(19) United States
(12) Patent Application Publication
(10) Pub. No.: US 2025/0295803 A1
(43) Pub. Date: Sep. 25, 2025

(54) 5T4 ANTIBODY-DRUG CONJUGATES AND USES THEREOF

(71) Applicant: EXELIXIS, INC., Alameda, CA (US)

(72) Inventors: Seema KANTAK, Pacifica, CA (US); Brian Alan MENDELSOHN, San Diego, CA (US); Robyn M. BARFIELD, Berkeley, CA (US); Stepan CHUPRAKOV, Walnut Creek, CA (US); Penelope M. DRAKE, Castro Valley, CA (US); Ayodele OGUNKOYA, Berkeley, CA (US)

(73) Assignee: EXELIXIS, INC., Alameda, CA (US)

(21) Appl. No.: 18/863,572

(22) PCT Filed: May 10, 2023
(86) PCT No.: PCT/US2023/066811

§ 371 Date: Nov. 6, 2024

Related U.S. Application Data
(60) Provisional application No. 63/341,957, filed on May 13, 2022.

(51) Int. Cl. A61K 47/68; A61P 35/00; C07K 16/30

(52) U.S. Cl. CPC A61K 47/6851; A61K 47/68033; A61K 47/6889; A61P 35/00; C07K 16/30; C07K 2317/33; C07K 2317/92

(57) ABSTRACT

The present disclosure provides 5T4 antibody-drug conjugates and uses thereof.

### CROSS-REFERENCE TO RELATED APPLICATIONS

This application claims the benefit of U.S. Provisional Patent Application No. 63/341,957, filed May 13, 2022, the disclosure of which is incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14529-099-228_SEQ_LISTING.xml”, was created on May 9, 2023 and is 125,376 bytes in size.

### FIELD

The present disclosure relates generally to antibody-drug conjugates (ADCs) that bind to 5T4 (e.g., human 5T4) and methods of their use.

### BACKGROUND

5T4 is an N-glycosylated transmembrane 72 kDa glycoprotein containing eight leucine-rich repeats. 5T4, also known as Wnt-Activated Inhibitory Factor 1 or WAIF1, is often referred to as an oncofetal antigen due to its expression in fetal trophoblast (where it was first discovered) or as trophoblast glycoprotein (TPBG). 5T4 is found in tumors such as colorectal, ovarian, and gastric tumors. Its expression is used as a prognostic aid. 5T4 has very limited expression in normal tissue but is widespread in malignant tumors throughout their development. Although its confined expression would appear to give 5T4 the potential to be a target in cancer therapy, therapeutic success with antibodies, ADCs, and vaccines targeting 5T4 has not yet been achieved.

There remains a need in the art for ADCs that can target 5T4 to treat, prevent, or alleviate 5T4-mediated diseases, disorders, or conditions, such as those involving tumor cells expressing 5T4.

### SUMMARY

The present disclosure provides ADCs comprising an antibody that binds to 5T4 (“5T4-ADC”). Such 5T4-ADCs, in some embodiments, bind to the same epitope of human 5T4 as an antibody comprising a heavy chain variable region (VH) and a light chain variable region (VL) described herein.

The present disclosure also provides pharmaceutical compositions comprising a 5T4-ADC that comprises an antibody or fragment thereof that binds to 5T4 and a drug conjugated (directly or indirectly) thereto. Such pharmaceutical compositions, in some embodiments, include 5T4-ADCs comprising an antibody or fragment thereof that binds to essentially the same epitope of human 5T4 as an antibody comprising a VH and a VL described herein.

The present disclosure also provides methods of treating, preventing, or alleviating a 5T4-mediated disease, disorder, or condition, such as alleviating one or more symptoms of the 5T4-mediated disease, disorder, or condition with a 5T4-ADC that binds to 5T4.

More specifically, the present disclosure provides a 5T4-ADC comprising (a) an antibody that binds to 5T4 and (b) one or more pyridazine-pyrrolo coupling moieties comprising a drug conjugated to the pyridazine-pyrrolo coupling moiety through a linker. In some embodiments, a 5T4-ADC is represented by Formula (IV):

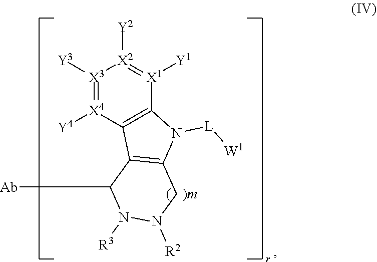

wherein
Ab represents the antibody that binds to 5T4;
R² and R³ are each independently hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or R² and R³ are cyclically linked to form a 5- or 6-membered heterocyclyl;
L is the linker and is represented by -(T¹V¹)ₐ-(T²V²)_{b}-(T³V³)_{c}-(T⁴V⁴)_{d}-(T⁵V⁵)ₑ—;
T¹, T², T³, T⁴, and T⁵ are each independently C₁-C₁₂ alkyl, substituted C₁-C₁₂ alkyl, (EDA)_{w}, (PEG)ₙ, (AA)ₚ, —(CR¹³OH)ₕ—, a piperidin-4-amine (P4A-R¹²), a meta-aminobenzyl carbamate (MABC) group, a meta-aminobenzyloxy (MABO) group, a para-aminiobenzyloxy (PABO) group, a para-aminobenzyl carbamate (PABC) group, a para-aminobenzyl (PAB) group, acetal, a disulfide, a hydrazine, (AA)ₚ-MABC-(AA)ₚ, (AA)ₚ-MABO-(AA)ₚ, (AA)ₚ-PABO-(AA)ₚ, or (AA)ₚ-PABC-(AA)ₚ;
V¹, V², V³, V⁴ and V⁵ are each independently a covalent bond, —C(═O)—, —NR¹¹—, —C(═O)NR¹¹—, —NR¹¹C(═O)—, —C(═O)O—, —OC(═O)—, —O—, —S—, —S(═O)—, —SO₂—, —SO₂NR¹¹—, —NR¹¹SO₂—, or —P(═O)OH—;
a, b, c, d, and e are each independently 0 or 1, wherein the sum of a, b, c, d, and e is 1 to 5;
h is independently an integer from 1 to 12;
m is 0 or 1;
n is independently an integer from 1 to 30;
p is independently zero or an integer from 1 to 20;
r is an integer from 1 to 10;
w is independently an integer from 1 to 20;
X¹, X², X³ and X⁴ are each independently C, N, O, or S;
Y¹, Y², Y³ and Y⁴ are each independently hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl, or Y¹ and Y², Y² and Y³, or Y³ and Y⁴ are cyclically linked;
W¹ is the drug;
R¹¹ is independently hydrogen, alkyl, substituted alkyl, PEG, aryl, or substituted aryl; and
R¹² is independently hydrogen, alkyl, substituted alkyl, a polyethylene glycol moiety, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; and
R¹³ is independently hydrogen, alkyl, substituted alkyl, aryl, or substituted aryl.

In some embodiments, the drug is a maytansinoid or an auristatin, such as MMAE or MMAF.

In some embodiments, a 5T4-ADC can be represented by Formula (IV), wherein R² and R³ are each alkyl, for example, methyl. In some embodiments, a 5T4-ADC can be represented by Formula (IV), wherein each of X¹, X², X³, and X⁴ are independently C or N.

In some embodiments, a 5T4-ADC can be represented by Formula (V):

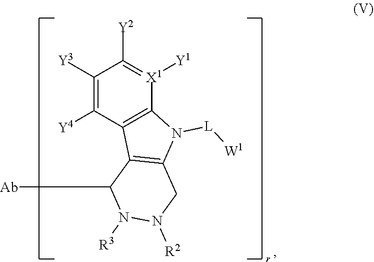

wherein X¹, Y¹, Y², Y³, Y⁴, R², R³, L, and W¹ are as defined herein, such as for Formula (IV).

In some embodiments, a 5T4-ADC can be represented by Formula (VI):

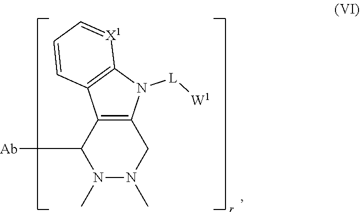

wherein X¹ is C or N, and L and W¹ are as defined herein, such as for Formula (IV).

In some embodiments, a 5T4-ADC can be represented by Formula (IV), (V), or (VI), wherein:
L is the linker and is represented by -(T¹V¹)ₐ-(T²V²)_{b}-(T³V³)_{c}-(T⁴V⁴)_{d}-(T⁵V⁵)ₑ—;
T¹, T², T³, T⁴ and T⁵ are each independently C₁-C₆ alkyl, P4A-R¹², (AA)ₚ, (AA)ₚ-PABC, or (PEG)ₙ;
V¹, V², V³, V⁴ and V⁵ are each independently selected from the group consisting of a covalent bond, —C(═O)—, —NR¹¹—, —C(═O)NR¹¹—, —NR¹¹C(═O)—, —C(═O)O—, —OC(═O)—, —O—, —S—, —S(═O)—, —SO₂—, —SO₂NR¹¹—, —NR¹¹SO₂— and —P(═O)OH—;
a, b, c, d, and e are each independently 0 or 1, wherein the sum of a, b, c, d, and e is 1 to 5;
p is an integer from 1 to 20;
n is an integer from 1 to 30;
R¹¹ is selected from the group consisting of hydrogen, alkyl, substituted alkyl, PEG, aryl, and substituted aryl; and
R¹² is independently a polyethylene glycol or carboxylic acid-modified polyethylene glycol.

In further embodiments, a 5T4-ADC can be represented by Formula (IV), (V), or (VI), wherein:
a, b, c, d, and e are each independently 0 or 1, wherein the sum of a, b, c, d, and e is 1 to 5;
p is an integer from 1 to 20;
n is an integer from 1 to 30;
L is the linker and is represented by -(T¹V¹)ₐ-(T²V²)_{b}-(T³V³)_{c}-(T⁴V⁴)_{d}-(T⁵V⁵)ₑ—;
T¹ is C₁-C₆ alkyl;
T², T³, T⁴, and T⁵ are each independently (PEG)ₙ, C₁-C₆ alkyl, (AA)ₚ, P4A-R¹², (AA)ₚ-(PABO-R¹⁶)-(AA)ₚ, (AA)ₚ-(PABC-R¹⁶)-(AA)ₚ, (AA)ₚ-(PABO-R¹⁶), or (AA)ₚ-(PABC-R¹⁶);
V¹, V², V³, V⁴, and V⁵ are each independently —CO— or —NR¹¹;
R¹¹ is independently hydrogen or alkyl;
R¹² is independently a carboxylic acid-modified polyethylene glycol; and
R¹⁶ is hydrogen.

In some embodiments, a 5T4-ADC can be represented by Formula (IV), (V), or (VI), wherein:
L is —(C₁-C₆ alkyl)-(V¹)—(P4A-R¹²)—(V²)—(C₁-C₅ alkyl)-(V³)—;
V¹, V², and V³ are each independently —CO— or —NR¹¹—;
R¹¹ is independently hydrogen or C₁-C₆ alkyl; and
R¹² is a carboxylic acid-modified polyethylene glycol moiety.

In some embodiments, a 5T4-ADC can be represented by Formula (IV), (V), or (VI), wherein L is:

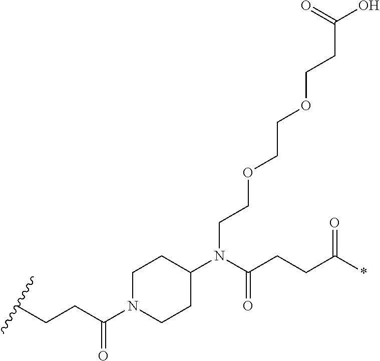

wherein [symbol] represents the point of attachment to the nitrogen of the pyridazine-pyrrolo coupling moiety and * represents the point of attachment to W¹.

In some embodiments, a 5T4-ADC can be represented by Formula (VI-1):

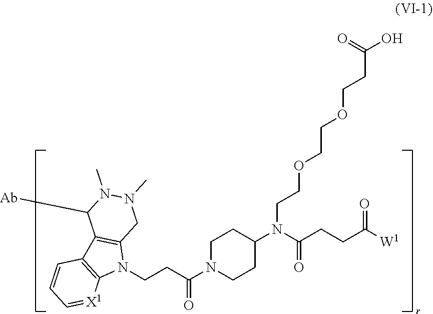

wherein:
Ab represents the antibody that binds to 5T4;
r is an integer from 1 to 10;
X¹ is C or N; and
W¹ is the drug.

In some embodiments, a 5T4-ADC can be represented by Formula (VI-1), wherein the drug is a maytansinoid or an auristatin, such as MMAE. In some embodiments, a 5T4-ADC can be represented by Formula (VI-1), wherein X¹ is C and W¹ is maytansine or deacylated maytansine. In some embodiments, a 5T4-ADC can be represented by Formula (VI-1), wherein X¹ is N and W¹ is MMAE.

In some embodiments, a 5T4-ADC can be represented by Formula (IV), (V), or (VI), wherein:
L is (C₁-C₆ alkyl)-V¹-(AA)-V²—(PEG)ₙ-V³-((AA)ₚ-(PABC-R¹⁶))—V⁴—(C₁-C₆ alkyl)-V⁵;
V¹, V², V³, V⁴ and V⁵ are each independently selected from the group consisting of —CO— and —NR¹¹—R¹¹ is independently hydrogen or C₁-C₆ alkyl;
R¹⁶ is hydrogen; and
n and p are each independently an integer from 1 to 20;

In some embodiments, a 5T4-ADC can be represented by Formula (IV), (V), or (VI), wherein L is:

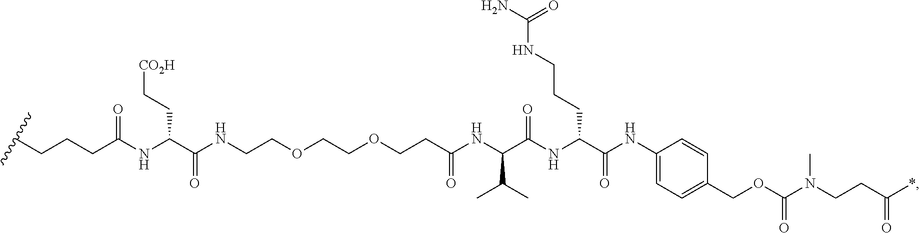

wherein [symbol] represents the point of attachment to the nitrogen of the pyridazine-pyrrolo coupling moiety; and * represents the point of attachment to W¹.

In some embodiments, a 5T4-ADC can be represented by Formula (VI-2):

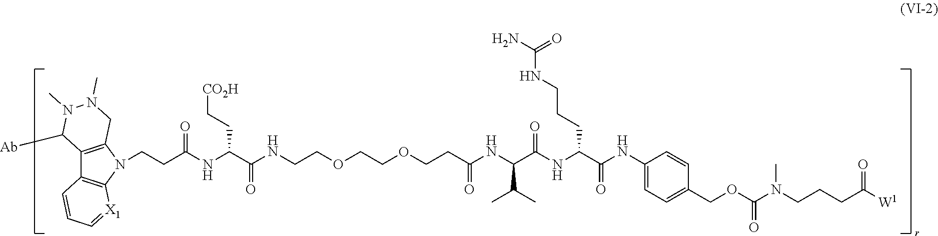

wherein:
Ab represents the antibody that binds to 5T4;
r is an integer from 1 to 10;
X¹ is C or N; and
W¹ is the drug.

In some embodiments, a 5T4-ADC can be represented by Formula (VI-2), wherein the drug is a maytansinoid or an auristatin, such as MMAE. In some embodiments, a 5T4-ADC can be represented by Formula (VI-2) wherein X¹ is C and W¹ is maytansine or deacylated maytansine.

The present disclosure also provides a 5T4-ADC of Formula (IV), (V), and (VI):

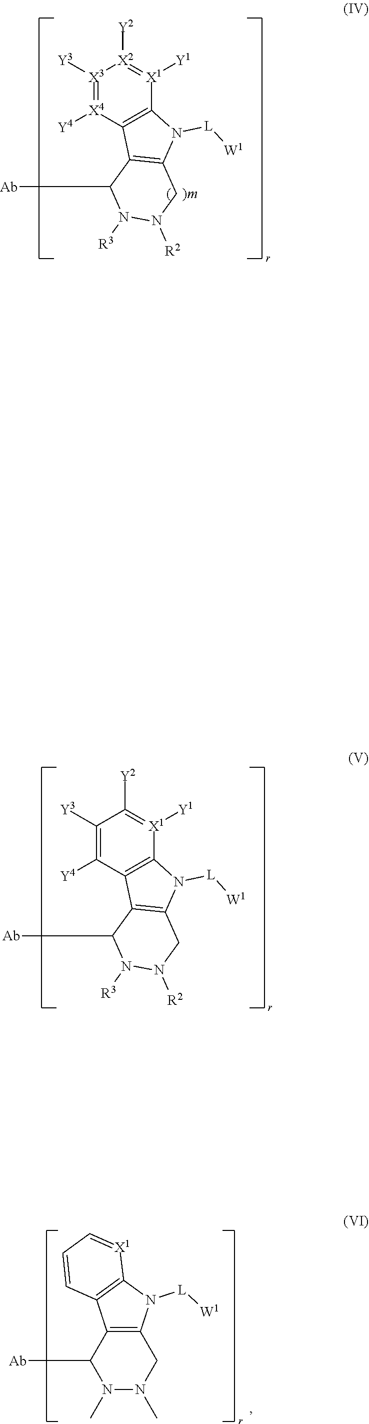

wherein:
Ab represents the antibody that binds to 5T4;
r is an integer from 1 to 10;
m is 0 or 1;
R² and R³ are each independently hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or R² and R³ are cyclically linked to form a 5- or 6-membered heterocyclyl;
X¹, X², X³, and X⁴ are each independently selected from the group consisting of C, N, O and S;
Y¹, Y², Y³, and Y⁴ are each independently hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or Y¹ and Y², Y² and Y³, or Y³ and Y⁴ are cyclically linked;
L is

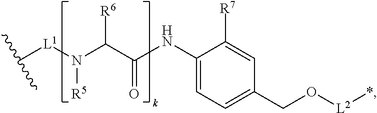

[symbol] represents the point of attachment to the nitrogen of the pyridazine-pyrrolo coupling moiety;
* represents the point of attachment to W¹;
R⁵ is independently hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl;
R⁶ is independently alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl;
R⁷ is a cleavable moiety;
k is an integer from 1 to 10;
L¹ comprises -(T¹-V¹)ₐ-(T²-V²)_{b}-(T³-V³)_{c}-(T⁴-V⁴)_{d}—;
L² comprises -(T⁵-V⁵)ₑ-(T⁶-V⁶)_{f}-(T⁷-V⁷)_{g}-(T⁸-V⁸)ₙ—;
T¹, T², T³, T⁴, T⁵, T⁶, T⁷, and T⁸ are each independently a covalent bond, C₁-C₁₂ alkyl, substituted C₁-C₁₂ alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, P4A-R¹², acetal, a hydrazine, a disulfide, and an ester;
V¹, V², V³, V⁴, V⁵, V⁶, V⁷, and V⁸ are each independently a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
a, b, c, d, e, f, g, and h are each independently 1 or 0;
m is independently an integer from 1 to 12;
n is independently an integer from 1 to 30;
p is independently an integer from 1 to 20;
q is independently an integer from 1 to 6;
w is independently an integer from 1 to 20;
R¹² is independently hydrogen, alkyl, substituted alkyl, a polyethylene glycol moiety, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl;
R¹³ is independently hydrogen, alkyl, substituted alkyl, aryl, and substituted aryl; and
R¹⁵ is independently hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, or substituted heterocyclyl.

In some embodiments, a 5T4-ADC can be represented by Formula (IV) wherein R² and R³ are each alkyl, for example, methyl. In some embodiments, a 5T4-ADC can be represented by Formula (IV) wherein each of X¹, X², X³, and X⁴ are independently C or N.

In some embodiments, a 5T4-ADC can be represented by Formula (VI-3):

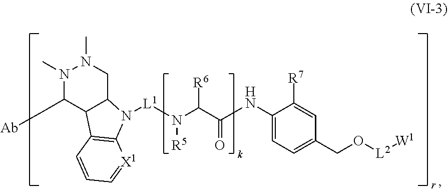

wherein:
Ab represents the antibody that binds to 5T4;
X¹ is C, N, O, or S;
R⁵ is independently hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl;
R⁶ is independently alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl;
R⁷ is a cleavable moiety;
L¹ comprises -(T¹-V¹)ₐ-(T²-V²)_{b}-(T³-V³)_{c}-(T⁴-V⁴)_{d}—;
L² comprises -(T⁵-V⁵)ₑ-(T⁶-V⁶)_{f}-(T⁷-V⁷)_{g}-(T⁸-V⁸)ₕ—;
T¹, T², T³, T⁴, T⁵, T⁶, T⁷, and T⁸ are each independently a covalent bond, C₁-C₁₂ alkyl, substituted C₁-C₁₂ alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, P4A-R¹², acetal, a hydrazine, a disulfide, or an ester;
V¹, V², V³, V⁴, V⁵, V⁶, V⁷, and V⁸ are each independently a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, or —P(O)OH—;
a, b, c, d, e, f, g, and h are each independently 1 or 0;
k is an integer from 1 to 10;
m is independently an integer from 1 to 12;
n is independently an integer from 1 to 30;
p is independently an integer from 1 to 20;
q is independently an integer from 1 to 6;
w is independently an integer from 1 to 20;
R¹² is independently hydrogen, alkyl, substituted alkyl, a polyethylene glycol moiety, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl;
R¹³ is independently hydrogen, alkyl, substituted alkyl, aryl, or substituted aryl; and
R¹⁵ is independently hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, or substituted heterocyclyl.

In some embodiments, a 5T4-ADC can be represented by Formula (IV), (V), or (IV), wherein:
L is

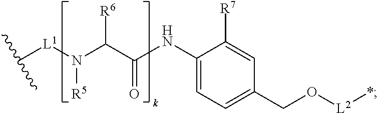

R⁵, R⁶, L¹, L², and k are as defined herein, such as for Formula (VI-3); and
R⁷ is

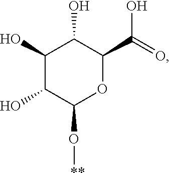

wherein ** represents the point of attachment to the phenyl group described herein, such as in Formula (VI-3), (V-4), or (VI-4).

In some embodiments, a 5T4-ADC can be represented by Formula (V-5):

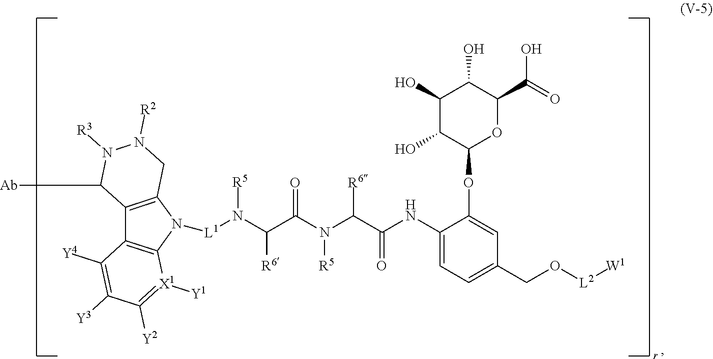

wherein Ab, L¹, L², R², R³, R⁵, X¹, Y¹, Y², Y³, Y⁴, W¹, and r are as defined herein, such as for Formula (VI-3) and R^{6′} and R^{6″} are each independently alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl.

In some embodiments, a 5T4-ADC can be represented by Formula (VI-5):

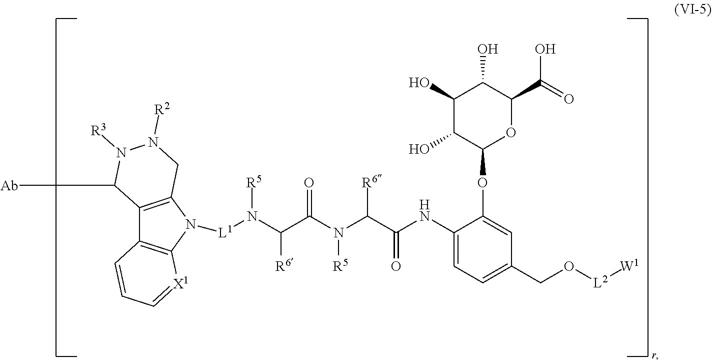

wherein Ab, L¹, L², R⁵, W¹, and r are as defined herein, such as for Formula (VI-3) and R^{6′} and R^{6″} are each independently alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl and wherein X¹ is C or N.

In some embodiments, a 5T4-ADC can be represented by Formula (VI-6):

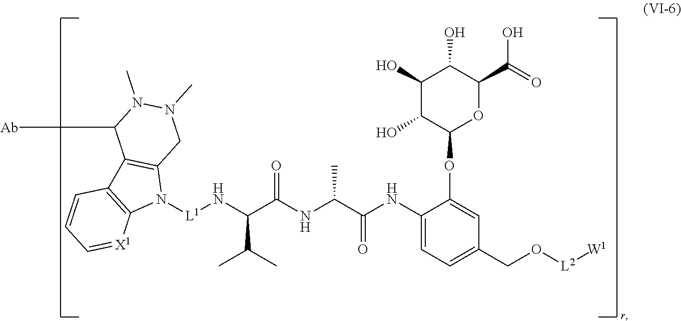

wherein Ab, L¹, L², W¹, and r are as defined herein, such as for Formula (VI-3) and wherein X¹ is C or N.

In some embodiments, a 5T4-ADC can be represented by Formula (VI-3), (V-5), (VI-5), or (VI-6), wherein:
L¹ comprises -(T¹-V¹)ₐ-(T²-V²)_{b}-(T³-V³)—;
T¹, T², and T³ are each independently selected from the group consisting of C₁-C₁₂ alkyl, (PEG)ₙ, and (AA)ₚ;
V¹, V², and V³ are each independently —C(═O)— or —NR¹¹—;
a, b, and c are each 1;
n is independently an integer from 1 to 10;
p is independently an integer from 1 to 10;
R¹¹ is independently hydrogen, alkyl, substituted alkyl, a polyethylene glycol moiety, aryl, or substituted aryl.

In some embodiments, a 5T4-ADC can be represented by Formula (VI-3), (V-5), (VI-5), or (VI-6), wherein L¹ is

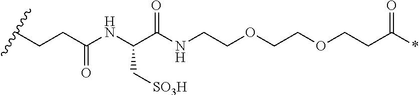

wherein [symbol] represents the point of attachment to the nitrogen of a pyridazine-pyrrolo coupling moiety, and wherein * represents the point of attachment to terminal nitrogen in the linker, as described herein, such as is shown in Formula (VI-6).

In some embodiments, a 5T4-ADC can be represented by Formula (VI-7):

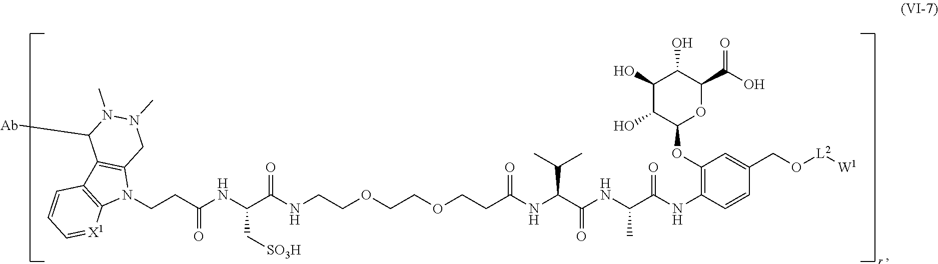

wherein Ab, L¹, L², W¹, and r are as defined herein, such as for Formula (VI-3) and wherein X¹ is C or N.

In some embodiments, a 5T4-ADC can be represented by Formula (VI-7) wherein L² is a carbonyl group. In some embodiments, a 5T4-ADC can be represented by Formula (VI-7) wherein X¹ is N and W¹ is MMAE.

In some embodiments, a 5T4-ADC can be represented by Formula (IV), (V), (VI), (VI-1), (VI-2), (VI-3), (V-5), (VI-5), (VI-6), or (VI-7) wherein the antibody (Ab) comprises: (i) a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26; (ii) a VH CDR1, a VH CDR2, and a VH CDR3 as set forth in a VH comprising the amino acid sequence of SEQ ID NO:44 and a VL CDR1, a VL CDR2, and a VL CDR3 as set forth in a VL comprising the amino acid sequence of SEQ ID NO:45; or (iii) a VH CDR1, a VH CDR2, and a VH CDR3 as set forth in a VH comprising the amino acid sequence of SEQ ID NO:62 and a VL CDR1, a VL CDR2, and a VL CDR3 as set forth in a VL comprising the amino acid sequence of SEQ ID NO:63.

In some embodiments, a 5T4-ADC can be represented by Formula (IV), (V), (VI), (VI-1), (VI-2), (VI-3), (V-5), (VI-5), (VI-6), or (VI-7) wherein the antibody (Ab) comprises: (a) a VH region comprising (1) a VH CDR1 having an amino acid sequence selected from the group consisting of SEQ ID NOs:1, 7, 12, 13, and 18; (2) a VH CDR2 having an amino acid sequence selected from the group consisting of SEQ ID NOs:2, 8, 14, 19, and 24; and (3) a VH CDR3 having an amino acid sequence selected from the group consisting of SEQ ID NOs:3, 9, 15, and 20; and (b) a VL region comprising (1) a VL CDR1 having an amino acid sequence selected from the group consisting of SEQ ID NOs:4, 10, 16, and 21; (2)ₐ VL CDR2 having an amino acid sequence selected from the group consisting of SEQ ID NOs:5, 11, and 22; and (3) a VL CDR3 having an amino acid sequence selected from the group consisting of SEQ ID NOs:6, 17, and 23.

In some embodiments, a 5T4-ADC can be represented by Formula (IV), (V), (VI), (VI-1), (VI-2), (VI-3), (V-5), (VI-5), (VI-6), or (VI-7) wherein the antibody (Ab) comprises: (i) a VH region comprising a VH CDR1 comprising the amino acid sequence of SEQ ID NO:1, a VH CDR2 comprising the amino acid sequence of SEQ ID NO:2, and a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; or (ii) a VH region comprising a VH CDR1 comprising the amino acid sequence of SEQ ID NO:7, a VH CDR2 comprising the amino acid sequence of SEQ ID NO:8, and a VH CDR3 comprising the amino acid sequence of SEQ ID NO:9; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:6; or (iii) a VH region comprising a VH CDR1 comprising the amino acid sequence of SEQ ID NO:12, a VH CDR2 comprising the amino acid sequence of SEQ ID NO:2, and a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; or (iv) a VH region comprising a VH CDR1 comprising the amino acid sequence of SEQ ID NO:13, a VH CDR2 comprising the amino acid sequence of SEQ ID NO:14, and a VH CDR3 comprising the amino acid sequence of SEQ ID NO:15; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:17; or (v) a VH region comprising a VH CDR1 comprising the amino acid sequence of SEQ ID NO:18, a VH CDR2 comprising the amino acid sequence of SEQ ID NO:19, and a VH CDR3 comprising the amino acid sequence of SEQ ID NO:20; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:23; or (vi) a VH region comprising a VH CDR1 comprising the amino acid sequence of SEQ ID NO:1, a VH CDR2 comprising the amino acid sequence of SEQ ID NO:24, and a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

In some embodiments, a 5T4-ADC can be represented by Formula (IV), (V), (VI), (VI-1), (VI-2), (VI-3), (V-5), (VI-5), (VI-6), or (VI-7) wherein the antibody (Ab) comprises: (a) a VH region comprising (1) a VH CDR1 having an amino acid sequence selected from the group consisting of SEQ ID NOs:27, 31, 34, 35, and 39; (2) a VH CDR2 having an amino acid sequence selected from the group consisting of SEQ ID NOs:28, 32, 36, 40, and 43; and (3) a VH CDR3 having an amino acid sequence selected from the group consisting of SEQ ID NOs:29, 33, 37, and 41; and (b) a VL region comprising (1) a VL CDR1 having an amino acid sequence selected from the group consisting of SEQ ID NOs:4, 10, 16, and 21; (2) a VL CDR2 having an amino acid sequence selected from the group consisting of SEQ ID NOs:5, 11, and 22; and (3) a VL CDR3 having an amino acid sequence selected from the group consisting of SEQ ID NOs:30, 38, and 42.

In some embodiments, a 5T4-ADC can be represented by Formula (IV), (V), (VI), (VI-1), (VI-2), (VI-3), (V-5), (VI-5), (VI-6), or (VI-7) wherein the antibody (Ab) comprises (i) a VH region comprising a VH CDR1 comprising the amino acid sequence of SEQ ID NO:27, a VH CDR2 comprising the amino acid sequence of SEQ ID NO:28, and a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30; or (ii) a VH region comprising a VH CDR1 comprising the amino acid sequence of SEQ ID NO:31, a VH CDR2 comprising the amino acid sequence of SEQ ID NO:32, and a VH CDR3 comprising the amino acid sequence of SEQ ID NO:33; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:30; or (iii) a VH region comprising a VH CDR1 comprising the amino acid sequence of SEQ ID NO:34, a VH CDR2 comprising the amino acid sequence of SEQ ID NO:28, and a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30; or (iv) a VH region comprising a VH CDR1 comprising the amino acid sequence of SEQ ID NO:35, a VH CDR2 comprising the amino acid sequence of SEQ ID NO:36, and a VH CDR3 comprising the amino acid sequence of SEQ ID NO:37; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:38; or (v) a VH region comprising a VH CDR1 comprising the amino acid sequence of SEQ ID NO:39, a VH CDR2 comprising the amino acid sequence of SEQ ID NO:40, and a VH CDR3 comprising the amino acid sequence of SEQ ID NO:41; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:42; or (vi) a VH region comprising a VH CDR1 comprising the amino acid sequence of SEQ ID NO:27, a VH CDR2 comprising the amino acid sequence of SEQ ID NO:43, and a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30.

In some embodiments, a 5T4-ADC can be represented by Formula (IV), (V), (VI), (VI-1), (VI-2), (VI-3), (V-5), (VI-5), (VI-6), or (VI-7) wherein the antibody (Ab) comprises: (a) a VH region comprising (1) a VH CDR1 having an amino acid sequence selected from the group consisting of SEQ ID NOs:46, 50, 53, 13, and 57; (2) a VH CDR2 having an amino acid sequence selected from the group consisting of SEQ ID NOs:47, 51, 54, 58, and 61; and (3) a VH CDR3 having an amino acid sequence selected from the group consisting of SEQ ID NOs:48, 52, 55, and 59; and (b) a VL region comprising (1) a VL CDR1 having an amino acid sequence selected from the group consisting of SEQ ID NOs:4, 10, 16, and 21; (2) VL CDR2 having an amino acid sequence selected from the group consisting of SEQ ID NOs:5, 11, and 22; and (3) a VL CDR3 having an amino acid sequence selected from the group consisting of SEQ ID NOs:49, 56, and 60.

In some embodiments, a 5T4-ADC can be represented by Formula (IV), (V), (VI), (VI-1), (VI-2), (VI-3), (V-5), (VI-5), (VI-6), or (VI-7) wherein the antibody (Ab) comprises: (i) a VH region comprising a VH CDR1 comprising the amino acid sequence of SEQ ID NO:46, a VH CDR2 comprising the amino acid sequence of SEQ ID NO:47, and a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49; or (ii) a VH region comprising a VH CDR1 comprising the amino acid sequence of SEQ ID NO:50, a VH CDR2 comprising the amino acid sequence of SEQ ID NO:51, and a VH CDR3 comprising the amino acid sequence of SEQ ID NO:52; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:49; or (iii) a VH region comprising a VH CDR1 comprising the amino acid sequence of SEQ ID NO:53, a VH CDR2 comprising the amino acid sequence of SEQ ID NO:47, and a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49; or (iv) a VH region comprising a VH CDR1 comprising the amino acid sequence of SEQ ID NO:13, a VH CDR2 comprising the amino acid sequence of SEQ ID NO:54, and a VH CDR3 comprising the amino acid sequence of SEQ ID NO:55; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:56; or (v) a VH region comprising a VH CDR1 comprising the amino acid sequence of SEQ ID NO:57, a VH CDR2 comprising the amino acid sequence of SEQ ID NO:58, and a VH CDR3 comprising the amino acid sequence of SEQ ID NO:59; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:60; or (vi) a VH region comprising a VH CDR1 comprising the amino acid sequence of SEQ ID NO:46, a VH CDR2 comprising the amino acid sequence of SEQ ID NO:61, and a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49.

In some embodiments, a 5T4-ADC can be represented by Formula (IV), (V), (VI), (VI-1), (VI-2), (VI-3), (V-5), (VI-5), (VI-6), or (VI-7) wherein the antibody (Ab) comprises a framework 1 (FR1), a framework 2 (FR2), a framework 3 (FR3) and/or a framework 4 (FR4) sequence as set forth in any one of SEQ ID NOs:25, 26, 44, 45, 62, and 63.

In some embodiments, a 5T4-ADC can be represented by Formula (IV), (V), (VI), (VI-1), (VI-2), (VI-3), (V-5), (VI-5), (VI-6), or (VI-7) wherein the antibody (Ab) comprises human framework sequences.

In some embodiments, a 5T4-ADC can be represented by Formula (IV), (V), (VI), (VI-1), (VI-2), (VI-3), (V-5), (VI-5), (VI-6), or (VI-7) wherein the antibody (Ab) comprises (i) a VH comprising the amino acid sequence of SEQ ID NO:25 and a VL comprising the amino acid sequence of SEQ ID NO:26; (ii) a VH comprising the amino acid sequence of SEQ ID NO:44 and a VL comprising the amino acid sequence of SEQ ID NO:45; or (iii) a VH comprising the amino acid sequence of SEQ ID NO:62 and a VL comprising the amino acid sequence of SEQ ID NO:63.

The present disclosure also provides a pharmaceutical composition comprising a 5T4-ADC, wherein the 5T4-ADC can be represented by Formula (IV), (V), (VI), (VI-1), (VI-2), (VI-3), (V-5), (VI-5), (VI-6), or (VI-7) and a pharmaceutically acceptable excipient, wherein the antibody (Ab) is as described in any embodiment described herein. In some embodiments, such a pharmaceutical composition can have a drug-to-antibody ratio (DAR) of the 5T4-ADC of about 1 to about 10, for example, a DAR of about 1 to about 4, about 1 to about 2, about 1.5 to about 2, or about 1.75 to about 1.9.

The present disclosure also provides a method for treating a cancer or a tumor in a subject comprising administering to the subject the 5T4-ADC, wherein the 5T4-ADC can be represented by Formula (IV), (V), (VI), (VI-1), (VI-2), (VI-3), (V-5), (VI-5), (VI-6), or (VI-7) or the pharmaceutical composition comprising a 5T4-ADC of Formula (IV), (V), (VI), (VI-1), (VI-2), (VI-3), (V-5), (VI-5), (VI-6), or (VI-7) and a pharmaceutically acceptable excipient, wherein the antibody (Ab) is as described in any embodiment herein.

### BRIEF DESCRIPTION OF THE DRAWINGS

FIG. 1 provides an exemplary synthesis of the linker-drug of Formula VIa-21 as Scheme 1.

FIG. 2 provides an exemplary synthesis of the linker-drug of Formula VIa-11 as Scheme 2.

FIG. 3 provides an exemplary synthesis of the linker-drug of Formula VIb-11 as Scheme 3.

FIG. 4 provides an exemplary synthesis of the linker-drug Formula VIb-82 as Scheme 4.

FIG. 5 provides an exemplary synthesis of the linker-drug of Formula VIb-81 as Scheme 5.

FIG. 6 reports the cell viability of MDA-MB-468 cells treated with a 5T4-ADC of Formula mAbA15-VIa-11 or mAbA17-VIa-11 ADCs.

FIG. 7 reports the cell viability of MCF-7 cells treated with a 5T4-ADC of Formula mAbA15-VIa-11 or mAbA17-VIa-11 ADCs.

FIG. 8 reports the cell viability of MDA-MB-468 cells treated with a 5T4-ADC of Formula mAbA15-VIb-82 or mAbA17-VIb-82.

FIG. 9 reports the cell viability of MCF-7 cells treated with a 5T4-ADC of Formula mAbA15-VIb-82 or mAbA17-VIb-82.

FIG. 10 reports the cell viability of MDA-MB-231 cells treated with a 5T4-ADC of Formula mAbA15-VIb-82 or mAbA17-VIb-82.

FIG. 11 reports the cell viability of MCI-H226 cells treated with a 5T4-ADC of Formula mAbA15-VIb-82 or mAbA17-VIb-82.

FIG. 12 reports the cell viability of BxPC3 cells treated with a 5T4-ADC of Formula mAbA15-VIb-82 or mAbA17-VIb-82.

FIG. 13 reports mean tumor volume in MCF-7.1 xenograft mice treated with a 5T4-ADC of Formula mAbA15-VIa-11 or mAbA17-VIa-11.

FIG. 14 reports mean tumor volume in MCF-7.1 xenograft mice treated with a 5T4-ADC of Formula mAbA15-VIb-82 or mAbA17-VIb-82.

FIG. 15 reports mean tumor volume in MCF-7.1 xenograft mice treated with a single dose of 0.5 mg/kg, 2.5 mg/kg, or 5.0 mg kg of a 5T4-ADC of Formula mAbA15-VIb-82.

FIG. 16 reports mean tumor volume in MCF-7.1 xenograft mice treated with two doses (separated by 1 week) of 0.25 mg/kg, 1.25 mg/kg, or 2.5 mg/kg of a 5T4-ADC of Formula mAbA15-VIb-82.

FIG. 17 reports mean tumor volume in MCF-7.1 xenograft mice treated with a single dose of 0.5 mg/kg, 2.5 mg/kg, or 5.0 mg kg of a 5T4-ADC of Formula mAbA17-VIb-82.

FIG. 18 reports mean tumor volume in MCF-7.1 xenograft mice treated with two doses (separated by 1 week) of 0.25 mg/kg, 1.25 mg/kg, or 2.5 mg/kg of a 5T4-ADC of Formula mAbA17-VIb-82.

FIG. 19 reports mean tumor volume in MDA-MB-468 xenograft mice treated with a 5T4-ADC of Formula mAbA15-VIa-11, mAbA15-VIb-81, or mAbA15-VIb-82.

FIG. 20 reports mean tumor volume in MDA-MB-231 xenograft mice treated with a 5T4-ADC of Formula mAbA15-VIb-82 or mAbA15-VIa-11.

FIG. 21 reports mean tumor volume in NCI-H₂₂₆ xenograft mice treated with a 5T4-ADC of Formula mAbA15-VIb-82 or mAbA15-VIa-11.

FIG. 22 reports mean tumor volume in BxPC3 xenograft mice treated with a 5T4-ADC of Formula mAbA15-VIb-82 or mAbA15-VIa-11.

FIG. 23 reports mean tumor volume in Calu-6 xenograft mice treated with a 5T4-ADC of Formula mAbA15-VIb-82.

FIGS. 24A-24C show total antibody and total ADC concentrations (μg/mL) quantified by two ELISA methods in toxicokinetic (TK) studies of rats treated with a 5T4-ADC of Formula mAbA15-VIb-82 at 30 mg/kg (FIG. 24A), 60 mg/kg (FIG. 24B), and 90 mg/kg (FIG. 24C).

### DETAILED DESCRIPTION

The present disclosure provides antibody-drug conjugates (ADCs) that bind to 5T4 and a drug conjugated (directly or indirectly) thereto. Such 5T4-ADCs are useful in compositions and in methods of treating, preventing, or alleviating a 5T4-mediated disease, disorder, or condition, including one or more symptoms of the disease, disorder, or condition. 5T4-mediated diseases, disorders, and conditions include a variety of cancers, including, but not limited to, any cancer wherein the tumor cells express or overexpress 5T4. In addition, 5T4-ADCs are useful for the killing and/or removal of tumor cells. 5T4-ADCs described herein are useful in compositions and in methods for treating cancer.

The term “alkyl” refers to monovalent saturated aliphatic hydrocarbyl groups having from one to twelve carbon atoms, such as one to 10, one to 6, one to 5, one to 4, or one to three carbon atoms. This term includes, by way of example, linear and branched hydrocarbyl groups such as methyl (—CH₃), ethyl (—CH₂CH₃), n-propyl (—CH₂CH₂CH₃), isopropyl (—CH(CH₃)₂), n-butyl (—CH₂CH₂CH₂CH₃), isobutyl (—CH₂CH(CH₃)₂), sec-butyl (—CH(CH₃)(CH₃CH₂)), t-butyl (—C(CH₃)₃), n-pentyl (—CH₂CH₂CH₂CH₂CH₃), and neopentyl (—CH₂C(CH₃)₃).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ-(where n is 0 to 2), —NR— (wherein R is hydrogen or alkyl) and having from one to five substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R^{a} and R^{b} can be the same or different and are chosen from hydrogen, optionally substituted alkyl, cycloalkyl, alkenyl, cycloalkenyl, alkynyl, aryl, heteroaryl, and heterocyclic.

The term “alkenyl” refers to divalent aliphatic hydrocarbyl groups preferably having from one to six and more preferably one to three carbon atoms that are either straight-chained or branched, and which are optionally substituted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

The term “substituted alkenyl” refers to an alkylene group having from one to three hydrogens replaced with substituents as described for carbons in the definition of “substituted” below.

The term “alkaryl” or “aralkyl” refers to the groups -alkylene-aryl and -substituted alkylene-aryl wherein alkylene, substituted alkylene, and aryl are defined herein.

The term “alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to alkenyl-O—, cycloalkyl-O—, cycloalkenyl-O—, and alkynyl-O—, wherein alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— wherein substituted alkyl, substituted alkenyl, substituted cycloalkyl, substituted cycloalkenyl, and substituted alkynyl are as defined herein.

The term “alkoxyamino” refers to the group —NH-alkoxy, wherein alkoxy is defined herein.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkenyl” refers to a straight chain or branched hydrocarbyl group having from two to six carbon atoms, such as two to four carbon atoms, and having at least one and preferably from one to two sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from one to five substituents, or from one to three substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl, and —SO₂-heteroaryl.

The term “alkynyl” refers to a straight or branched monovalent hydrocarbyl group having from two to six carbon atoms and preferably two to three carbon atoms and having at least one and preferably from one to two sites of triple bond unsaturation. Examples of such alkynyl groups include acetylenyl (—C≡CH), and propargyl (—CH₂C—CH).

The term “substituted alkynyl” refers to an alkynyl group as defined herein having from one to five substituents, or from one to three substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl, and —SO₂-heteroaryl.

The term “acylamino” refers to a group selected from —NR²⁰C(O)alkyl, —NR²⁰C(O)substituted alkyl, NR²⁰C(O)cycloalkyl, —NR²⁰C(O)substituted cycloalkyl, —NR²⁰C(O)-cycloalkenyl, —NR²⁰C(O)-substituted cycloalkenyl, —NR²⁰C(O)alkenyl, —NR²⁰C(O)-substituted alkenyl, —NR²⁰C(O)alkynyl, —NR²⁰C(O)-substituted alkynyl, —NR²⁰C(O)aryl, —NR²⁰C(O)-substituted aryl, —NR²⁰C(O)-heteroaryl, —NR²⁰C(O)substituted heteroaryl, —NR²⁰C(O)-heterocyclic, and —NR²⁰C(O)-substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “acyl” refers to a group selected from H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—.

The term “aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and wherein R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “acyloxy” refers to a group selected from alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

The term “aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and wherein R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

The term “sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and wherein R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aryl” refers to a monovalent aromatic carbocyclic group having six to eighteen carbon atoms as a single ring (such as is present in a phenyl group) or a ring system having multiple condensed rings (examples of such aromatic ring systems include naphthyl, anthryl, and indanyl) which condensed rings may or may not be aromatic, provided that the point of attachment is through an atom of an aromatic ring. This term includes, by way of example, phenyl, and naphthyl. Unless otherwise constrained by the definition for the aryl substituent, such aryl groups can optionally be substituted with from one to five substituents, or from one to three substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and trihalomethyl.

The term “aryloxy” refers to the group —O-aryl, wherein aryl is as defined herein, including, by way of example, phenoxy, naphthoxy, and the like, including optionally substituted aryl groups as also defined herein.

The term “amino” refers to the group —NH₂.

The term “substituted amino” refers to the group —NRR wherein each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

The term “carboxyl,” “carboxy” or “carboxylate” refers to —CO₂H or salts thereof.

The term “carboxyl ester” or “carboxy ester” or the terms “carboxyalkyl” or “carboxylalkyl” refers to a group selected from —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “cyano” or “nitrile” refers to the group —CN.

The term “cycloalkyl” refers to a cyclic alkyl group of three to ten carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl, and the like.

The term “substituted cycloalkyl” refers to a cycloalkyl group having one to five substituents, or one to three substituents, wherein the substituents are independently selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

The term “cycloalkenyl” refers to a non-aromatic cyclic alkyl group of three to ten carbon atoms having a single or multiple rings and having at least one double bond, preferably one or two double bonds.

The term “substituted cycloalkenyl” refers to a cycloalkenyl group having one to five substituents, or one to three substituents, wherein the substituents are independently selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-substituted alkyl, —SO-aryl, —SO-heteroaryl, —SO₂-alkyl, —SO₂-substituted alkyl, —SO₂-aryl, and —SO₂-heteroaryl.

The term “halo” and “halogen” refers to fluoro, chloro, bromo, or iodo.

The term “hydroxy” and “hydroxyl” refers to the group —OH.

The term “heteroaryl” refers to an aromatic group of one to fifteen carbon atoms, such as one to ten carbon atoms, and one to ten heteroatoms selected from the group consisting of oxygen, nitrogen, and sulfur within the ring. Such a heteroaryl group can have a single ring (such as, pyridinyl, imidazolyl, or furyl) or multiple condensed rings in a ring system (for example as in groups such as, indolizinyl, quinolinyl, benzofuran, benzimidazolyl, or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In some embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with one to five substituents, or one to three substituents, wherein the substituents are independently selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO— substituted alkyl, —SO-aryl, —SO-heteroaryl, —SO₂-alkyl, —SO₂-substituted alkyl, —SO₂-aryl, and —SO₂-heteroaryl, and trihalomethyl.

The term “heteroaralkyl” refers to a group comprising an alkylene bound to a heteroaryl, wherein alkylene and heteroaryl are defined herein. This term includes, by way of example, pyridylmethyl, pyridylethyl, indolylmethyl, and the like.

The term “heteroaryloxy” refers to —O-heteroaryl.

The terms “heterocycle,” “heterocyclic,” “heterocycloalkyl,” and “heterocyclyl” are used herein interchangeably to refer to a saturated or unsaturated group having a single ring or multiple condensed rings, including fused bridged and spiro ring systems, and having from three to twenty ring atoms, including one to ten hetero atoms. These ring atoms are selected from nitrogen, sulfur, and oxygen, where, in fused ring systems, one or more of the rings can be cycloalkyl, aryl, or heteroaryl, provided that the point of attachment is through the non-aromatic ring. In some embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with one to five, or from one to three substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

The term “hydroxyamino” refers to the group —NHOH.

The term “nitro” refers to the group —NO₂.

The term “oxo” refers to a double-bonded oxygen atom (═O).

The term “sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

The term “thiol” refers to the group —SH.

The term “thioxo” or the term “thioketo” refers to a double bonded sulfur atom (═S).

The term “alkylthio” or the term “thioalkoxy” refers to the group —S-alkyl, wherein alkyl is as defined herein. In some embodiments, sulfur can be oxidized to —S(O)—. The sulfoxide can exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O³¹)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, wherein R⁶⁰ is selected from the group consisting of optionally substituted alkyl, cycloalkyl, heteroalkyl, heterocycloalkylalkyl, cycloalkylalkyl, aryl, arylalkyl, heteroaryl, and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which can optionally include from one to four of the same or different additional heteroatoms selected from the group consisting of 0, N and S, of which N can have —H or C₁-C₆ alkyl substitution; and each M⁺ is a counter ion with a net single positive charge. Each M⁺ can independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl, and N-morpholinyl.

In addition to the disclosure herein, substituent groups for hydrogens on unsaturated carbon atoms in “substituted” alkenyl, alkynyl, aryl, and heteroaryl groups are, unless otherwise specified, —R⁶⁰, halo, —O⁻M⁺, —OR⁷⁰, —SR⁷⁰, —S—, M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸OR⁸⁰, —C(NR⁷⁰)NR⁸OR⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸OR⁸⁰, wherein R⁶⁰, R⁷⁰, R⁸⁰ and M⁺ are as defined herein, provided that in case of substituted alkenyl or alkynyl, the substituents are not —O⁻M⁺, —OR⁷⁰, —SR⁷⁰, or —S-M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸OR⁸⁰, —C(NR⁷⁰)NR⁸OR⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸OR⁸⁰, wherein R⁶⁰, R⁷⁰, R⁸⁰ and M⁺ are as defined herein.

A group that is substituted can have one, two, three, or four substituents, such as one, two, or three substituents, or one or two substituents, or one substituent.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “subject” refers to human and non-human subjects, especially mammalian subjects.

The term “treating” or “treatment” as used herein means the treating or treatment of a disease or medical condition in a subject, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a subject; (c) suppressing the disease or medical condition, for example by, slowing or arresting the development of the disease or medical condition in a subject; or (d) alleviating a symptom of the disease or medical condition in a subject. In some embodiments, the term “treating,” or “treatment” excludes a prophylactic treatment.

The terms “native amino acid sequence” as used herein refers to the amino acid sequence of a polypeptide prior to modification to include a modified amino acid residue.

The terms “amino acid analog,” “unnatural amino acid,” and the like is used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some embodiments, the amino acid analogs share backbone structures, and/or the side chain structures of one or more natural amino acids, with difference(s) being one or more modified groups in the molecule. Such modification can include, but is not limited to, substitution of an atom (such as N) for a related atom (such as S), addition of a group (such as methyl, or hydroxyl, and the like) or an atom (such as Cl or Br, and the like), deletion of a group, substitution of a covalent bond (single bond for double bond, and the like), or combinations thereof. For example, amino acid analogs can include α-hydroxy acids, and α-amino acids, and the like.

The term “amino acid side chain” is used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

The term “carbohydrate” is used to refer to monomer units and/or polymers of monosaccharides, disaccharides, oligosaccharides, and polysaccharides. The term sugar is be used to refer to the smaller carbohydrates, such as monosaccharides, disaccharides. The term “carbohydrate derivative” includes compounds where one or more functional groups of a carbohydrate of interest are substituted (replaced by any convenient substituent), modified (converted to another group using any convenient chemistry) or absent (e.g., eliminated or replaced by H). A variety of carbohydrates and carbohydrate derivatives are available and can be adapted for use in the subject compounds and conjugates.

The term “glycoside” or “glycosyl” refers to a sugar molecule or group bound to a moiety via a glycosidic bond. For example, the moiety that the glycoside is bound to can be a cleavable linker as described herein. A glycosidic bond can link the glycoside to the other moiety through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some cases, glycosides can be cleaved from the moiety they are attached to, such as by chemically-mediated hydrolysis or enzymatically-mediated hydrolysis.

The term “5T4” refers to a polypeptide (“polypeptide” and “protein” are used interchangeably herein) or any native 5T4 from any vertebrate source, including mammals such as primates (e.g., humans, cynomolgus monkey (cyno)), dogs, and rodents (e.g., mice and rats), unless otherwise indicated. 5T4, also known as “5T4 Oncofetal Trophoblast Glycoprotein” or “Wnt-Activated Inhibitory Factor 1” or “5T4 Oncotrophoblast Glycoprotein” or “5T4 Oncofetal Antigen” or “WAIF1” or “M6P1” or “5T4-AG” or “5T4AG” or “TPBG” is a glycoprotein encoded by the TPBG gene. The term 5T4 encompasses “full-length,” 5T4, as well as any form of 5T4 or any fragment thereof that results from processing in the cell. In some embodiments, the 5T4 comprises a signal sequence. In some embodiments, the 5T4 does not include a signal sequence. In some embodiments, the term 5T4 refers to a fragment of the full-length 5T4, which comprises a 5T4 extracellular domain. The term 5T4 also encompasses naturally occurring variants of 5T4, such as SNP variants, splice variants and allelic variants. The full-length amino acid sequence of human 5T4 is provided below (exemplary signal sequence=italic text; exemplary extracellular domain=underline text):

(SEQ ID NO: 64)
MPGGCSRGPA AGDGRLRLAR LALVLLGWVS SSSPTSSASS FSSSAPFLAS AVSAQPPLPD
QCPALCECSE AARTVKCVNR NLTEVPTDLP AYVRNLFLTG NQLAVLPAGA FARRPPLAEL
AALNLSGSRL DEVRAGAFEH LPSLRQLDLS HNPLADLSPF AFSGSNASVS APSPLVELIL
NHIVPPEDER QNRSFEGMVV AALLAGRALQ GLRRLELASN HFLYLPRDVL AQLPSLRHLD
LSNNSLVSLT YVSFRNLTHL ESLHLEDNAL KVLHNGTLAE LOGLPHIRVF LDNNPWVCDC
HMADMVTWLK ETEVVQGKDR LTCAYPEKMR NRVLLELNSA DLDCDPILPP SLQTSYVFLG
IVLALIGAIF LLVLYLNRKG IKKWMHNIRD ACRDHMEGYH YRYEINADPR LTNLSSNSDV

The full-length amino acid sequence of cynomolgus monkey (cyno) 5T4 is provided below (exemplary signal sequence=italic text; exemplary extracellular domain=underline text):

(SEQ ID NO: 65)
MPGGCSRGPA AGDGRLRLAR LALVLLGWVS SSSSTSSASS SSSSAPFLAS AASAQPPLPD
QCPALCECSE AARTVKCVNR NLTEVPTDLP LYVRNLFLTG NQLAVLPAGA FARRPPLAEL
AALNLSGSRL DEVRGGAFEH LPSLRQLDLS HNPLAYLSPF AFSGSNASIS APSPLVELIL
NHIVPPDDKR QNRSFEGMVA AALVAGRALQ GLHLLELASN HFLYLPRDVL AQLPSLRYLD
LSNNSLVSLT YVSFRNLTHL ESLHLEDNAL KVLHNGTLAE LQGLPHVRVF LDNNPWVCDC
HMADMVTWLK QTGVVQGKDR LTCAFPEKMR NRVLLELNSA DLDCDPILPP SLQTSYVFLG
IVLALIGAIF LLVLYLNRKG IKKWMHNIRD ACRDHMEGYH YRYEINADPR LTNLSSNSDV

The full-length amino acid sequence of mouse 5T4 is provided below (exemplary signal sequence=italic text; exemplary extracellular domain=underline text):

(SEQ ID NO: 66)
MPGAGSRGPS AGDGRLRLAR LALVLLGWVS ASAPSSSVPS SSTSPAAFLA SGSAQPPPAE
RCPAACECSE AARTVKCVNR NLLEVPADLP PYVRNLFLTG NQMTVLPAGA FARQPPLADL
EALNLSGNHL KEVCAGAFEH LPGLRRLDLS HNPLTNLSAF AFAGSNASVS APSPLEELIL
NHIVPPEDQR QNGSFEGMVA FEGMVAAALR SGLALRGLTR LELASNHFLF LPRDLLAQLP
SLRYLDLRNN SLVSLTYASF RNLTHLESLH LEDNALKVLH NSTLAEWHGL AHVKVFLDNN
PWVCDCYMAD MVAWLKETEV VPDKARLTCA FPEKMRNRGL LDLNSSDLDC DAVLPQSLQT
SYVFLGIVLA LIGAIFLLVL YLNRKGIKKW MHNIRDACRD HMEGYHYRYE INADPRLTNL
SSNSDV

The terms “antibody,” “immunoglobulin,” or “Ig” are used interchangeably herein and are used in the broadest sense and specifically covers, for example polyclonal antibodies, monoclonal antibodies (including agonist, antagonist, neutralizing antibodies, full length monoclonal antibodies), antibody compositions with polyepitopic or monoepitopic specificity, recombinantly produced antibodies, single domain antibodies, monospecific antibodies, multispecific antibodies (including bispecific antibodies), synthetic antibodies, chimeric antibodies, humanized antibodies, or human versions of antibodies having full length heavy and/or light chains. Antibodies also include single antibody domains as well as antibody fragments (and/or polypeptides that comprise antibody fragments) that retain 5T4 binding characteristics. Non-limiting examples of antibody fragments include antigen-binding regions and/or effector regions of the antibody, e.g., Fab, Fab′, F(ab′)₂, Fv, scFv, (scFv)₂, single-chain antibody molecule, dual variable domain antibody, single variable domain antibody, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). In general terms, a variable (V) region can be any suitable arrangement of immunoglobulin heavy (VH) and/or light (VL) variable regions. For example, antibodies also include tetrameric antibodies comprising two heavy chain and two light chain molecules, an antibody light chain monomer, and an antibody heavy chain monomer. Thus, for example, the V region can be dimeric and contain VH—VH, VH-VL, or VL-VL dimers that bind 5T4. In any embodiment, a VH region and a VL region can be covalently coupled either directly or through a linker to form a single-chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable regions”) can be obtained by constructing polynucleotides that encode one or more CDRs of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments can be incorporated, for example, into single domain antibodies, maxibodies, minibodies, intrabodies, diabodies, triabodies, tetrabodies, variable regions of new antigen receptors (v-NAR), and bis-single-chain Fv regions (see, e.g., Hollinger and Hudson, Nature Biotechnology, 23(9):1126-1136, 2005). In some embodiments, antibodies comprising a VH and/or VL contain a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “monospecific,” as used herein denotes an antibody that has one or more binding sites each of which binds to the same epitope of the same antigen.

The term “bispecific” means that the antibody can specifically bind to at least two distinct antigenic determinants, for example two binding sites each formed by a pair of an antibody heavy chain variable region (VH) and an antibody light chain variable region (VL) binding to different antigens or to different epitopes on the same antigen. Such a bispecific antibody can have a 1+1 format (comprising one binding site for a first antigen or epitope and one binding site for a second antigen or epitope). Other bispecific antibody formats can be 2+1 or 1+2 formats (comprising two binding sites for a first antigen or epitope and one binding site for a second antigen or epitope) or 2+2 format (comprising two binding sites for a first antigen or epitope and two binding sites for a second antigen or epitope). When a bispecific antibody comprises two antigen binding sites, each can bind to a different antigenic determinant. Such a bispecific antibody can bind to two different epitopes on the same antigen (e.g., epitopes on 5T4).

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well known in the art. These include, but are not limited to, basic local alignment search tool (BLAST), ALIGN, MegAlign, BestFit, GCG Wisconsin Package, and variants thereof. In some embodiments, two nucleic acids or polypeptides are substantially identical, meaning they have at least 70%, at least 75%, at least 80%, at least 85%, or at least 90%, and in some embodiments, at least 95%, 96%, 97%, 98%, or 99% nucleotide or amino acid residue identity, when compared and aligned for maximum correspondence, as measured using a sequence comparison algorithm or by visual inspection. In some embodiments, identity exists over a region of the amino acid sequences that is at least about ten residues, at least about 20 residues, at least about 40-60 residues, at least about 60-80 residues in length or any integral value there between. In some embodiments, identity exists over a longer region than 60-80 residues, such as at least about 80-100 residues, and in some embodiments, the sequences are substantially identical over the full-length of the sequences being compared, such as the coding region of a target protein or an antibody. In some embodiments, identity exists over a region of the nucleotide sequences that is at least about ten bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments, the sequences are substantially identical over the full length of the sequences being compared, such as a nucleotide sequence encoding a protein of interest.

A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions that do not eliminate binding are well known in the art.

The term “polypeptide” refers to a polymer of amino acids of any length. The polymer can be linear or branched, it can comprise modified amino acids, and it can include (e.g., be substituted with) non-amino acids. The terms also encompass an amino acid polymer that has been modified naturally or by intervention; for example, disulfide bond formation, glycosylation, lipidation, acetylation, phosphorylation, or any other manipulation or modification, such as linkage to or conjugation with (directly or indirectly) a moiety such as a labeling component or a drug (e.g., toxin). Also included within the definition are, for example, polypeptides containing one or more analogs of an amino acid (including, for example, unnatural amino acids), as well as other modifications known in the art. It is understood that, because the polypeptides of this disclosure can be based upon antibodies or other members of the immunoglobulin superfamily, in some embodiments, the polypeptides can occur as single chains or dimers of single chains.

As used herein, an “antigen” is a moiety or molecule that contains an epitope to which an antibody can bind. As such, an antigen can be bound by an antibody. In some embodiments, the antigen to which an antibody described herein binds is 5T4 (e.g., human 5T4), or a fragment thereof, such as a fragment that comprises one or more regions of 5T4.

As used herein, an “epitope” is a term in the art and refers to a localized region of an antigen to which an antibody can bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope), e.g., human 5T4. It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three-dimensional protein structure. In other embodiments, an antibody requires amino acid residues making up the epitope to exhibit a particular conformation (e.g., bend, twist, turn or fold) in order to recognize and bind the epitope.

An antibody binds “an epitope,” “essentially the same epitope,” or “the same epitope” as a reference antibody. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels.

“Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities. Additional details regarding methods for epitope binning and determining epitope binding of antibodies are described herein, as shown in Example 5.

As used herein, the terms “specifically binds,” “specifically recognizes,” “immunospecifically binds,” “selectively binds,” “immunospecifically recognizes” and “immunospecific” are analogous terms in the context of antibodies and refer to molecules that bind to an antigen (e.g., epitope) as understood by one skilled in the art.

In some embodiments, “specifically binds” means, for instance, that a polypeptide or molecule interacts more frequently, more rapidly, with greater duration, with greater affinity, or with some combination of the above to the epitope, protein, or target molecule than with alternative substances, including related and unrelated proteins. For example, a molecule that specifically binds to an antigen can bind to other peptides or polypeptides, generally with lower affinity as determined by, e.g., immunoassays, BIACORE™, KinExA 3000 instrument (Sapidyne Instruments, Boise, ID), the OctetQK384 system (ForteBio, Menlo Park, CA), or other assays known in the art. In some embodiments, an antibody or antigen binding region binds to or specifically binds to an antigen when it binds to the antigen with higher affinity than to any cross-reactive antigen as determined using experimental techniques, such as radioimmunoassays (RIAs) and enzyme linked immunosorbent assays (ELISAs). Typically, a specific or selective reaction will be at least twice background signal or noise and can be more than ten times background. See, e.g., Fundamental Immunology 332-36 (Paul ed., 2d ed. 1989) for a discussion regarding binding specificity. In some embodiments, the extent of binding of an antibody or antigen-binding region to a “non-target” protein is less than about 10% of the binding of the antibody or antigen-binding region to its target antigen, for example, as determined by fluorescence activated cell sorting (FACS) analysis or RIAs. In some embodiments, molecules that specifically bind to an antigen bind to the antigen with a K_{A} that is at least 2 logs, 2.5 logs, 3 logs, 4 logs or greater than the K_{A} when the molecules bind to another antigen. In some embodiments, molecules that specifically bind to an antigen do not cross react with other proteins. In another specific embodiment, molecules that specifically bind to an antigen do not cross react with other non-5T4 proteins. In some embodiments, “specifically binds” means, for instance, that a polypeptide or molecule binds a protein or target with a K_{D} of about 0.1 mM or less, but more usually less than about 1 μM. In some embodiments, “specifically binds” means that a polypeptide or molecule binds a target with a K_{D} of at least about 0.1 μM or less, at least about 0.01 μM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a polypeptide or molecule that specifically binds a first target may or may not specifically bind a second target. As such, “specific binding” does not necessarily require (although it can include) exclusive binding, e.g., binding to a single target. Thus, in some embodiments, a polypeptide or molecule can specifically bind more than one target. In some embodiments, multiple targets can be bound by the same antigen-binding site on the polypeptide or molecule. For example, in some embodiments, an antibody can comprise two identical antigen-binding sites, each of which specifically binds the same epitope on two or more proteins. In alternative embodiments, an antibody can be bispecific and comprise at least two antigen-binding sites with differing specificities. Generally, but not necessarily, reference to “binding” means “specific binding.”

The term “binding affinity” generally refers to the strength of the sum of noncovalent interactions between a single binding site of a molecule (e.g., antibody) and its binding partner (e.g., an antigen such as 5T4). Unless indicated otherwise, as used herein, “binding affinity” refers to intrinsic binding affinity that reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity is known in the art, any of which can be used for purposes of the present disclosure. In one embodiment, the “K_{D}” or “K_{D} value” can be measured by biolayer interferometry (BLI) using, for example, the OctetQK384 system (ForteBio, Menlo Park, CA). Alternatively, the K_{D} may also be measured in a radiolabeled antigen binding assay (RIA), for example, performed with the Fab version of an antibody of interest and its antigen (Chen et al., (1999) J. Mol Biol 293:865-881) or using surface plasmon resonance (SPR) assays by BIACORE™ using, for example, a BIACORE™-2000 or a BIACORE™-3000 (BIAcore, Inc., Piscataway, NJ). An “on-rate” or “rate of association” or “association rate” or “kₒₙ,” as well as an “off-rate” or “rate of dissociation” or “dissociation rate” or “k_{off},” can also be determined with the same SPR or BLI techniques described herein using, for example, the OctetQK384 system (ForteBio, Menlo Park, CA) or a BIACORE™-2000 or a BIACORE™-3000 (BIACORE™, Inc., Piscataway, NJ), respectively.

The term “compete,” when used in the context of a 5T4 antibody, describes a binding agent that, in the presence of another binding agent, is at least partially inhibited from binding to an epitope or binding site due to binding of the other binding agent. Competition can be determined by an assay in which the binding agent under study prevents or inhibits the specific binding of a reference molecule (e.g., a reference ligand, or reference antigen binding protein, such as a reference antibody) to a common antigen (e.g., 5T4). Numerous types of competitive binding assays can be used to determine if a test binding agent competes with a reference molecule for binding to 5T4 (e.g., human 5T4). Examples of assays that can be employed include solid phase direct or indirect radioimmunoassay (RIA), solid phase direct or indirect enzyme immunoassay (EIA), sandwich competition assay (see, e.g., Stahli et al., (1983) Methods in Enzymology 9:242-253); solid phase direct biotin-avidin EIA (see, e.g., Kirkland et al., (1986) J. Immunol. 137:3614-3619 or Cheung et al., (1990) Virology 176:546-552); solid phase direct labeled assay, solid phase direct labeled sandwich assay (see, e.g., Harlow and Lane, (1988) Antibodies, A Laboratory Manual, Cold Spring Harbor Press); solid phase direct label RIA using 1-125 label (see, e.g., Morel et al., (1988) Molec. Immunol. 25:7-15); and direct labeled RIA (Moldenhauer et al., (1990) Scand. J. Immunol. 32:77-82). Typically, such an assay involves the use of a purified antigen (e.g., 5T4, such as human 5T4) bound to a solid surface or cells bearing either of an unlabeled test antigen binding protein (e.g., test 5T4 antibody or ADC) or a labeled reference antigen binding protein (e.g., reference 5T4 antibody or ADC). Competitive inhibition can be measured by determining the amount of label bound to the solid surface or cells in the presence of the test antigen binding protein. Usually, the test antigen binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur (e.g., similar epitope or overlapping epitope). Usually, when a competing antibody is present in excess, it will inhibit specific binding of a reference antibody to a common antigen by at least 20%, for example, at least 20%, 25%, 30%, 35%, 40%, 45%, 50%, 55%, 60%, 65%, 70%, or 75%. In some embodiments, binding is inhibited by at least 80%, 85%, 90%, 95%, 96%, 97%, 98%, 99% or more.

The terms “constant region” and “constant domain” are used interchangeably herein, are well-known antibody terms of art, and refer to an antibody portion, for example, a carboxyl terminal portion of a light and/or heavy chain which is not directly involved in binding of an antibody to an antigen, but which can exhibit various effector functions, such as interaction with an Fc receptor. The term includes the portion of an immunoglobulin molecule having a generally more conserved amino acid sequence relative to an immunoglobulin variable region.

Antibody “effector functions” refer to those biological activities attributable to the Fc region (e.g., a native sequence Fc region or amino acid sequence variant Fc region) of an antibody and which vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226 (of the EU numbering system) or from Pro230 (of the EU numbering system) to the carboxyl-terminus thereof. The C-terminal lysine (residue 447 of the EU numbering system) of the Fc region can be removed, for example, during production or purification of the antibody, or by recombinantly engineering the nucleic acid encoding a heavy chain of the antibody. An exemplary Fc region sequence is provided below (CH2 domain=bold text; CH3 domain=underline text):

(SEQ ID NO: 67)
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPGK.

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), and the like. Such effector functions generally require the Fc region to be combined with a binding region or binding domain (e.g., an antibody variable region or domain) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including, or combining with other sequences such as variable region sequences) by a human. Native sequence human Fc regions include a native sequence human IgG1 Fc region (non-A and A allotypes); native sequence human IgG2 Fc region; native sequence human IgG3 Fc region; and native sequence human IgG4 Fc region; as well as naturally occurring variants thereof.

A “variant Fc region” comprises an amino acid sequence that differs from that of a native sequence Fc region by virtue of at least one amino acid modification, (e.g., substituting, addition, or deletion) preferably one or more amino acid substitution(s). In some embodiments, the variant Fc region has at least one amino acid substitution compared to a native sequence Fc region or to the Fc region of a parent polypeptide, for example, from about one to about ten amino acid substitutions, and preferably from about one to about five amino acid substitutions in a native sequence Fc region or in the Fc region of the parent polypeptide. The variant Fc region described herein can possess at least about 80% homology with a native sequence Fc region and/or with an Fc region of a parent polypeptide, or at least about 90% homology therewith, for example, at least about 95% homology therewith. The variant Fc region herein described herein can have a loss of effector function (e.g., silent Fc). An exemplary variant Fc region (“silent Fc”) sequence is provided below (CH2 domain=bold text with amino acid changes underlined; CH3 domain=underline text):

(SEQ ID NO: 68)
CPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALKAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPGK.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes one or more constant regions. The “heavy chain” can refer to any distinct types, e.g., for example, alpha (α), delta (δ), epsilon (ε), gamma (γ) and mu (μ), based on the amino acid sequence of the constant region, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, e.g., kappa (κ) or lambda (λ) based on the amino acid sequence of the constant regions. Light chain amino acid sequences are well known in the art.

The terms “antigen binding fragment,” “antigen binding domain,” “antigen binding region,” and similar terms refer to a portion of an antibody that comprises amino acid residues that interact with an antigen and confer on the binding fragment or region its specificity and affinity for the antigen (e.g., the CDRs). “Antigen binding fragment” as used herein includes “antibody fragment,” which comprises a portion of an antibody including one or more CDRs, such as the antigen binding or variable region of the antibody.

Antibodies described herein include, but are not limited to, synthetic antibodies, monoclonal antibodies, recombinantly produced antibodies, multispecific antibodies (e.g., including bispecific antibodies), human antibodies, humanized antibodies, chimeric antibodies, intrabodies, single-chain Fvs (scFv) (e.g., including monospecific, bispecific, and the like), camelized antibodies, Fab fragments, F(ab′) fragments, disulfide-linked Fvs (sdFv), anti-idiotypic (anti-Id) antibodies, and epitope-binding fragments of any of the above.

In some embodiments, antibodies described herein include immunoglobulin molecules and immunologically active portions of immunoglobulin molecules, such as molecules that contain one or more antigen binding sites that bind to 5T4.

An 5T4 antibody, as described herein, can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In some embodiments, a 5T4 antibody, as described herein, is an IgG antibody (e.g., human IgG), or a class (e.g., human IgG1, IgG2, IgG3, or IgG4) or a subclass thereof.

In some embodiments, a 5T4 antibody is a 4-chain antibody unit comprising two heavy (H) chain/light (L) chain pairs, wherein the amino acid sequences of the H chains are identical and the amino acid sequences of the L chains are identical. In some embodiments, the H and/or L chains comprise constant regions, for example, human constant regions. In some embodiments, the L chain constant region of a 5T4 antibody is a kappa or lambda light chain constant region, for example, a human kappa or lambda light chain constant region. In some embodiments, the H chain constant region of a 5T4 antibody comprises a gamma heavy chain constant region, for example, a human gamma heavy chain constant region. In some embodiments, a 5T4 antibody comprise an IgG constant region, for example, human IgG constant regions (e.g., IgG1, IgG2, IgG3, and/or IgG4 constant regions).

As used herein, “5T4 antibody” and “antibody that binds to 5T4” are used interchangeably and refer to an antibody that preferentially binds to 5T4. An antibody or fragment thereof can preferentially bind to 5T4, such as human 5T4, which means that the antibody or fragment thereof binds to 5T4, such as human 5T4, with greater affinity than it binds to an unrelated control protein. For example, the antibody or fragment thereof can specifically recognize and bind to 5T4 or a portion thereof. “Specific binding” means that the 5T4 antibody or fragment thereof binds to 5T4 with an affinity that is at least 5, 10, 15, 20, 25, 50, 100, 250, 500, 1000, or 10,000 times greater than the affinity for an unrelated control protein (e.g., hen egg white lysozyme). In some embodiments, the 5T4 antibody or fragment thereof can bind 5T4 substantially exclusively (e.g., is able to distinguish 5T4 from other known polypeptides, for example, by virtue of measurable differences in binding affinity). In some embodiments, a 5T4 antibody can react with 5T4 sequences other than human 5T4 sequences (e.g., cynomolgus 5T4 sequences).

The terms “variable region” and “variable domain” are used interchangeably to refer to a portion of the light and heavy chains of an antibody that are generally located at the amino-terminal of the light and heavy chain, has a length of about 120 to 130 amino acids in the heavy chain, about 100 to 110 amino acids in the light chain, and is used in the binding and specificity of each antibody for its antigen. The variable region of the heavy chain is referred to herein as “VH.” The variable region of the light chain is referred to herein as “VL.” The term “variable” refers to the fact that certain segments of the variable regions differ extensively in sequence among antibodies. The V region mediates antigen binding and defines specificity of a particular antibody for its antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” or alternatively called “complementarity determining regions.” The variable regions of heavy and light chains each comprise four frameworks (FR1, FR2, FR3 and FR4), largely adopting a p sheet configuration, connected by three hypervariable regions, which form loops connecting, and in some cases forming part of, the p sheet structure. The hypervariable regions in each chain are held together in proximity by the frameworks and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, MD, (1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “hypervariable region,” “HVR,” “HV,” “complementarity determining region, “and “CDR” when used herein refers to the regions of an antibody variable region that are hypervariable in sequence and/or form structurally defined loops. Generally, antibodies comprise six hypervariable regions: three in the VH (H1 or VH CDR1, H2 or VH CDR2, and H3 or VH CDR3), and three in the VL (L1 or VL CDR1, L2 or VL CDR2, and L3 or VL CDR3). Several hypervariable region delineations are in use and are encompassed herein. The Kabat CDRs are based on sequence variability and are the most used (see, e.g., Kabat et al., Sequences of Proteins of Immunological Interest, 5th Ed. Public Health Service, National Institutes of Health, Bethesda, MD. (1991)). Chothia refers instead to the location of the structural loops (see, e.g., Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

A universal numbering system has been developed and widely adopted, ImMunoGeneTics (IMGT®) Information System (Lefranc et al., Dev. Comp. Immunol. 27(1):55-77 (2003)). IMGT© is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable region is conserved between species and present in structures called loops, by using numbering systems that align variable region sequences of structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Pluckthun, J. Mol. Biol. 309: 657-670 (2001). Correspondence between the numbering system, including, for example, the Kabat numbering and the IMGT® unique numbering system, is well known to one skilled in the art (see, e.g., Kabat, supra; Chothia and Lesk, supra; Martin, supra; Lefranc et al., supra) and is also illustrated below. Various systems known in the art or described herein represent different ways of delineating CDRs, and when they are used to define the same antibody, they are often considered equivalent. An exemplary system, shown herein, combines Kabat and Chothia.

| | Exemplary | IMGT ® | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions can comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-102, or 95-102 (H3) in the VH. As used herein, the terms “hypervariable region,” “HVR,” “HV,” “complementarity determining region,” or “CDR” are used interchangeably.

The term “vector” refers to a substance that is used to carry or include a nucleic acid sequence, for example, to introduce a nucleic acid sequence into a host cell. Examples of vectors include expression vectors, plasmids, phage vectors, viral vectors, episomes and artificial chromosomes, which can include selection sequences or markers operable for stable integration into a host cell's chromosome. Additionally, a vector can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g., both an antibody heavy and light chain or an antibody VH and VL region) both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a 5T4 antibody), and it is further understood that expression levels can be optimized to obtain sufficient expression using methods well known in the art.

The terms “5T4-mediated disease,” “5T4-mediated disorder”, and “5T4-mediated condition” are used interchangeably and refer to any disease, disorder or condition associated with or characterized by 5T4-expressing cells, such as 5T4-expressing tumor cells. A 5T4-mediated disease includes a cancer including, but not limited to, cancers that express or overexpress 5T4.

The term “tumor,” in any embodiment herein, refers to any neoplastic cell growth or proliferation, whether malignant or benign, and to all pre-cancerous and cancerous cells and tissues.

The terms “cancer” and “cancerous” refer to or describe the physiological condition in mammals that is typically characterized by unregulated cell growth.

The term “ADC” refers to an antibody-drug conjugate, which in the context of the present invention refers to a 5T4 antibody, which is coupled to another moiety which includes a drug, as described herein.

As used herein, “drug” refers to a compound that has biological activity, such as a cytotoxic compound (e.g., a cytotoxic small molecule, a cytotoxic synthetic peptide, and the like).

Examples of drugs include small molecule drugs, such as a cancer chemotherapeutic agent. For example, where the polypeptide is an antibody (or fragment thereof) that has specificity for a tumor cell, the antibody can be modified as described herein to include a modified amino acid, which can be subsequently conjugated to a cancer chemotherapeutic agent. Cancer chemotherapeutic agents include non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones. Peptidic compounds can also be used.

Suitable cancer chemotherapeutic agents include dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. For example, dolastatin 10 or auristatin PE can be included in a 5T4-ADC of the present disclosure. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD)).

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (CYNOTAN™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U®), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatin, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (TAXOL®), docetaxel (TAXOTERE®), deoxycoformycin, mitomycin-C, L-asparaginase, azathioprine; brequinar; alkaloids, e.g. vincristine, vinblastine, vinorelbine, vindesine, and the like; podophyllotoxins, e.g. etoposide, teniposide, and the like; antibiotics, e.g. anthracycline, daunorubicin hydrochloride (daunomycin, rubidomycin, cerubidine), idarubicin, doxorubicin, epirubicin and morpholino derivatives, and the like; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, and the like; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (TAXOL®), TAXOL® derivatives, docetaxel (TAXOTERE®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, and the like; estrogens and pregestins, e.g. hydroxyprogesterone caproate, medroxyprogesterone acetate, megestrol acetate, estradiol, clomiphene, tamoxifen; and the like; adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, flutamide (DROGENIL®), toremifene (FARESTON®), and goserelin (ZOLADEX®), and the like. Estrogens stimulate proliferation and differentiation; therefore compounds that bind to the estrogen receptor are used to block this activity. Corticosteroids can inhibit T cell proliferation.

Other suitable chemotherapeutic agents include metal complexes, e.g. cisplatin (cis-DDP), carboplatin, and the like; ureas, e.g. hydroxyurea; hydrazines, e.g. N-methylhydrazine; epidophyllotoxin; a topoisomerase inhibitor; procarbazine; mitoxantrone; leucovorin; tegafur; and the like Other anti-proliferative agents of interest include immunosuppressants, e.g. mycophenolic acid, thalidomide, desoxyspergualin, azasporine, leflunomide, mizoribine, azaspirane (SKF 105685); gefitinib (IRESSA®, ZD 1839, 4-(3-chloro-4-fluorophenylamino)-7-methoxy-6-(3-(4-morpholinyl)propoxy)quinazoline); and the like.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL®, TAXOTERE® (a formulation of docetaxel), 10-desacetyl analogs of paclitaxel and 3′N-desbenzoyl-3′N-t-butoxycarbonyl analogs of paclitaxel) can be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis). Paclitaxel should be understood to refer to not only the common chemically available form of paclitaxel, but analogs and derivatives (e.g., TAXOTERE® docetaxel, as noted herein) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ; (8) colony-stimulating factors; and (9) inhibitors of angiogenesis.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition. In some embodiments, the effective amount is a therapeutically effective amount.

The term “therapeutically effective amount” as used herein refers to the amount of an antibody or ADC described herein that is sufficient to reduce and/or ameliorate the severity and/or duration of a given disease, disorder, or condition, and/or a symptom related thereto. A therapeutically effective amount of an agent, including a therapeutic agent, can be an amount necessary for (i) reduction or amelioration of the advancement or progression of a given disease, disorder, or condition, (ii) reduction or amelioration of the recurrence, development or onset of a given disease, disorder or conditions, and/or (iii) to improve or enhance the therapeutic effect of another therapy (e.g., a therapy other than the administration of an antibody or ADC described herein). A “therapeutically effective amount” of a substance/molecule/agent of the present disclosure (e.g., a 5T4 antibody or ADC) can vary based on a number of factors such as the disease state, age, sex, and weight of the individual, and the ability of the substance/molecule/agent, to elicit a desired response in the individual. A therapeutically effective amount encompasses an amount in which any toxic or detrimental effects of the substance/molecule/agent are outweighed by the therapeutically beneficial effects. In some embodiments, the term “therapeutically effective amount” refers to an amount of an antibody or other agent (e.g., or drug) effective to “treat” a disease, disorder, or condition, in a subject or mammal.

In some embodiments, the drug is a microtubule affecting agent that has anti-proliferative activity, such as a maytansinoid. In some embodiments, the drug is an antimitotic agent, such as an auristatin or an active auristatin analog or derivative thereof. In some embodiments, the drug is a DNA alkylating agent.

The term “pharmaceutically acceptable” as used herein means being approved by a regulatory agency of the federal or a state government, or listed in the U.S. Pharmacopeia, European Pharmacopeia, or other generally recognized Pharmacopeia for use in animals, and more particularly in humans.

“Excipients” include carriers, excipients, preservatives, or stabilizers that are nontoxic to the cell or mammal being exposed thereto at the dosages and concentrations employed and can be included, for example, to affect stability, bulk up formulations, or to confer a therapeutic enhancement on the active ingredient in the final dosage form (e.g., facilitating absorption, reducing viscosity, enhancing solubility). An “excipient” can be an organic or inorganic ingredient, natural or synthetic with which the active ingredient is combined to facilitate the use of the active ingredient, e.g., the administration of the active ingredient to a subject. Examples of excipients include buffers such as phosphate, citrate, and other organic acids; antioxidants such as ascorbic acid; low molecular weight (e.g., less than about ten amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates such as glucose, mannose, or dextrins; chelating agents such as EDTA; sugar alcohols such as mannitol or sorbitol; salt-forming counterions such as sodium; and/or nonionic surfactants such as TWEEN™ polyethylene glycol (PEG), and PLURONICS™. The term “excipient” can also refer to a diluent, adjuvant (e.g., Freund's adjuvant (complete or incomplete)), excipient, or vehicle with which the therapeutic is administered. Such excipients can be sterile liquids, such as water and oils, such as those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, in any embodiment, can also contain minor amounts of wetting or emulsifying agents, or pH buffering agents. Compositions can take the form of solutions, suspensions, emulsion, tablets, pills, capsules, powders, sustained-release formulations, and the like. Oral compositions, such as formulations, can include standard excipients such as pharmaceutical grades of mannitol, lactose, starch, magnesium stearate, sodium saccharine, cellulose, magnesium carbonate, and the like. Examples of suitable excipients are described in Remington: The Science and Practice of Pharmacy (2020) (Elsevier Science, Amsterdam, Netherlands), including pharmaceutical compounds, can contain an effective amount or therapeutically effective amount of a 5T4-ADC, for example, in isolated or purified form, together with a suitable amount of excipient to provide the form for proper administration to the subject. The formulation should suit the mode of administration.

### 5T4 Antibodies

5T4-ADCs described herein comprise a drug and 5T4 antibody conjugated thereto. In some embodiments, a 5T4 antibody refers to an antibody, specifically binding to 5T4, such as a 5T4 protein, a 5T4 polypeptide, a 5T4 polypeptide fragment, a 5T4 peptide, or a 5T4 epitope. In some embodiments, the 5T4 antibody is a human or humanized antibody (e.g., comprising human constant regions) that binds to 5T4. In some embodiments, a 5T4 antibody can bind to 5T4 which is expressed on the surface of a mammalian (e.g., human) cell, including a 5T4-expressing tumor cell. In some embodiments, a 5T4 antibody binds a 5T4 extracellular epitope expressed on a cell such as a tumor cell (e.g., an extracellular 5T4 epitope). In some embodiments, 5T4 is a human 5T4. An exemplary amino acid sequence of human 5T4 is described herein.

In some embodiments, the 5T4 antibody competes for binding to 5T4 with a reference 5T4 antibody that comprises a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 as described in any one of Tables 1-3. Accordingly, in some embodiments, the 5T4 antibody competes for binding to 5T4 with a reference 5T4 antibody that comprises one, two, and/or three VH CDRs and/or one, two, and/or three VL CDRs from: (a) the antibody designated as mAbA4; or (b) the antibody designated as mAbA15; or (c) the antibody designated as mAbA17. In some embodiments, a 5T4-ADC comprises a drug conjugated (directly or indirectly) to a 5T4 antibody that competes for binding to 5T4 with a reference 5T4 antibody that comprises one, two, and/or three VH CDRs and one, two, and/or three VL CDRs from: (a) the antibody designated as mAbA4; or (b) the antibody designated as mAbA15; or (c) the antibody designated as mAbA17. In some embodiments, the 5T4 antibody competes for binding to 5T4 with a reference 5T4 antibody that comprises a VH region and VL region from: (a) the antibody designated as mAbA4; or (b) the antibody designated as mAbA15; or (c) the antibody designated as mAbA17. In some embodiments, the 5T4 antibody competes for binding to 5T4 with a reference 5T4 antibody that comprises: (a) a VH region comprising the amino acid sequence of SEQ ID NO:25 and a VL region comprising the amino acid sequence of SEQ ID NO:26; or (b) a VH region comprising the amino acid sequence of SEQ ID NO:44 and a VL region comprising the amino acid sequence of SEQ ID NO:45; or (c) a VH region comprising the amino acid sequence of SEQ ID NO:62 and a VL region comprising the amino acid sequence of SEQ ID NO:63.

In some embodiments, the 5T4 antibody comprises a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 as described in any one of Tables 1-3. Accordingly, in some embodiments, the 5T4 antibody comprises one, two, and/or three heavy chain CDRs and/or one, two, and/or three light chain CDRs from: (a) the antibody designated as mAbA4; or (b) the antibody designated as mAbA15; or (c) the antibody designated as mAbA17. In some embodiments, the 5T4 antibody comprises one, two, and/or three heavy chain CDRs and one, two, and/or three light chain CDRs from: (a) the antibody designated as mAbA4; or (b) the antibody designated as mAbA15; or (c) the antibody designated as mAbA17.

In some embodiments, the 5T4 antibody comprises a VH region, which comprises one or more (such as one, two, or three) of VH CDR1, VH CDR2, and VH CDR3 as described herein, such as in any one of Tables 1-3; and/or a VL region, which comprises one or more (such as one, two, or three) of VL CDR1, VL CDR2, and VL CDR3 as described herein, such as in any one of Tables 1-3. In some embodiments, the 5T4 antibody is bispecific and comprises a first binding region that comprises one, two, and/or three heavy chain CDRs and/or one, two, and/or three light chain CDRs as described in any one of Tables 1-3 and a second region that comprises one, two, and/or three heavy chain CDRs and/or one, two, and/or three light chain CDRs from a binding agent that binds to a second target antigen that is not 5T4. In some embodiments, the 5T4 antibody is bispecific and comprises a first binding domain that comprises one, two, and/or three heavy chain CDRs and/or one, two, and/or three light chain CDRs as described in any one of Tables 1-3 and a second binding domain that comprises one, two, and/or three heavy chain CDRs and/or one, two, and/or three light chain CDRs from a binding agent that binds to a second 5T4 epitope.

The antibody designated as mAbA4 comprises a VH amino acid sequence of SEQ ID NO:25 and a VL amino acid sequence of SEQ ID NO:26.

The antibody designated as mAbA15 comprises a VH amino acid sequence of SEQ ID NO:44 and a VL amino acid sequence of SEQ ID NO:45.

The antibody designated as mAbA17 comprises a VH amino acid sequence of SEQ ID NO:62 and a VL amino acid sequence of SEQ ID NO:63.

TABLE 1
Antibody mAbA4

| | | Exemplary | IMGT ® | Kabat | Chothia | Contact | AbM |
|---|---|---|---|---|---|---|---|
| VH | VH | GFTFSRYYIH | GFTFSRYY | RYYIH (SEQ | GFTFSRY (SEQ | SRYYIH | GFTFSRYYIH |
| CDR | CDR1 | (SEQ ID NO: 1) | (SEQ ID NO: 7) | ID NO: 12) | ID NO: 13) | (SEQ ID NO: 18) | (SEQ ID NO: 1) |
| Seq. | VH | FISPGGSYTYYA | ISPGGSYT | FISPGGSYTYYA | PGGS (SEQ | WVAFISPGGSYT | FISPGGSYTY |
| | CDR2 | DSVKG | (SEQ ID NO: 8) | DSVKG | ID NO: 14) | Y | (SEQ ID NO: 24) |
| | | (SEQ ID NO: 2) | | (SEQ ID NO: 2) | | (SEQ ID NO: 19) | |
| | VH | EYYSMGVLDY | AREYYSMGVLDY | EYYSMGVLDY | YYSMGVLD (SEQ | AREYYSMGVLD | EYYSMGVLDY |
| | CDR3 | (SEQ ID NO: 3) | (SEQ ID NO: 9) | (SEQ ID NO: 3) | ID NO: 15) | (SEQ ID NO: 20) | (SEQ ID NO: 3) |
| VL | VL | RASQSVSSAVA | QSVSSA | RASQSVSSAVA | SQSVSSA (SEQ | SSAVAWY | RASQSVSSAVA |
| CDR | CDR1 | (SEQ ID NO: 4) | (SEQ ID NO: 10) | (SEQ ID NO: 4) | ID NO: 16) | (SEQ ID NO: 21) | (SEQ ID NO: 4) |
| Seq. | VL | SASSLYS | SAS | SASSLYS | SAS (SEQ | LLIYSASSLY | SASSLYS |
| | CDR2 | (SEQ ID NO: 5) | (SEQ ID NO: 11) | (SEQ ID NO: 5) | ID NO: 11) | (SEQ ID NO: 22) | (SEQ ID NO: 5) |
| | VL | QQAYESPYT | QQAYESPYT | QQAYESPYT | AYESPY (SEQ | QQAYESPY | QQAYESPYT |
| | CDR3 | (SEQ ID NO: 6) | (SEQ ID NO: 6) | (SEQ ID NO: 6) | ID NO: 17) | (SEQ ID NO: 23) | (SEQ ID NO: 6) |

VH Amino Acid Sequence:
EVQLVESGGGLVQPGGSLRLSCAASGFTFSRYYIHWVRQAPGKGLEWVAFISPGGSYTYYADSVKGRFTISADTSKNTAYLQMNSLRAEDTAV
YYCAREYYSMGVLDYWGQGTLVTVSS (SEQ ID NO: 25)
VL Amino Acid Sequence:
DIQMTQSPSSLSASVGDRVTITCRASQSVSSAVAWYQQKPGKAPKLLIYSASSLYSGVPSRFSGSRSGTDFTLTISSLQPEDFATYYCQQAYES
PYTFGQGTKVEIK (SEQ ID NO: 26)

TABLE 2
Antibody mAbA15

| | | Exemplary | IMGT ® | Kabat | Chothia | Contact | AbM |
|---|---|---|---|---|---|---|---|
| VH | VH | GFTFSSYYIH | GFTFSSYY | SYYIH (SEQ | GFTFSSY | SSYYIH | GFTFSSYYIH |
| CDR | CDR1 | (SEQ ID NO: 27) | (SEQ ID NO: 31) | ID NO: 34) | (SEQ ID | (SEQ ID NO: 39) | (SEQ ID NO: 27) |
| Seq. | | | | | NO: 35) | | |
| | VH | YITSTGSYTEYAD | ITSTGSYT | YITSTGSYTEYA | STGS | WVAYITSTGSYT | YITSTGSYTE |
| | CDR2 | SVKG | (SEQ ID NO: 32) | DSVKG (SEQ | (SEQ ID | E | (SEQ ID NO: 43) |
| | | (SEQ ID NO: 28) | | ID NO: 28) | NO: 36) | (SEQ ID NO: 40) | |
| | VH | YDRSTRYSGLDY | ARYDRSTRYSGL | YDRSTRYSGLDY | DRS | ARYDRSTRYSGL | YDRSTRYSGLDY |
| | CDR3 | (SEQ ID NO: 29) | DY | (SEQ ID | TRYSGLD | D | (SEQ ID NO: 29) |
| | | | (SEQ ID NO: 33) | NO: 29) | (SEQ ID | (SEQ ID NO: 41) | |
| | | | | | NO: 37) | | |
| VL | VL | RASQSVSSAVA | QSVSSA | RASQSVSSAVA | SQSVSSA | SSAVAWY | RASQSVSSAVA |
| CDR | CDR1 | (SEQ ID NO: 4) | (SEQ ID NO: 10) | (SEQ ID | (SEQ ID | (SEQ ID NO: 21) | (SEQ ID NO: 4) |
| Seq. | | | | NO: 4) | NO: 16) | | |
| | VL | SASSLYS | SAS | SASSLYS (SEQ | SAS | LLIYSASSLY | SASSLYS |
| | CDR2 | (SEQ ID NO: 5) | (SEQ ID NO: 11) | ID NO: 5) | (SEQ ID | (SEQ ID NO: 22) | (SEQ ID NO: 5) |
| | | | | | NO: 11) | | |
| | VL | QQAYATPVT | QQAYATPVT | QQAYATPVT | AYATPV | QQAYATPV | QQAYATPVT |
| | CDR3 | (SEQ ID NO: 30) | (SEQ ID NO: 30) | (SEQ ID | (SEQ ID | (SEQ ID NO: 42) | (SEQ ID NO: 30) |
| | | | | NO: 30) | NO: 38) | | |

VH Amino Acid Sequence:
EVQLVESGGGLVQPGGSLRLSCAASGFTFSSYYIHWVRQAPGKGLEWVAYITSTGSYTEYADSVKGRFTISADTSKNTAYLQMNSLRAEDTAV
YYCARYDRSTRYSGLDYWGQGTLVTVSS (SEQ ID NO: 44)
VL Amino Acid Sequence:
DIQMTQSPSSLSASVGDRVTITCRASQSVSSAVAWYQQKPGKAPKLLIYSASSLYSGVPSRFSGSRSGTDFTLTISSLQPEDFATYYCQQAYAT
PVTFGQGTKVEIK (SEQ ID NO: 45)

TABLE 3
Antibody mAbA17

| | | Exemplary | IMGT ® | Kabat | Chothia | Contact | AbM |
|---|---|---|---|---|---|---|---|
| VH | VH | GFTFSRYWIH | GFTFSRYW | RYWIH | GFTFSRY | SRYWIH | GFTFSRYWIH |
| CDR | CDR1 | (SEQ ID NO: 46) | (SEQ ID | (SEQ ID NO: 53) | (SEQ ID NO: 13) | (SEQ ID NO: 57) | (SEQ ID NO: 46) |
| Seq. | | | NO: 50) | | | | |
| | VH | AIFPYSGSTLYAD | IFPYSGST | AIFPYSGSTLYAD | PYSG | WVAAIFPYSGST | AIFPYSGSTL |
| | CDR2 | SVKG | (SEQ ID | SVKG | (SEQ ID NO: 54) | L | (SEQ ID NO: 61) |
| | | (SEQ ID NO: 47) | NO: 51) | (SEQ ID NO: 47) | | (SEQ ID NO: 58) | |
| | VH | SPSRSILPSGLDY | ARSPSR | SPSRSILPSGLDY | PSRSILPSGLD | ARSPSRSILPSGL | SPSRSILPSGLDY |
| | CDR3 | (SEQ ID NO: 48) | SILPSGL | (SEQ ID NO: 48) | (SEQ ID NO: 55) | D | (SEQ ID NO: 48) |
| | | | DY (SEQ | | | (SEQ ID NO: 59) | |
| | | | ID NO: 52) | | | | |
| VL | VL | RASQSVSSAVA | QSVSSA | RASQSVSSAVA | SQSVSSA | SSAVAWY | RASQSVSSAVA |
| CDR | CDR1 | (SEQ ID NO: 4) | (SEQ | (SEQ ID NO: 4) | (SEQ ID NO: 16) | (SEQ ID NO: 21) | (SEQ ID NO: 4) |
| Seq. | | | ID NO: 10) | | | | |
| | VL | SASSLYS | SAS (SEQ | SASSLYS | SAS | LLIYSASSLY | SASSLYS |
| | CDR2 | (SEQ ID NO: 5) | ID NO: 11) | (SEQ ID NO: 5) | (SEQ ID NO: 11) | (SEQ ID NO: 22) | (SEQ ID NO: 5) |
| | VL | QQGDGSPYT | QQGDGSPYT | QQGDGSPYT | GDGSPY | QQGDGSPY | QQGDGSPYT |
| | CDR3 | (SEQ ID NO: 49) | (SEQ ID | (SEQ ID NO: 49) | (SEQ ID NO: 56) | (SEQ ID NO: 60) | (SEQ ID NO: 49) |
| | | | NO: 49) | | | | |

VH Amino Acid Sequence:
EVQLVESGGGLVQPGGSLRLSCAASGFTFSRYWIHWVRQAPGKGLEWVAAIFPYSGSTLYADSVKGRFTISADTSKNTAYLQMNSLRAEDTAV
YYCARSPSRSILPSGLDYWGQGTLVTVSS (SEQ ID NO: 62)
VL Amino Acid Sequence:
DIQMTQSPSSLSASVGDRVTITCRASQSVSSAVAWYQQKPGKAPKLLIYSASSLYSGVPSRFSGSRSGTDFTLTISSLQPEDFATYYCQQGDGS
PYTFGQGTKVEIK (SEQ ID NO: 63)

In some embodiments, the 5T4 antibody comprises a VH region. In some embodiments, the 5T4 antibody comprises a VL region. In some embodiments, the 5T4 antibody has a combination of (i) a VH region; and (ii) a VL region.

In some embodiments, the 5T4 antibody comprises a heavy chain having a combination of (i) a VH as described herein, such as in any one of Tables 1-3, and (ii) one or more heavy chain constant regions (e.g., CH1, hinge, CH2, and CH3). An exemplary IgG heavy chain can comprise any VH amino acid sequence as described herein and the following CH1, hinge, CH2, and CH3 amino acid sequence:

(SEQ ID NO: 69)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

Another exemplary IgG heavy chain can comprise any VH amino acid sequence as described herein and the following CH1, hinge, CH2, and CH3 amino acid sequence

(SEQ ID NO: 70)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALKAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

In some embodiments, a 5T4 antibody comprises a light chain having a combination of (i) a VL region as described herein, such as in any one of Tables 1-3; and (ii) a light chain constant region (CL). An exemplary light chain (e.g., for pairing with an IgG heavy chain) can comprise any VL amino acid sequence described herein and the following CL amino acid sequence:

(SEQ ID NO: 7)
RTVAAPSVFIFPPSDSQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In some embodiments, the 5T4 antibody comprises (a) a heavy chain having a combination of (i) a VH as described herein, such as in any one of Tables 1-3, and (ii) one or more heavy chain constant regions (e.g., CH1, hinge, CH2, and CH3); and (b) a light chain having a combination of (i) a VL as described herein, such as in any one of Tables 1-3, and (ii) a light chain constant region in an IgG format (CL1).

In some embodiments, the antibody that binds to 5T4 can comprise a heavy chain having an amino acid sequence of EVQLVESGGGLVQPGGSLRLSCAASGFTFSRYYIHWVRQAPGKGLEWVAFISPGG SYTYYADSVKGRFTISADTSKNTAYLQMNSLRAEDTAVYYCAREYYSMGVLDYWG QGTLVTVSSASTKGPSVFPLAPSSKSTSGGTAALGCLVKDYFPEPVTVSWNSGALT SGVHTFPAVLQSSGLYSLSSVVTVPSSSLGTQTYICNVNHKPSNTKVDKKVEPKSC DKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALPA PIEKTISKAKGQPREPQVYTLPPSREEMTKNQVSLTCLVKGFYPSDIAVEWESNGQ PENNYKTTPPVLDSDGSFFLYSKLTVDKSRWQQGNVFSCSVMHEALHNHYTQKSL SLSPGSLCTPSRGS (SEQ ID NO:90); and a light chain having an amino acid sequence of DIQMTQSPSSLSASVGDRVTITCRASQSVSSAVAWYQQKPGKAPKLLIYSASSLYS GVPSRFSGSRSGTDFTLTISSLQPEDFATYYCQQAYESPYTFGQGTKVEIKRTVAA PSVFIFPPSDEQLKSGTASVVCLLNNFYPREAKVQWKVDNALQSGNSQESVTEQD SKDSTYSLSSTLTLSKADYEKHKVYACEVTHQGLSSPVTKSFNRGEC (SEQ ID NO:91).

In some embodiments, the antibody that binds to 5T4 can a heavy chain which has been modified to include a formyl glycine residue and therefore have the amino acid sequence of EVQLVESGGGLVQPGGSLRLSCAASGFTFSRYYIHWVRQAPGKGLEWVAFISPGG SYTYYADSVKGRFTISADTSKNTAYLQMNSLRAEDTAVYYCAREYYSMGVLDYWG QGTLVTVSSASTKGPSVFPLAPSSKSTSGGTAALGCLVKDYFPEPVTVSWNSGALT SGVHTFPAVLQSSGLYSLSSVVTVPSSSLGTQTYICNVNHKPSNTKVDKKVEPKSC DKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALPA PIEKTISKAKGQPREPQVYTLPPSREEMTKNQVSLTCLVKGFYPSDIAVEWESNGQ PENNYKTTPPVLDSDGSFFLYSKLTVDKSRWQQGNVFSCSVMHEALHNHYTQKSL SLSPGSL(fGly)TPSRGS (SEQ ID NO:92), wherein f(Gly) is the formyl glycine residue.

In some embodiments, a 5T4-ADC comprises a heavy chain having the amino acid sequence of EVQLVESGGGLVQPGGSLRLSCAASGFTFSRYYIHWVRQAPGKGLEWVAFISPGG SYTYYADSVKGRFTISADTSKNTAYLQMNSLRAEDTAVYYCAREYYSMGVLDYWG QGTLVTVSSASTKGPSVFPLAPSSKSTSGGTAALGCLVKDYFPEPVTVSWNSGALT SGVHTFPAVLQSSGLYSLSSVVTVPSSSLGTQTYICNVNHKPSNTKVDKKVEPKSC DKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALPA PIEKTISKAKGQPREPQVYTLPPSREEMTKNQVSLTCLVKGFYPSDIAVEWESNGQ PENNYKTTPPVLDSDGSFFLYSKLTVDKSRWQQGNVFSCSVMHEALHNHYTQKSL SLSPGSLnTPSRGS (SEQ ID NO:93), wherein n is an amino acid having its side chain replaced by a linker-drug as disclosed herein, for example, a linker-drug of Formula (XIV), (XV), or (XVI).

In some embodiments, an antibody that binds to 5T4 comprises a heavy chain having an amino acid sequence of EVQLVESGGGLVQPGGSLRLSCAASGFTFSSYYIHWVRQAPGKGLEWVAYITSTG SYTEYADSVKGRFTISADTSKNTAYLQMNSLRAEDTAVYYCARYDRSTRYSGLDY WGQGTLVTVSSASTKGPSVFPLAPSSKSTSGGTAALGCLVKDYFPEPVTVSWNSG ALTSGVHTFPAVLQSSGLYSLSSVVTVPSSSLGTQTYICNVNHKPSNTKVDKKVEP KSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKA LPAPIEKTISKAKGQPREPQVYTLPPSREEMTKNQVSLTCLVKGFYPSDIAVEWESN GQPENNYKTTPPVLDSDGSFFLYSKLTVDKSRWQQGNVFSCSVMHEALHNHYTQ KSLSLSPGSLCTPSRGS (SEQ ID NO:94), and a light chain having an amino acid sequence of DIQMTQSPSSLSASVGDRVTITCRASQSVSSAVAWYQQKPGKAPKLLIYSASSLYS GVPSRFSGSRSGTDFTLTISSLQPEDFATYYCQQAYATPVTFGQGTKVEIKRTVAA PSVFIFPPSDEQLKSGTASVVCLLNNFYPREAKVQWKVDNALQSGNSQESVTEQD SKDSTYSLSSTLTLSKADYEKHKVYACEVTHQGLSSPVTKSFNRGEC (SEQ ID NO:95).

In some embodiments, the antibody that binds to 5T4 can a heavy chain which has been modified to include a formyl glycine residue and therefore have the amino acid sequence of EVQLVESGGGLVQPGGSLRLSCAASGFTFSSYYIHWVRQAPGKGLEWVAYITSTG SYTEYADSVKGRFTISADTSKNTAYLQMNSLRAEDTAVYYCARYDRSTRYSGLDY WGQGTLVTVSSASTKGPSVFPLAPSSKSTSGGTAALGCLVKDYFPEPVTVSWNSG ALTSGVHTFPAVLQSSGLYSLSSVVTVPSSSLGTQTYICNVNHKPSNTKVDKKVEP KSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKA LPAPIEKTISKAKGQPREPQVYTLPPSREEMTKNQVSLTCLVKGFYPSDIAVEWESN GQPENNYKTTPPVLDSDGSFFLYSKLTVDKSRWQQGNVFSCSVMHEALHNHYTQ KSLSLSPGSL(fGly)TPSRGS (SEQ ID NO:96), wherein f(Gly) is the formyl glycine residue.

In some embodiments, a 5T4-ADC comprises a heavy chain having an amino acid sequence of EVQLVESGGGLVQPGGSLRLSCAASGFTFSSYYIHWVRQAPGKGLEWVAYITSTG SYTEYADSVKGRFTISADTSKNTAYLQMNSLRAEDTAVYYCARYDRSTRYSGLDY WGQGTLVTVSSASTKGPSVFPLAPSSKSTSGGTAALGCLVKDYFPEPVTVSWNSG ALTSGVHTFPAVLQSSGLYSLSSVVTVPSSSLGTQTYICNVNHKPSNTKVDKKVEP KSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKA LPAPIEKTISKAKGQPREPQVYTLPPSREEMTKNQVSLTCLVKGFYPSDIAVEWESN GQPENNYKTTPPVLDSDGSFFLYSKLTVDKSRWQQGNVFSCSVMHEALHNHYTQ KSLSLSPGSLnTPSRGS (SEQ ID NO:97), wherein n is an amino acid having its side chain replaced by a linker-drug as disclosed herein, for example, a linker-drug of Formula (XIV), (XV), or (XVI).

In some embodiments, an antibody that binds to 5T4 comprises a heavy chain having the amino acid sequence of EVQLVESGGGLVQPGGSLRLSCAASGFTFSRYWIHWVRQAPGKGLEWVAAIFPYS GSTLYADSVKGRFTISADTSKNTAYLQMNSLRAEDTAVYYCARSPSRSILPSGLDY WGQGTLVTVSSASTKGPSVFPLAPSSKSTSGGTAALGCLVKDYFPEPVTVSWNSG ALTSGVHTFPAVLQSSGLYSLSSVVTVPSSSLGTQTYICNVNHKPSNTKVDKKVEP KSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKA LPAPIEKTISKAKGQPREPQVYTLPPSREEMTKNQVSLTCLVKGFYPSDIAVEWESN GQPENNYKTTPPVLDSDGSFFLYSKLTVDKSRWQQGNVFSCSVMHEALHNHYTQ KSLSLSPGSLCTPSRGS (SEQ ID NO:98), and a light chain having the amino acid sequence of DIQMTQSPSSLSASVGDRVTITCRASQSVSSAVAWYQQKPGKAPKLLIYSASSLYS GVPSRFSGSRSGTDFTLTISSLQPEDFATYYCQQGDGSPYTFGQGTKVEIKRTVAA PSVFIFPPSDEQLKSGTASVVCLLNNFYPREAKVQWKVDNALQSGNSQESVTEQD SKDSTYSLSSTLTLSKADYEKHKVYACEVTHQGLSSPVTKSFNRGEC (SEQ ID NO:99).

In some embodiments, the antibody that binds to 5T4 can a heavy chain which has been modified to include a formyl glycine residue and therefore have the amino acid sequence of EVQLVESGGGLVQPGGSLRLSCAASGFTFSRYWIHWVRQAPGKGLEWVAAIFPYS GSTLYADSVKGRFTISADTSKNTAYLQMNSLRAEDTAVYYCARSPSRSILPSGLDY WGQGTLVTVSSASTKGPSVFPLAPSSKSTSGGTAALGCLVKDYFPEPVTVSWNSG ALTSGVHTFPAVLQSSGLYSLSSVVTVPSSSLGTQTYICNVNHKPSNTKVDKKVEP KSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKA LPAPIEKTISKAKGQPREPQVYTLPPSREEMTKNQVSLTCLVKGFYPSDIAVEWESN GQPENNYKTTPPVLDSDGSFFLYSKLTVDKSRWQQGNVFSCSVMHEALHNHYTQ KSLSLSPGSL(fGly)TPSRGS (SEQ ID NO:100), wherein f(Gly) is the formyl glycine residue.

In some embodiments, a 5T4-ADC comprises a heavy chain having an amino acid sequence of EVQLVESGGGLVQPGGSLRLSCAASGFTFSRYWIHWVRQAPGKGLEWVAAIFPYS GSTLYADSVKGRFTISADTSKNTAYLQMNSLRAEDTAVYYCARSPSRSILPSGLDY WGQGTLVTVSSASTKGPSVFPLAPSSKSTSGGTAALGCLVKDYFPEPVTVSWNSG ALTSGVHTFPAVLQSSGLYSLSSVVTVPSSSLGTQTYICNVNHKPSNTKVDKKVEP KSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKA LPAPIEKTISKAKGQPREPQVYTLPPSREEMTKNQVSLTCLVKGFYPSDIAVEWESN GQPENNYKTTPPVLDSDGSFFLYSKLTVDKSRWQQGNVFSCSVMHEALHNHYTQ KSLSLSPGSLnTPSRGS (SEQ ID NO:101), wherein n is an amino acid having its side chain replaced by a linker-drug as disclosed herein, for example, a linker-drug of Formula (XIV), (XV), or (XVI).

In some embodiments, a 5T4 antibody comprises one or more CDRs (e.g., one, two, three, four, five, or six CDRs), for example, a VH CDR1, VH CDR2, VH CDR3, VL CDR1, VL CDR2, and/or VL CDR3 as described in Table 1. In some embodiments, a 5T4 antibody comprises one or more CDRs, (e.g., one, two, three, four, five, or six CDRs), for example, a VH CDR1, VH CDR2, VH CDR3, VL CDR1, VL CDR2, and/or VL CDR3 as described in Table 2. In some embodiments, a 5T4 antibody comprises one or more CDRs, (e.g., one, two, three, four, five, or six CDRs), for example, a VH CDR1, VH CDR2, VH CDR3, VL CDR1, VL CDR2, and/or VL CDR3 as described in Table 3.

In some embodiments, a 5T4 antibody comprises one or more CDRs, (e.g., one, two, or three VH CDRs), for example, a VH CDR1, VH CDR2, VH CDR3, as described in Table 1. In some embodiments, a 5T4 antibody comprises one or more CDRs, (e.g., one, two, or three VL CDRs), for example, a VL CDR1, VL CDR2, and/or VL CDR3, as described in Table 1. In some embodiments, a 5T4 antibody comprises one or more CDRs, (e.g., one, two, or three VH CDRs), for example, a VH CDR1, VH CDR2, VH CDR3, as described in Table 1 and one or more CDRs, (e.g., one, two, or three VL CDRs), for example, a VL CDR1, VL CDR2, and/or VL CDR3, as described in Table 1.

In some embodiments, a 5T4 antibody comprises one or more CDRs, (e.g., one, two, or three VH CDRs), for example, a VH CDR1, VH CDR2, VH CDR3, as described in Table 2. In other embodiments, a 5T4 antibody comprises one or more CDRs, (e.g., one, two, or three VL CDRs), for example, a VL CDR1, VL CDR2, and/or VL CDR3, as described in Table 2. In some embodiments, a 5T4 antibody comprises one or more CDRs, (e.g., one, two, or three VH CDRs), for example, a VH CDR1, VH CDR2, VH CDR3, as described in Table 2 and one or more CDRs, (e.g., one, two, or three VL CDRs), for example, a VL CDR1, VL CDR2, and/or VL CDR3, as described in Table 2.

In some embodiments, a 5T4 antibody comprises one or more CDRs, (e.g., one, two, or three VH CDRs), for example, a VH CDR1, VH CDR2, VH CDR3, as described in Table 3. In some embodiments, a 5T4 antibody comprises one or more CDRs, (e.g., one, two, or three VL CDRs), for example, a VL CDR1, VL CDR2, and/or VL CDR3, as described in Table 3. In some embodiments, a 5T4 antibody comprises one or more CDRs, (e.g., one, two, or three VH CDRs), for example, a VH CDR1, VH CDR2, VH CDR3, as described in Table 3 and one or more CDRs, (e.g., one, two, or three VL CDRs), for example, a VL CDR1, VL CDR2, and/or VL CDR3, as described in Table 3.

In some embodiments, a 5T4 antibody comprises one or more (e.g., one, two, or three) VH CDRs as described herein, such as in Tables 1-3. In other embodiments, a 5T4 antibody comprises one or more (e.g., one, two, or three) VL CDRs as described herein, such as in Tables 1-3. In some embodiments, a 5T4 antibody comprises one or more (e.g., one, two, or three) VH CDRs as described herein, such as in Tables 1-3 and one or more (e.g., one, two, or three) VL CDRs as described herein, such as in Tables 1-3. Accordingly, in some embodiments, a 5T4 antibody comprises a VH CDR1 comprising an amino acid sequence comprising any one of SEQ ID NOs:1, 7, 12, 13, 18, 27, 31, 34, 35, 39, 46, 50, 53, and 57. In some embodiments, a 5T4 antibody comprises a VH CDR2 comprising an amino acid sequence comprising any one of SEQ ID NOs:2, 8, 14, 19, 24, 28, 32, 36, 40, 43, 47, 51, 54, 58, and 61. In some embodiments, a 5T4 antibody comprises a VH CDR3 comprising an amino acid sequence of any one of SEQ ID NOs:3, 9, 15, 20, 29, 33, 37, 41, 48, 52, 55, and 59. In some embodiments, a 5T4 antibody comprises a VH CDR1 and/or a VH CDR2 and/or a VH CDR3 independently selected from a VH CDR1, VH CDR2, VH CDR3 as described in any one of Tables 1-3. In some embodiments, a 5T4 antibody comprises a VL CDR1 comprising an amino acid sequence of any one of SEQ ID NOs:4, 10, 16, and 21. In some embodiments, a 5T4 antibody comprises a VL CDR2 comprising an amino acid sequence of any one of SEQ ID NOs:5, 11, and 22. In some embodiments, a 5T4 antibody comprises a VL CDR3 comprising an amino acid sequence of any one of SEQ ID NOs:6, 17, 23, 30, 38, 42, 49, 56, and 60. In some embodiments, a 5T4 antibody comprises a VL CDR1 and/or a VL CDR2 and/or a VL CDR3 independently selected from a VL CDR1, VL CDR2, VL CDR3 as described herein, such as in any one of Tables 1-3.

In some embodiments, a 5T4 antibody comprises a heavy chain variable region (VH) comprising: (1) a VH CDR1 comprising an amino acid sequence of any one of: (i) SEQ ID NO:1, 27, or 46, (ii) SEQ ID NO:7, 31, or 50, (iii) SEQ ID NO:12, 34, or 53, (iv) SEQ ID NO:13 or 53, and (v) SEQ ID NO:18, 39, or 57; (2) a VH CDR2 comprising an amino acid sequence of any one of: (i) SEQ ID NO:2, 28, or 47, (ii) SEQ ID NO:8, 32, or 51, (iii) SEQ ID NO:14, 36, or 54, (iv) SEQ ID NO:19, 40, or 58, and (v) SEQ ID NO:24, 43, or 61; and (3) a VH CDR3 comprising an amino acid sequence of any one of: (i) SEQ ID NO:3, 29, or 48, (ii) SEQ ID NO:9, 33, or 52, (iii) SEQ ID NO:15, 37, or 55, and (iv) SEQ ID NO:20, 41, or 59; and/or a VL region comprising: (1) a VL CDR1 comprising an amino acid sequence of any one of: (i) SEQ ID NO:4, (ii) SEQ ID NO:10, (iii) SEQ ID NO:16, and (iv) SEQ ID NO:21; (2) a VL CDR2 comprising an amino acid sequence of any one of: (i) SEQ ID NO:5, (ii) SEQ ID NO:11, and (iii) SEQ ID NO:22; and (3) a VL CDR3 comprising an amino acid sequence of any one of: (i) SEQ ID NO:6, 30, or 49, (ii) SEQ ID NO:17, 38, or 56, and (iii) SEQ ID NO:23, 42, or 60.

In some embodiments, a 5T4 antibody comprises a VH region comprising: (1) a VH CDR1 comprising an amino acid sequence of any one of: (i) SEQ ID NO:1, 27, or 46, (ii) SEQ ID NO:7, 31, or 50, (iii) SEQ ID NO:12, 34, or 53, (iv) SEQ ID NO:53, and (v) SEQ ID NO:18, 39, or 57; (2) a VH CDR2 comprising an amino acid sequence of any one of: (i) SEQ ID NO:2, 28, or 47, (ii) SEQ ID NO:8, 32, or 51, (iii) SEQ ID NO:14, 36, or 54, (iv) SEQ ID NO:19, 40, or 58, and (v) SEQ ID NO:24, 43, or 61; and (3) a VH CDR3 comprising an amino acid sequence of any one of: (i) SEQ ID NO:3, 29, or 48, (ii) SEQ ID NO:9, 33, or 52, (iii) SEQ ID NO:15, 37, or 55, and (iv) SEQ ID NO:20, 41, or 59.

In some embodiments, a 5T4 antibody comprises a VL region comprising: (1) a VL CDR1 comprising an amino acid sequence of any one of: (i) SEQ ID NO:4, (ii) SEQ ID NO:10, (iii) SEQ ID NO:16, and (iv) SEQ ID NO:21; (2) a VL CDR2 comprising an amino acid sequence of any one of: (i) SEQ ID NO:5, (ii) SEQ ID NO:11, and (iii) SEQ ID NO:22; and (3) a VL CDR3 comprising an amino acid sequence of any one of: (i) SEQ ID NO:6, 30, or 49, (ii) SEQ ID NO:17, 38, or 56, and (iii) SEQ ID NO:23, 42, or 60.

In some embodiments, a 5T4-ADC comprises an antibody that binds to 5T4, wherein the antibody comprises: (a) a VH region comprising: (1) a VH CDR1 comprising an amino acid sequence of any one of: (i) SEQ ID NO:1, 27, or 46, (ii) SEQ ID NO:7, 31, or 50, (iii) SEQ ID NO:12, 34, or 53, (iv) SEQ ID NO:53, and (v) SEQ ID NO:18, 39, or 57; (2) a VH CDR2 comprising an amino acid sequence of any one of: (i) SEQ ID NO:2, 28, or 47, (ii) SEQ ID NO:8, 32, or 51, (iii) SEQ ID NO:14, 36, or 54, (iv) SEQ ID NO:19, 40, or 58, and (v) SEQ ID NO:24, 43, or 61; and (3) a VH CDR3 comprising an amino acid sequence of any one of: (i) SEQ ID NO:3, 29, or 48, (ii) SEQ ID NO:9, 33, or 52, (iii) SEQ ID NO:15, 37, or 55, and (iv) SEQ ID NO:20, 41, or 59; and/or a VL region comprising: (1) a VL CDR1 comprising an amino acid sequence of any one of: (i) SEQ ID NO:4, (ii) SEQ ID NO:10, (iii) SEQ ID NO:16, and (iv) SEQ ID NO:21; (2) a VL CDR2 comprising an amino acid sequence of any one of: (i) SEQ ID NO:5, (ii) SEQ ID NO:11, and (iii) SEQ ID NO:22; and (3) a VL CDR3 comprising an amino acid sequence of any one of: (i) SEQ ID NO:6, 30, or 49, (ii) SEQ ID NO:17, 38, or 56, and (iii) SEQ ID NO:23, 42, or 60.

In some embodiments, a 5T4-ADC comprises an antibody that binds to 5T4, wherein the antibody comprises a VH region comprising: (1) a VH CDR1 comprising an amino acid sequence of any one of: (i) SEQ ID NO:1, 27, or 46, (ii) SEQ ID NO:7, 31, or 50, (iii) SEQ ID NO:12, 34, or 53, (iv) SEQ ID NO:13, 35, or 53, and (v) SEQ ID NO:18, 39, or 57; (2) a VH CDR2 comprising an amino acid sequence of any one of: (i) SEQ ID NO:2, 28, or 47, (ii) SEQ ID NO:8, 32, or 51, (iii) SEQ ID NO:14, 36, or 54, (iv) SEQ ID NO:19, 40, or 58, and (v) SEQ ID NO:24, 43, or 61; and (3) a VH CDR3 comprising an amino acid sequence of any one of: (i) SEQ ID NO:3, 29, or 48, (ii) SEQ ID NO:9, 33, or 52, (iii) SEQ ID NO:15, 37, or 55, and (iv) SEQ ID NO:20, 41, or 59.

In some embodiments, a 5T4-ADC comprises an antibody that binds to 5T4, wherein the antibody comprises a VL region comprising: (1) a VL CDR1 comprising an amino acid sequence of any one of: (i) SEQ ID NO:4, (ii) SEQ ID NO:10, (iii) SEQ ID NO:16, and (iv) SEQ ID NO:21; (2) a VL CDR2 comprising an amino acid sequence of any one of: (i) SEQ ID NO:5, (ii) SEQ ID NO:11, and (iii) SEQ ID NO:22; and (3) a VL CDR3 comprising an amino acid sequence of any one of: (i) SEQ ID NO:6, 30, or 49, (ii) SEQ ID NO:17, 38, or 56, and (iii) SEQ ID NO:23, 42, or 60.

In some embodiments, a 5T4-ADC comprises an antibody that binds to 5T4, wherein the antibody comprises all three heavy chain CDRs and/or all three light chain CDRs from: (a) the antibody designated as mAbA4 that comprises a VH amino acid sequence of SEQ ID NO:25 and a VL amino acid sequence of SEQ ID NO:26; or (b) the antibody designated as mAbA15 that comprises a VH amino acid sequence of SEQ ID NO:44 and a VL amino acid sequence of SEQ ID NO:45; or (c) the antibody designated as mAbA17 that comprises a VH amino acid sequence of SEQ ID NO:62 and a VL amino acid sequence of SEQ ID NO:63. In some embodiments, a 5T4-ADC comprises a 5T4 antibody, wherein the 5T4 antibody comprises all three heavy chain CDRs and/or all three light chain CDRs from the antibody designated as mAbA4. In some embodiments, a 5T4-ADC comprises a 5T4 antibody, wherein the 5T4 antibody comprises all three heavy chain CDRs and/or all three light chain CDRs from the antibody designated as mAbA15. In some embodiments, a 5T4-ADC comprises a 5T4 antibody, wherein the 5T4 antibody comprises all three heavy chain CDRs and/or all three light chain CDRs from the antibody designated as mAbA17.

In some embodiments, a 5T4-ADC comprises an antibody that binds to 5T4, wherein the antibody comprises: (a) a VH region comprising a VH CDR1, a VH CDR2, and a VH CDR3 amino acid sequence as described herein, such as in Tables 1-3; and/or (b) a VL region comprising a VL CDR1, a VL CDR2, and a VL CDR3 amino acid sequence as described herein, such as in Tables 1-3. In some embodiments, a 5T4-ADC comprises an antibody that binds to 5T4 wherein the antibody comprises: a VH region comprising a VH CDR1, a VH CDR2, and a VH CDR3 amino acid sequence as described herein, such as in Tables 1-3. In some embodiments, a 5T4-ADC comprises an antibody that binds to 5T4, wherein the antibody comprises a VL region comprising a VL CDR1, a VL CDR2, and a VL CDR3 amino acid sequence as described herein, such as in Tables 1-3.

In some embodiments, a 5T4-ADC comprises an antibody that binds to 5T4, wherein the antibody comprises: a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises a VL region comprising: (1) a VL CDR1 comprising an amino acid sequence of SEQ ID NO:4; and (2) a VL CDR2 comprising an amino acid sequence of SEQ ID NO:5.

In some embodiments, a 5T4-ADC comprises an antibody that binds to 5T4, wherein the antibody comprises: a VL region comprising: (1) a VL CDR1 comprising an amino acid sequence of SEQ ID NO:10; and (2) a VL CDR2 comprising an amino acid sequence of SEQ ID NO:11.

In some embodiments, a 5T4-ADC comprises an antibody that binds to 5T4, wherein the antibody comprises: a VL region comprising: (1) a VL CDR1 comprising an amino acid sequence of SEQ ID NO:16; and (2) a VL CDR2 comprising an amino acid sequence of SEQ ID NO:11

In some embodiments, a 5T4-ADC comprises an antibody that binds to 5T4, wherein the antibody comprises: a VL region comprising: (1) a VL CDR1 comprising an amino acid sequence of SEQ ID NO:21; and (2) a VL CDR2 comprising an amino acid sequence of SEQ ID NO:22.

In some embodiments, a 5T4-ADC comprises an antibody that binds to 5T4, wherein the antibody comprises: (a) a VH region comprising: (1) a VH CDR1 comprising an amino acid sequence of SEQ ID NO:1; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: (a) a VH region comprising: (1) a VH CDR1 comprising an amino acid sequence of SEQ ID NO:1; and (b) a VL region comprising: (1) a VL CDR1 comprising an amino acid sequence of SEQ ID NO:4; and (2) a VL CDR2 comprising an amino acid sequence of SEQ ID NO:5.

In some embodiments, a 5T4-ADC comprises an antibody that binds to 5T4, wherein the antibody comprises: (a) a VH region comprising: (1) a VH CDR1 comprising an amino acid sequence of SEQ ID NO:7; and (b) a VL region comprising: (1) a VL CDR1 comprising an amino acid sequence of any of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: (a) a VH region comprising: a VH CDR1 comprising the amino acid sequence of SEQ ID NO:7; and (b) a VL region comprising: (1) a VL CDR1 comprising an amino acid sequence of SEQ ID NO:10; and (2) a VL CDR2 comprising an amino acid sequence of SEQ ID NO:11.

In some embodiments, a 5T4-ADC comprises an antibody that binds to 5T4, wherein the antibody comprises: (a) a VH region comprising: (1) a VH CDR1 comprising an amino acid sequence of SEQ ID NO:12; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: (a) a VH region comprising: a VH CDR1 comprising an amino acid sequence of SEQ ID NO:12; and (b) a VL region comprising: (1) a VL CDR1 comprising an amino acid sequence of SEQ ID NO:10; and (2) a VL CDR2 comprising an amino acid sequence of SEQ ID NO:11.

In some embodiments, a 5T4-ADC comprises an antibody that binds to 5T4, wherein the antibody comprises: (a) a VH region comprising: (1) a VH CDR1 comprising an amino acid sequence of SEQ ID NO:18; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: (a) a VH region comprising: a VH CDR1 comprising an amino acid sequence of SEQ ID NO:18; and (b) a VL region comprising: (1) a VL CDR1 comprising an amino acid sequence of SEQ ID NO:21; and (2) a VL CDR2 comprising an amino acid sequence of SEQ ID NO:22.

In some embodiments, a 5T4-ADC comprises an antibody that binds to 5T4, wherein the antibody comprises: (a) a VH region comprising: (1) a VH CDR1 comprising an amino acid sequence of SEQ ID NO: 27; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: a VH region comprising: (1) a VH CDR1 comprising an amino acid sequence of SEQ ID NO:27; and a VL region comprising: (1) a VL CDR1 comprising an amino acid sequence of SEQ ID NO:4; and (2) a VL CDR2 comprising an amino acid sequence of SEQ ID NO:5.

In some embodiments, a 5T4-ADC comprises an antibody that binds to 5T4, wherein the antibody comprises: (a) a VH region comprising: (1) a VH CDR1 comprising an amino acid sequence of SEQ ID NO:31; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: (a) a VH region comprising: a VH CDR1 comprising an amino acid sequence of SEQ ID NO:31; and (b) a VL region comprising: (1) a VL CDR1 comprising an amino acid sequence of SEQ ID NO:10; and (2) a VL CDR2 comprising an amino acid sequence of SEQ ID NO:11.

In some embodiments, a 5T4-ADC comprises an antibody that binds to 5T4, wherein the antibody comprises: (a) a VH region comprising: (1) a VH CDR1 comprising an amino acid sequence of SEQ ID NO:34; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: (a) a VH region comprising: a VH CDR1 comprising an amino acid sequence of SEQ ID NO:34; and (b) a VL region comprising: (1) a VL CDR1 comprising an amino acid sequence of SEQ ID NO:4; and (2) a VL CDR2 comprising an amino acid sequence of SEQ ID NO:5.

In some embodiments, a 5T4-ADC comprises an antibody that binds to 5T4, wherein the antibody comprises: (a) a VH region comprising: (1) a VH CDR1 comprising an amino acid sequence of SEQ ID NO:35; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: (a) a VH region comprising: (1) a VH CDR1 comprising an amino acid sequence of SEQ ID NO:35; and (b) a VL region comprising: (1) a VL CDR1 comprising an amino acid sequence of SEQ ID NO:16; and (2) a VL CDR2 comprising an amino acid sequence of SEQ ID NO:11.

In some embodiments, a 5T4-ADC comprises an antibody that binds to 5T4, wherein the antibody comprises: (a) a VH region comprising: (1) a VH CDR1 comprising an amino acid sequence of SEQ ID NO:39; and (b) a VL region comprising: (1) a VL CDR1 comprising an amino acid sequence of any one of SEQ ID NOs:4, 10, 16, and 21; and (2) a VL CDR2 comprising an amino acid sequence of any of SEQ ID NOs:5, 11, and 22.

In some embodiments, a 5T4-ADC comprises an antibody that binds to 5T4, wherein the antibody comprises: (a) a VH region comprising: a VH CDR1 comprising an amino acid sequence of SEQ ID NO:39; and (b) a VL region comprising: (1) a VL CDR1 comprising an amino acid sequence of SEQ ID NO:21; and (2) a VL CDR2 comprising an amino acid sequence of SEQ ID NO:22.

In some embodiments, a 5T4-ADC comprises an antibody that binds to 5T4, wherein the antibody comprises: (a) a VH region comprising: (1) a VH CDR1 comprising an amino acid sequence of SEQ ID NO:46; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: (a) a VH region comprising: (1) a VH CDR1 comprising an amino acid sequence of SEQ ID NO:46; and (b) a VL region comprising: (1) a VL CDR1 comprising an amino acid sequence of SEQ ID NO:4; and (2) a VL CDR2 comprising an amino acid sequence of SEQ ID NO:5.

In some embodiments, a 5T4-ADC comprises an antibody that binds to 5T4, wherein the antibody comprises: (a) a VH region comprising: (1) a VH CDR1 comprising an amino acid sequence of SEQ ID NO:50; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: (a) a VH region comprising: a VH CDR1 comprising an amino acid sequence of SEQ ID NO:50; and (b) a VL region comprising: (1) a VL CDR1 comprising an amino acid sequence of SEQ ID NO:10; and (2) a VL CDR2 comprising an amino acid sequence of SEQ ID NO:11.

In some embodiments, a 5T4-ADC comprises an antibody that binds to 5T4, wherein the antibody comprises: (a) a VH region comprising: (1) a VH CDR1 comprising an amino acid sequence of SEQ ID NO:53; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: (a) a VH region comprising: a VH CDR1 comprising an amino acid sequence of SEQ ID NO:53; and (b) a VL region comprising: (1) a VL CDR1 comprising an amino acid sequence of SEQ ID NO:4; and (2) a VL CDR2 comprising an amino acid sequence of SEQ ID NO:5.

In some embodiments, a 5T4-ADC comprises an antibody that binds to 5T4, wherein the antibody comprises: (a) a VH region comprising: (1) a VH CDR1 comprising an amino acid sequence of SEQ ID NO:57; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises: (a) a VH region comprising: a VH CDR1 comprising an amino acid sequence of SEQ ID NO:57; and (b) a VL region comprising: (1) a VL CDR1 comprising an amino acid sequence of SEQ ID NO:21; and (2) a VL CDR2 comprising an amino acid sequence of SEQ ID NO:22.

In some embodiments, a 5T4-ADC comprises an antibody that binds to 5T4, wherein the antibody comprises: (a) a VH region comprising: (1) a VH CDR1 comprising an amino acid sequence of any one of SEQ ID NOs:1, 7, 12, 13, and 18; (2) a VH CDR2 comprising an amino acid sequence of any one of SEQ ID NOs:2, 8, 14, 19, and 24; and (3) a VH CDR3 comprising an amino acid sequence of any one of SEQ ID NOs:3, 9, 15, and 20; and (b) a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22; and (3) a VL CDR3 comprising an amino acid sequence of any one of SEQ ID NOs:6, 17, and 23.

In some embodiments, a 5T4-ADC comprises an antibody that binds to 5T4, wherein the antibody comprises: (a) a VH region comprising: (1) a VH CDR1 comprising an amino acid sequence of SEQ ID NO:1; (2) a VH CDR2 comprising an amino acid sequence of SEQ ID NO:2; and (3) a VH CDR3 comprising an amino acid sequence of SEQ ID NO:3; and (b) a VL region comprising: (1) a VL CDR1 comprising an amino acid sequence of SEQ ID NO:4; (2) a VL CDR2 comprising an amino acid sequence of SEQ ID NO:5; and (3) a VL CDR3 comprising an amino acid sequence of SEQ ID NO:6.

In some embodiments, a 5T4-ADC comprises an antibody that binds to 5T4, wherein the antibody comprises: (a) a VH region comprising: (1) a VH CDR1 comprising the amino acid sequence of SEQ ID NO:7; (2) a VH CDR2 comprising an amino acid sequence of SEQ ID NO:8; and (3) a VH CDR3 comprising an amino acid sequence of SEQ ID NO:9; and (b) a VL region comprising: (1) a VL CDR1 comprising an amino acid sequence of SEQ ID NO:10; (2) a VL CDR2 comprising an amino acid sequence of SEQ ID NO:11; and (3) a VL CDR3 comprising an amino acid sequence of SEQ ID NO:6.

In some embodiments, a 5T4-ADC comprises an antibody that binds to 5T4, wherein the antibody comprises: (a) a VH region comprising: (1) a VH CDR1 comprising an amino acid sequence of SEQ ID NO:12; (2) a VH CDR2 comprising an amino acid sequence of SEQ ID NO:2; and (3) a VH CDR3 comprising an amino acid sequence of SEQ ID NO:3; and (b) a VL region comprising: (1) a VL CDR1 comprising an amino acid sequence of SEQ ID NO:4; (2) a VL CDR2 comprising an amino acid sequence of SEQ ID NO:5; and (3) a VL CDR3 comprising an amino acid sequence of SEQ ID NO:6.

In some embodiments, a 5T4-ADC comprises an antibody that binds to 5T4, wherein the antibody comprises: (a) a VH region comprising: (1) a VH CDR1 comprising an amino acid sequence of SEQ ID NO:13; (2) a VH CDR2 comprising an amino acid sequence of SEQ ID NO:14; and (3) a VH CDR3 comprising an amino acid sequence of SEQ ID NO:15; and (b) a VL region comprising: (1) a VL CDR1 comprising an amino acid sequence of SEQ ID NO:16; (2) a VL CDR2 comprising an amino acid sequence of SEQ ID NO:11; and (3) a VL CDR3 comprising an amino acid sequence of SEQ ID NO:17.

In some embodiments, a 5T4-ADC comprises an antibody that binds to 5T4, wherein the antibody comprises: (a) a VH region comprising: (1) a VH CDR1 comprising an amino acid sequence of SEQ ID NO:18; (2) a VH CDR2 comprising an amino acid sequence of SEQ ID NO:19; and (3) a VH CDR3 comprising an amino acid sequence of SEQ ID NO:20; and (b) a VL region comprising: (1) a VL CDR1 comprising an amino acid sequence of SEQ ID NO:21; (2) a VL CDR2 comprising an amino acid sequence of SEQ ID NO:22; and (3) a VL CDR3 comprising an amino acid sequence of SEQ ID NO:23.

In some embodiments, a 5T4-ADC comprises an antibody that binds to 5T4, wherein the antibody comprises: (a) a VH region comprising: (1) a VH CDR1 comprising an amino acid sequence of SEQ ID NO:1; (2) a VH CDR2 comprising an amino acid sequence of SEQ ID NO:24; and (3) a VH CDR3 comprising an amino acid sequence of SEQ ID NO:3; and (b) a VL region comprising: (1) a VL CDR1 comprising an amino acid sequence of SEQ ID NO:4; (2) a VL CDR2 comprising an amino acid sequence of SEQ ID NO:5; and (3) a VL CDR3 comprising an amino acid sequence of SEQ ID NO:6.

In some embodiments, a 5T4-ADC comprises an antibody that binds to 5T4, wherein the antibody comprises: (a) a VH region comprising: (1) a VH CDR1 comprising an amino acid sequence of any one of SEQ ID NOs:27, 31, 34, 35, and 39; (2) a VH CDR2 comprising an amino acid sequence of any one of SEQ ID NOs:28, 32, 36, 40, and 43; and (3) a VH CDR3 comprising an amino acid sequence of any one of SEQ ID NOs:29, 33, 37, and 41; and (b) a VL region comprising: (1) a VL CDR1 comprising an amino acid sequence of any one of SEQ ID NOs:4, 10, 16, and 21; (2) a VL CDR2 comprising an amino acid sequence of any one of SEQ ID NOs:5, 11, and 22; and (3) a VL CDR3 comprising an amino acid sequence of any one of SEQ ID NOs:30, 38, and 42.

In some embodiments, a 5T4-ADC comprises an antibody that binds to 5T4, wherein the antibody comprises: (a) a VH region comprising: (1) a VH CDR1 comprising an amino acid sequence of SEQ ID NO:27; (2) a VH CDR2 comprising an amino acid sequence of SEQ ID NO:28; and (3) a VH CDR3 comprising an amino acid sequence of SEQ ID NO:29; and (b) a VL region comprising: (1) a VL CDR1 comprising an amino acid sequence of SEQ ID NO:4; (2) a VL CDR2 comprising an amino acid sequence of SEQ ID NO:5; and (3) a VL CDR3 comprising an amino acid sequence of SEQ ID NO:30.

In some embodiments, a 5T4-ADC comprises an antibody that binds to 5T4, wherein the antibody comprises: (a) a VH region comprising: (1) a VH CDR1 comprising an amino acid sequence of SEQ ID NO:31; (2) a VH CDR2 comprising an amino acid sequence of SEQ ID NO:32; and (3) a VH CDR3 comprising an amino acid sequence of SEQ ID NO:33; and (b) a VL region comprising: (1) a VL CDR1 comprising an amino acid sequence of SEQ ID NO:10; (2) a VL CDR2 comprising an amino acid sequence of SEQ ID NO:11; and (3) a VL CDR3 comprising an amino acid sequence of SEQ ID NO:30.

In some embodiments, a 5T4-ADC comprises an antibody that binds to 5T4, wherein the antibody comprises: (a) a VH region comprising: (1) a VH CDR1 comprising an amino acid sequence of SEQ ID NO:34; (2) a VH CDR2 comprising an amino acid sequence of SEQ ID NO:28; and (3) a VH CDR3 comprising an amino acid sequence of SEQ ID NO:29; and (b) a VL region comprising: (1) a VL CDR1 comprising an amino acid sequence of SEQ ID NO:4; (2) a VL CDR2 comprising an amino acid sequence of SEQ ID NO:5; and (3) a VL CDR3 comprising an amino acid sequence of SEQ ID NO:30.

In some embodiments, a 5T4-ADC comprises an antibody that binds to 5T4, wherein the antibody comprises: (a) a VH region comprising: (1) a VH CDR1 comprising an amino acid sequence of SEQ ID NO:35; (2) a VH CDR2 comprising an amino acid sequence of SEQ ID NO:36; and (3) a VH CDR3 comprising an amino acid sequence of SEQ ID NO:37; and (b) a VL region comprising: (1) a VL CDR1 comprising an amino acid sequence of SEQ ID NO:16; (2) a VL CDR2 comprising an amino acid sequence of SEQ ID NO:11; and (3) a VL CDR3 comprising an amino acid sequence of SEQ ID NO:38.

In some embodiments, a 5T4-ADC comprises an antibody that binds to 5T4, wherein the antibody comprises: (a) a VH region comprising: (1) a VH CDR1 comprising an amino acid sequence of SEQ ID NO:39; (2) a VH CDR2 comprising an amino acid sequence of SEQ ID NO:40; and (3) a VH CDR3 comprising an amino acid sequence of SEQ ID NO:41; and (b) a VL region comprising: (1) a VL CDR1 comprising an amino acid sequence of SEQ ID NO:21; (2) a VL CDR2 comprising an amino acid sequence of SEQ ID NO:22; and (3) a VL CDR3 comprising an amino acid sequence of SEQ ID NO:42.

In some embodiments, a 5T4-ADC comprises an antibody that binds to 5T4, wherein the antibody comprises: (a) a VH region comprising: (1) a VH CDR1 comprising an amino acid sequence of SEQ ID NO:27; (2) a VH CDR2 comprising an amino acid sequence of SEQ ID NO:43; and (3) a VH CDR3 comprising an amino acid sequence of SEQ ID NO:29; and (b) a VL region comprising: (1) a VL CDR1 comprising an amino acid sequence of SEQ ID NO:4; (2) a VL CDR2 comprising an amino acid sequence of SEQ ID NO:5; and (3) a VL CDR3 comprising an amino acid sequence of SEQ ID NO:30.

In some embodiments, a 5T4-ADC comprises an antibody that binds to 5T4, wherein the antibody comprises: (a) a VH region comprising: (1) a VH CDR1 comprising an amino acid sequence of any one of SEQ ID NOs:13, 46, 50, 53, and 57; (2) a VH CDR2 comprising an amino acid sequence of any one of SEQ ID NOs:47, 51, 54, 58, and 61; and (3) a VH CDR3 comprising an amino acid sequence of any one of SEQ ID NOs:48, 52, 55, and 59; and (b) a VL region comprising: (1) a VL CDR1 comprising an amino acid sequence of any one of SEQ ID NOs:4, 10, 16, and 21; (2) a VL CDR2 comprising an amino acid sequence of any one of SEQ ID NOs:5, 11, and 22; and (3) a VL CDR3 comprising an amino acid sequence of any one of SEQ ID NOs:49, 56, and 60.

In some embodiments, a 5T4-ADC comprises an antibody that binds to 5T4, wherein the antibody comprises: (a) a VH region comprising: (1) a VH CDR1 comprising an amino acid sequence of SEQ ID NO:46; (2) a VH CDR2 comprising an amino acid sequence of SEQ ID NO:47; and (3) a VH CDR3 comprising an amino acid sequence of SEQ ID NO:48; and (b) a VL region comprising: (1) a VL CDR1 comprising an amino acid sequence of SEQ ID NO:4; (2) a VL CDR2 comprising an amino acid sequence of SEQ ID NO:5; and (3) a VL CDR3 comprising an amino acid sequence of SEQ ID NO:49.

In some embodiments, a 5T4-ADC comprises an antibody that binds to 5T4, wherein the antibody comprises: (a) a VH region comprising: (1) a VH CDR1 comprising an amino acid sequence of SEQ ID NO:50; (2) a VH CDR2 comprising an amino acid sequence of SEQ ID NO:51; and (3) a VH CDR3 comprising an amino acid sequence of SEQ ID NO:52; and (b) a VL region comprising: (1) a VL CDR1 comprising an amino acid sequence of SEQ ID NO:10; (2) a VL CDR2 comprising an amino acid sequence of SEQ ID NO:11; and (3) a VL CDR3 comprising an amino acid sequence of SEQ ID NO:49.

In some embodiments, a 5T4-ADC comprises an antibody that binds to 5T4, wherein the antibody comprises: (a) a VH region comprising: (1) a VH CDR1 comprising an amino acid sequence of SEQ ID NO:53; (2) a VH CDR2 comprising an amino acid sequence of SEQ ID NO:47; and (3) a VH CDR3 comprising an amino acid sequence of SEQ ID NO:48; and (b) a VL region comprising: (1) a VL CDR1 comprising an amino acid sequence of SEQ ID NO:4; (2) a VL CDR2 comprising an amino acid sequence of SEQ ID NO:5; and (3) a VL CDR3 comprising an amino acid sequence of SEQ ID NO:49.

In some embodiments, a 5T4-ADC comprises an antibody that binds to 5T4, wherein the antibody comprises: (a) a VH region comprising: (1) a VH CDR1 comprising an amino acid sequence of SEQ ID NO:13; (2) a VH CDR2 comprising an amino acid sequence of SEQ ID NO:54; and (3) a VH CDR3 comprising an amino acid sequence of SEQ ID NO:55; and (b) a VL region comprising: (1) a VL CDR1 comprising an amino acid sequence of SEQ ID NO:16; (2) a VL CDR2 comprising an amino acid sequence of SEQ ID NO:11; and (3) a VL CDR3 comprising an amino acid sequence of SEQ ID NO:56.

In some embodiments, a 5T4-ADC comprises an antibody that binds to 5T4, wherein the antibody comprises: (a) a VH region comprising: (1) a VH CDR1 comprising an amino acid sequence of SEQ ID NO:57; (2) a VH CDR2 comprising an amino acid sequence of SEQ ID NO:58; and (3) a VH CDR3 comprising an amino acid sequence of SEQ ID NO:59; and (b) a VL region comprising: (1) a VL CDR1 comprising an amino acid sequence of SEQ ID NO:21; (2) a VL CDR2 comprising an amino acid sequence of SEQ ID NO:22; and (3) a VL CDR3 comprising an amino acid sequence of SEQ ID NO:60.

In some embodiments, a 5T4-ADC comprises an antibody that binds to 5T4, wherein the antibody comprises: (a) a VH region comprising: (1) a VH CDR1 comprising an amino acid sequence of SEQ ID NO:46; (2) a VH CDR2 comprising an amino acid sequence of SEQ ID NO:61; and (3) a VH CDR3 comprising an amino acid sequence of SEQ ID NO:48; and (b) a VL region comprising: (1) a VL CDR1 comprising an amino acid sequence of SEQ ID NO:4; (2) a VL CDR2 comprising an amino acid sequence of SEQ ID NO:5; and (3) a VL CDR3 comprising an amino acid sequence of SEQ ID NO:49.

In some embodiments, a 5T4-ADC comprises an antibody that binds to 5T4, wherein the antibody comprises: a VH region comprising: a VH CDR1 comprising an amino acid sequence selected from the group consisting of SEQ ID NOs:27, 31, 34, 35, and 39; and a VL region comprising: (1) a VL CDR1 comprising an amino acid sequence of any one of SEQ ID NOs:4, 10, 16, and 21; and (2) a VL CDR2 comprising an amino acid sequence of any one of SEQ ID NOs:5, 11, and 22.

In some embodiments, a 5T4-ADC comprises an antibody that binds to 5T4, wherein the antibody comprises a VH region and/or VL region described herein, wherein an VH and/or VL comprises human framework sequences. In some embodiments, an VH region and/or VL region comprises a framework 1 (FR1), a framework 2 (FR2), a framework 3 (FR3) and/or a framework 4 (FR4) sequence, such as a human FR1, a human FR2, a human FR3 and/or a human FR4.

In some embodiments, an CDRs of a 5T4 antibody can be determined by the Kabat system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242).

In some embodiments, CDRs of a 5T4 antibody can be determined by the Chothia system, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A. et al., 1990, J. Mol. Biol. 215(1):175-82; and U.S. Pat. No. 7,709,226).

In some embodiments, CDRs of a 5T4 antibody can be determined by the ImMunoGeneTics (IMGT®) system, for example, as described in Lefranc, M.-P., 1999, The Immunologist, 7:132-136 and Lefranc, M.-P. et al., 1999, Nucleic Acids Res., 27:209-212 (“IMGT® CDRs”).

In some embodiments, CDRs of a 5T4 antibody can be determined by the AbM system, which will be referred to herein as the “AbM CDRs,” for example as described in MacCallum et al., 1996, J. Mol. Biol., 262:732-745. See also, e.g., Martin, A., “Protein Sequence and Structure Analysis of Antibody Variable Domains,” in Antibody Engineering, Kontermann and Dubel, eds., Chapter 31, pp. 422-439, Springer-Verlag, Berlin (2001).

In some embodiments, CDRs of a 5T4 antibody can be determined by the Contact system, which will be referred to herein as the “Contact CDRs” (see, e.g., MacCallum R M et al., 1996, J Mol Biol 5: 732-745). The Contact CDRs are based on an analysis of the available complex crystal structures.

In some embodiments, the position of one or more (e.g., one, two, three) CDRs along the VH (e.g., CDR1, CDR2, or CDR3) and/or VL (e.g., CDR1, CDR2, or CDR3) region of a 5T4 antibody can vary by one, two, three, four, five, or six amino acid positions provided that binding to 5T4 (e.g., human 5T4) is maintained (e.g., substantially maintained, for example, at least 50%, at least 60%, at least 70%, at least 80%, at least 90%, or at least 95%). For example, in some embodiments, the position defining a CDR of any of Table 1, 2, or 3 can vary by shifting the N-terminal and/or C-terminal boundary of the CDR by one, two, three, four, five, or six amino acids, relative to the current CDR position, provided that binding to 5T4 (e.g., human 5T4) is maintained (e.g., substantially maintained, for example, at least 50%, at least 60%, at least 70%, at least 80%, at least 90%, or at least 95%). Additionally or alternatively, in some embodiments, the length of one or more (e.g., one, two, three) CDRs along the VH (e.g., CDR1, CDR2, or CDR3) and/or VL (e.g., CDR1, CDR2, or CDR3) region of a 5T4 antibody (e.g., a human 5T4 antibody) described herein can vary (e.g., be shorter or longer) by one, two, three, four, five, or more amino acids, provided that binding to 5T4 (e.g., human 5T4) is maintained (e.g., substantially maintained, for example, at least 50%, at least 60%, at least 70%, at least 80%, at least 90%, or at least 95%). For example, in some embodiments, a VH and/or VL CDR1, CDR2, and/or CDR3 can be one, two, three, four, five or more amino acids shorter than one or more of the CDRs described by SEQ ID NOs:1-24, 27-43, or 46-61, provided that binding to 5T4 (e.g., human 5T4) is maintained (e.g., substantially maintained, for example, at least 50%, at least 60%, at least 70%, at least 80%, at least 90%, or at least 95%). In other embodiments, a VH and/or VL CDR1, CDR2, and/or CDR3 can be one, two, three, four, five or more amino acids longer than one or more of the CDRs described by SEQ ID NOs:1-24, 27-43, or 46-61, provided that binding to 5T4 (e.g., human 5T4) is maintained (e.g., substantially maintained, for example, at least 50%, at least 60%, at least 70%, at least 80%, at least 90%, or at least 95%). Additionally or alternatively, in some embodiments, the amino terminus of a VH and/or VL CDR1, CDR2, and/or CDR3 can be extended by one, two, three, four, five or more amino acids compared to one or more of the CDRs described by SEQ ID NOs:1-24, 27-43, or 46-61, provided that binding to 5T4 (e.g., human 5T4) is maintained (e.g., substantially maintained, for example, at least 50%, at least 60%, at least 70%, at least 80%, at least 90%, or at least 95%). Additionally or alternatively, in some embodiments, the carboxy terminus of a VH and/or VL CDR1, CDR2, and/or CDR3 can be extended by one, two, three, four, five or more amino acids compared to one or more of the CDRs described by SEQ ID NOs:1-24, 27-43, or 46-61, provided that binding to 5T4 (e.g., human 5T4) is maintained (e.g., substantially maintained, for example, at least 50%, at least 60%, at least 70%, at least 80%, at least 90%, or at least 95%). Additionally or alternatively, in some embodiments, the amino terminus of a VH and/or VL CDR1, CDR2, and/or CDR3 can be shortened by one, two, three, four, five or more amino acids compared to one or more of the CDRs described by SEQ ID NOs:1-24, 27-43, or 46-61, provided that binding to 5T4 (e.g., human 5T4) is maintained (e.g., substantially maintained, for example, at least 50%, at least 60%, at least 70%, at least 80%, at least 90%, or at least 95%). In some embodiments, the carboxy terminus of a VH and/or VL CDR1, CDR2, and/or CDR3 can be shortened by one, two, three, four, five or more amino acids compared to one or more of the CDRs described by SEQ ID NOs:1-24, 27-43, or 46-61, provided that binding to 5T4 (e.g., human 5T4) is maintained (e.g., substantially maintained, for example, at least 50%, at least 60%, at least 70%, at least 80%, at least 90%, or at least 95%). Any method known in the art can be used to ascertain whether binding to 5T4 (e.g., human 5T4) is maintained, for example, the binding assays and conditions described in the “Examples” section described herein. For example, Example 2 described herein describes an assay for measuring binding to 5T4 (e.g., human 5T4).

In some embodiments, a 5T4-ADC can comprise a 5T4 antibody that comprises one or more (e.g., one, two, three, four, or more) conservative sequence modifications. With respect to polypeptides that are 5T4 antibodies, such as human 5T4 antibodies, conservative sequence modifications include conservative amino acid substitutions in which an amino acid residue is replaced with an amino acid residue having a similar side chain. Families of amino acid residues having similar side chains have been defined in the art. These families are disclosed herein. Thus, in some embodiments, a predicted nonessential amino acid residue in a 5T4 antibody can be replaced with another amino acid residue from the same side chain family. Methods of identifying amino acid conservative substitutions which do not eliminate antigen binding and nucleotides encoding thereof are well known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). In some embodiments, the conservative sequence modifications described herein modify the amino acid sequences of a 5T4 antibody (e.g., a human 5T4 antibody) by 50%, 55%, 60%, 65%, 70%, 75%, 80%, 85%, 90%, 95%, 98%, or 99%. In some embodiments, the amino acid sequence modifications refer to at most one, two, three, four, five, or six amino acid substitutions to the CDRs, such as those described in any one of Tables 1-3. Thus, for example, each such CDR can contain up to five conservative amino acid substitutions, for example up to (not more than) four conservative amino acid substitutions, for example up to (not more than) three conservative amino acid substitutions, for example up to (not more than) two conservative amino acid substitutions, or no more than one conservative amino acid substitution. In some embodiments, a 5T4-ADC comprises a 5T4 antibody that contains one or more, (e.g., one, two, three, four, five, or six CDRs) having at least 75%, 80%, 85%, 90%, 95%, 96%, 97%, 98%, or 99% identity to the CDRs of mAbA4, mAbA15, or mAbA17 (see, e.g., Tables 1, 2, or 3). In some embodiments, a 5T4-ADC comprises a 5T4 antibody that contains a VH and a VL comprising CDRs identical to those of mAbA4, mAbA15, or mAbA17 (see, e.g., Tables 1, 2, or 3). In some embodiments, the amino acid sequence modifications do not include any modification within a specificity determining residue (SDR). In some embodiments, the amino acid sequence modifications do not include any modification within a CDR (such as CDR1, CDR2, CDR3, or any combination thereof). In further embodiments, the amino acid sequence modifications are in the framework or constant region.

In some embodiments, the antibody in a 5T4-ADC comprises a VH comprising an amino acid sequence having at least 75%, 80%, 85%, 90%, 95%, 96%, 97%, 98%, or 99% identity to SEQ ID NO:25 and a VL comprising an amino acid sequence having at least 75%, 80%, 85%, 90%, 95%, 96%, 97%, 98%, or 99% identity to SEQ ID NO:26, and the binding of the antibody to 5T4 (e.g., human 5T4) is maintained (e.g., substantially maintained, for example, at least 50%, at least 60%, at least 70%, at least 80%, at least 90%, or at least 95%).

In some embodiments, the antibody in a 5T4-ADC comprises a VH comprising an amino acid sequence having at least 75%, 80%, 85%, 90%, 95%, 96%, 97%, 98%, or 99% identity to SEQ ID NO:44 and a VL comprising an amino acid sequence having at least 75%, 80%, 85%, 90%, 95%, 96%, 97%, 98%, or 99% identity to SEQ ID NO:45, and the binding of the antibody to 5T4 (e.g., human 5T4) is maintained (e.g., substantially maintained, for example, at least 50%, at least 60%, at least 70%, at least 80%, at least 90%, or at least 95%).

In some embodiments, the antibody in a 5T4-ADC comprises a VH comprising an amino acid sequence having at least 75%, 80%, 85%, 90%, 95%, 96%, 97%, 98%, or 99% identity to SEQ ID NO:62 and a VL comprising an amino acid sequence having at least 75%, 80%, 85%, 90%, 95%, 96%, 97%, 98%, or 99% identity to SEQ ID NO:63, and the binding of the antibody to 5T4 (e.g., human 5T4) is maintained (e.g., substantially maintained, for example, at least 50%, at least 60%, at least 70%, at least 80%, at least 90%, or at least 95%).

Additionally provided is a nucleic acid encoding a 5T4 binding agent (e.g., antibody or antibody fragment) or a fusion polypeptide as disclosed herein, a nucleic acid complementary thereto, a vector comprising a nucleic acid as disclosed herein, a cell comprising a nucleic acid or a vector as disclosed herein. In some embodiments, the cell expresses the 5T4 binding agent. In some embodiments, the cell replicates the nucleic acid or the vector. In some embodiments, further provided are materials for generating 5T4 antibodies, including human 5T4 antibodies, useful for the preparation of ADCs. In this regard, a cell (e.g., an isolated cell) can produce an antibody comprising a VH and a VL as described herein. In some embodiments, a polynucleotide can comprise one or more nucleic acid sequences encoding a 5T4 antibody or antibody fragment. In some embodiments, the polynucleotide is an isolated and/or recombinant polynucleotide. In some embodiments, the isolated polynucleotide comprises a nucleotide sequence that encodes an antibody heavy chain variable region (VH) and/or an antibody light chain variable region (VL), wherein the VH and the VL comprise CDRs identical to CDRs as described herein.

In some embodiments, one or more vectors (e.g., expression vectors) can comprise one or more polynucleotides for expression of the one or more polynucleotides in a suitable host cell. Such vectors are useful, for example, for amplifying the polynucleotides in host cells to create useful quantities thereof, and for expressing binding agents, such as antibodies or antibody fragments, using recombinant techniques.

In some embodiments, one or more vectors are expression vectors wherein one or more polynucleotides are operatively linked to one or more polynucleotides comprising expression control sequences. Autonomously replicating recombinant expression constructs such as plasmid and viral DNA vectors incorporating one or more polynucleotides encoding antibody sequences that bind 5T4 are specifically contemplated. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs can also include sequences encoding one or more selectable markers that permit identification of host cells bearing the construct. Expression constructs can also include sequences that facilitate, e.g., promote, homologous recombination in a host cell. In some embodiments, expression constructs also include sequences necessary for replication in a host cell.

In some embodiments, an expression control sequence may include a promoter/enhancer sequence, e.g., cytomegalovirus promoter/enhancer (Lehner et al., J. Clin. Microbiol., 29: 2494-2502, 1991; Boshart et al., Cell, 41: 521-530, 1985); Rous sarcoma virus promoter (Davis et al., Hum. Gene Ther., 4: 151, 1993); Tie promoter (Korhonen et al., Blood, 86(5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointest. Liver Physiol., G977-G979. 2005); and Math1 (mouse atonal homolog 1; Shroyer et al., Gastroenterology, 132: 2477-2478, 2007), for expression in mammalian cells, the promoter being operatively linked upstream (e.g., 5′) of a polypeptide coding sequence. In some embodiments, a promoter can be an epithelial-specific promoter or endothelial-specific promoter. A polynucleotide can also optionally include a suitable polyadenylation sequence (e.g., the SV40 or human growth hormone gene polyadenylation sequence) operably linked downstream (e.g., 3′) of the polypeptide coding sequence.

In any embodiment, one or more polynucleotides can optionally additionally comprise one or more nucleotide sequences encoding one or more secretory signal peptides fused in frame with the polypeptide sequences. The one or more secretory signal peptides can direct secretion of the antibody polypeptides by the cells that express the one or more polynucleotides and can be cleaved by the cell from the secreted polypeptide. In any embodiment, one or more polynucleotides can further optionally comprise one or more sequences whose only intended function is to facilitate large scale production of the vector.

Expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, viral vectors are rendered replication-deficient by, e.g., deleting or disrupting select genes required for viral replication.

A cell can comprise one or more polynucleotides and/or one or more vectors. For example, in any embodiment a cell can be transformed or transfected with one or more polynucleotides encoding a 5T4 antibody (e.g., a human 5T4 antibody) or one or more vectors comprising the one or more polynucleotides encoding a 5T4 antibody (e.g., a human 5T4 antibody). In some embodiments, a cell can express a 5T4 antibody (e.g., a human 5T4 antibody) containing one or more, (e.g., one, two, three, four, five, or six) CDRs having at least 75%, 80%, 85%, 90%, 95%, 96%, 97%, 98%, or 99% identity to CDRs as described herein, such as mAbA4, mAbA15, and/or mAbA17 (see, e.g., Tables 1, 2, and/or 3). In some embodiments, a cell can express a 5T4 antibody (e.g., a human 5T4 antibody) containing VH regions and VL regions comprising CDRs identical to those as described herein, such as of mAbA4, mAbA15, and/or mAbA17 (see, e.g., Tables 1, 2, and/or 3). Suitable cells for generating a 5T4 antibody as described herein include prokaryotic cells, such as Escherichia coli (see, e.g., Pluckthun et al., Methods Enzymol., 178: 497-515, 1989), and eukaryotic cells, such as an animal cell (e.g., a myeloma cell, Chinese Hamster Ovary (CHO) cell, or hybridoma cell), yeast (e.g., Saccharomyces cerevisiae), or a plant cell (e.g., a tobacco, corn, soybean, or rice cell). Use of mammalian host cells can provide for translational modifications (e.g., glycosylation, truncation, lipidation, and phosphorylation) that can be desirable to confer optimal biological activity on recombinant expression products. Similarly, in any embodiment, a polypeptide (e.g., 5T4 antibodies, including human 5T4 antibodies) can be glycosylated or non-glycosylated and/or have been covalently modified to include one or more water-soluble polymer attachments such as polyethylene glycol, polyoxyethylene glycol, or polypropylene glycol.

Methods for introducing DNA or RNA into a host cell are well known and include, but are not limited to, transformation, transfection, electroporation, nuclear injection, and fusion with carriers such as liposomes, micelles, ghost cells, and protoplasts. Such host cells are useful for amplifying polynucleotides and for expressing polypeptides encoded by the polynucleotides. In this regard, a process to produce a 5T4 antibody can comprise introducing RNA or DNA that encodes for a 5T4 antibody, as described herein, into a host cell, culturing the host cell, and isolating the 5T4 antibody thus produced.

A variety of methods for producing an antibody from a polynucleotide is generally well known. For example, basic molecular biology procedures are described by Maniatis et al., Molecular Cloning, A Laboratory Manual, 2nd ed., Cold Spring Harbor Laboratory, New York, 1989 (see also Maniatis et al, 3rd ed., Cold Spring Harbor Laboratory, New York, 2001). Additionally, numerous publications describe techniques suitable for the preparation of antibodies by manipulation of DNA, creation of expression vectors, and transformation and culture of appropriate cells (see, e.g., Mountain and Adair, Chapter 1 in Biotechnology and Genetic Engineering Reviews, Tombs ed., Intercept, Andover, U K, 1992); and Current Protocols in Molecular Biology, Ausubel ed., Wiley Interscience, New York, 1999).

### 5T4-ADCS

An antibody that binds to 5T4 and a drug can be linked directly or indirectly to each other via a pyridazine-pyrrolo coupling moiety to form a 5T4-ADC as described herein. In some embodiments, a 5T4-ADC can be represented by Formula (IV):

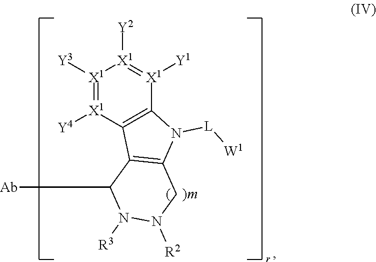

wherein:
Ab is an antibody that binds to 5T4;
r is an integer from 1 to 10;
m is 0 or 1;
R² and R³ are each independently hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or R² and R³ are cyclically linked to form a 5- or 6-membered heterocyclyl;
X¹, X², X³, and X⁴ are each independently C, N, O, or S;
Y¹, Y², Y³, and Y⁴ are each independently hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or Y¹ and Y², Y² and Y³, or Y³ and Y⁴ are cyclically linked;
L is a linker as described herein; and
W¹ is a drug.

Formula (IV) has been previously described in WO 2015/081282, which is herein incorporated by reference in its entirety.

In some embodiments, m is 0 or 1 and R² and R³ are each alkyl. For example, R² and R³ can independently each be C₁-C₁₀ alkyl (e.g., C₁-C₆ alkyl). In some embodiments, R² and R³ are each independently methyl, ethyl, n-propyl, iso-propyl, n-butyl, sec-butyl, isobutyl, or t-butyl. In some embodiments, at least one of R² and R³ is methyl. In some embodiments, both of R² and R³ are methyl. In some embodiments, each of X¹, X², X³, and X⁴ is independently C or N. In some embodiments, X¹ is C. In some embodiments, X¹ is N. In some embodiments, X² is C or N. In some embodiments, X² is C. In some embodiments, X³ is C or N. In some embodiments, X³ is C. In some embodiments, X⁴ is C or N. In some embodiments, X⁴ is C. Various combinations of X¹, X², X³, and X⁴ are possible. For example, in some embodiments, each of X¹, X², X³, and X⁴ is C. In other embodiments, three of X¹, X², X³, and X⁴ are C and one of X¹, X², X³, and X⁴ is N. In some embodiments, each of Y¹, Y², Y³, and Y⁴ is hydrogen.

In the context of a 5T4-ADC of Formula (IV), the linker-drug of Formula (IV) can be represented by Formula (XIV), below:

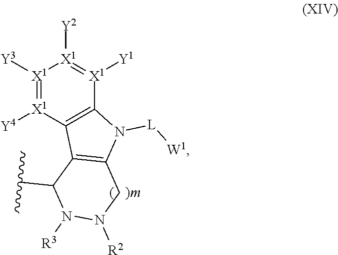

wherein [symbol] represents the point of attachment to the antibody that binds to 5T4, for example, residue n of SEQ ID NO:93, 97, or 101. All other variables of the linker-drug of Formula (XIV) can be as defined herein, such as for Formula (IV) and/or as described herein.

In some embodiments, a 5T4-ADC can be represented by Formula (V):

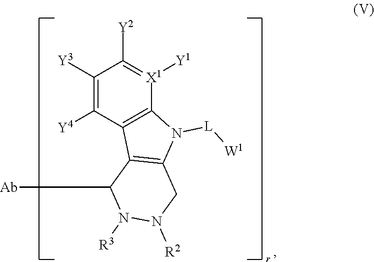

wherein:
Ab is an antibody that binds to 5T4;
r is an integer from 1 to 10;
R² and R³ are each independently hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or R² and R³ are cyclically linked to form a 5- or 6-membered heterocyclyl;
X¹ is C or N;
Y¹, Y², Y³, and Y⁴ are each independently hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or Y¹ and Y², Y² and Y³, or Y³ and Y⁴ are cyclically linked;
L is a linker as described herein; and
W¹ is a drug.

Formula (V) has been previously described in WO 2015/081282, which is herein incorporated by reference in its entirety.

In some embodiments, R² and R³ are each alkyl. For example, R² and R³ can independently each be C₁-C₁₀ alkyl (e.g., C₁-C₆ alkyl). In some embodiments, R² and R³ are each independently methyl, ethyl, n-propyl, iso-propyl, n-butyl, sec-butyl, isobutyl, or t-butyl. In some embodiments, at least one of R² and R³ is methyl. In some embodiments, both of R² and R³ are methyl. In some embodiments, each of Y¹, Y², Y³, and Y⁴ is hydrogen.

In the context of a 5T4-ADC of Formula (V), the linker-drug of Formula (V) can be represented by Formula (XV), below:

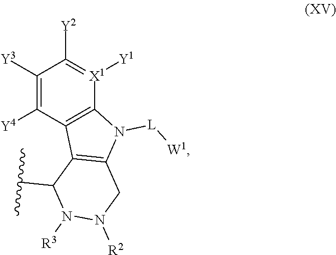

wherein [symbol] represents the point of attachment to the antibody that binds to 5T4, for example, residue n of SEQ ID NO:93, 97, or 101. All other variables of the linker-drug of Formula (XV) can be as defined herein, such as for Formula (V) and/or as described herein.

In some embodiments, a 5T4-ADC can be represented by Formula (VI):

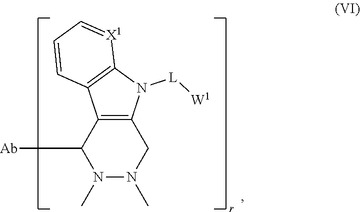

wherein:
Ab is an antibody that binds to 5T4;
r is an integer from 1 to 10;
X¹ is C or N;
L is a linker as described herein; and
W¹ is a drug.

Formula (IV) has been previously described in WO 2015/081282, which is herein incorporated by reference in its entirety.

In the context of a 5T4-ADC of Formula (VI), the linker-drug of Formula (VI) can be represented by Formula (XVI), below:

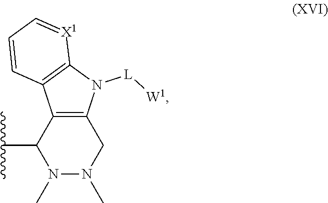

wherein [symbol] represents the point of attachment to an antibody that binds to 5T4, for example, residue n of SEQ ID NO:93, 97, or 101. All other variables of the linker-drug of Formula (XVI) can be as defined herein, such as for Formula (VI) and/or as described herein.

### Linkers

In some embodiments, a 5T4-ADC can comprise a linker (L) moiety that links a pyridazine-pyrrolo coupling moiety, as described herein, to a drug. In any embodiment, linker L can be represented by the following structure:

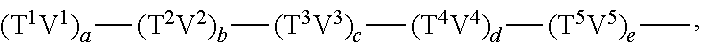

wherein a, b, c, d, and e are each independently 0 or 1 and wherein the sum of a, b, c, d, and e is 1 to 5. In some embodiments, the sum of a, b, c, d, and e is 3. In some embodiments, the sum of a, b, c, d, and e is 5.

In a linker L, T¹, T², T³, T⁴, and T⁵ are each independently C₁-C₁₂ alkyl, substituted C₁-C₁₂ alkyl, (EDA)_{w}, (PEG)ₙ, (AA)ₚ, —(CR¹³OH)ₙ—, a piperidin-4-amine (P4A), a meta-aminobenzyl carbamate (MABC) group, a meta-aminobenzyloxy (MABO) group, a para-aminobenzyloxy (PABO) group, a para-aminobenzyl carbamate (PABC) group, a para-aminobenzyl (PAB) group, acetal, a disulfide, a hydrazine, a protease-cleavable moiety, a glucuronidase cleavable moiety, a beta-lactamase cleavable moiety, an ester, (AA)ₚ-MABC-(AA)ₚ, (AA)ₚ-MABO-(AA)ₚ, (AA)ₚ-PABO-(AA)ₚ, or (AA)ₚ-PABC-(AA)ₚ, wherein w is an integer from 1 to 20, n is an integer from 1 to 30, each p is independently zero or an integer from 1 to 20, h is an integer from 1 to 12, and each R¹³ is independently hydrogen, alkyl, substituted alkyl, aryl, or substituted aryl.

As used herein, “(EDA)_{w}” is an ethylene diamine moiety that has the following structure:

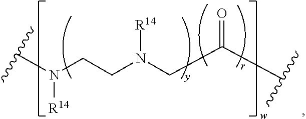

wherein y is an integer from 1 to 6, r is 0 or 1, w is an integer from 1 to 20, and each R¹⁴ is independently hydrogen, alkyl, substituted alkyl, PEG, aryl, or substituted aryl and wherein any two adjacent R¹⁴ groups can be cyclically linked to form a piperazinyl ring, wherein each [symbol] represents a point of attachment in any direction to an adjacent chemical moiety.

A piperidin-4-amine moiety can optionally be substituted at one or more positions with any substituent, e.g., alkyl, substituted alkyl, a polyethylene glycol moiety, acyl, substituted acyl, aryl, or substituted aryl. As such, as used herein, piperidin-4-amine, as used herein, can be described by the term “P4A-R¹²” which corresponds to the following structure:

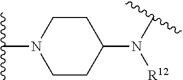

wherein R¹² is independently hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl, wherein each [symbol] represents a point of attachment in any direction to an adjacent chemical moiety

As used herein, (PEG)ₙ is polyethylene glycol having the following structure:

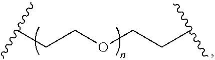

wherein n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19, or 20, wherein each [symbol] represents a point of attachment in any direction to an adjacent chemical moiety.

As used herein “AA” refers to an amino acid as described herein, including, but not limited to, any naturally occurring amino acids, naturally occurring amino acid analogs, enantiomers of naturally occurring amino acids, and non-naturally occurring amino acids. In (AA)ₚ, each amino acid residue can independently be chosen from those described herein and p can be an integer from 1 to 50, such as from 1 to 40, from 1 to 30, from 1 to 20, from 1 to 12, or from 1 to 6, such as 1, 2, 3, 4, 5, 6, 7, 8, 9, 10, 11, 12, 13, 14, 15, 16, 17, 18, 19, or 20.

As used herein, an MABC group can be described by the term “MABC-R¹⁶” and by following structure:

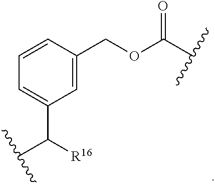

As used herein, a MABO group can be described by the term “MABO-R¹⁶” and by the following structure:

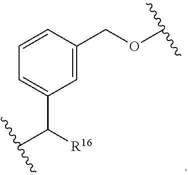

As used herein, a PABC group can be described by the term “PABC-R¹⁶” and by the following structure:

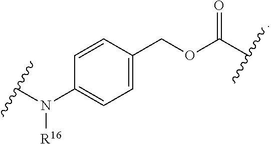

As used herein, a PABO group can be described by the term “PABO-R¹⁶” and by the following structure:

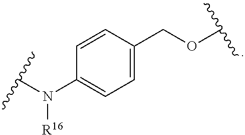

As used herein, a para-aminobenzyl (PAB) group can be described by the term “PAB-R¹⁶” and by the following structure:

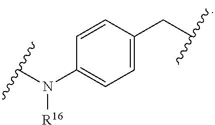

In MABC-R¹⁶, MABO-R¹⁶, PABC-R¹⁶, PABO-R¹⁶, and PAB-R¹⁶, each [symbol] represents a point of attachment in any direction to an adjacent chemical moiety, and R¹⁶ is independently hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl.

In some embodiments, one or more of T¹, T², T³, T⁴, and T⁵ is C₁-C₆ alkyl.

In some embodiments, T¹ independently is C₁-C₆ alkyl. In some embodiments, T¹ is ethyl. In some embodiments, each of T¹ and T³ is C₁-C₆ alkyl. In some embodiments, each of T¹ and T³ is ethyl.

In some embodiments, one or more of T¹, T², T³, T⁴, and T⁵ is P4A-R¹². In some embodiments, each R¹² is independently a polyethylene glycol moiety. In some embodiments, each R¹² is independently a carboxylic acid-modified polyethylene glycol. In some embodiments, T² is P4A-R¹² wherein R¹² is a carboxylic acid-modified polyethylene glycol.

In some embodiments, one or more of T¹, T², T³, T⁴, and T⁵ is (PEG)ₙ. In some embodiments, n is 2. In some embodiments, T³ is (PEG)ₙ wherein n is 2.

In some embodiments, one or more of T¹, T², T³, T⁴, and T⁵ is (AA)ₚ. In some embodiments, p is 1. In some embodiments, T² is (AA)ₚ wherein p is 1. In some embodiments, AA of (AA)ₚ is an amino acid with a polar uncharged side chain (e.g., serine, threonine, asparagine, or glutamine). In some embodiments, AA of (AA)ₚ is glutamine.

In some embodiments, one or more of T¹, T², T³, T⁴, and T⁵ is (AA)ₚ-(PABC-R¹⁶)-(AA)ₚ. In some embodiments, R¹⁶ is hydrogen. In some embodiments, p is 0. In some embodiments, R¹⁶ is hydrogen, and p is 0. In some embodiments, therefore, one or more of T¹, T², T³, T⁴, and T⁵ is an AA-PABC described by the following structure:

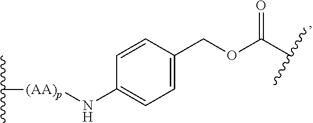

wherein each [symbol] represents a point of attachment in any direction to an adjacent chemical moiety in the linker.

In some embodiments, p of AA-PABC is 2. In some embodiments, T⁴ is AA-PABC, wherein p is 2. In some embodiments, the two amino acids of (AA)₂ are valine and citrulline.

V¹, V², V³, V⁴, and V⁵ of linker L can each independently be a covalent bond, amino, carbonyl, amido, oxycarbonyl, carboxy, sulfonyl, sulfoxide, sulfonylamino, aminosulfonyl, thio, oxy, phospho, phosphoramidate, or thiophosphoraidate. In some embodiments, V¹, V², V³, V⁴, and V⁵ are each independently a covalent bond, —C(═O)—, —NR¹¹—, —C(═O)NR¹¹—, —NR¹¹C(═O)—, —C(═O)O—, —OC(═O)—, —O—, —S—, —S(═O)—, —SO₂—, —SO₂NR¹¹—, —NR¹¹SO₂— and —P(═O)OH—, wherein R¹¹ is hydrogen, alkyl, substituted alkyl, a PEG, aryl, or substituted aryl. In some embodiments, V¹, V², and V³ are independently C(═O) or —NR¹¹—, wherein R¹¹ is independently hydrogen or C₁-C₆ alkyl.

In some embodiments, linker L can be represented by the following structure:

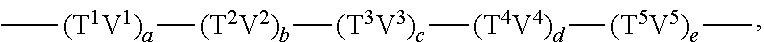

wherein:
a, b, c, d, and e are each independently 0 or 1, wherein the sum of a, b, c, d, and e is 1 to 5;
T¹ is C₁-C₆ alkyl;
T², T³, T⁴, and T⁵ are each independently (PEG)ₙ, C₁-C₆ alkyl, (AA)ₚ, P4A-R¹², (AA)ₚ-(PABO-R¹⁶)-(AA)ₚ, (AA)ₚ-(PABC-R¹⁶)-(AA)ₚ, (AA)ₚ-(PABO-R¹⁶), or (AA)ₚ-(PABC-R¹⁶);
V¹, V², V³, V⁴, and V⁵ are each independently a covalent bond, —CO—, or —NR¹¹—;
each R¹¹ is independently hydrogen, alkyl, substituted alkyl, a polyethylene glycol, aryl, or substituted aryl;
each R¹² is independently hydrogen, alkyl, substituted alkyl, or a modified or unmodified polyethylene glycol moiety;
R¹⁶ is hydrogen;
n is an integer from 1 to 30; and
p is an integer from 1 to 20.

In some embodiments, R¹² is (PEG)ₙ. In some embodiments, R¹² is (PEG)ₙ terminally modified with a carboxylic acid. In some embodiments, R¹¹ is hydrogen or C₁-C₁₂ alkyl. In some embodiments, p is 1. In some embodiments, p is 2. In some embodiments, p is 1 at one position in the linker and p is 2 at another position in the linker. In some embodiments, n is an integer from 1 to 5. In some embodiments, n is 2.

### Non-Cleavable Linkers

In some embodiments, linker L can be represented by the following structure:

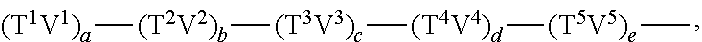

wherein:
a, b, c, d, and e are each independently 0 or 1, wherein the sum of a, b, c, d, and e is 1 to 5;
T¹ is C₁-C₆ alkyl;
T² and T³ are each independently C₁-C₆ alkyl or P4A-R¹²;
T⁴ and T⁵ are each independently (PEG)ₙ, C₁-C₆ alkyl, (AA)ₚ, P4A-R¹², (AA)ₚ-(PABO-R¹⁶)-(AA)ₚ, (AA)ₚ-(PABC-R¹⁶)-(AA)ₚ, (AA)ₚ-(PABO-R¹⁶), or (AA)ₚ-(PABC-R¹⁶);
V¹, V², V³, V⁴, and V⁵ are each independently a covalent bond, —CO—, or —NR¹¹—; and
each R¹¹ is independently hydrogen or C₁-C₆ alkyl; and
each R¹² is independently a modified or unmodified polyethylene glycol moiety

In some embodiments, each of T¹ and T³ is independently C₁-C₆ alkyl, such as independently methyl, ethyl, or propyl. In some embodiments, each of T¹ and T³ is ethyl. In some embodiments, T² is P4A-R¹², wherein R¹² is a modified or unmodified polyethylene glycol moiety. In some embodiments, d and e are 0.

In some embodiments, linker L can be represented by the following structure:

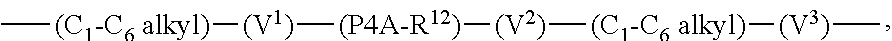

wherein:
V¹, V², and V³ are each independently —CO— or —NR¹¹—;
each R¹¹ is independently hydrogen or C₁-C₆ alkyl; and
each R¹² is independently a carboxylic acid-modified polyethylene glycol moiety.

In some embodiments, linker L is linker (L-1):

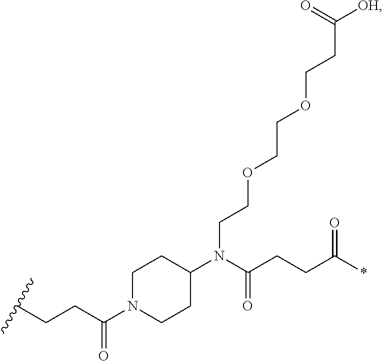

wherein [symbol] represents the point of attachment to the nitrogen of a pyridazine-pyrrolo coupling moiety, as described herein and * represents the point of attachment to W¹.

Accordingly, in some embodiments, a 5T4-ADC can be represented by Formula (VI-1):

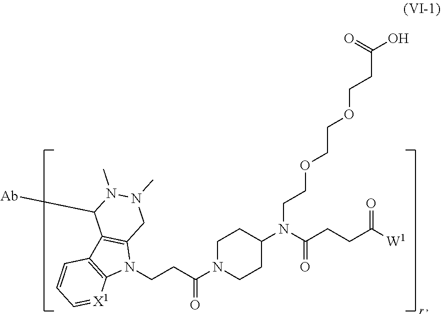

wherein:
Ab is an antibody that binds to 5T4;
r is an integer from 1 to 10; and
W¹ is a drug.

In some embodiments, r is an integer from 1 to 4. In some embodiments, r is 1 or 2.

In some embodiments, a 5T4-ADC can be represented by Formula (VI-1), wherein X¹ is C. Accordingly, in some embodiments, a 5T4-ADC can be represented by Formula (VIa-1):

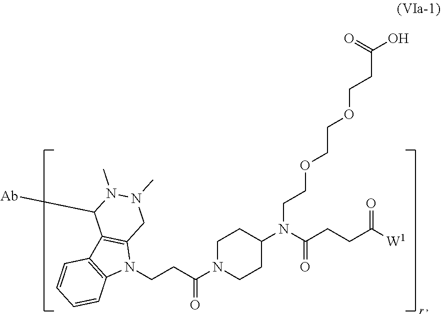

wherein:
Ab is an antibody that binds to 5T4;
r is an integer from 1 to 10; and
W¹ is a drug.
   In some embodiments, r is an integer from 1 to 4. In some embodiments, r is 1 or 2.

In some embodiments, a 5T4-ADC can be represented by Formula (VI-1), wherein X¹ is N. Accordingly, in some embodiments, a 5T4-ADC can be represented by Formula (VIb-1):

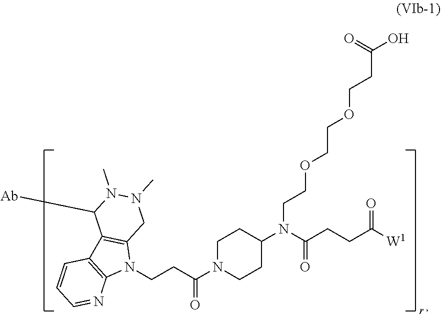

wherein:
Ab is an antibody that binds to 5T4;
r is an integer from 1 to 10; and
W¹ is a drug.

In some embodiments, r is an integer from 1 to 4. In some embodiments, r is 1 or 2.

### Cleavable Linkers

In some embodiments, the antibody and drug are conjugated to each other through a cleavable linker. A cleavable linker is a linker that includes one or more cleavable moieties, wherein the cleavable moiety includes one or more bonds that can dissociate under some conditions, thus separating the cleavable linker into two or more separable portions. For example, the cleavable moiety can include one or more covalent bonds, which under some conditions, can dissociate or break apart to separate the cleavable linker into two or more portions. As such, a cleavable linker can be included in a 5T4-ADC, such that under certain conditions, the cleavable linker is cleaved to separate or release the drug from the antibody at a desired target site of action for the drug.

In some embodiments, the cleavable linker can be an enzymatically cleavable linker. An enzymatically cleavable linker can be separated into two or more portions as described above through the enzymatic action of an enzyme. The enzymatically cleavable linker can comprise any cleavable moiety that can be cleaved through the enzymatic action of an enzyme, such as, but not limited to, a peptide, a glycoside, and the like. In some instances, the enzyme that cleaves the enzymatically cleavable linker is present at a desired target site of action, such as the desired target site of action of the drug that is to be released from the antibody-drug conjugate. In some cases, the enzyme that cleaves the enzymatically cleavable linker is not present in a significant amount in other areas, such as in whole blood, plasma, or serum. As such, the cleavage of an enzymatically cleavable moiety can be controlled such that substantial cleavage occurs at the desired site of action, whereas cleavage does not significantly occur in other areas or before the antibody-drug conjugate reaches the desired site of action.

In some embodiments, an enzymatically cleavable linker comprises a peptide. The peptide can be any peptide suitable for use in the cleavable linker and that can be cleaved through the enzymatic action of an enzyme. Non-limiting examples of peptides that can be used as an enzymatically cleavable moiety include, for example, Val-Ala; Phe-Lys; and the like.

In some embodiments, linker L is cleavable and can be represented by the following structure:

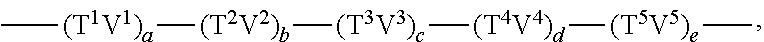

wherein:
a, b, c, d, and e are each independently 0 or 1, wherein the sum of a, b, c, d, and e is 1 to 5;
T¹ and T⁵ are each C₁-C₆ alkyl;
T², T³, and T⁴ are each independently (PEG)ₙ, (AA)ₚ, or (AA)ₚ-PABC-R¹⁶;
V¹, V², V³, V⁴, and V⁵ are each independently a covalent bond, —CO—, or —NR¹¹—;
R¹¹ is as described herein;
R¹⁶ is as described herein;
each n is independently an integer from 1 to 30; and each p is independently an integer from 1 to 20.

In some embodiments, n is an integer from 1 to 5. In some embodiments, n is 2.

In some embodiments, p is 1. In some embodiments, p is 2. In some embodiments, at one position in linker L, p is 1 and at another position in linker L, p is 2.

In some embodiments, T² is an amino acid residue. In some embodiments, T² is an amino acid residue with a polar uncharged side chain. In some embodiments, T² is a glutamine residue.

In some embodiments, T³ is (PEG)ₙ, wherein n is an integer from 1 to 30, such as from 1 to 5. In some embodiments, n is 2.

In some embodiments, T⁴ is (AA)ₚ-PABC-R¹⁶, wherein p is an integer from 1 to 20, such as from 1 to 5, and R¹⁶ is hydrogen. In some embodiments, p is 2. In some embodiments, the amino acids (AA) are valine and citrulline.

In some embodiments, linker L can be represented by the following structure:

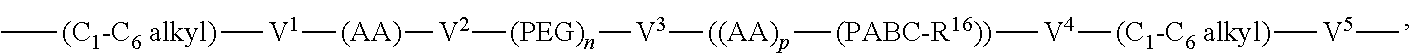

wherein:
R¹⁶ is hydrogen;
each of n and p is independently an integer from 1 to 30, 1 to 20, or 1 to 5; V¹, V², V³, V⁴, and V⁵ are each independently a covalent bond, —CO—, or —NR¹¹—; and
each R¹¹ is independently hydrogen or C₁-C₆ alkyl.

In some embodiments, n and p are both 2.

In some embodiments, linker L can be linker (L-2):

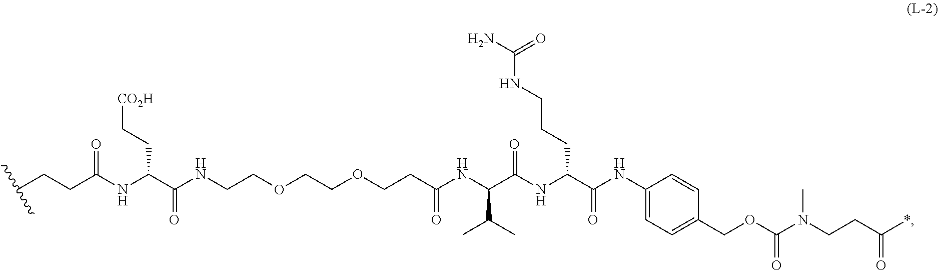

wherein [symbol] represent attachment to a pyridazine-pyrrolo coupling moiety as described herein and * represents the point of attachment to W¹.

In some embodiments, a 5T4-ADC can be represented by Formula (VI-2):

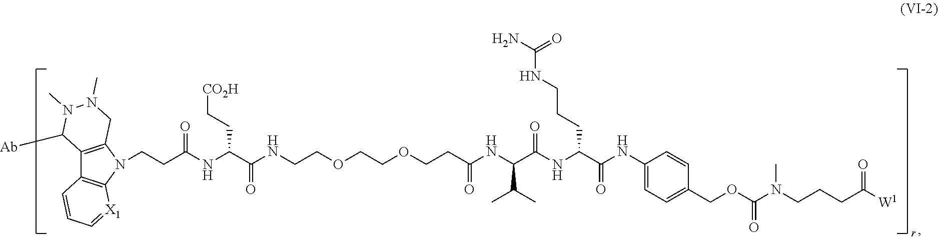

wherein:
Ab is an antibody that binds to 5T4,
r is an integer from 1 to 10; and
W¹ is a drug.

In some embodiments, X¹ is N or C.

In some embodiments, r is an integer from 1 to 4. In some embodiments, r is 1 or 2.

In some embodiments, a 5T4-ADC can be represented by Formula (VI-2), wherein X¹ is C. Accordingly, in some embodiments, a 5T4-ADC can be represented by Formula (VIa-2):

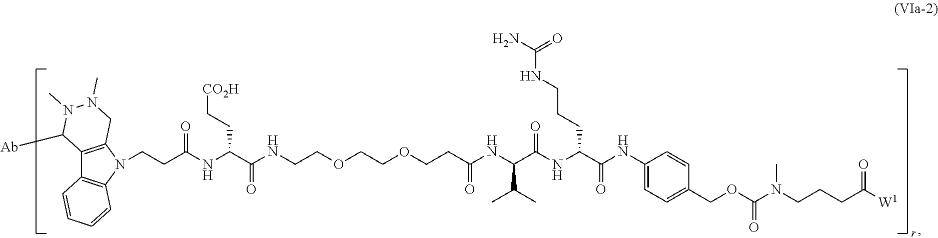

wherein:
Ab is an antibody that binds to 5T4;
r is an integer from 1 to 10; and
W¹ is a drug.

In some embodiments, r is an integer from 1 to 4. In some embodiments, r is 1 or 2.

In some embodiments, a 5T4-ADC can be represented by Formula (VI-2), wherein X¹ is N. Accordingly, in some embodiments, a 5T4-ADC can be represented by Formula (VIb-2):

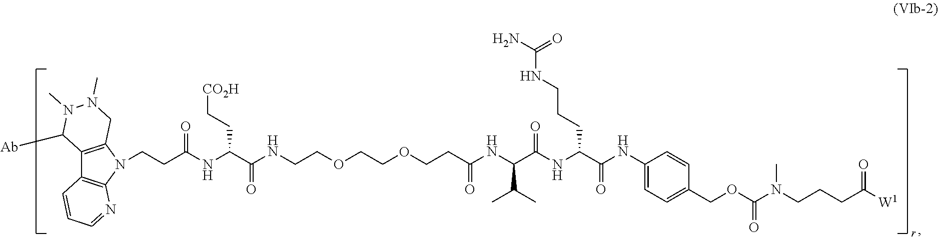

wherein:
Ab is an antibody that binds to 5T4;
r is an integer from 1 to 10; and
W¹ is a drug.

In some embodiments, r is an integer from 1 to 4. In some embodiments, r is 1 or 2.

### Dual Cleavage Linkers

In some embodiments, a cleavable linker includes two cleavable moieties, such as a first cleavable moiety and a second cleavable moiety. The cleavable moieties can be configured such that cleavage of both cleavable moieties is needed in order to separate or release the drug from the antibody, for example, at a desired target site of action for the drug. For example, cleavage of the cleavable linker can be achieved by initially cleaving one of the two cleavable moieties and then cleaving the other of the two cleavable moieties. In some embodiments, the cleavable linker includes a first cleavable moiety and a second cleavable moiety that hinders cleavage of the first cleavable moiety. By “hinders cleavage” is meant that the presence of an uncleaved second cleavable moiety reduces the likelihood or substantially inhibits the cleavage of the first cleavable moiety, thus substantially reducing the amount or preventing the cleavage of the cleavable linker. For embodiment, the presence of uncleaved second cleavable moiety can hinder enzymatic and/or chemical cleavage of the first cleavable moiety. The hindrance of cleavage of the first cleavable moiety by the presence of the second cleavable moiety, in turn, substantially reduces the amount or prevents the release of the drug from the antibody. For example, the premature release of the drug from the antibody can be substantially reduced or prevented until the 5T4-ADC is at or near the desired target site of action for the drug.

In some embodiments, the second cleavable moiety hinders cleavage of the first cleavable moiety, and cleavage of the cleavable linker can be achieved by initially cleaving the second cleavable moiety and then cleaving the first cleavable moiety. Cleavage of the second cleavable moiety can reduce or eliminate the hindrance on the cleavage of the first cleavable moiety, thus allowing the first cleavable moiety to be cleaved. Cleavage of the first cleavable moiety can result in the cleavable linker dissociating or separating into two or more portions as described herein to release the drug from the 5T4-ADC. In some embodiments, cleavage of the first cleavable moiety does not substantially occur in the presence of an uncleaved second cleavable moiety. The term “substantially” in this context, means that about 10% or less cleavage of the first cleavable moiety occurs in the presence of an uncleaved second cleavable moiety, such as about 9% or less, or about 8% or less, or about 7% or less, or about 6% or less, or about 5% or less, or about 4% or less, or about 3% or less, or about 2% or less, or about 1% or less, or about 0.5% or less, or about 0.1% or less cleavage of the first cleavable moiety occurs in the presence of an uncleaved second cleavable moiety.

Stated another way, the second cleavable moiety can protect the first cleavable moiety from cleavage. For embodiment, the presence of uncleaved second cleavable moiety can protect the first cleavable moiety from cleavage, and thus substantially reduce or prevent premature release of the drug from the 5T4 antibody until the 5T4-ADC is at or near the desired target site of action for the drug. As such, cleavage of the second cleavable moiety exposes the first cleavable moiety (e.g., deprotects the first cleavable moiety), thus allowing the first cleavable moiety to be cleaved, which results in cleavage of the cleavable linker, which, in turn, separates or releases the drug from the antibody at a desired target site of action for the drug as described herein. In some embodiments, cleavage of the second cleavable moiety exposes the first cleavable moiety to subsequent cleavage, but cleavage of the second cleavable moiety does not in and of itself result in cleavage of the cleavable linker (e.g., cleavage of the first cleavable moiety is still needed in order to cleave the cleavable linker).

Examples of cleavable moieties that are suitable for use in a cleavable linker include chemically cleavable moieties and enzymatically cleavable moieties. For example, a first cleavable moiety can be a chemically cleavable moiety and a second cleavable moiety can be a chemically cleavable moiety, which can be the same or different from the first cleavable moiety. In other embodiments, a first cleavable moiety can be an enzymatically cleavable moiety and a second cleavable moiety can be a chemically cleavable moiety. In yet other embodiments, a first cleavable moiety can be a chemically cleavable moiety and a second cleavable moiety can be an enzymatically cleavable moiety. In yet further embodiments, a first cleavable moiety can be an enzymatically cleavable moiety and a second cleavable moiety can be an enzymatically cleavable moiety, which is the same or different from the first cleavable moiety.

Chemically cleavable moieties include cleavable moieties that can be cleaved in the presence of certain chemical conditions. In some embodiments, a chemically cleavable moiety includes one or more bonds that can dissociate in the presence of certain chemical conditions, thus separating the cleavable moiety into two or more separable portions. For example, a chemically cleavable moiety can be cleaved in the presence of chemical conditions, such as acidic conditions or alkali conditions, which can lead to hydrolysis of the chemically cleavable moiety. In some embodiments, the chemical conditions under which the chemically cleavable moiety is cleaved can be present at a desired target site of action, such as the desired target site of action of the drug that is to be released from the 5T4-ADC. In some embodiments, the chemical conditions found at a desired site of cleavage of the chemically cleavable moiety are not significantly present in other areas, such as in whole blood, plasma, or serum. As such, cleavage of a chemically cleavable moiety can be controlled such that substantial cleavage occurs at a desired site of action, whereas cleavage does not significantly occur in other areas or before the 5T4-ADC reaches the desired site of action.

In some embodiments, the cleavable moiety can be an enzymatically cleavable moiety. An enzymatically cleavable moiety is a cleavable moiety that can be separated into two or more portions as described herein through the enzymatic action of an enzyme. The enzymatically cleavable moiety can be any cleavable moiety that can be cleaved through the enzymatic action of an enzyme, such as, but not limited to, a peptide, a glycoside, and the like. In some embodiments, the enzyme that cleaves the enzymatically cleavable moiety is present at a desired target site of action, such as the desired target site of action of the drug that is to be released from a 5T4-ADC. In some embodiments, the enzyme that cleaves the enzymatically cleavable moiety is not present in a significant amount in other areas, such as in whole blood, plasma, or serum. As such, the cleavage of an enzymatically cleavable moiety can be controlled such that substantial cleavage occurs at the desired site of action, whereas cleavage does not significantly occur in other areas or before the 5T4-ADC reaches the desired site of action, for example, a cancer cell.

In some embodiments, linker L can be linker (L-3):

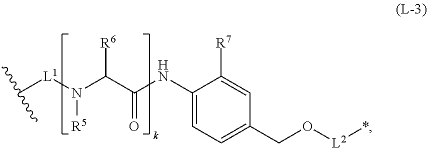

wherein:
[symbol] represents the point of attachment to a pyridazine-pyrrolo coupling moiety as described herein (e.g., any of Formulas IV, V, and VI);
* represents the point of attachment to W¹;
each R⁵ is independently hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl;
each R⁶ is independently alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl;
R⁷ is a cleavable moiety;
k is an integer from 1 to 10;
L¹ comprises -(T¹-V¹)ₐ-(T²-V²)_{b}-(T³-V³)_{c}-(T⁴-V⁴)_{d}—, L² comprises -(T⁵-V⁵)ₑ-(T⁶-V⁶)_{f}-(T⁷-V⁷)_{g}-(T⁸-V⁸)ₙ—;
T¹, T², T³, T⁴, T⁵, T⁶, T⁷, and T⁸ are each independently a covalent bond, C₁-C₁₂ alkyl, substituted C₁-C₁₂ alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, P4A-R¹², acetal, a hydrazine, a disulfide, or an ester;
V¹, V², V³, V⁴, V⁵, V⁶, V⁷, and V⁸ are each independently a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, or —P(O)OH—;
each of a, b, c, d, e, f, g, and h is independently 1 or 0;
m is independently an integer from 1 to 12;
n is independently an integer from 1 to 30;
p is independently an integer from 1 to 20;
q is independently an integer from 1 to 6;
w is independently an integer from 1 to 20;
each R¹² is independently hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl;
each R¹³ is independently hydrogen, alkyl, substituted alkyl, aryl, or substituted aryl; and
each R¹⁵ is independently hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, or substituted heterocyclyl.

In some embodiments, a 5T4-ADC of Formula (V) includes linker (L-3). Accordingly, in some embodiments, a 5T4-ADC can be represented by Formula (V-3)

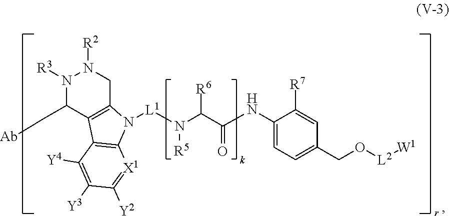

wherein:
Ab is an antibody that binds to 5T4;
r is an integer from 1 to 10;
X¹ is C or N;
W¹ is a drug;
R², R³, Y¹, Y², Y³, and Y⁴ are as defined herein, such as for Formula (V); and
L¹, L², R⁵, R⁶, R⁷, and k are as defined herein, such as for linker (L-3).

In some embodiments, r is an integer from 1 to 4. In some embodiments, r is an integer from 1 to 2.

In some embodiments, a 5T4-ADC of Formula (VI) includes linker (L-3). Accordingly, in some embodiments, a 5T4-ADC can be represented by Formula (VI-3)

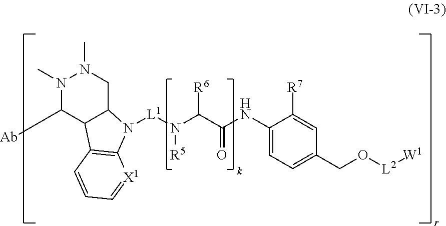

wherein:
Ab is an antibody that binds to 5T4;
r is an integer from 1 to 10;
X¹ is C or N;
W¹ is a drug; and
L¹, L², R⁵, R⁶, R⁷, and k are as defined herein, such as for linker (L-3).

In some embodiments, r is an integer from 1 to 4. In some embodiments, r is 1 or 2.

In some embodiments of linker (L-3), k is 2. Accordingly, linker L can be linker (L-4):

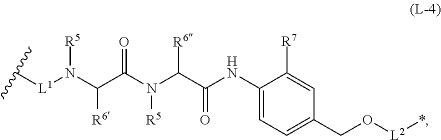

wherein:
[symbol] represents the point of attachment to a pyridazine-pyrrolo coupling moiety as described herein (e.g., any of Formulas IV, V, and VI);
* represents the point of attachment to W;
each R⁵ is independently hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl;
R^{6′} and R^{6″} are each independently alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl;
R⁷ is a cleavable moiety; and
L¹ and L² are as defined herein, such as for linker (L-3).

In some embodiments, a 5T4-ADC of Formula (V) includes linker (L-4). Accordingly, in some embodiments, a 5T4-ADC can be represented by Formula (V-4):

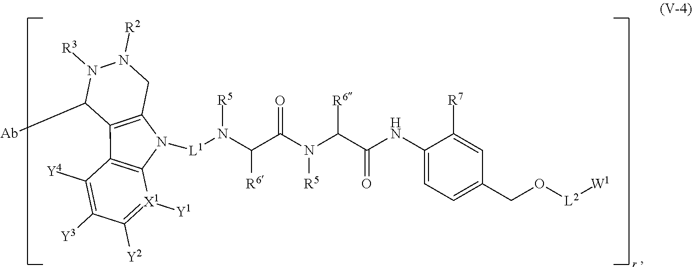

wherein:
Ab is an antibody that binds to 5T4;
r is an integer from 1 to 10;
X¹ is C or N;
W¹ is a drug;
R², R³, Y¹, Y², Y³, and Y⁴ are as defined herein, such as for Formula (V);
L¹, L², R⁵, R^{6′}, R^{6″}, and R⁷ are as defined herein, such as for linker (L-4).

In some embodiments, r is an integer from 1 to 4. In some embodiments, r is 1 or 2.

In some embodiments, a 5T4-ADC of Formula (VI) includes linker (L-4). In some embodiments, a 5T4-ADC can be represented by Formula (VI-4)

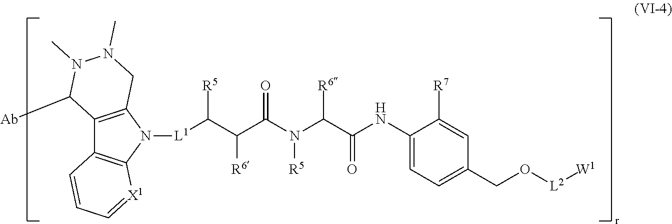

wherein:
Ab is an antibody that binds to 5T4;
r is an integer from 1 to 10;
X¹ is C or N;
W¹ is a drug;
L¹, L², R⁵, R^{6′}, R^{6″}, and R⁷ are as defined herein, such as for linker (L-4).

In some embodiments, r is an integer from 1 to 4. In some embodiments, r is 1 or 2.

In some embodiments, R⁷ is an enzymatically cleavable moiety comprising a sugar moiety, such as a glycoside or glycosyl. In some embodiments, the glycoside can facilitate an increase in the hydrophilicity of the cleavable linker as compared to a cleavable linker that does not include the glycoside. The glycoside can be any glycoside suitable for use in the cleavable linker and that can be cleaved through the enzymatic action of an enzyme.

A glycoside of a R⁷ moiety can be attached to the rest of the 5T4-ADC through a glycosidic bond. The glycosidic bond can link the glycoside to the 5T4-ADC through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some embodiments, the glycosidic bond is an O-glycosidic bond (an O-glycoside).

In some embodiments, R⁷ is

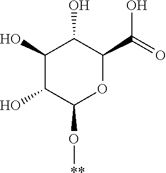

wherein ** represents the point of attachment to the phenyl group described herein, such as in Formula (VI-3), (V-4), or VI-4). Accordingly, linker L can be linker (L-5):

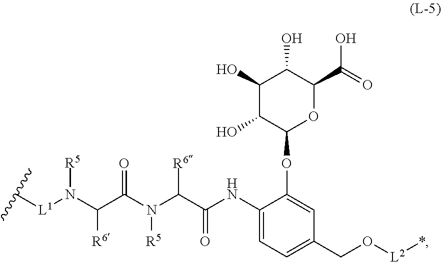

wherein: [symbol]
represents the point of attachment to a pyridazine-pyrrolo coupling moiety as described herein (e.g., any of Formulas IV, V, and VI);
* represents the point of attachment to W¹;
each R⁵ is independently hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl;
R^{6′} and R^{6″} are independently alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl; and
L¹ and L² are as defined herein, such as for linker (L-3).

In some embodiments, linker (L-5) is attached to Formula (V). In some embodiments, a 5T4-ADC can be represented by Formula (V-5):

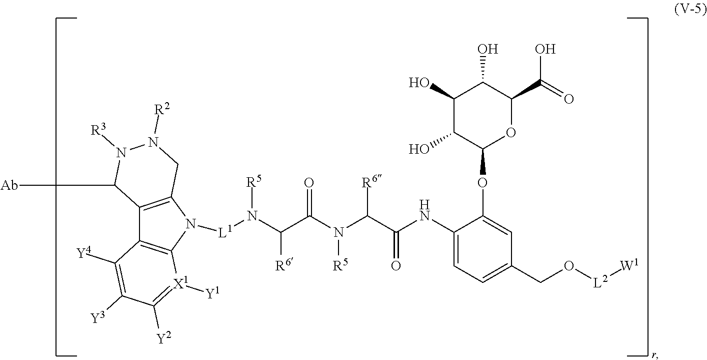

wherein:
Ab is an antibody that binds to 5T4;
r is an integer from 1 to 10;
X¹ is C or N;
W¹ is a drug;
R², R³, Y¹, Y², Y³, and Y⁴ are as defined herein, such as for Formula (V);
L¹, L², R⁵, R^{6′}, and R^{6″} are as defined herein, such as for linker (L-5).

In some embodiments, r is an integer from 1 to 4. In some embodiments, r is 1 or 2.

In some embodiments, a 5T4-ADC of Formula (VI) can include linker (L-5). Accordingly, in some embodiments, a 5T4-ADC can be represented by Formula (VI-5):

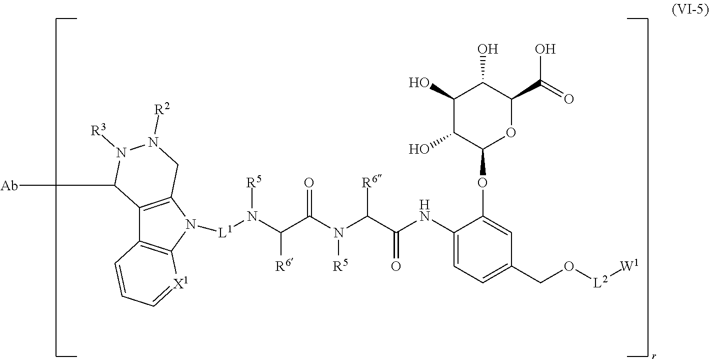

wherein:
Ab is an antibody that binds to 5T4;
r is an integer from 1 to 10;
X¹ is C or N;
W¹ is a drug;
L¹, L², R⁵, R^{6′}, and R^{6″} are as defined herein, such as for linker (L-5).

In some embodiments, r is an integer from 1 to 4. In some embodiments, r is 1 or 2.

In some embodiments of linker (L-5), R⁵ is hydrogen, R^{6′} is isopropyl and R^{6″} is methyl. Accordingly, in some embodiments, linker L can be linker (L-6):

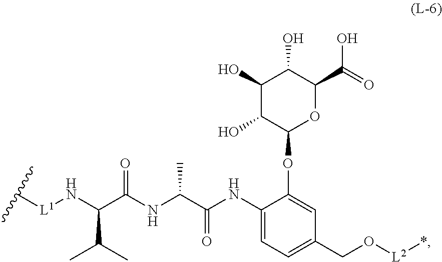

wherein:
[symbol] represents the point of attachment to a pyridazine-pyrrolo coupling moiety (e.g., any of Formulas IV, V, and VI);
* represents the point of attachment to W¹; and
L¹ and L² are as defined herein, such as for linker (L-5).

In some embodiments, a 5T4-ADC of Formula (V) includes linker (L-6). Accordingly, in some embodiments, a 5T4-ADC can be represented by Formula (V-6):

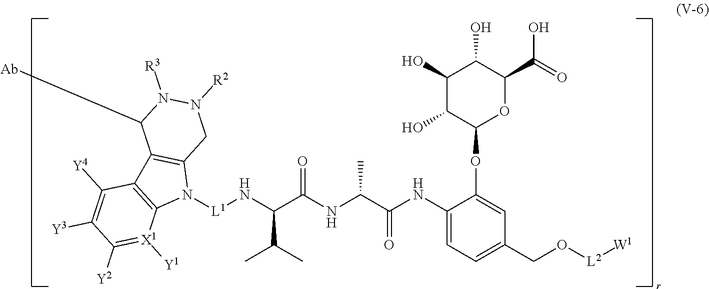

wherein:
Ab is an antibody that binds to 5T4;
r is an integer from 1 to 10;
X¹ is C or N;
R², R³, Y¹, Y², Y³, and Y⁴ are as defined herein, such as for Formula (V);
W¹ is a drug; and
L¹ and L² are as defined herein, such as for linker (L-6).

In some embodiments, r is an integer from 1 to 4. In some embodiments, r is 1 or 2.

In some embodiments, a 5T4-ADC of Formula (VI) includes linker (L-6). Accordingly, in some embodiments, a 5T4-ADC can be represented by Formula (VI-6):

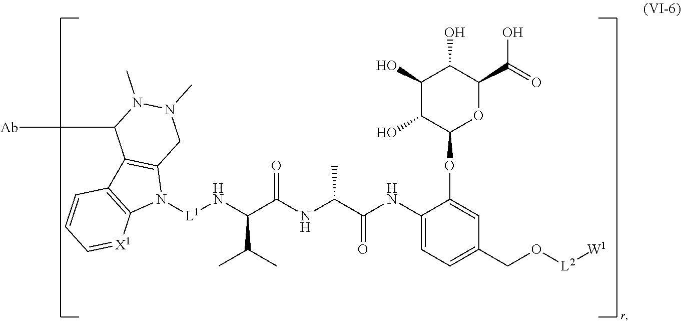

wherein:
Ab is an antibody that binds to 5T4;
r is an integer from 1 to 10;
X¹ is C or N;
W¹ is a drug; and
L¹ and L² are as defined herein, such as for linker (L-6).

In some embodiments, r is an integer from 1 to 4. In some embodiments, r is 1 or 2.

In some embodiments, a 5T4-ADC can be represented by Formula (V-3), (V-4), (V-5), (V-6), (VI-3), (VI-4), (VI-5), or (VI-6), wherein:
L¹ comprises -(T¹-V¹)ₐ-(T²-V²)_{b}-(T³-V³)_{c}-(T⁴-V⁴)_{d}—;
L² comprises -(T⁵-V⁵)ₑ-(T⁶-V⁶)_{f}-(T⁷-V⁷)_{g}-(T⁸-V⁸)ₕ—;
T¹, T², and T³ are each independently C₁-C₁₂ alkyl, (PEG)ₙ, or (AA)ₚ;
T⁵, T⁶, T⁷, and T⁸ are each independently a covalent bond, C₁-C₁₂ alkyl, substituted C₁-C₁₂ alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, P4A-R¹², acetal, a hydrazine, a disulfide, and an ester;
V¹, V², V³, V⁴, V⁵, V⁶, V⁷, and V⁸ are each independently-C(═O)—or —NR¹¹—;
a, b, and c are each 1;
d is 0 and therefore T⁴ and V⁴ are absent;
each of e, f, g, and h is independently 1 or 0;
n is an integer from 1 to 10;
p is an integer from 1 to 10; and
R¹¹ is hydrogen, alkyl, substituted alkyl, a polyethylene glycol moiety, aryl, or substituted aryl.

In some embodiments, a 5T4-ADC can be represented by Formula (V-3), (V-4), (V-5), (V-6), (VI-3), (VI-4), (VI-5), or (VI-6), wherein T¹, T², and T³ are C₁-C₆ alkyl, (AA)ₚ, or (PEG)ₙ, and p and n are independently integers from 1 to 10. In some embodiments, p is 1. In some embodiments, n is 2. In some embodiments, (AA)ₚ can include a non-naturally occurring amino acid, such as a cysteine sulfonic acid amino acid. In some embodiments, V¹, V², and V³ are independently —C(═O)—or —NR¹⁵—, wherein R¹⁵ is hydrogen.

In any embodiment described herein, L¹ can be as follows:

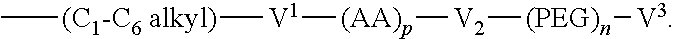

wherein V¹, V², V³, AA, p, and n are as described herein, such as for linker (L-3). In some embodiments, V¹, V², and V³ are —C(═O) or —NR¹⁵—, wherein R¹⁵ is hydrogen. In some embodiments, p is 1 and n is 2. In some embodiments, (AA)ₚ can include a non-naturally occurring amino acid, such as a cysteine sulfonic acid amino acid.

In some embodiments, L¹ is

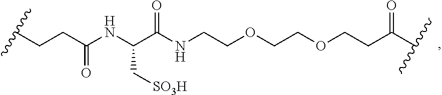 [SMILES: *CCC(=O)N[C@@H](CS(=O)(=O)O)C(=O)NCCOCCOCCC(*)=O]

wherein each [symbol] represents a point of attachment in any direction to an adjacent chemical moiety in the linker. Accordingly, in some embodiments, linker L can be linker (L-7):

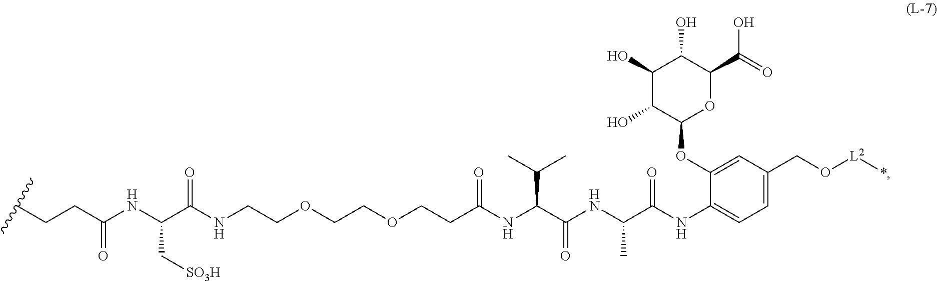

wherein:
[symbol] represents the point of attachment to a pyridazine-pyrrolo coupling moiety as described herein (e.g., any of Formulas IV, V, and VI);
* represents the point of attachment to W¹; and
L² is as defined herein, such as for linker (L-3).

In some embodiments, a 5T4-ADC can be represented by Formula (V-7):

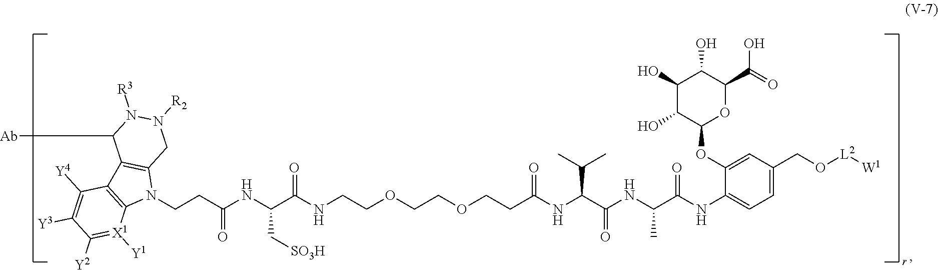

wherein:
Ab is an antibody that binds to 5T4;
r is an integer from 1 to 10;
X¹ is C or N;
R², R³, Y¹, Y², Y³, and Y⁴ are as defined herein, such as for Formula (V);
W¹ is a drug; and
L² is as defined herein, such as for linker (L-7).

In some embodiments, r is an integer from 1 to 4. In some embodiments, r is 1 or 2.

In some embodiments, a 5T4-ADC can be represented by Formula (VI-7):

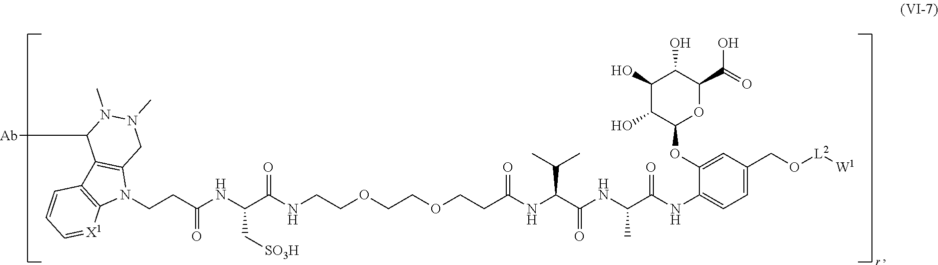

wherein:
Ab is an antibody that binds to 5T4;
r is an integer from 1 to 10;
X¹ is C or N;
W¹ is a drug; and
L² is as defined herein, such as for linker (L-7).

In some embodiments, r is an integer from 1 to 4. In some embodiments, r is 1 or 2.

In some embodiments of L², e is 1, T⁵ is a covalent bond, V⁵ is carbonyl, and each of f, g, and h is 0. In some embodiments, L² is carbonyl (C═O). Accordingly, in some embodiments, linker L can be linker (L-8):

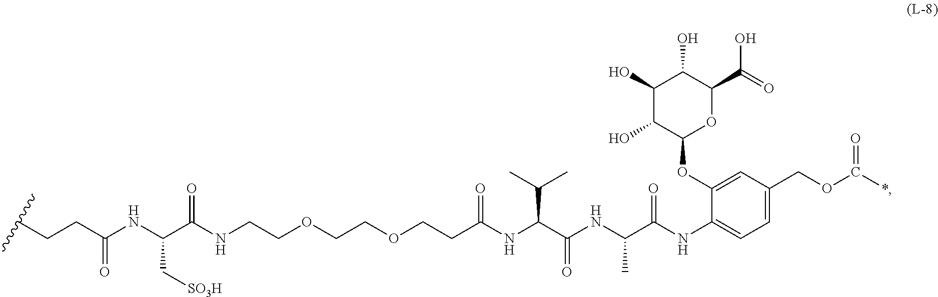

wherein:
[symbol] represents the point of attachment to a pyridazine-pyrrolo coupling moiety as described herein (e.g., any of Formulas IV, V, and VI); and
* represents the point of attachment to W.

In some embodiments, linker (L) in the 5T4-ADC of Formula (V) can be linker (L-8). Accordingly, in some embodiments, a 5T4-ADC can be represented by Formula (V-8):

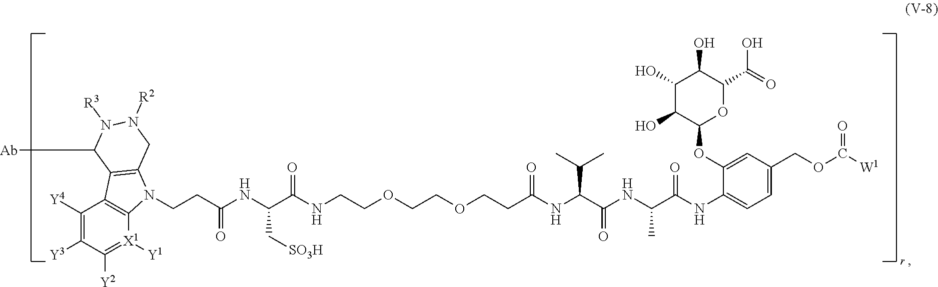

wherein:
Ab is an antibody that binds to 5T4;
r is an integer from 1 to 10;
X¹ is C or N;
R², R³, Y¹, Y², Y³, and Y⁴ are as defined herein, such as for Formula (V); and
W¹ is a drug.

In some embodiments, r is an integer from 1 to 4. In some embodiments, r is 1 or 2.

In some embodiments, linker (L) in the 5T4-ADC of Formula (VI) can be linker (L-8). Accordingly, in some embodiments, a 5T4-ADC can be represented by Formula (VI-8):

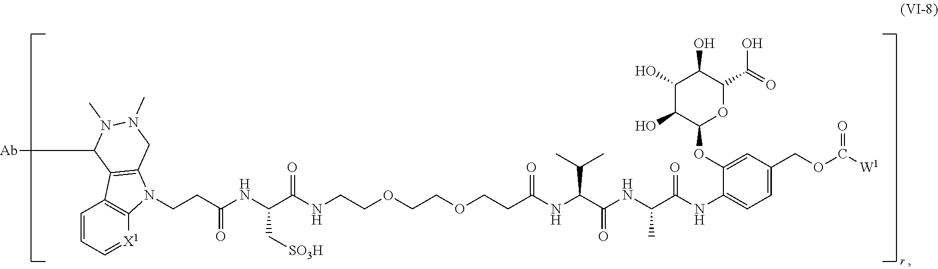

wherein:
Ab is an antibody that binds to 5T4;
r is an integer from 1 to 10;
X¹ is C or N; and
W¹ is a drug.

In some embodiments, r is an integer from 1 to 4. In some embodiments, r is 1 or 2.

In some embodiments, a 5T4-ADC can be represented by Formula (VI-8), wherein X¹ is C. Accordingly, in some embodiments, a 5T4-ADC can be represented by Formula (VIa-8):

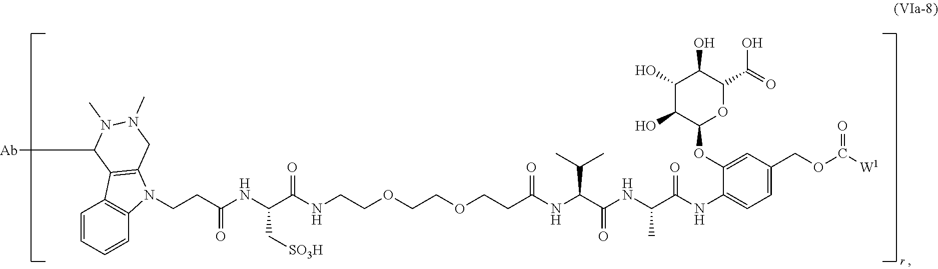

wherein:
Ab is an antibody that binds to 5T4;
r is an integer from 1 to 10; and
W¹ is a drug.

In some embodiments, r is an integer from 1 to 4. In some embodiments, r is 1 or 2.

In some embodiments, a 5T4-ADC can be represented by Formula (VI-8), wherein X¹ is N. Accordingly, in some embodiments, a 5T4-ADC can be represented by Formula (VIb-8):

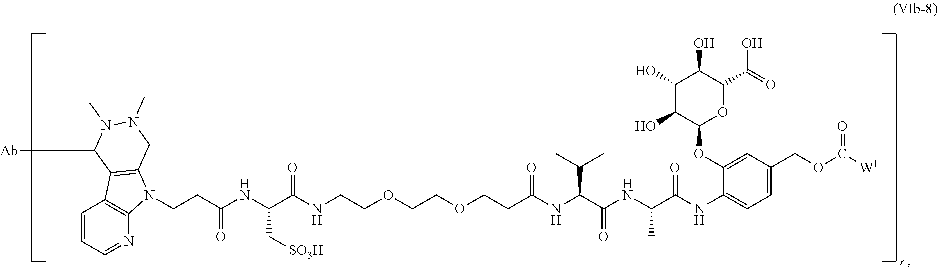

wherein:
Ab is an antibody that binds to 5T4;
r is an integer from 1 to 10; and
W¹ is a drug.

In some embodiments, r is an integer from 1 to 4. In some embodiments, r is 1 or 2.

### Drugs

The drug useful for use in a 5T4-ADC as described herein, represented as W¹ in each of the formulas provided herein, is not particularly limited. Examples of suitable drugs include, but are not limited to, small molecule drugs, such as cancer chemotherapeutic agents. For example, the drug can be a microtubule-affecting agent. In some embodiments, the drug is a microtubule-affecting agent that has antiproliferative activity, such as a maytansinoid or an auristatin.

### Maytansine

In some embodiments, the drug is a maytansinoid. For example, the drug can be maytansine, which has the following structure (W-1):

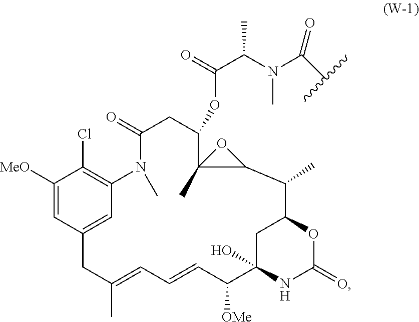

or a derivative thereof, such as deacylated maytansine (W-1′):

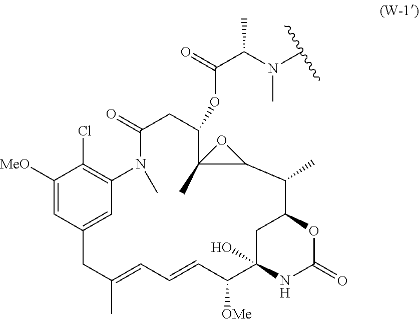

wherein [symbol] indicates the point of attachment of the maytansinoid to a linker in a 5T4-ADC.

For example, in any of the 5T4-ADCs represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), and (VIb-8), W¹ can be a maytansinoid, such as a maytansinoid of W-1 or W-1′, wherein [symbol] indicates the point of attachment between the maytansinoid and L or L².

In some embodiments, a 5T4-ADC can be represented by Formula (VI-11), Formula (VI-21), or Formula (VI-81):

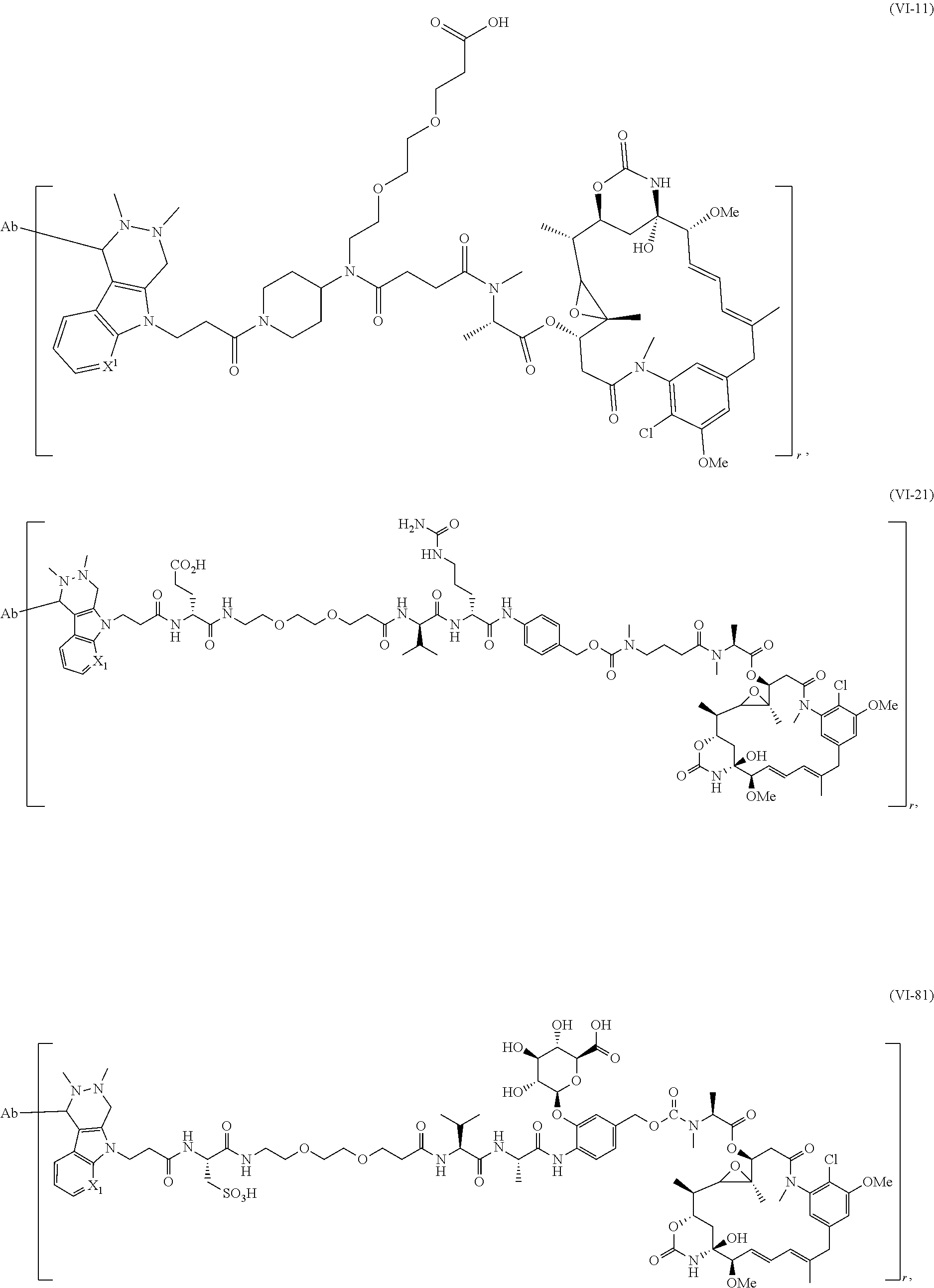

wherein:
Ab is an antibody that binds to 5T4;
r is an integer from 1 to 10; and
X¹ is C or N.

In some embodiments, r is an integer from 1 to 4. In some embodiments, r is 1 or 2.

In some embodiments, X¹ is C, and a 5T4-ADC can be represented by Formula (VIa-11) or Formula (VIa-21):

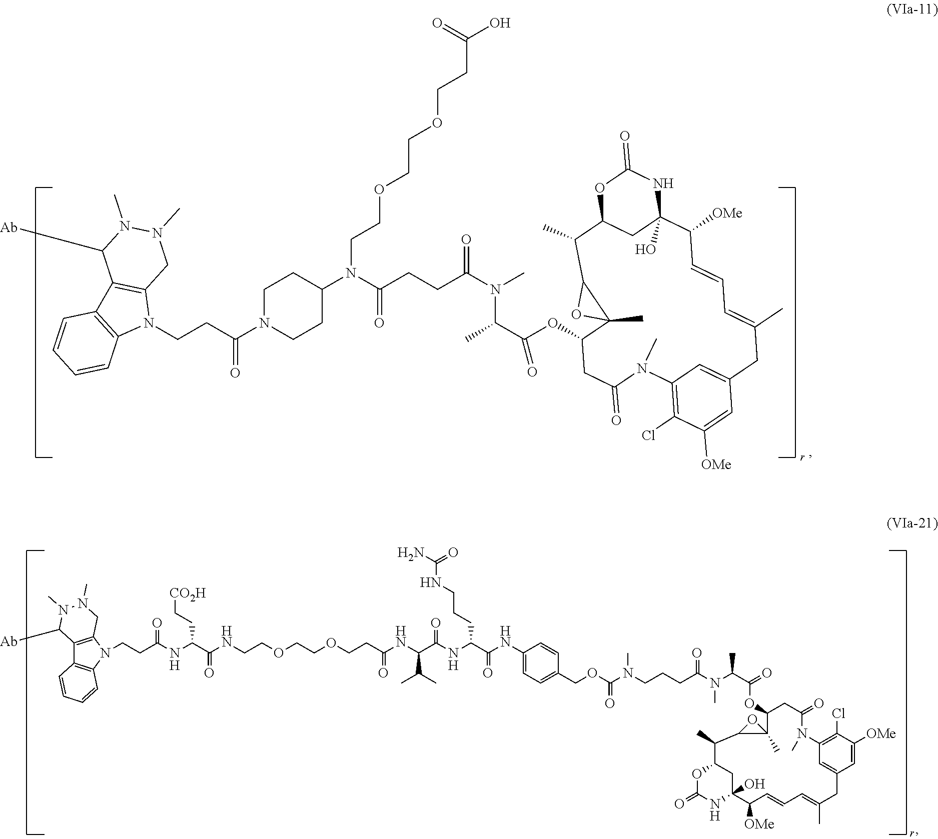

wherein:
Ab is an antibody that binds to 5T4; and
r is an integer from 1 to 10.

In some embodiments, r is an integer from 1 to 4. In some embodiments, r is 1 or 2.

In some embodiments, X¹ is N, and a 5T4-ADC can be represented by Formula (VIb-81):

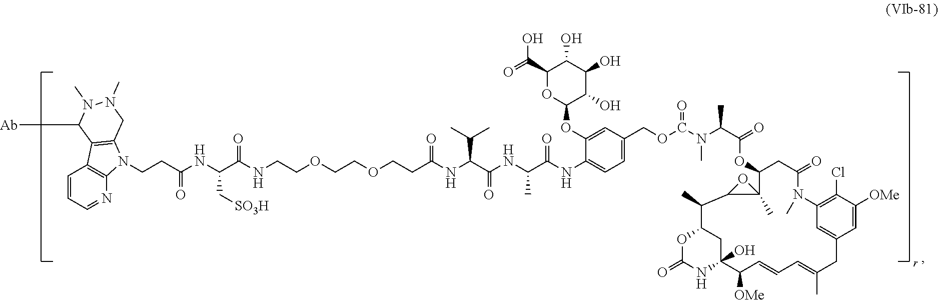

wherein:
Ab is an antibody that binds to 5T4; and
r is an integer from 1 to 10.

In some embodiments, r is an integer from 1 to 4. In some embodiments, r is 1 or 2.

In some embodiments, the maytansinoid of the structure above can be referred to as a deacylated maytansine or a deacyl maytansine.

### Auristatin

In some embodiments, the drug is an antimitotic agent, such as an auristatin or an active auristatin analog or derivative thereof (e.g., monomethyl auristatin D (MMAD), monomethyl auristatin E (MMAE), monomethyl auristatin F (MMAF), and the like). For example, in any of the formulas representing 5T4-ADCs described herein, W¹ can be MMAE, MMAF, or MMAD.

In some embodiments, W¹ is MMAE, which has the following structure (W-2):

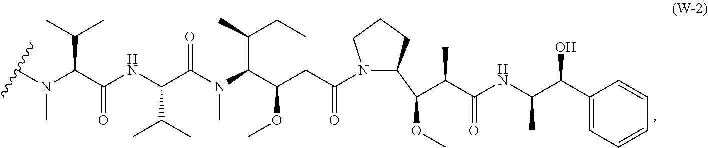 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein [symbol] indicates the point of attachment between MMAE and a linker L (such as [symbol] an L² moiety as disclosed herein) in a 5T4-ADC.

For example, MMAE can be included in a 5T4-ADC that comprises linker (L-1). Accordingly in some embodiments, a 5T4-ADC can be represented by Formula (VI-12):

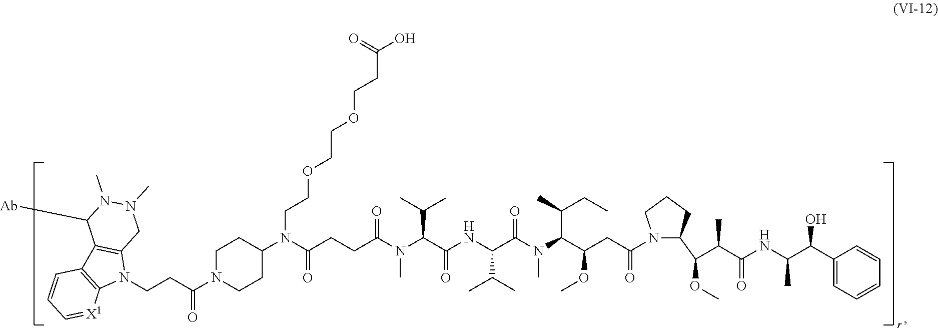

wherein:
Ab is an antibody that binds to 5T4;
r is an integer from 1 to 10; and
X¹ is C or N.

In some embodiments, r is an integer from 1 to 4. In some embodiments, r is 1 or 2.

In some embodiments, MMAE can be included in a 5T4-ADC comprising linker (L-5). Accordingly in some embodiments, a 5T4-ADC can be represented by Formula (VI-52):

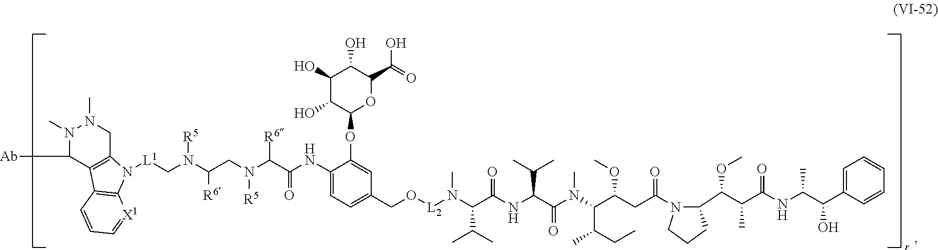

wherein:
Ab is an antibody that binds to 5T4;
r is an integer from 1 to 10;
X¹ is C or N; and
L¹ and L² are as defined herein, such as for linker (L-3).

In some embodiments, r is an integer from 1 to 4. In some embodiments, r is 1 or 2.

In some embodiments, MMAE can be included in a 5T4-ADC comprising linker (L-6). Accordingly in some embodiments, a 5T4-ADC can be represented by Formula (VI-62):

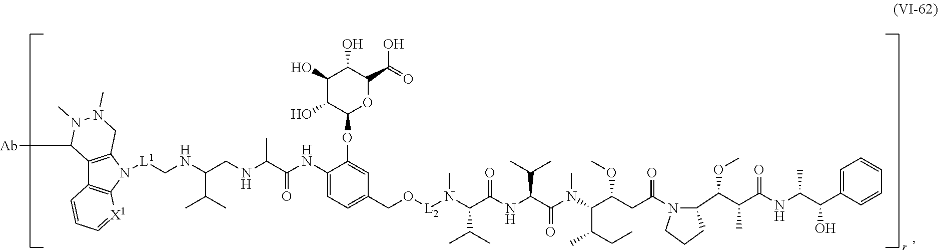

wherein:
Ab is an antibody that binds to 5T4;
r is an integer from 1 to 10;
X¹ is C or N; and
L¹ and L² are as defined herein, such as for linker (L-3).
   In some embodiments, r is an integer from 1 to 4. In some embodiments, r is 1 or 2.

In some embodiments, a 5T4-ADC can be represented by Formula (VI-12), (VI-52), and (VI-62) wherein X¹ is N. In some embodiments, a 5T4-ADC can be represented by Formula (VIb-12), Formula (VIb-52), or Formula (VIb-62):

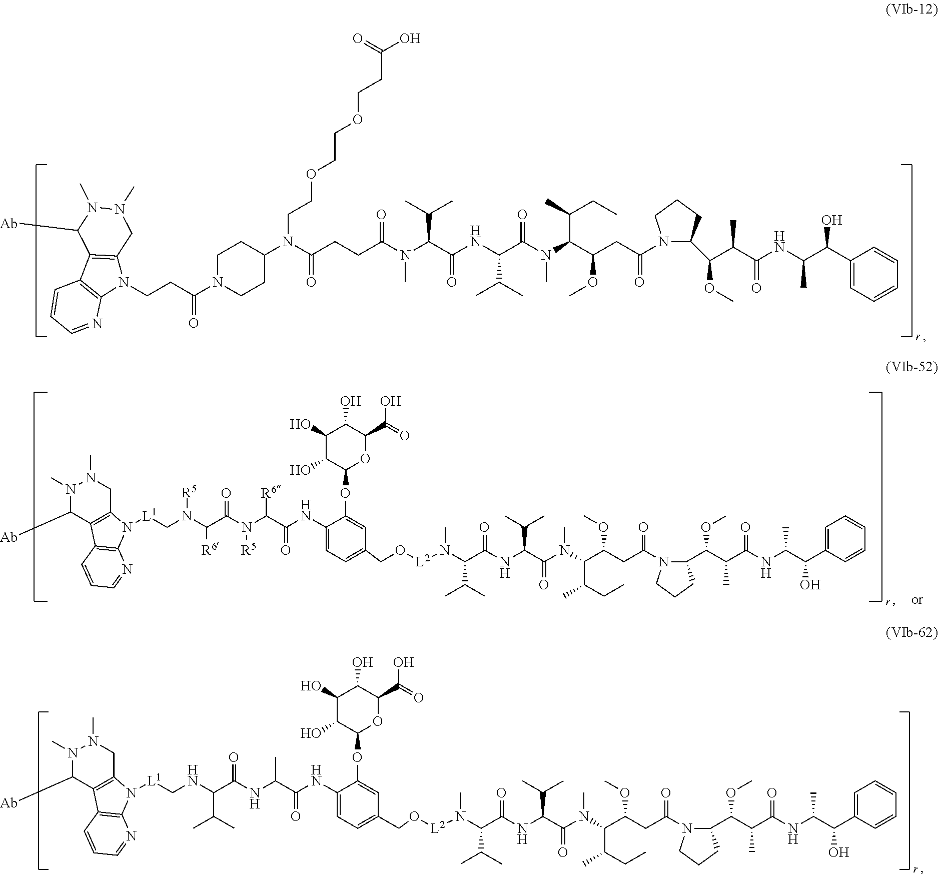

wherein
Ab is an antibody that binds to 5T4;
r is an integer from 1 to 10;
R⁵, R^{6′}, R^{6″}, L¹, and L² are as defined herein, such as for linker (L-5); and
r is an integer from 1 to 10.

In some embodiments, r is an integer from 1 to 4. In some embodiments, r is 1 or 2.

In some embodiments, MMAE can be included in a 5T4-ADC comprising linker (L-7). Accordingly, a 5T4-ADC can be represented by Formula (VIb-72):

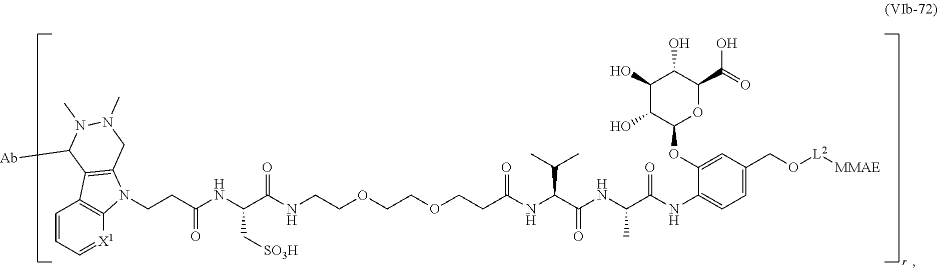

wherein:
Ab is an antibody that binds to 5T4;
r is an integer from 1 to 10; and
L² is as defined herein, such as for linker (L-6).

In some embodiments, r is an integer from 1 to 4. In some embodiments, r is 1 or 2.

In some embodiments, MMAE can be included in a 5T4-ADC comprising linker (L-8). Accordingly, a 5T4-ADC can be represented by Formula (VIb-82):

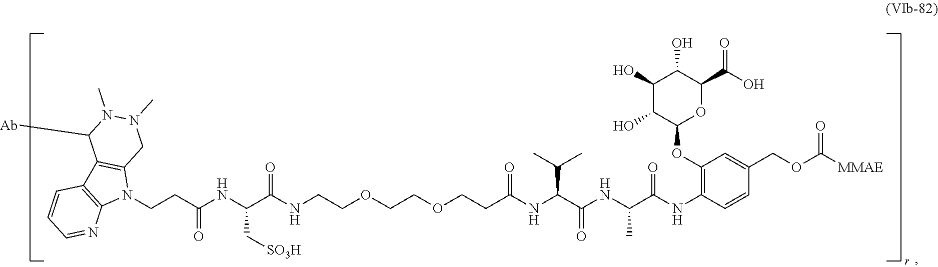

wherein
Ab is an antibody that binds to 5T4; and
r is an integer from 1 to 10.

In some embodiments, r is an integer from 1 to 4. In some embodiments, r is 1 or 2.

In some embodiments, W¹ is MMAF, which has the following structure (W-3):

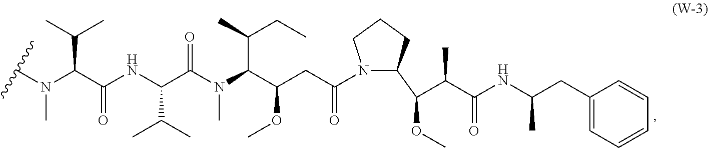 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)Cc1ccccc1)OC)C(C)C)C(C)C]

wherein [symbol] indicates the point of attachment between MMAF and a linker in a 5T4-ADC.

For example, MMAF can be included in a 5T4-ADC, for example, as follows:

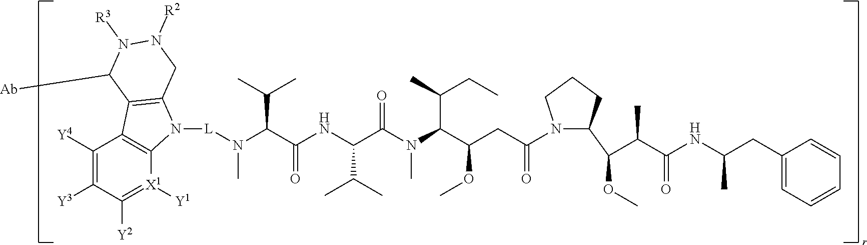

wherein:
Ab is an antibody that binds to 5T4;
r is an integer from 1 to 10; and
Y¹, Y², Y³, Y⁴, X¹, R², R³, and L are as defined herein, such as for Formula (V).

In some embodiments, L is chosen from linkers (L-1), (L-2), (L-3), (L-4), (L-5), (L-6), (L-7), and (L-8). In some embodiments, X¹ is C or N. In some embodiments, each of Y¹, Y², Y³ and Y⁴ is hydrogen. In some embodiments, R² and R³ are independently C₁-C₆ alkyl, such as methyl. In some embodiments, r is an integer from 1 to 10. In some embodiments, r is an integer from 1 to 4. In some embodiments, r is 1 or 2.

### SUMMARY SECTION

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82) wherein Ab is an antibody that binds to 5T4 as disclosed herein. In some embodiments, the antibody comprises a VH comprising one or more (such as one, two, or three) CDRs identical or substantially identical to the CDRs as described herein, such as in any one of Tables 1-3. Additionally or alternatively, in some embodiments, the antibody comprises a VL comprising one or more (such as one, two, or three) CDRs identical or substantially identical to the CDRs as described herein, such as in any one of Tables 1-3.

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82), wherein Ab is an antibody that binds to 5T4 as disclosed herein. In some embodiments, the antibody comprises a VH identical or substantially identical to a VH as described herein, such as in in any one of Tables 1-3. Additionally or alternatively, in some embodiments, the antibody comprises a VL identical or substantially identical to a VL as described herein, such as in any one of Tables 1-3.

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82), wherein Ab is an antibody that binds to 5T4. In some embodiments, the antibody comprises (i) a VH comprising the amino acid sequence of SEQ ID NO:25 and a VL comprising the amino acid sequence of SEQ ID NO:26; or (ii) a VH comprising the amino acid sequence of SEQ ID NO:44 and a VL comprising the amino acid sequence of SEQ ID NO:45; or (iii) a VH comprising the amino acid sequence of SEQ ID NO:62 and a VL comprising the amino acid sequence of SEQ ID NO:63.

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82), wherein Ab is an antibody that binds to 5T4 as disclosed herein. In some embodiments, the antibody comprises (a) a VH region comprising: (1) a VH CDR1 comprising an amino acid sequence of any one SEQ ID NOs:1, 7, 12, 13, and 18; (2) a VH CDR2 comprising an amino acid sequence of any one SEQ ID NOs:2, 8, 14, 19, and 24; and (3) a VH CDR3 comprising an amino acid sequence of any one SEQ ID NOs:3, 9, 15, and 20; and (b) a VL region comprising: (1) a VL CDR1 comprising an amino acid sequence of any one SEQ ID NOs:4, 10, 16, and 21; (2) a VL CDR2 comprising an amino acid sequence of any one SEQ ID NOs:5, 11, and 22; and (3) a VL CDR3 comprising an amino acid sequence of any one SEQ ID NOs:6, 17, and 23.

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82), wherein Ab is an antibody that binds to 5T4. In some embodiments, the antibody comprises (i) a VH region comprising a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; or (ii) a VH region comprising a VH CDR1 comprising the amino acid sequence of SEQ ID NO:7, a VH CDR2 comprising the amino acid sequence of SEQ ID NO:8, and a VH CDR3 comprising the amino acid sequence of SEQ ID NO:9; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:6; or (iii) a VH region comprising a VH CDR1 comprising the amino acid sequence of SEQ ID NO:12, a VH CDR2 comprising the amino acid sequence of SEQ ID NO:2, and a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; or (iv) a VH region comprising a VH CDR1 comprising the amino acid sequence of SEQ ID NO:13, a VH CDR2 comprising the amino acid sequence of SEQ ID NO:14, and a VH CDR3 comprising the amino acid sequence of SEQ ID NO:15; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:17; or (v) a VH region comprising a VH CDR1 comprising the amino acid sequence of SEQ ID NO:18, a VH CDR2 comprising the amino acid sequence of SEQ ID NO:19, and a VH CDR3 comprising the amino acid sequence of SEQ ID NO:20; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:23; or (vi) a VH region comprising a VH CDR1 comprising the amino acid sequence of SEQ ID NO:1, a VH CDR2 comprising the amino acid sequence of SEQ ID NO:24, and a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82), wherein Ab is an antibody that binds to 5T4 and comprises (a) a VH region comprising: (1) a VH CDR1 comprising an amino acid sequence of any one SEQ ID NOs:27, 31, 34, 35, and 39; (2) a VH CDR2 comprising an amino acid sequence of any one SEQ ID NOs:28, 32, 36, 40, and 43; and (3) a VH CDR3 comprising an amino acid sequence of any one SEQ ID NOs:29, 33, 37, and 41; and (b) a VL region comprising: (1) a VL CDR1 comprising an amino acid sequence of any one SEQ ID NOs:4, 10, 16, and 21; (2) a VL CDR2 comprising an amino acid sequence of any one SEQ ID NOs:5, 11, and 22; and (3) a VL CDR3 comprising an amino acid sequence of any one SEQ ID NOs:30, 38, and 42.

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82), wherein Ab is an antibody that binds to 5T4. In some embodiments, the antibody comprises (i) a VH region comprising a VH CDR1 comprising the amino acid sequence of SEQ ID NO:27, a VH CDR2 comprising the amino acid sequence of SEQ ID NO:28, and a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30; or (ii) a VH region comprising a VH CDR1 comprising the amino acid sequence of SEQ ID NO:31, a VH CDR2 comprising the amino acid sequence of SEQ ID NO:32, and a VH CDR3 comprising the amino acid sequence of SEQ ID NO:33; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:30; or (iii) a VH region comprising a VH CDR1 comprising the amino acid sequence of SEQ ID NO:34, a VH CDR2 comprising the amino acid sequence of SEQ ID NO:28, and a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30; or (iv) a VH region comprising a VH CDR1 comprising the amino acid sequence of SEQ ID NO:35, a VH CDR2 comprising the amino acid sequence of SEQ ID NO:36, and a VH CDR3 comprising the amino acid sequence of SEQ ID NO:37; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:38; or (v) a VH region comprising a VH CDR1 comprising the amino acid sequence of SEQ ID NO:39, a VH CDR2 comprising the amino acid sequence of SEQ ID NO:40, and a VH CDR3 comprising the amino acid sequence of SEQ ID NO:41; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:42; or (vi) a VH region comprising a VH CDR1 comprising the amino acid sequence of SEQ ID NO:27, a VH CDR2 comprising the amino acid sequence of SEQ ID NO:43, and a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30.

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82), wherein Ab is an antibody that binds to 5T4. In some embodiments, the antibody comprises (a) a VH region comprising (1) a VH CDR1 comprising an amino acid sequence of any one SEQ ID NOs:46, 50, 53, 13, and 57; (2) a VH CDR2 comprising an amino acid sequence of any one SEQ ID NOs:47, 51, 54, 58, and 61; and (3) a VH CDR3 comprising an amino acid sequence of any one SEQ ID NOs:48, 52, 55, and 59; and (b) a VL region comprising: (1) a VL CDR1 comprising an amino acid sequence of any one SEQ ID NOs:4, 10, 16, and 21; (2) a VL CDR2 comprising an amino acid sequence of any one SEQ ID NOs:5, 11, and 22; and (3) a VL CDR3 comprising an amino acid sequence of any one SEQ ID NOs:49, 56, and 60.

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82), wherein Ab is an antibody that binds to 5T4 and comprises (i) a VH region comprising a VH CDR1 comprising the amino acid sequence of SEQ ID NO:46, a VH CDR2 comprising the amino acid sequence of SEQ ID NO:47, and a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49; (ii) a VH region comprising a VH CDR1 comprising the amino acid sequence of SEQ ID NO:50, a VH CDR2 comprising the amino acid sequence of SEQ ID NO:51, a VH CDR3 comprising the amino acid sequence of SEQ ID NO:52; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:49; or (iii) a VH region comprising a VH CDR1 comprising the amino acid sequence of SEQ ID NO:53, a VH CDR2 comprising the amino acid sequence of SEQ ID NO:47, and a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49; or (iv) a VH region comprising a VH CDR1 comprising the amino acid sequence of SEQ ID NO:13, and a VH CDR2 comprising the amino acid sequence of SEQ ID NO:54, and a VH CDR3 comprising the amino acid sequence of SEQ ID NO:55; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:56; or (v) a VH region comprising a VH CDR1 comprising the amino acid sequence of SEQ ID NO:57, a VH CDR2 comprising the amino acid sequence of SEQ ID NO:58, and a VH CDR3 comprising the amino acid sequence of SEQ ID NO:59; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:60; or (vi) a VH region comprising a VH CDR1 comprising the amino acid sequence of SEQ ID NO:46, a VH CDR2 comprising the amino acid sequence of SEQ ID NO:61, and a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49.

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82), wherein Ab is an antibody that binds to 5T4. In some embodiments, the antibody further comprises a framework 1 (FR1), a framework 2 (FR2), a framework 3 (FR3) and/or a framework 4 (FR4) sequence as set forth in any one of SEQ ID NOs:25, 26, 44, 45, 62, and 63.

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82), wherein Ab is an antibody that binds to 5T4 and comprises a human framework sequence.

In some embodiments, a 5T4-ADC can be represented by Formula (IV), (V), (VI), (VI-1), (VIa-1), (VIb-1), (VI-2), (VIa-2), (VIb-2), (V-3), (VI-3), (V-4), (VI-4), (V-5), (VI-5), (V-6), (VI-6), (V-7), (VI-7), (V-8), (VI-8), (VIa-8), (VIb-8), (VI-11), (VI-21), (VI-81), (VIa-11), (VIa-21), (VIb-81), (VI-12), (VI-52), (VI-62), (VIb-12), (VIb-52), (VIb-62), (VIb-72), or (VIb-82), wherein Ab is an antibody that binds to 5T4. In some embodiments, the antibody comprises (i) a VH comprising the amino acid sequence of SEQ ID NO:25 and a VL comprising the amino acid sequence of SEQ ID NO:26; or (ii) a VH comprising the amino acid sequence of SEQ ID NO:44 and a VL comprising the amino acid sequence of SEQ ID NO:45; or (iii) a VH comprising the amino acid sequence of SEQ ID NO:62 and a VL comprising the amino acid sequence of SEQ ID NO:63.

### Antibody-Linker-Drug Conjugation

In some embodiments, the antibody can be modified before conjugation to the linker-drug. Modification of the antibody can produce a modified antibody that contains one or more reactive groups suitable for conjugation to the linker-drug.

In some embodiments, the antibody can be modified at one or more amino acid residues to provide one or more reactive groups suitable for conjugation to the linker-drug. For example, carbonyl groups introduced into a polypeptide can be selectively reacted with α-nucleophiles, such as aminooxy- and hydrazide-bearing compounds. Chemistries selective for carbonyl functional groups on a protein with enhanced kinetics, site selectivity and conjugate stability can result in improved bioconjugates.

In some embodiments, the antibody can be modified to include a reactive aldehyde group (e.g., a reactive aldehyde). A reactive aldehyde can be included in an “aldehyde tag” or “ald-tag,” which is meant to refer to an amino acid sequence derived from a sulfatase motif that has been converted by action of a formylglycine generating enzyme (FGE) to contain a 2-formylglycine residue (referred to herein as “fGly”). The fGly residue generated by an FGE is also referred to in the literature as a “formylglycine.” Stated differently, the term “aldehyde tag” is used herein to refer to an amino acid sequence comprising a “converted” sulfatase motif (i.e., a sulfatase motif in which the cysteine or the serine residue has been converted to fGly by action of an FGE, e.g., L(fGly)TPSR (SEQ ID NO:102)). A converted sulfatase motif can be derived from an amino acid sequence comprising an “unconverted” sulfatase motif (i.e., a sulfatase motif in which the cysteine or serine residues has not been converted to fGly by an FGE, but is capable of being converted, e.g., an unconverted sulfatase motif with the sequence: L(C/S)TPSR (SEQ ID NO:103)). “Conversion,” used in the context of action of a formylglycine generating enzyme (FGE) on a sulfatase motif, refers to biochemical modification of a cysteine or serine residue in a sulfatase motif to a formylglycine (fGly) residue (e.g., Cys to fGly, or Ser to fGly). As used herein, an aldehyde-tagged antibody refers to an antibody where a cysteine or serine residue has been modified to form a formylglycine residue. Additional aspects of aldehyde tags and uses thereof in site-specific protein modification are described in U.S. Pat. No. 7,985,783 and U.S. Application Publication No. 2011/0117621, the disclosures of which are each incorporated herein by reference.

Conversion of a polypeptide to include fGly can be accomplished by cell-based (in vivo) or cell-free methods (in vitro). Similarly, modification of a polypeptide to produce a polypeptide suitable for conjugation (e.g., modification to produce a polypeptide containing a reactive group suitable for conjugation) can be accomplished by cell-based (in vivo) or cell-free methods (in vitro).

The amino acid sequence of an antibody can be modified to include a sulfatase motif that contains a serine or cysteine residue that is capable of being converted (oxidized) to an FGly residue by action of an FGE either in vivo (e.g., at the time of translation of an aldehyde tag-containing protein in a cell) or in vitro (e.g., by contacting an aldehyde tag-containing protein with an FGE in a cell-free system). A 5T4 antibody used to generate a 5T4-ADC includes at least an Ig constant region, e.g., an Ig heavy chain constant region (e.g., at least a CH1 domain; at least a CH1 and a CH2 domain; a CH1, a CH2, and a CH3 domain; or a CH1, a CH2, a CH3, and a CH4 domain), or an Ig light chain constant region.

A sulfatase motif can be introduced into any convenient site in the antibody. As noted herein, in some embodiments, the extent of modification of the native amino acid sequence of the target polypeptide is minimized, to minimize the number of amino acid residues that are inserted, deleted, substituted (replaced), and/or added (e.g., to the N- or C-terminus). Minimizing the extent of amino acid sequence modification of the target antibody can minimize the impact such modifications can have upon antibody function and/or structure.

In some embodiments, an aldehyde-tagged antibody comprises an aldehyde-tagged Ig heavy chain constant region (e.g., at least a CH1 domain; at least a CH1 and a CH2 domain; a CH1, a CH2, and a CH3 domain; or a CH1, a CH2, a CH3, and a CH4 domain). The aldehyde-tagged Ig heavy chain constant region can include heavy chain constant region sequences of an IgA, IgM, IgD, IgE, IgG1, IgG2, IgG3, or IgG4 isotype heavy chain or any allotypic variant of same, e.g., human heavy chain constant region sequences or mouse heavy chain constant region sequences, a hybrid heavy chain constant region, a synthetic heavy chain constant region, or a consensus heavy chain constant region sequence, and the like, modified to include at least one sulfatase motif that can be modified by an FGE to generate an fGly-modified Ig polypeptide. Allotypic variants of Ig heavy chains are known in the art. See, e.g., Jefferis and Lefranc (2009) MAbs 1:4.

In some embodiments, an aldehyde-tagged antibody comprises an aldehyde-tagged Ig light chain constant region. The aldehyde-tagged Ig light chain constant region can include constant region sequences of a kappa light chain, a lambda light chain, e.g., kappa or lambda light chain constant regions, a hybrid light chain constant region, a synthetic light chain constant region, or a consensus light chain constant region sequence, and the like, modified to include at least one sulfatase motif that can be modified by an FGE to generate an fGly-modified antibody.

Alternatively, isolated, unmodified polypeptides can be isolated following recombinant production in a host cell lacking a suitable enzyme or by synthetic production. The isolated polypeptide can then be contacted with a suitable enzyme under conditions to provide for the desired modification of the polypeptide to include fGly. The polypeptide can be unfolded by methods known in the art (e.g., using heat, adjustment of pH, chaotropic agents, (e.g., urea, and the like), organic solvents (e.g., hydrocarbons: octane, benzene, chloroform), and the like) and the denatured protein contacted with a suitable enzyme. The modified polypeptide can then be refolded under suitable conditions.

In some embodiments, the modified antibody containing the fGly residue can be conjugated to a linker-drug by reaction of the fGly with a coupling moiety on the linker-drug, such as a hydrazinyl-pyrrolo coupling moiety as described herein. For example, an fGly-containing antibody can be isolated from a production source (e.g., recombinant host cell production, synthetic production), and contacted with a reactive partner-containing drug or other moiety (e.g., detectable label) under conditions suitable to provide for conjugation of the drug or other moiety to the 5T4 antibody. For example, the reactive partner-containing drug or other moiety can include a conjugating moiety (e.g., a hydrazinyl-pyrrolo coupling moiety as described herein). The hydrazinyl-pyrrolo-containing drug can be reacted with the antibody to produce a 5T4-ADC, as described herein.

A general scheme for coupling an antibody to a pyridazine-pyrrolo coupling moiety is shown below.

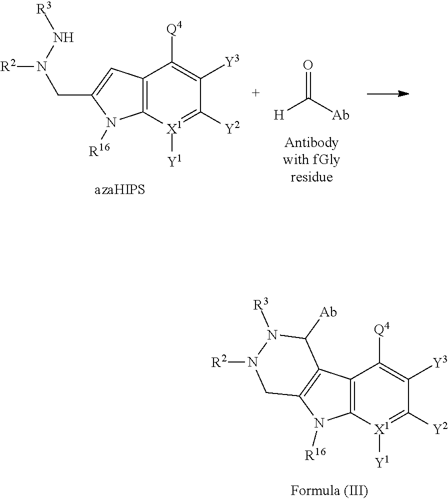

A hydrazinyl-pyrrolo coupling moiety, which can interchangeably be referred to herein as an aza-hydrazino-iso-Pictet-Spengler (azaHIPS) coupling moiety, upon conjugation to a formyl-glycine, forms a pyridazine-pyrrolo coupling moiety, such as that shown as Formula (III). A 5T4 antibody can include a 2-formylglycine residue (fGly) that is reacted with azaHIPS coupling moiety, thus conjugating the two together. To generate a 5T4-ADC, a drug can be coupled directly or indirectly (e.g., through a linker moiety) to the azaHIPS moiety at any location of the azaHIPs moiety (e.g., R¹⁶, Y¹, Y², Y³, or Q⁴). R² and R³ can each independently be any desired substituent, such as, but not limited to, hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl. R¹⁶, Y¹, Y², Y³, and Q⁴ can be as described herein, such as for Formula (I) or Formula (II).

Formula (I) below represents a hydrazinyl-pyrrolo coupling moiety that can be used to link an antibody that binds to 5T4 and a drug in any 5T4-ADC described herein.

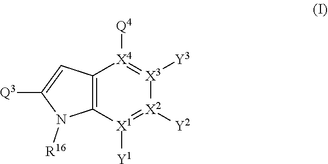

wherein:
one of R¹⁶, Y¹, Y², Y³, and Q⁴ is -L-W¹,
wherein one of Q³ and Q⁴ is —(CH₂)ₘNR³NHR²;
m is 0 or 1;
R² and R³ are each independently hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or R² and R³ are cyclically linked to form a 5- or 6-membered heterocyclyl;
if Q³ is not —(CH₂)ₘNR³NHR², then Q³ is Y⁴;
if Q⁴ is not -L-W¹ or —(CH₂)ₘNR³NHR², then Q⁴ is Y⁴;
X¹, X², X³, and X⁴ are each independently C, N, O, or S, wherein one of X¹, X², X³, and X⁴ is optionally absent;
Y¹, Y², Y³, if not -L-W¹, and Y⁴, are each independently hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or Y¹ and Y², Y² and Y³, or if Q⁴ is Y⁴, Y⁴ and Y³ may be optionally cyclically linked;
if R¹⁶ is not L-W¹, R¹⁶ is hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl;
L is a linker as described herein; and
W¹ is a drug.

Formula (I) has been previously described in WO 2015/081282, which is incorporated in its entirety herein by reference.

In some embodiments, a hydrazinyl-pyrrolo coupling moiety can be represented by Formula (II):

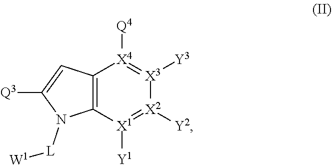

wherein:
one of Q³ and Q⁴ is [—(CH₂)ₘNR³NR²];
the other of Q³ and Q⁴ is Y⁴;
m is 0 or 1; and
R² and R³ are each independently hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or R² and R³ are cyclically linked to form a 5- or 6-membered heterocyclyl;
X¹, X², X³, and X⁴ are each independently C, N, O, or S, wherein one of X¹,
X², X³, and X⁴ is optionally absent;
Y¹, Y², Y³, and Y⁴ are each independently hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or Y¹ and Y², Y² and Y³, or if Q⁴ is Y⁴, Y³ and Y⁴ may optionally be cyclically linked;
L is a linker as described herein; and
W¹ is a drug.

Formula (II) has been previously described in WO 2015/081282 and in WO 2020/154437, each of which is incorporated herein by reference in its entirety.

In some embodiments, m is 0 or 1 and R² and R³ are each alkyl. For example, R² and R³ can each independently be C₁-C₁₀ alkyl (e.g., C₁-C₆ alkyl). In some embodiments, R² and R³ are each independently methyl, ethyl, n-propyl, iso-propyl, n-butyl, sec-butyl, isobutyl, or t-butyl. In some embodiments, at least one of R² and R³ is methyl. In some embodiments, both of R² and R³ are methyl. In some embodiments, each of X¹, X², X³, and X⁴ are each independently C or N. In some embodiments, X¹ is C. In some embodiments, X¹ is N. In some embodiments, X² is C or N. In some embodiments, X² is C. In some embodiments, X³ is C or N. In some embodiments, X³ is C. In some embodiments, X⁴ is C or N. In some embodiments, X⁴ is C. Various combinations of X¹, X², X³, and X⁴ are possible. For example, in some embodiments, each of X¹, X², X³, and X⁴ is C. In other embodiments, three of X¹, X², X³, and X⁴ are C and one of X¹, X², X³, and X⁴ is N. In some embodiments, each of Y¹, Y², Y³, and Y⁴ are hydrogen.

An exemplary scheme for making a 5T4-ADC is shown in Scheme A:

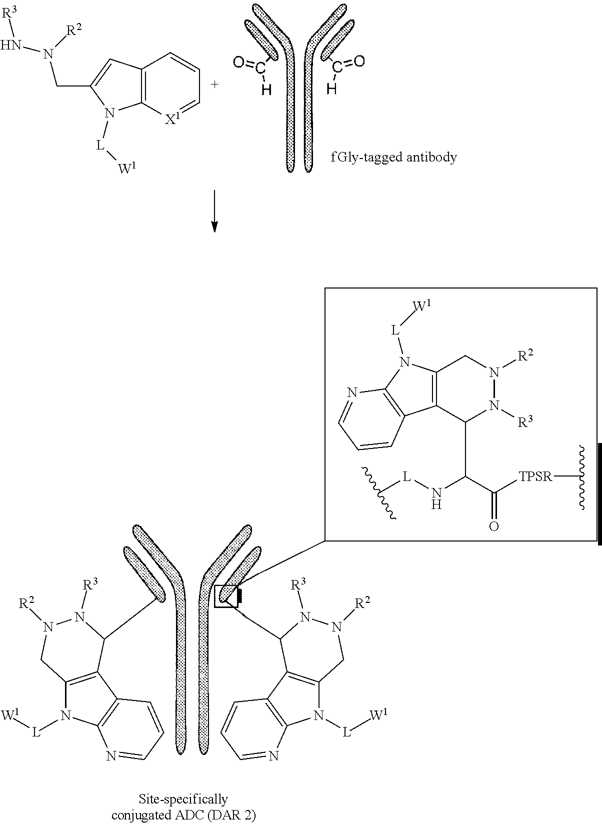

In Scheme A, the linker-drug is conjugated to an antibody that binds to 5T4. The 5T4 antibody has a recognition motif, for example, a L(C/S)TPSR (SEQ ID NO:103) recognition motif (e.g., such as in SEQ ID NO: 90, 94, 98) in the constant region of the light chain. The cysteine residue of a L(C/S)TPSR (SEQ ID NO:103) recognition motif can be converted to a formyl glycine (fGly) and subsequently conjugated to an azaHIPs moiety through a Pictet-Spengler reaction.

An exemplary scheme for making a 5T4-ADC is shown in Scheme B:

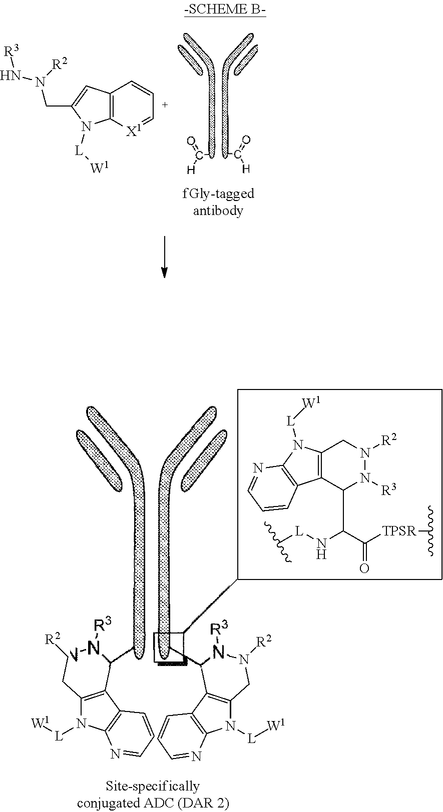

In Scheme B, the linker-drug is conjugated to an antibody that binds to 5T4. The 5T4 antibody has a recognition motif, for example, a L(C/S)TPSR (SEQ ID NO:103) recognition motif (e.g., SEQ ID NO: 90, 94, 98) in the constant region of the heavy chain. The cysteine residue of a L(C/S)TPSR (SEQ ID NO:103) recognition motif can be converted to a formyl glycine (fGly) and subsequently conjugated to an azaHIPs moiety through a Pictet-Spengler reaction.

In some embodiments, a 5T4-ADC includes a linker-drug conjugated to the C-terminus of each heavy chain constant region of an 5T4 antibody via a pyridazine-pyrrolo coupling moiety and can be characterized by a stoichiometric ratio of antibody to linker-drug of 1 to 4, 1 to 2, 1.75 to 2, or 1.9 to 2. In another embodiment, a 5T4-ADC includes a pyridazine-pyrrolo coupling moiety linked to only the C-terminus of one heavy chain constant region has a stoichiometric ratio between antibody and drug of 0.5 to 1, 0.75 to 1, or 0.9 to 1.

In some embodiments, a 5T4-ADC includes a linker-drug conjugated to an 5T4 antibody via a pyridazine-pyrrolo coupling moiety, wherein the linker-drug comprises maytansine conjugated to the pyridazine-pyrrolo coupling moiety via linker (L-1) as shown in Scheme C below:

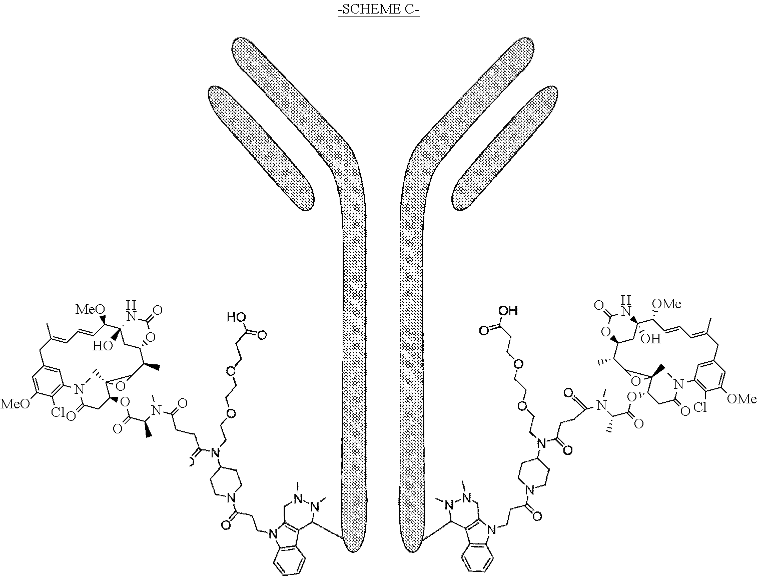 [SMILES: CCC(=O)N(C)[C@@H](C)C(=O)O[C@H]1CC(=O)N(C)c2cc(cc(OC)c2Cl)C/C(C)=C/C=C/[C@@H](OC)[C@]2(O)C[C@H](OC(=O)N2)[C@@H](C)C2O[C@]21C ; C=C(C)N(C)[C@@H](C)C(=O)O[C@H]1CC(=O)N(C)c2cc(cc(OC)c2Cl)C/C(C)=C/C=C/[C@@H](OC)[C@]2(O)C[C@H](OC(=O)N2)[C@@H](C)C2O[C@]21C]

In some embodiments, the 5T4 antibody has a recognition motif, for example, a L(C/S)TPSR (SEQ ID NO:103) recognition motif (e.g., SEQ ID NO: 90, 94, 98), in the constant region of the heavy chain. The cysteine residue of a L(C/S)TPSR (SEQ ID NO:103) recognition motif can be converted to a formyl glycine (fGly) and subsequently conjugated via a Pictet-Spengler reaction to an azaHIPs moiety conjugated to MMAE via linker (L-1).

In some embodiments, a 5T4-ADC includes a linker-drug conjugated to an 5T4 antibody via a pyridazine-pyrrolo coupling moiety, wherein the linker-drug comprises MMAE conjugated to the pyridazine-pyrrolo coupling moiety via linker (L-2) as shown in Scheme D below:

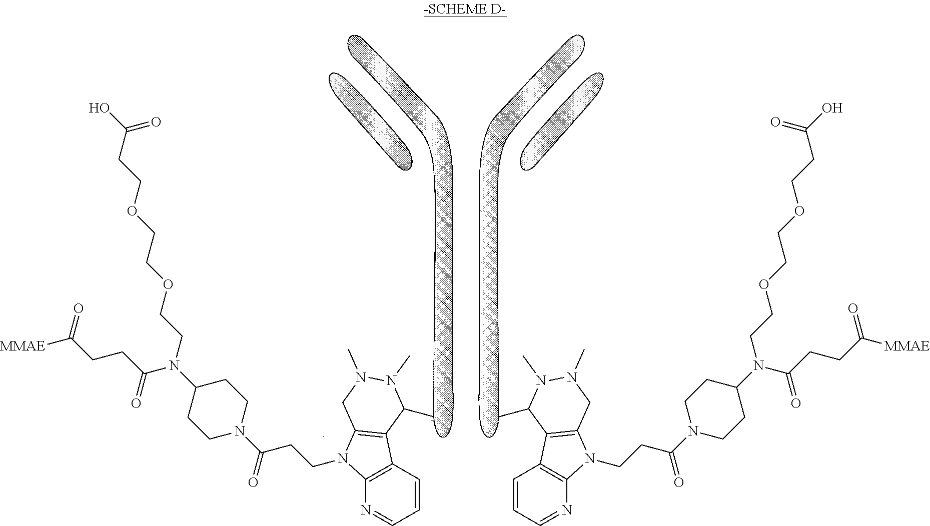

In some embodiments, the 5T4 antibody has a recognition motif, for example, a L(C/S)TPSR (SEQ ID NO:103) recognition motif, in the constant region of the heavy chain. The cysteine residue of a L(C/S)TPSR (SEQ ID NO:103) recognition motif can be converted to a formyl glycine (fGly) and subsequently conjugated via a Pictet-Spengler reaction to an azaHIPs moiety conjugated to MMAE via linker (L-2).

In some embodiments, a 5T4-ADC includes a linker-drug conjugated to an 5T4 antibody via a pyridazine-pyrrolo coupling moiety, wherein the linker-drug comprises MMAE conjugated to the pyridazine-pyrrolo coupling moiety via linker (L-7) as shown in Scheme E below:

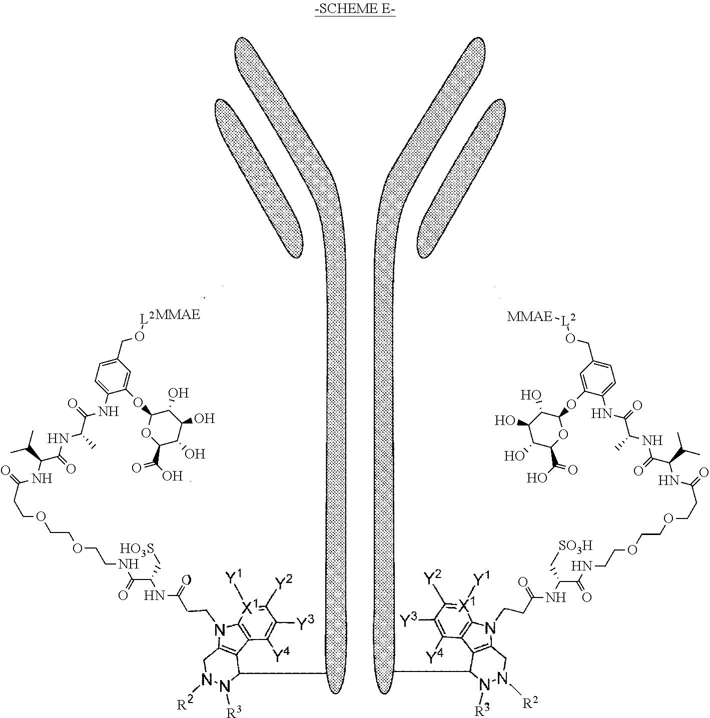

In some embodiments, the 5T4 antibody has a recognition motif, for example, a L(C/S)TPSR (SEQ ID NO:103) recognition motif (e.g., SEQ ID NO: 90, 94, 98), in the constant region of the heavy chain. The cysteine residue of a L(C/S)TPSR (SEQ ID NO:103) recognition motif can be converted to a formyl glycine (fGly) and subsequently conjugated via a Pictet-Spengler reaction to an azaHIPs moiety conjugated to MMAE via linker (L-7).

In some embodiments, a 5T4-ADC includes a linker-drug conjugated to an 5T4 antibody via a pyridazine-pyrrolo coupling moiety, wherein the linker-drug comprises MMAE conjugated to the pyridazine-pyrrolo coupling moiety via linker (L-8) as shown in Scheme F below:

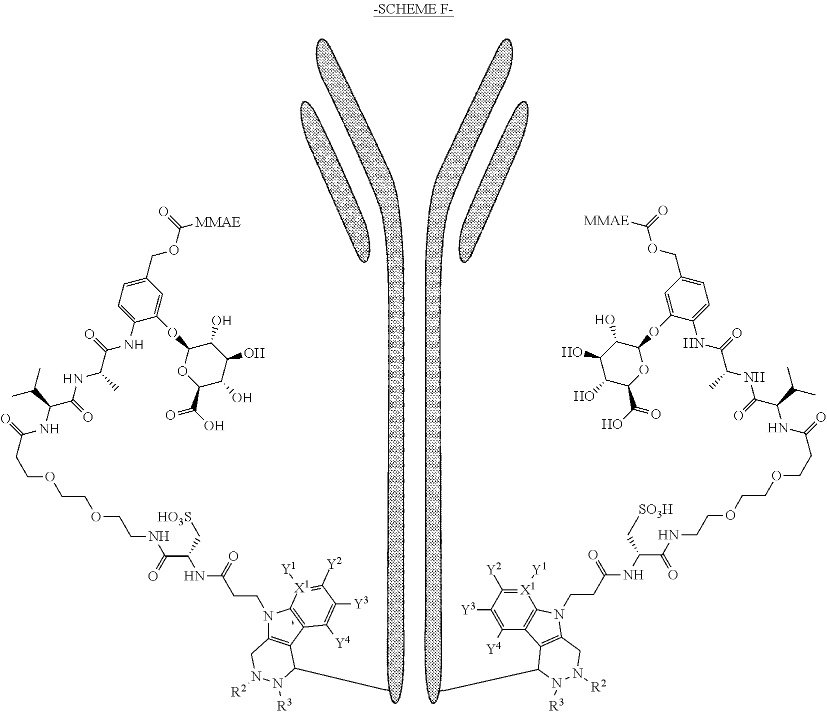

In some embodiments, the antibody that binds to 5T4 has a recognition motif, for example, a L(C/S)TPSR (SEQ ID NO:103) recognition motif (e.g., SEQ ID NO: 90, 94, 98), in the constant region of the heavy chain. The cysteine residue of a L(C/S)TPSR (SEQ ID NO:103) recognition motif can be converted to a formyl glycine (fGly) and subsequently conjugated via a Pictet-Spengler reaction to an azaHIPs moiety conjugated to MMAE via linker (L-8).

### Formulations & Compositions

5T4-ADCs can be included in a pharmaceutical composition for administration, e.g., to a subject for treating a disease, disorder, or condition. A pharmaceutical composition can comprise 5T4-ADCs which, on average, can exhibit a drug-to-antibody ratio (“DAR”) of about 1 to about 2. In other words, each antibody is conjugated to one or two linker-drug conjugates. Methods to determine DAR are well known to the skilled person and include methods using Reverse Phase Chromatography, or HPLC-MS.

For example, in any embodiment, a pharmaceutical composition comprising a 5T4-ADC can exhibit a DAR of about 1 to about 1.9 or about 1 to about 1.8, about 1 to about 1.7, about 1.5 to about 1.9, about 1.5 to about 1.8, about 1.5 to about 1.7, about 1.7 to about 1.9, about 1.7 to about 1.8, or about 1.8 to about 1.9. In some embodiments, a pharmaceutical composition comprises a 5T4-ADC comprising an antibody that binds to 5T4 as described herein, including mAbA4, mAbA15, and mAbA17, as described herein, such as in any one of Tables 1-3, and a pharmaceutically acceptable excipient. In some embodiments, a pharmaceutical composition comprises a 5T4-ADC of Formula (IV), (V), or (VI), and L is chosen from linkers (L-1), (L-2), (L-3), (L-4), (L-5), (L-6), (L-7), and (L-8). In some embodiments, a pharmaceutical composition comprises a 5T4-ADC comprising MMAE, MMAF, maytansine, or any derivative thereof. In some embodiments, a pharmaceutical composition comprises a 5T4-ADC of Formula (VI-1), (VIa-1), or (VIb-1) and a pharmaceutically acceptable excipient. In some embodiments, a pharmaceutical composition comprises a 5T4-ADC of Formula (VI-2), (VIa-2), or (VIb-2) and a pharmaceutically acceptable excipient. In some embodiments, a pharmaceutical composition comprises a 5T4-ADC of Formula (V-3), (VI-3), (V-4), (VI-4), (V-5), (VI-5), (V-6), (VI-6), (V-7), (VI-7), (V-8), (VI-8), (VIa-8), or (VIb-8) and a pharmaceutically acceptable excipient. In some embodiments, a pharmaceutical composition comprises a 5T4-ADC of Formula (VI-11), (VI-12), (VIa-11), or (VIb-12) and a pharmaceutically acceptable excipient. In some embodiments, a pharmaceutical composition comprises a 5T4-ADC of Formula (VI-21) or (VIa-21) and a pharmaceutically acceptable excipient. In some embodiments, a pharmaceutical composition comprises a 5T4-ADC of Formula (VI-52), (VI-62), (VI-81), (VIb-81), (VIb-52), (VIb-62), (VIb-72), or (VIb-82) and a pharmaceutically acceptable excipient. In some embodiments, a pharmaceutical composition comprises a 5T4-ADC of mAbA4-VIa-21 (i.e., a linker-drug of Formula VIa-21 conjugated to antibody mAbA4), mAbA15-VIa-21 (i.e., a linker-drug of Formula VIa-21 conjugated to antibody mAbA15), mAbA15-VIb-82 (i.e., a linker-drug of Formula VIb-82 conjugated to antibody mAbA15), or mAbA17-VIb-82 (i.e., a linker-drug of Formula VIb-82 conjugated to antibody mAbA17) and a pharmaceutically acceptable excipient.

A 5T4-ADC can be formulated in any of a variety of different ways. A 5T4-ADC of the present disclosure can be provided in any suitable form, e.g., in the form of a pharmaceutically acceptable salt, and can be formulated for any suitable route of administration, e.g., oral, topical, or parenteral administration. Where a 5T4-ADC is provided as a liquid injectable (such as in those embodiments where they it can be administered intravenously or directly into a tissue), a 5T4-ADC can be provided as a ready-to-use dosage form, or as a storage-stable powder for reconstitution or liquid composed of pharmaceutically acceptable excipients and excipients.

Methods for formulating a 5T4-ADC can be adapted from those available in the art. For example, 5T4-ADCs can be provided in a pharmaceutical composition comprising an effective amount of a 5T4-ADC and a pharmaceutically acceptable excipient (e.g., saline). The pharmaceutical composition can optionally include other additives (e.g., buffers, stabilizers, preservatives, and the like). In some embodiments, the formulations are suitable for administration to a mammal, such as those that are suitable for administration to a human.

Also provided herein are pharmaceutical compositions that contain an effective amount of a 5T4-ADC described herein and a pharmaceutically acceptable excipient. In some embodiments, a 5T4-ADC comprises an antibody that binds to 5T4 as described herein, including mAbA4, mAbA15, and mAbA17, as described in any one of Tables 1-3. In some embodiments, a pharmaceutical composition comprises an effective amount of a 5T4-ADC of Formula (IV), (V), or (VI), and L is chosen from linkers (L-1), (L-2), (L-3), (L-4), (L-5), (L-6), (L-7), or (L-8). In some embodiments, a pharmaceutical composition comprises an effective amount of a 5T4-ADC comprising MMAE, MMAF, maytansine, or any derivative thereof. In some embodiments, a pharmaceutical composition comprises an effective amount of a 5T4-ADC of Formula (VI-1), (VIa-1), or (VIb-1) and a pharmaceutically acceptable excipient. In some embodiments, a pharmaceutical composition comprises an effective amount of a 5T4-ADC of Formula (VI-2), (VIa-2), or (VIb-2) and a pharmaceutically acceptable excipient. In some embodiments, a pharmaceutical composition comprises an effective amount of a 5T4-ADC of Formula (V-3), (VI-3), (V-4), (VI-4), (V-5), (VI-5), (V-6), (VI-6), (V-7), (VI-7), (V-8), (VI-8), (VIa-8), or (VIb-8) and a pharmaceutically acceptable excipient. In some embodiments, a pharmaceutical composition comprises an effective amount of a 5T4-ADC of Formula (VI-11), (VI-12), (VIa-11), or (VIb-12) and a pharmaceutically acceptable excipient. In some embodiments, a pharmaceutical composition comprises an effective amount of a 5T4-ADC of Formula (VI-21) or (VIa-21) and a pharmaceutically acceptable excipient. In some embodiments, a pharmaceutical composition comprises an effective amount of a 5T4-ADC of Formula (VI-52), (VI-62), (VI-81), (VIb-81), (VIb-52), (VIb-62), (VIb-72), or (VIb-82) and a pharmaceutically acceptable excipient. In some embodiments, a pharmaceutical composition comprises an effective amount of a 5T4-ADC of mAbA4-VIa-21, mAbA15-VIa-21, mAbA15-VIb-82, or mAbA17-VIb-82 and a pharmaceutically acceptable excipient.

The pharmaceutically acceptable excipient can be one or more compatible solid or liquid fillers, diluents, other excipients, or encapsulating substances that are suitable for administration into a human or veterinary subject (e.g., a physiologically acceptable and/or pharmacologically acceptable). The pharmaceutically acceptable excipient can be co-mingled with one or more of the active components, e.g., a hybrid molecule, and with each other, when more than one pharmaceutically acceptable excipient is present in the pharmaceutical composition, in a manner so as not to substantially impair the desired pharmaceutical efficacy. Pharmaceutically acceptable materials typically are capable of administration to a subject without the production of significant undesirable physiological effects such as nausea, dizziness, rash, or gastric upset. It is, for example, desirable for a composition comprising a pharmaceutically acceptable excipient not to be immunogenic when administered to a human subject for therapeutic purposes.

Pharmaceutical compositions of the invention can additionally contain suitable buffering agents, including, for example, acetic acid in a salt, citric acid in a salt, boric acid in a salt, and phosphoric acid in a salt. The pharmaceutical compositions can also optionally contain suitable preservatives, such as benzalkonium chloride, chlorobutanol, parabens, and thimerosal. Pharmaceutical compositions of the invention can be presented in unit dosage form and can be prepared by any suitable method, many of which are well known in the art of pharmacy. Such methods include the step of bringing the antibody or antigen-binding fragment of the invention into association with an excipient that constitutes one or more accessory ingredients. In general, the pharmaceutical composition is prepared by uniformly and intimately bringing the active agent into association with a liquid excipient, a finely divided solid excipient, or both, and then, if necessary, shaping the product.

A composition suitable for parenteral administration conveniently comprises a sterile aqueous preparation of the composition, which preferably is isotonic with the blood of the recipient. This aqueous preparation can be formulated of known methods using suitable dispersing or wetting agents and suspending agents. The sterile injectable preparation also can be a sterile injectable solution or suspension in a non-toxic parenterally acceptable diluent or solvent, for example, as a solution in 1,3-butane diol. Among the acceptable vehicles and solvents that can be employed are water, Ringer's solution, and isotonic sodium chloride solution. In addition, sterile, fixed oils are conventionally employed as a solvent or suspending medium. For this purpose, any bland fixed oil can be employed, such as synthetic mono- or di-glycerides. In addition, fatty acids such as oleic acid can be used in the preparation of injectables. Excipient formulations suitable for oral, subcutaneous, intravenous, intramuscular, and the like, administrations can be found in Remington's Pharmaceutical Sciences, Mack Publishing Co., Easton, PA.

Preparation of pharmaceutical compositions of the invention and their various routes of administration can be carried out in accordance with methods well known in the art. The delivery systems useful in the context of the invention include time-released, delayed release, and sustained release delivery systems such that the delivery of the composition occurs prior to, and with sufficient time to cause, sensitization of the site to be treated. The composition can be used in conjunction with other therapeutic agents or therapies. Such systems can avoid repeated administrations of the composition, thereby increasing convenience to the subject and the physician, and can be particularly suitable for some compositions of the invention.

Many types of release delivery systems are available and known to those of ordinary skill in the art. Suitable release delivery systems include polymer base systems such as poly(lactide-glycolide), copolyoxalates, polycaprolactones, polyesteramides, polyorthoesters, polyhydroxybutyric acid, and polyanhydrides.

Microcapsules of the foregoing polymers containing drugs are described in, for example, U.S. Pat. No. 5,075,109. Delivery systems also include non-polymer systems that are lipids such as sterols such as cholesterol, cholesterol esters, and fatty acids or neutral fats such as mono-di- and triglycerides; hydrogel release systems; sylastic systems; peptide-based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Generally, a 5T4-ADC or pharmaceutical composition is suitably packaged, e.g., in a vial, pouch, ampoule, and/or any container appropriate for a therapeutic method. Components can be provided as concentrates (including lyophilized compositions), which can be further diluted prior to use, or they can be provided at the concentration of use. For use of a 5T4-ADC in vivo, single dosages can be provided in sterilized containers having the desired amount and concentration of components.

### Methods of Treatment

Also provided herein are methods of treating, preventing, or alleviating a 5T4-mediated disease, disorder, or condition, including one or more symptoms of the 5T4-mediated disease, disorder, or condition with a 5T4-ADC comprising an antibody that binds to 5T4, such as human 5T4, and a drug conjugated directly or indirectly thereto. Also provided herein are methods of killing tumor cells with a 5T4-ADC comprising an antibody that binds to 5T4, such as human 5T4, and a drug conjugated directly or indirectly thereto.

The antibody that binds to 5T4 can include any as described herein, such as mAbA4, mAbA15, and mAbA17, as described in any one of Tables 1-3. In some embodiments, a method of killing a tumor cell comprises contacting the tumor cell with a 5T4-ADC of Formula (IV), (V), or (VI), wherein L is linker (L-1), (L-2), (L-3), (L-4), (L-5), (L-6), (L-7), or (L-8). In some embodiments, a method of killing a tumor cell comprises contacting the tumor cell with a 5T4-ADC comprising MMAE, MMAF, maytansine, or any derivative thereof. In some embodiments, a method of killing a tumor cell comprises contacting the tumor cell with a 5T4-ADC of Formula (VI-1), (VIa-1), or (VIb-1). In some embodiments, a method of killing a tumor cell comprises contacting the tumor cell with a 5T4-ADC of Formula (VI-2), (VIa-2), or (VIb-2). In some embodiments, a method of killing a tumor cell comprises contacting the tumor cell with a 5T4-ADC of Formula (V-3), (VI-3), (V-4), (VI-4), (V-5), (VI-5), (V-6), (VI-6), (V-7), (VI-7), (V-8), (VI-8), (VIa-8), or (VIb-8). In some embodiments, a method of killing a tumor cell comprises contacting the tumor cell with a 5T4-ADC of Formula (VI-11), (VI-12), (VIa-11), or (VIb-12) and a pharmaceutically acceptable excipient. In some embodiments, a method of killing a tumor cell comprises contacting the tumor cell with a 5T4-ADC of Formula (VI-21) or (VIa-21). In some embodiments, a method of killing a tumor cell comprises contacting the tumor cell with a 5T4-ADC of Formula (VI-52), (VI-62), (VI-81), (VIb-81), (VIb-52), (VIb-62), (VIb-72), or (VIb-82). In some embodiments, a method of killing a tumor cell comprises contacting the tumor cell with a 5T4-ADC of mAbA4-VIa-21, mAbA15-VIa-21, mAbA15-VIb-82, or mAbA17-VIb-82. In some embodiments, the contacting is in vivo or in vitro.

In any embodiment, the method of contacting the tumor cell with a 5T4-ADC can comprise contacting the tumor cell with a composition comprising the 5T4-ADC and one or more a pharmaceutically acceptable excipients. In some embodiments, the 5T4-ADC in the composition can be characterized by a DAR of about 1 to about 10, about 1 to about 4, about 1 to about 2, about 1.5 to about 2, or about 1.75 to about 1.9.

Also provided herein is a method of treating a cancer in a subject in need thereof comprising administering an effective amount of a 5T4-ADC to the subject, wherein the 5T4-ADC comprises an antibody that binds to 5T4 and a drug conjugated thereto via a linker, as described herein. In some embodiments, the antibody that binds to 5T4 is mAbA4, mAbA15, or mAbA17, as described herein, such as in any one of Tables 1-3.

In some embodiments, a method of treating cancer in a subject in need thereof comprises administering to the subject an effective amount of a 5T4-ADC of Formula (IV), (V), or (VI), wherein L is (L-1), (L-2), (L-3), (L-4), (L-5), (L-6), (L-7), or (L-8). In some embodiments, a method of treating cancer in a subject in need thereof comprises administering to the subject an effective amount of a 5T4-ADC, as described herein, comprising MMAE, MMAF, maytansine, or any derivative thereof.

In some embodiments, a method of treating cancer in a subject in need thereof comprises administering to the subject an effective amount of a 5T4-ADC of Formula (VI-1), (VIa-1), or (VIb-1). In some embodiments, a method of treating cancer in a subject in need thereof comprises administering to the subject an effective amount of a 5T4-ADC of Formula (VI-2), (VIa-2), or (VIb-2). In some embodiments, a method of treating cancer in a subject in need thereof comprises to the subject administering an effective amount of a 5T4-ADC of Formula (V-3), (VI-3), (V-4), (VI-4), (V-5), (VI-5), (V-6), (VI-6), (V-7), (VI-7), (V-8), (VI-8), (VIa-8), or (VIb-8). In some embodiments, a method of treating cancer in a subject in need thereof comprises administering to the subject an effective amount of a 5T4-ADC of Formula (VI-11), (VI-12), (VIa-11), or (VIb-12) and a pharmaceutically acceptable excipient. In some embodiments, a method of treating cancer in a subject in need thereof comprises administering to the subject an effective amount of a 5T4-ADC of Formula (VI-21) or (VIa-21). In some embodiments, a method of treating cancer in a subject in need thereof comprises administering to the subject an effective amount of a 5T4-ADC of Formula (VI-52), (VI-62), (VI-81), (VIb-81), (VIb-52), (VIb-62), (VIb-72), or (VIb-82). In some embodiments, a method of treating cancer in a subject in need thereof comprises administering to the subject an effective amount of a 5T4-ADC of mAbA4-VIa-21, mAbA15-VIa-21, mAbA15-VIb-82, or mAbA17-VIb-82.

In any embodiment, the method of treating cancer in a subject in need thereof can comprising administering to the subject a pharmaceutical composition comprising the 5T4-ADC and a pharmaceutically acceptable excipient. In some embodiments, the 5T4-ADC in the pharmaceutical composition can be characterized by a DAR of about 1 to about 10, about 1 to about 4, about 1 to about 2, about 1.5 to about 2, or about 1.75 to about 1.9.

In some embodiments, the method of treating cancer in a subject in need thereof is effective to enhance the removal of the cancer cells, for example, tumor cells, in the subject.

“Enhancing” the removal of cancer or tumor cells does not require a 100% enhancement of removal. Any enhancement in the rate of removal is contemplated. Similarly, “modulating” tumor growth refers to reducing the size of the tumor, slowing tumor growth, or inhibiting an increase in the size of an existing tumor. Complete abolition of a tumor is not required; any decrease in tumor size or slowing of tumor growth constitutes a beneficial biological effect in a subject. In this regard, tumor cell removal can be enhanced by, for example, at least about 5%, at least about 10% or at least about 20% compared to levels of removal observed in the absence of the method (e.g., in a biologically matched control subject or specimen that is not exposed to the agent of the method). The effect is detected by, for example, a reduction in tumor size, a decrease or maintenance of the levels of tumor markers, or reduction or maintenance of a tumor cell population. In some embodiments, removal of tumor cells is enhanced by, for example, at least about 30%, at least about 40%, at least about 50%, at least about 60%, at least about 70%, at least about 80%, at least about 90%, or more (about 100%) compared to the removal of tumor cells in the absence of a 5T4 binding agent (e.g., an antibody) of the method.

The present disclosure also provides a method of modulating (e.g., inhibiting, reducing, preventing) tumor growth in a subject in need thereof, the method comprising administering to the subject a 5T4-ADC comprising an antibody that binds to 5T4 and a drug linked thereto via a linker, as described herein, in an amount effective to modulate the tumor growth in the subject. In some embodiments, a method of modulating tumor growth in a subject in need thereof comprising administering an effective amount of a 5T4-ADC comprising an antibody that binds to 5T4 and a drug conjugated thereto via a linker, as described herein. In some embodiments, the antibody that binds to 5T4 is mAbA4, mAbA15, or mAbA17, as described in any one of Tables 1-3. In some embodiments, a method of modulating tumor growth in a subject in need thereof comprises administering to the subject an effective amount of a 5T4-ADC of Formula (IV), (V), or (VI), wherein L is (L-1), (L-2), (L-3), (L-4), (L-5), (L-6), (L-7), or (L-8). In some embodiments, a method of modulating tumor growth in a subject in need thereof comprises administering to the subject an effective amount of a 5T4-ADC comprising MMAE, MMAF, maytansine, or any derivative thereof. In some embodiments, a method of modulating tumor growth in a subject in need thereof comprises administering to the subject an effective amount of a 5T4-ADC of Formula (VI-1), (VIa-1), or (VIb-1). In some embodiments, a method of modulating tumor growth in a subject in need thereof comprises administering to the subject an effective amount of a 5T4-ADC of Formula (VI-2), (VIa-2), or (VIb-2). In some embodiments, a method of modulating tumor growth in a subject in need thereof comprises administering to the subject an effective amount of a 5T4-ADC of Formula (V-3), (VI-3), (V-4), (VI-4), (V-5), (VI-5), (V-6), (VI-6), (V-7), (VI-7), (V-8), (VI-8), (VIa-8), or (VIb-8). In some embodiments, a method of modulating tumor growth in a subject in need thereof comprises administering to the subject an effective amount of a 5T4-ADC of Formula (VI-11), (VI-12), (VIa-11), or (VIb-12). In some embodiments, a method of modulating tumor growth in a subject in need thereof comprises administering to the subject an effective amount of a 5T4-ADC of Formula (VI-21) or (VIa-21). In some embodiments, a method of modulating tumor growth in a subject in need thereof comprises administering to the subject an effective amount of a 5T4-ADC of Formula (VI-52), (VI-62), (VI-81), (VIb-81), (VIb-52), (VIb-62), (VIb-72), or (VIb-82). In some embodiments, a method of modulating tumor growth in a subject in need thereof comprises administering to the subject an effective amount of a 5T4-ADC of mAbA4-VIa-21, mAbA15-VIa-21, mAbA15-VIb-82, or mAbA17-VIb-82.

In any embodiment, the method of modulating tumor growth in a subject in need thereof can comprising administering to the subject a pharmaceutical composition comprising the 5T4-ADC and one or more pharmaceutically acceptable excipients. In some embodiments, the 5T4-ADC in the pharmaceutical composition can be characterized by a DAR of about 1 to about 10, about 1 to about 4, about 1 to about 2, about 1.5 to about 2, or about 1.75 to about 1.9.

In some embodiments, the cancer and tumor cells described herein that may be treated and/or killed by the methods described herein express 5T4, for example, as on a surface of the cancer or tumor cell. In some embodiments, a tumor or cancer cell may overexpress 5T4. As used herein, the term “overexpress” means to transcribe and translate more genetic product than normal (such as in normal cells), the process of which is often a characteristic of cancer cells.

Examples of cancers that can be treated with a 5T4-ADC described herein include, but are not limited to: breast cancer, colon cancer, renal cancer, lung cancer, squamous cell myeloid leukemia, hemangiomas, melanomas, astrocytomas, and glioblastomas as well as other cellular-proliferative disease states, including but not limited to proliferative diseases of the heart, such as sarcoma (angiosarcoma, fibrosarcoma, rhabdomyosarcoma, liposarcoma), myxoma, rhabdomyoma, fibroma, lipoma, and teratoma; of the lung, such as bronchogenic carcinoma (e.g., squamous cell, undifferentiated small cell, undifferentiated large cell, adenocarcinoma), alveolar (e.g., bronchiolar) carcinoma, bronchial adenoma, sarcoma, lymphoma, chondromatous hanlartoma, inesothelioma; of the gastrointestinal system such as of the esophagus (e.g., squamous cell carcinoma, adenocarcinoma, leiomyosarcoma, lymphoma), of the stomach (e.g., carcinoma, lymphoma, leiomyosarcoma), of the pancreas (e.g., ductal adenocarcinoma, insulinorna, glucagonoma, gastrinoma, carcinoid tumors, vipoma), of the small bowel (e.g., adenocarcinoma, lymphoma, carcinoid tumors, Karposi's sarcoma, leiomyoma, hemangioma, lipoma, neurofibroma, fibroma), of the large bowel (e.g., adenocarcinoma, tubular adenoma, villous adenoma, hamartoma, leiomyoma); of the Genitourinary tract such as of the kidney (e.g., adenocarcinoma, Wilm's tumor (e.g., nephroblastoma) lymphoma, leukemia, renal cell carcinoma), of the bladder and urethra (e.g., squamous cell carcinoma, transitional cell carcinoma, adenocarcinoma), of the prostate (e.g., adenocarcinoma, sarcoma, small cell carcinoma of the prostate), of the testis (e.g., seminoma, teratoma, embryonal carcinoma, teratocarcinoma, choriocarcinoma, sarcoma, interstitial cell carcinoma, fibroma, fibroadenoma, adenomatoid tumors, lipoma); of the liver: hepatoma (e.g., hepatocellular carcinoma), cholangiocarcinoma, hepatoblastoma, angiosarcoma, hepatocellular adenoma, hemangioma; proliferative diseases of the bone such as osteogenic sarcoma (e.g., osteosarcoma), fibrosarcoma, malignant fibrous histiocytoma, chondrosarcoma, Ewing's sarcoma, malignant lymphoma (e.g., reticulum cell sarcoma), malignant giant cell tumor chordoma, osteochronfroma (e.g., osteocartilaginous exostoses), benign chondroma, chondroblastoma, chondromyxofibroma, osteoid osteoma, and giant cell tumors; of the nervous system such as of the skull (e.g., osteoma, hemangioma, granuloma, xanthoma, osteitis defornians), meninges (e.g., meningioma, meningiosarcoma, gliomatosis), brain (e.g., astrocytoma, medulloblastoma, glioma, ependymoma, germinoma (e.g., pinealoma), glioblastoma multiform, oligodendroglioma, schwannoma, retinoblastoma, congenital tumors), spinal cord neurofibroma, meningioma, glioma, sarcoma); of the gynecological system such as of the uterus (e.g., endometrial carcinoma), cervix (e.g., cervical carcinoma, pre-tumor cervical dysplasia), ovaries (e.g., ovarian carcinoma) serous cystadenocarcinoma, mucinous cystadenocarcinoma, unclassified carcinoma, granulosa-thecal cell tumors, Sertoli-Leydig cell tumors, dysgerminoma, malignant teratoma), vulva (e.g., squamous cell carcinoma, intraepithelial carcinoma, adenocarcinoma, fibrosarcoma, melanoma), vagina (e.g., clear cell carcinoma, squamous cell carcinoma, botryoid sarcoma (embryonal rhabdomyosarcoma], fallopian tubes (e.g., carcinoma); of the blood (e.g., myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (e.g., malignant lymphoma); of the skin such as malignant melanoma, basal cell carcinoma, squamous cell carcinoma, Karposi's sarcoma, moles dysplastic nevi, lipoma, angioma, dermatofibroma, keloids, psoriasis; of the adrenal glands such as neuroblastoma; and of the thyroid including medullary thyroid cancer. In some embodiments, the tumor or cancer is a relapsed tumor or cancer. In some embodiments, the tumor or cancer is a metastatic tumor or cancer. In some embodiments, the tumor or cancer is a primary tumor or cancer. Additionally or alternatively, the tumor or cancer is resistant to a chemotherapy or other non-5T4-target anti-cancer therapy.

Additionally, a 5T4-ADC comprising an antibody that binds to 5T4 can be used to alleviate or reduce side effects associated with cancer such as, for example, bone deterioration, vertebral collapse, and paralysis. In one embodiment, the subject suffers from or is at risk of suffering from bone metastases and a 5T4-ADC comprising an antibody that binds to 5T4 is administered in an amount to reduce deterioration of surrounding bone. Accordingly, in some embodiments, a 5T4-ADC comprising an antibody that binds to 5T4 prevents bone deterioration due to bone metastases, wherein tumor cell proliferation is or is not reduced. In some embodiments, a 5T4 binding agent (e.g., an antibody or ADC) a 5T4-ADC comprising an antibody that binds to 5T4 both prevents bone deterioration due to bone metastases and reduces tumor cell proliferation. In general, the effect on tumor cell proliferation (e.g., inhibition of proliferation or no effect on proliferation) depends on the microenvironment of a particular metastasis. For example, proliferation of metastases located in microenvironments with substantial amounts of type 1 collagen can be inhibited. In contrast, proliferation of metastases located in microenvironments lacking substantial amounts of type 1 collagen cannot be inhibited, yet bone deterioration near the metastasis is reduced or prevented.

Therefore, the present disclosure also provides a method of alleviating or reducing side effects associated with cancer comprising administering an effective amount of a 5T4-ADC to a subject in need thereof. In some embodiments, a method of alleviating or reducing side effects associated with cancer comprises administering an effective amount of a 5T4-ADC comprising an antibody that binds to 5T4 and a drug conjugated thereto via a linker, as described herein. In some embodiments, the antibody that binds to 5T4 is mAbA4, mAbA15, or mAbA17, as described in any one of Tables 1-3. In some embodiments, a method of alleviating or reducing side effects associated with cancer comprises administering to the subject an effective amount of a 5T4-ADC of Formula (IV), (V), or (VI), wherein L is (L-1), (L-2), (L-3), (L-4), (L-5), (L-6), (L-7), or (L-8). In some embodiments, a method of alleviating or reducing side effects associated with cancer comprises administering to the subject an effective amount of a 5T4-ADC comprising MMAE, MMAF, maytansine, or any derivative thereof. In some embodiments, a method of alleviating or reducing side effects associated with cancer comprises administering to the subject an effective amount of a 5T4-ADC of Formula (VI-1), (VIa-1), or (VIb-1). In some embodiments, a method of alleviating or reducing side effects associated with cancer comprises administering to the subject an effective amount of a 5T4-ADC of Formula (VI-2), (VIa-2), or (VIb-2). In some embodiments, a method of alleviating or reducing side effects associated with cancer comprises administering to the subject an effective amount of a 5T4-ADC of Formula (V-3), (VI-3), (V-4), (VI-4), (V-5), (VI-5), (V-6), (VI-6), (V-7), (VI-7), (V-8), (VI-8), (VIa-8), or (VIb-8). In some embodiments, a method of alleviating or reducing side effects associated with cancer comprises administering to the subject an effective amount of a 5T4-ADC of (VI-11), (VI-12), (VIa-11), or (VIb-12). In some embodiments, a method of alleviating or reducing side effects associated with cancer comprises administering to the subject an effective amount of a 5T4-ADC of Formula (VI-21) and (VIa-21). In some embodiments, a method of alleviating or reducing side effects associated with cancer comprises administering to the subject an effective amount of a 5T4-ADC of Formula (VI-52), (VI-62), (VI-81), (VIb-81), (VIb-52), (VIb-62), (VIb-72), or (VIb-82). In some embodiments, a method of alleviating or reducing side effects associated with cancer comprises administering to the subject an effective amount of a 5T4-ADC of mAbA4-VIa-21, mAbA15-VIa-21, mAbA15-VIb-82, or mAbA17-VIb-82.

In any embodiment, the method of alleviating or reducing side effects associated with cancer in a subject in need thereof can comprising administering to the subject a pharmaceutical composition comprising a 5T4-ADC as disclosed herein and one or more pharmaceutically acceptable excipients. In some embodiments, the 5T4-ADC in the pharmaceutical composition can be characterized by a DAR of about 1 to about 10, about 1 to about 4, about 1 to about 2, about 1.5 to about 2, or about 1.75 to about 1.9.

It is understood that modifications that do not substantially affect the activity of the various embodiments described herein are also provided within the definition of the subject matter described herein. Accordingly, the following examples are intended to illustrate but not limit the present disclosure.

### EXAMPLES

### Example 1. Antibody Generation

To obtain binders for human 5T4, antibody discovery was conducted by phage display.

To obtain binders for human 5T4, antibody discovery was conducted by phage display of human Fab libraries and was carried out using standard protocols. The extracellular domain of human 5T4 was purchased from Acro Biosystems (biotinylated human 5T4 His-AVITAG™ Acro Cat No. TPG-H83Eb) or produced. Antigens produced consisted of human 5T4, cynomolgus 5T4, and mouse 5T4, all cloned into a vector containing a C-terminal 6-his-Avi tag and biotinylated in vitro using a co-expressed biotin ligase enzyme using standard procedures where possible.

Phage clones were screened for the ability to bind to biotinylated human 5T4. Briefly, Fab-formatted phage libraries were constructed using expression vectors capable of replication and expression in phage (also referred to as a phagemid). Both the heavy chain and the light chain were encoded in the same expression vector, where the heavy chain was fused to a truncated variant of the phage coat protein pill. The light chain and heavy chain-pill fusion were expressed as separate polypeptides and assembled in the bacterial periplasm, where the redox potential enables disulfide bond formation, to form the antigen binding domain (Fab) of the candidate antibody.

The library was created using sequences derived from a specific human heavy chain variable domain and a specific human light chain variable domain. Light chain variable domains within the screened library were generated with diversity was introduced into the VL CDR3 (L3), and where the light chain VL CDR1 (L1) and CDR2 (L2) remained the human germline sequence. For the screened library, all three CDRs of the VH region were diversified to match the positional amino acid frequency by CDR length found in the human antibody repertoire.

The phage display heavy chain (SEQ ID NO:72) and light chain (SEQ ID NO:73) scaffolds used in the library are listed below, where “x” represents CDR amino acids that were varied to create the library, and bold italic represents the CDR sequences that were constant.

The sequence for SEQ ID NO:72 was

EVQLVESGGGLVQPGGSLRLSCAASGFTFSXXXXXWVRQAPGKGLEWVAX
XXXXXXXXXXXXXXXXRFTISADTSKNTAYLQMNSLRAEDTAVYYCARXX
XXXXXXXXXXXWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSC.

The sequence for SEQ ID NO:73 was

DIQMTQSPSSLSASVGDRVTITCRASQSVSSAVAWYQQKPGKAPKLLIY
SASSLYSGVPSRFSGSRSGTDFTLTISSLQPEDFATYYCXXXXXXXXXF
GQGTKVEIKRTVAAPSVFIFPPSDSQLKSGTASVVCLLNNFYPREAKVQ
WKVDNALQSGNSQESVTEQDSKDSTYSLSSTLTLSKADYEKHKVYACEV
THQGLSSPVTKSFNRGEC.

Diversity was create through mutagenesis using degenerate DNA oligonucleotide primers to introduce diversity into VL CDR3 (L3) and VH CDR1 (H₁), CDR2 (H₂) and CDR3 (H₃) to mimic the diversity found in the natural antibody repertoire, as described in more detail in Kunkel, TA (PNAS Jan. 1, 1985. 82 (2) 488-492), herein incorporated by reference in its entirety. Briefly, uracil-incorporated single-stranded circular DNA were prepared from isolated phage using standard procedures and Kunkel mutagenesis was carried out to introduce diversity to the four CDRs. Chemically-synthesized DNA was then electroporated into TG1 cells, followed by recovery. Recovered cells were sub-cultured and infected with M13K07 helper phage to produce the phage library.

Phage panning was performed using standard procedures. Briefly, the first round of phage panning was performed with target immobilized on streptavidin magnetic beads which were subjected to approximately 1×10¹² phages from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD₆₀₀ of approximately 0.6. After 20 minutes incubation at room temperature, infected cells were sub-cultured in 25 mL 2×YT+Ampicillin and M13K07 helper phage (final concentration of approximately 1×¹⁰ pfu/mL) and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with 50 or 100 nM bead-immobilized 5T4 antigen using standard procedures (100 nM 5T4 for round 3, 50 nM 5T4 for round 4). In total, 3-4 rounds of phage panning were performed to enrich in phage displaying Fabs specific for the 5T4 antigen. 5T4-specific enrichment was confirmed using polyclonal ELISA and individual clones were isolated and further verified by performing monoclonal phage ELISA. DNA sequencing was used to determine the sequence of the CDRs of isolated Fab clones containing a candidate antibody.

The genes encoding heavy chain and light chain variable domains of the candidate antibodies were cloned separately into mammalian expression vectors for expression as full length IgGs in mammalian cells.

For the full length IgGs, the heavy chain constant regions (e.g., CH1=regular text; hinge=italicized text; CH2=bold text; and CH3=underline text) included the following amino acid sequence:

(SEQ ID NO: 74)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

For the full length IgGs, the light chain constant region (e.g., CL) included the following amino acid sequence:

(SEQ ID NO: 75)
RTVAAPSVFIFPPSDSQLKSGTASVVCLLNNFYPREAKVQWKVDNALQSG
NSQESVTEQDSKDSTYSLSSTLTLSKADYEKHKVYACEVTHQGLSSPVTK
SFNRGEC.

The IgG antibodies were purified from culture supernatant using Protein A resin.

### Example 2. Screening Assays

Bivalent binding assays were employed to provide data on whether antibodies with VH/VL obtained by the phage display in Example 1 bound immobilized 5T4 from all three clinically relevant species: human, cynomolgus monkey (cyno), and mouse. For the bivalent binding and cross-reactivity assays the 5T4 human, cyno, and mouse antigens were synthesized using sequences from the protein database and expressed as biotinylated proteins using a his-avi tag and in vivo biotinylation with co-expressed BirA enzyme.

A standard Octet-based assay was used to determine qualitatively if each antibody bound to each antigen. For these assays, 200 nM biotinylated antigens were immobilized on streptavidin biosensors in 1× kinetic buffer (ForteBio). The loaded sensors were then dipped in wells containing an antibody of interest at 500 nM and the association binding signal was monitored. An increase in signal during the association step above the negative control was considered as binding to the antigen.

For qualitative binding affinity using Octet, all twenty-seven antibodies tested showed binding to human 5T4, thirteen of the antibodies also showed binding to cyno 5T4, and thirteen of the antibodies also showed binding to mouse 5T4. Data for exemplary antibodies are shown in Table 4 below.

TABLE 4
Qualitative bivalent binding affinity by Octet

| | Human 5T4 | Cyno 5T4 | Mouse 5T4 |
|---|---|---|---|
| | Octet | Octet | Octet |
| Antibody | (bivalent) | (bivalent) | (bivalent) |
| mAbA5 | + | + | − |
| mAbA15 | + | + | + |
| mAbA17 | + | + | + |

Monovalent binding assays were employed to determine the relative affinity of each 5T4 binder obtained by the phage display in Example 1 to human 5T4. Human 5T4 was purchased from Acro Biosystems (Acro Catalog No. TPGH52E) and also produced separately. The monovalent interaction of binders to human 5T4 was monitored using an Octet (Pall ForteBio) instrument. For these assays, 36 nM antibody was immobilized on anti-human Fc (AHC) sensors in 1× kinetic buffer (ForteBio). The sensors were then dipped in wells containing a 3× serial dilution of human 5T4 ranging from 1 μM to 1.37 nM. The association was allowed to reach equilibrium. Sensors were then moved to wells containing only 1× kinetic buffer and allowed to dissociate to equilibrium. Association and dissociation rates were measured and from these, monovalent K_{D}S were calculated using the onboard analysis software (ForteBio).

Among all twenty-seven tested antibodies, the majority of them showed weak binding in the assay of Monovalent Binding Affinity—Internal (Lot 20-019-62). mAbA4, mAbA15, and mAbA17 are among those exhibiting strong or moderate binding in all three lots. The data are shown in Table 5 below.

TABLE 5
Qualitative monovalent binding affinity by Octet

| | Monovalent | Monovalent | Monovalent |
|---|---|---|---|
| | Binding | Binding | Binding |
| | Affinity - Acro | Affinity - | Affinity - Acro |
| | TPGH52E5 (Lot | Internal | TPGH52E5 (Lot |
| | B465-87AF1-K2) | (Lot 20-019-62) | B465-87AF1-JP) |
| | Octet | Octet | Octet |
| Antibody | K_{D} [nM] | K_{D} [nM] | K_{D} [nM] |
| mAbA4 | 0.01 | 83 | 30.2 |
| mAbA15 | 3.62 | 10.4 | 11.2 |
| mAbA17 | 2.81 | 9.34 | 21.1 |

### Example 3: Additional Screening and Selection

Antibodies that were selected for binding to 5T4, for example, such as those described in Example 2, were evaluated for binding to cells that express 5T4. Cells were harvested at 70-90% confluence on the day of the assay. In a v-bottom 96-well plate (Costar 3897), 100,000 cells per well were incubated with 50 μL per well 8- or 12-point antibody dilution series for 30 minutes at +4° C. After incubation, cells were washed once with PBS by centrifugation at 200×g for 5 minutes. Cells were then incubated with 50 μL per well of Alexa Fluor 488 Goat anti-human IgG Fab (Jackson Immuno Research 109-547-003) at 1:100 dilution for 30 minutes at +4° C. Cells were washed twice with PBS by centrifugation at 200×g for 5 minutes and were acquired on iQue Screener Plus (Sartorius). Mean fluorescence intensities of BL1 (Alexa-Fluor-488) channel were plotted against antibody concentrations to generate a dose response curve and EC50 values.

Twenty-six antibodies among twenty-seven tested showed binding affinity to HEK-5T4 cells, sixteen antibodies showed binding affinity to MCF-7 cells, and twenty-six antibodies showed binding affinity to HEK-cyno-5T4 cells.

Antibodies that selected for binding to all three HEK-5T4, MCF-7, and HEK-cyno-5T4 cell lines (including mAbA4, mAbA15, and mAbA17 were further evaluated in internalization assays with HEK-5T4 and MCF-7 cells.

Adherent cells were harvested at 70-90% confluence and were plated overnight at 37° C. in a 96-well black, clear/flat bottom half-area plate (Corning 3882) at 20,000 cells/well in 50 μL culture media. For suspension cell lines, 96-well plates were coated with poly-L-ornithine solution (Millipore-Sigma) for 1 hour at 37° C. and cells were seeded at 40,000 cells per/well density in 50 μL culture media, about 1 to 2 hours prior to the assay. Antibodies were diluted in cell culture media, mixed with 3× molar concentration of Incucyte human FabFluor-pH red antibody labelling reagent and incubated for 15 minutes at room temperature. 2× final concentration of all dilutions of antibody/FabFluor mix was prepared. 50 μL of 2× antibody/Fab-Fluor mix was then directly added to every well with pre-seeded cells in duplicates and plate was quickly transferred to Incucyte. Plates were then scanned every 30 minutes for up to 24 hours. Care was taken to begin the first scanning (0 hour) within 2 to 3 minutes of adding antibody/FabFluor mix to the cells. Plates were analyzed by reading the red signal generated overtime, normalized to the % confluence of cells in that well. Few antibodies were tested at different concentrations on target cell lines, and the best concentration was selected based on a good signal:noise ratio.

In the internalization assays, six antibodies (including mAbA4 and mAbA15 showed strong internalization in HEK-5T4 cells, one antibody showed moderate internalization in HEK-5T4 cells, and seven antibodies showed no internalization or non-specific internalization in HEK-5T4 cells. In additional assays, two antibodies showed strong internalization in MCF-7 cells, four antibodies showed moderate internalization in HEK-5T4 cells, and eight antibodies showed no internalization or non-specific internalization in MCF-7 cells.

### Example 4: Developability Assays

Antibodies that were selected for binding to 5T4, such as those described in Examples 2 and 3, were tested by various methods to assess developability. For example, various chromatographic methods, including size exclusion chromatography (SEC), hydrophobic interaction chromatography (HIC), and standup monolayer adsorption chromatography (SMAC) were employed to assess factors, such as monomer percentage, solubility, and antibody aggregation or precipitation.

Size exclusion chromatography (SEC) analysis was performed using a 7.8 mm ID×30 cm TSKgel G3000SWXL column (Tosoh Bioscience LLC, PN 08541) with a 6 mm×4 cm guard column (PN 08543) on an Agilent 1100 HPLC. Antibodies were normalized to 1 mg/mL concentration in Dulbecco's PBS (pH 7.4, without Ca²⁺/Mg²⁺) and clarified via centrifugation to pellet particulates while still retaining soluble aggregates. The mobile phase buffer was Dulbecco's PBS (pH 7.4, without Ca²⁺/Mg²⁺) at 2× concentration (diluted from 10× stock concentration. For each sample, 10 μL was loaded and isocratically eluted at 1.0 mL/minute over 20 minutes at ambient temperature. Absorbance was monitored at 280 nm. Chromatographic peaks were integrated to determine percentage of homogeneity and retention time. The column stationary phase along with choice of mobile phase supports hydrophobic and electrostatic interactions in addition to molecular sizing (secondary interactions much milder compared to SMAC). Data analysis was performed using Agilent ChemStation B.04.03 SP1.

Results showed that mAbA4, mAbA15, and mAbA17 (among other tested antibodies) had strong developability based on SEC analysis.

Hydrophobic interaction chromatography (HIC) analysis was performed using a 4.6 mm ID×3.5 cm TSKgel Butyl-NPR column (Tosoh Bioscience LLC, PN 14947) on an Agilent 1100 HPLC. Antibodies were normalized to 1 mg/mL concentration in Dulbecco's PBS (pH 7.4, without Ca²⁺/Mg²⁺). The column was equilibrated with 100% mobile phase Buffer A (2 M ammonium sulfate/20 mM sodium phosphate, pH 7.0) at a flow rate of 1 mL/minute at ambient temperature. For each sample, 10 μL was loaded and eluted using a gradient from 100% mobile phase buffer A to 100% mobile phase buffer B (20 mM sodium phosphate, pH 7.0) at 1.0 mL/minute over 15 minutes, held at 100% B for 3 minutes to wash the column, and returned 100% A for 2 minutes for equilibration. Absorbance was monitored at 280 nm. Sample retention time was calculated and compared to a set of standard controls to identify antibodies with increased retention time (increased hydrophobicity) and the presence of multiple species. Data analysis was performed using Agilent ChemStation B.04.03 SP1

Results from the hydrophobic interaction chromatography (HIC) analysis showed that certain antibodies had strong developability and others had moderate developability. mAbA4, mAbA15, and mAbA17 are among those having strong developability based on HIC analysis. Antibody hydrophobicity can impact antibody aggregation, solubility, and viscosity. Results indicate a low propensity for aggregation and precipitation of these antibodies.

Standup monolayer adsorption chromatography (SMAC) analysis was performed using a 4.6 mm ID×300 mm Zenix SEC 300 column (Sepax Technologies, PN 213300P-4630) with a 4.6 mm×50 mm guard column (PN 213300P-4605) on an Agilent 1100 HPLC. Antibodies were normalized to 1 mg/mL concentration in Dulbecco's PBS (pH 7.4, without Ca²⁺/Mg²⁺) and clarified via centrifugation to pellet particulates while still retaining soluble aggregates. The mobile phase buffer was Dulbecco's PBS (pH 7.4, without Ca²⁺/Mg²⁺) at 2× concentration (diluted from 10× stock concentration). For each sample, 10 μL was loaded and isocratically eluted at 0.4 mL/minute over 32 minutes at ambient temperature. Absorbance was monitored at 280 nm. Sample retention time was calculated and compared to a set of standard controls to identify antibodies with increased retention time (increased propensity to form aggregates and/or increased hydrophobic/electrostatic interactions) as the column stationary phase along with mobile phase selection promotes secondary interactions in addition to molecular sizing. Data analysis was performed using Agilent ChemStation B.04.03 SP1.

Results from the standup monolayer adsorption chromatography (SMAC) analysis showed that certain antibodies had strong developability and others had moderate developability. mAbA4, mAbA15, and mAbA17 are among those having strong developability based on SMAC analysis. The determination was based on good retention times, which indicate colloidal stability and low propensity to aggregate.

### Example 5: Epitope Binning Assays

Epitope binning assays were employed to determine which epitopes of 5T4 each antibody obtained by phage display in Example 1 bound to.

In order to maintain overall structure of the 5T4 protein while still enabling epitope binding determination, mouse-human chimeras were generated by cloning each of seven contiguous sequences of human 5T4 (Epitopes 1-7) in to replace each of the corresponding mouse sequences. For chimera generation, the full-length human and mouse 5T4 genes were synthesized by GenScript and cloned into a mammalian vector with a 6-His-Avi tag. Each of the seven sequences (Epitopes 1-7) as described herein were then removed from the human gene and separately cloned into the mouse gene using appropriate primers. Epitope 1 of human 5T4 (H1) corresponds to SSPTSSASSFSSSAPFLASAVSAQPPLPDQCPALCECSEAART (SEQ ID NO:76); Epitope 2 of human 5T4 (H2) corresponds to VKCVNRNLTEVPTDLPAYVRNLFLTGNQLAVLPAGAFARRPPLAELAALNLSGSRL DEVR (SEQ ID NO:77); Epitope 3 of human 5T4 (H3) corresponds to AGAFEHLPSLRQLDLSHNPLADLSPFAFSG (SEQ ID NO:78); Epitope 4 of human 5T4 (H4) corresponds to SNASVSAPSPLVELILNHIVPPEDERQNRSFEGMVVAALLAGRALQGLRRLE (SEQ ID NO:79); Epitope 5 of human 5T4 (H5) corresponds to LASNHFLYLPRDVLAQLPSLRHLDLSNNSLVSLTYVSFRNLTHLESL (SEQ ID NO:80); Epitope 6 of human 5T4 (H6) corresponds to HLEDNALKVLHNGTLAELQGLPHIRVFL (SEQ ID NO:81); and Epitope 7 of human 5T4 (H7) corresponds to DNNPWVCDCHMADMVTWLKETEVVQGKDRLTCAYPEKMRNRVLLELNSADLDCD PILPPSLQTS (SEQ ID NO:82). Antigens were transiently expressed using the Expi293 protein expression system (Thermo Fisher) and biotinylated in vitro using co-expressed BirA biotin ligase and purified using Ni-NTA affinity chromatography. As shown in Table 6, the seven chimeric constructs of human (H) and mouse (M) sequences generated were: (1) H1 M2 M3 M4 M5 M6 M7 (SEQ ID NO:83); (2) M1 H2 M3 M4 M5 M6 M7 (SEQ ID NO:84); (3) M1 M2 H3 M4 M5 M6 M7 (SEQ ID NO:85); (4) M1 M2 M3 H4 M5 M6 M7 (SEQ ID NO:86); (5) M1 M2 M3 M4 H5 M6 M7 (SEQ ID NO:87); (6) M1 M2 M3 M4 M5 H6 M7 (SEQ ID NO:88); and (7) M1 M2 M3 M4 M5 M6 H7 (SEQ ID NO:89). Each antibody was then tested for binding to each of the chimeric constructs and full-length human protein. Any significant binding signal to a chimera indicates the region to which the antibody specifically binds.

TABLE 6
5T4 Chimeric Constructs

| | | |
|---|---|---|
| Human | H1 M2 M3 M4 M5 | SSPTSSASS FSSSAPFLAS AVSAQPPLPD QCPALCECSE |
| 5T4 | M6 M7 | AARTVKCVNR NLLEVPADLP PYVRNLFLTG NQMTVLPAGA |
| Epitope 1 | (SEQ ID NO: 83) | FARQPPLADL EALNLSGNHL KEVCAGAFEH LPGLRRLDLS |
| | | HNPLTNLSAF AFAGSNASVS APSPLEELIL NHIVPPEDQR |
| | | QNGSFEGMVA FEGMVAAALR SGLALRGLTR LELASNHFLF |
| | | LPRDLLAQLP SLRYLDLRNN SLVSLTYASF RNLTHLESLH |
| | | LEDNALKVLH NSTLAEWHGL AHVKVFLDNN PWVCDCYMAD |
| | | MVAWLKETEV VPDKARLTCA FPEKMRNRGL LDLNSSDLDC |
| | | DAVLPQSLQT S |
| Human | M1 H2 M3 M4 M5 | SAPSSSVPS SSTSPAAFLA SGSAQPPPAE RCPAACECSE |
| 5T4 | M6 M7 | AARTVKCVNR NLTEVPTDLP AYVRNLFLTG NQLAVLPAGA |
| Epitope 2 | (SEQ ID NO: 84) | FARRPPLAEL AALNLSGSRL DEVRAGAFEH LPGLRRLDLS |
| | | HNPLTNLSAF AFAGSNASVS APSPLEELIL NHIVPPEDQR |
| | | QNGSFEGMVA FEGMVAAALR SGLALRGLTR LELASNHFLF |
| | | LPRDLLAQLP SLRYLDLRNN SLVSLTYASF RNLTHLESLH |
| | | LEDNALKVLH NSTLAEWHGL AHVKVFLDNN PWVCDCYMAD |
| | | MVAWLKETEV VPDKARLTCA FPEKMRNRGL LDLNSSDLDC |
| | | DAVLPQSLQT S |
| Human | M1 M2 H3 M4 M5 | SAPSSSVPS SSTSPAAFLA SGSAQPPPAE RCPAACECSE |
| 5T4 | M6 M7 | AARTVKCVNR NLLEVPADLP PYVRNLFLTG NQMTVLPAGA |
| Epitope 3 | (SEQ ID NO: 85) | FARQPPLADL EALNLSGNHL KEVCAGAFEH LPSLRQLDLS |
| | | HNPLADLSPF AFSGSNASVS APSPLEELIL NHIVPPEDQR |
| | | QNGSFEGMVA FEGMVAAALR SGLALRGLTR LELASNHFLF |
| | | LPRDLLAQLP SLRYLDLRNN SLVSLTYASF RNLTHLESLH |
| | | LEDNALKVLH NSTLAEWHGL AHVKVFLDNN PWVCDCYMAD |
| | | MVAWLKETEV VPDKARLTCA FPEKMRNRGL LDLNSSDLDC |
| | | DAVLPQSLQT S |
| Human | M1 M2 M3 H4 M5 | SAPSSSVPS SSTSPAAFLA SGSAQPPPAE RCPAACECSE |
| 5T4 | M6 M7 | AARTVKCVNR NLLEVPADLP PYVRNLFLTG NQMTVLPAGA |
| Epitope 4 | (SEQ ID NO: 86) | FARQPPLADL EALNLSGNHL KEVCAGAFEH LPGLRRLDLS |
| | | HNPLTNLSAF AFAGSNASVS APSPLVELIL NHIVPPEDER |
| | | QNRSFEGMVV AALLAGRALQ GLRRLELASN HFLFLPRDLL |
| | | AQLPSLRYLD LRNNSLVSLT YASFRNLTHL ESLHLEDNAL |
| | | KVLHNSTLAE WHGLAHVKVF LDNNPWVCDC YMADMVAWLK |
| | | ETEVVPDKAR LTCAFPEKMR NRGLLDLNSS DLDCDAVLPQ |
| | | SLQTS |
| Human | M1 M2 M3 M4 H5 | SAPSSSVPS SSTSPAAFLA SGSAQPPPAE RCPAACECSE |
| 5T4 | M6 M7 | AARTVKCVNR NLLEVPADLP PYVRNLFLTG NQMTVLPAGA |
| Epitope 5 | (SEQ ID NO: 87) | FARQPPLADL EALNLSGNHL KEVCAGAFEH LPGLRRLDLS |
| | | HNPLTNLSAF AFAGSNASVS APSPLEELIL NHIVPPEDQR |
| | | QNGSFEGMVA FEGMVAAALR SGLALRGLTR LELASNHFLY |
| | | LPRDVLAQLP SLRHLDLSNN SLVSLTYVSF RNLTHLESLH |
| | | LEDNALKVLH NSTLAEWHGL AHVKVFLDNN PWVCDCYMAD |
| | | MVAWLKETEV VPDKARLTCA FPEKMRNRGL LDLNSSDLDC |
| | | DAVLPQSLQT S |
| Human | M1 M2 M3 M4 M5 | SAPSSSVPS SSTSPAAFLA SGSAQPPPAE RCPAACECSE |
| 5T4 | H6 M7 | AARTVKCVNR NLLEVPADLP PYVRNLFLTG NQMTVLPAGA |
| Epitope 6 | (SEQ ID NO: 88) | FARQPPLADL EALNLSGNHL KEVCAGAFEH LPGLRRLDLS |
| | | HNPLTNLSAF AFAGSNASVS APSPLEELIL NHIVPPEDQR |
| | | QNGSFEGMVA FEGMVAAALR SGLALRGLTR LELASNHFLF |
| | | LPRDLLAQLP SLRYLDLRNN SLVSLTYASF RNLTHLESLH |
| | | LEDNALKVLH NGTLAELQGL PHIRVFLDNN PWVCDCYMAD |
| | | MVAWLKETEV VPDKARLTCA FPEKMRNRGL LDLNSSDLDC |
| | | DAVLPQSLQT S |
| Human | M1 M2 M3 M4 M5 | SAPSSSVPS SSTSPAAFLA SGSAQPPPAE RCPAACECSE |
| 5T4 | M6 H7 | AARTVKCVNR NLLEVPADLP PYVRNLFLTG NQMTVLPAGA |
| Epitope 7 | (SEQ ID NO: 89) | FARQPPLADL EALNLSGNHL KEVCAGAFEH LPGLRRLDLS |
| | | HNPLTNLSAF AFAGSNASVS APSPLEELIL NHIVPPEDQR |
| | | QNGSFEGMVA FEGMVAAALR SGLALRGLTR LELASNHFLF |
| | | LPRDLLAQLP SLRYLDLRNN SLVSLTYASF RNLTHLESLH |
| | | LEDNALKVLH NSTLAEWHGL AHVKVFLDNN PWVCDCHMAD |
| | | MVTWLKETEV VQGKDRLTCA YPEKMRNRVL LELNSADLDC |
| | | DPILPPSLQT S |

A standard Octet-based assay was used to for the epitope binning assay to establish an epitope bin for each antibody. For these assays, 50 nM biotinylated antigens were immobilized on streptavidin sensors in 1× kinetic buffer (ForteBio). The sensors were then dipped in wells containing the antibody in question at 100 nM and the association signal was monitored. The chimera(s) which effectively knocked out binding of an antibody then corresponds to the specific epitope(s) to which the antibody binds. Full-length human 5T4 was included in the assay for reference.

The results show that nine antibodies specifically bind to Epitope 4 of 5T4, three antibodies specifically bind to Epitope 7 of 5T4, and three antibodies specifically bind to Epitope 2 of 5T4, one antibody specifically binds to both Epitope 4 and Epitope 6 of 5T4, and eleven antibodies bind to full length 5T4 (FL). Exemplary epitope binning results (for mAbA4, mAbA15, and mAbA17) are summarized in Table 7.

TABLE 7
Epitope Binning Assays

| Antibody | Epitope Bin |
|---|---|
| mAbA4 | 4 |
| mAbA15 | 2 |
| mAbA17 | 7 |

As described herein, among all the antibodies screened from the phage library as described in Example 1, twenty-seven antibodies that were still able to bind to 5T4 after formatting into IgG were further tested as described in Examples 2-5. Three antibodies designated mAbA4, mAbA15, and mAbA17 were selected based on a multiplicity of activities, including their specificity for human 5T4 binding, their binding to cyno 5T4, their cell surface binding signal, and their strong developability as assessed by a variety of methods such as SEC, HIC, and SMAC. The VH, VL, and CDR sequences of these antibodies are described herein, e.g., in Tables 1-3.

### Example 6: Preparation of ADCs

### A. Preparation of Tagged Antibodies

Tagged antibodies were prepared according to methods known in the art, such as those described in Rabuka D. et al., “Site-specific chemical protein conjugation using genetically encoded aldehyde tags,” Nat Protoc., 2012; 7(6):1052-1067 and in U.S. Pat. No. 7,985,783 B2, each of which is incorporated in its entirety herein by reference. Briefly, an antibody containing a heterologous sulfatase motif can be contacted with a formylglycine-generating enzyme to convert a cysteine or serine in the motif to a formylglycine having an aldehyde moiety, thereby generating an “aldehyde-tagged antibody.” Any formylglycine-generating enzyme can be used, such as Mycobacterium tuberculosis or Mycobacterium tuberculosis.

### B. Preparation of Linker-Payloads

### 1. Synthesis of Linker-Payload VIa-21.

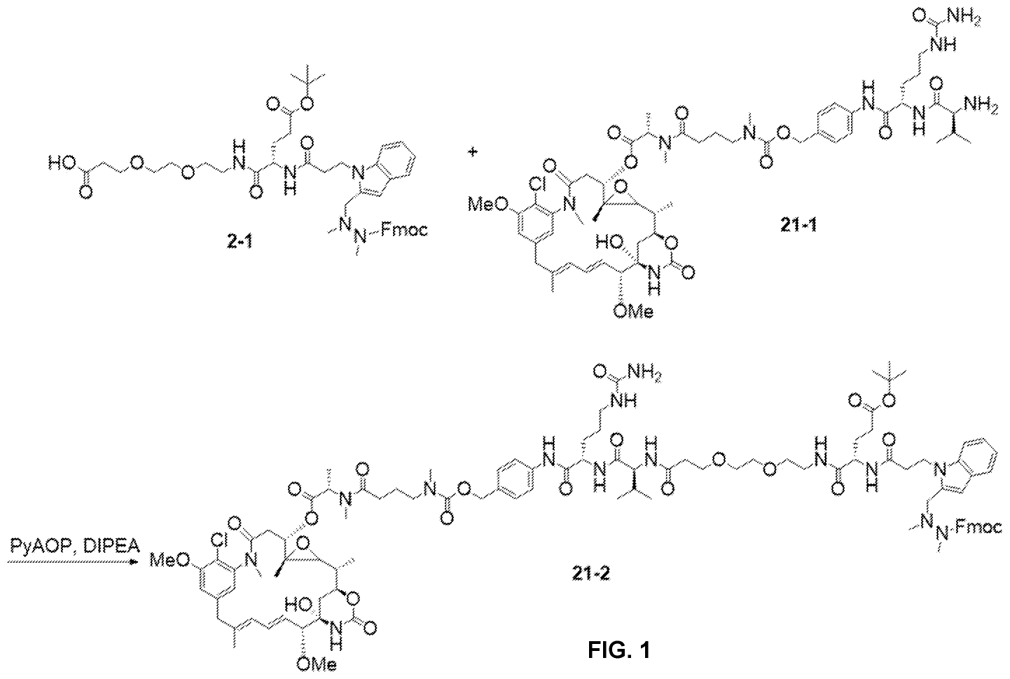

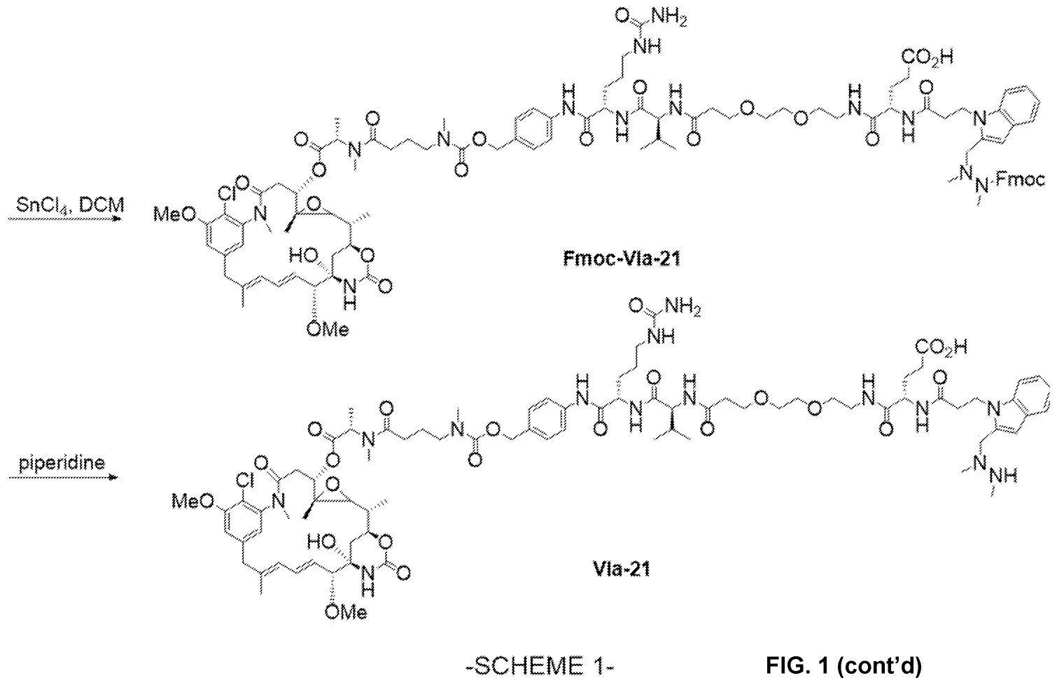

The synthesis of linker-drug VIa-21 is shown in Scheme 1 in FIG. 1. Compound 2-1 was obtained commercially from Shanghai Medicilon Inc. (Shanghai, China) and used as received. Compound 2-1 can also be prepared according to synthetic procedures disclosed in international patent application publication WO 2015/081282, which is incorporated herein by reference in its entirety. Compound 21-1 was prepared according to the procedures disclosed in, e.g., Kudirka et al. ACS Med. Chem. Lett. 2016, 7(11):94-998 and in international patent application publication WO 2015/081282, each of which are incorporated herein by reference in its entirety. All other reagents used were obtained from commercial sources and used without purification.

Compound 21-1 (62 mg, 54 μmol) and 30 μL of DIPEA were added to a stirred solution of Compound 2-1 (45 mg, 54 μmol) in 2 mL of anhydrous DMF at room temperature. (7-Azabenzotriazol-1-yloxy)tripyrrolidinophosphonium hexafluorophosphate (“PyAOP,” 28 mg, 54 μmol) was added at room temperature. After 30 minutes, the reaction mixture was poured into 20 mL of brine and extracted with DCM (2×25 mL). The organic layer was dried over sodium sulfate, concentrated under vacuum, and purified by silica gel chromatography (5-10% MeOH in DCM) to obtain 70 mg (36 μmol, 67% yield) of intermediate 3 as a colorless solid. LRMS (ESI) analysis of the solid revealed a peak at m/z 1986.0 [M+Na]⁺, as expected for C₁₀₁H₁₃₅ClN₁₄O₂₄.

SnCl₄ in DCM (1 M, 0.36 mL) was slowly added to a solution of intermediate Fmoc-VIa-21 (70 mg, 36 μmol) in anhydrous DCM (2 mL) at room temperature. After addition, reaction mixture was allowed to stir for 30 minutes at room temperature and the reaction was quenched with 2 mL of acetonitrile with two drops of water. The resulting solution was concentrated under vacuum and the residue was purified by reversed-phase chromatography (C18 column, 0-100% acetonitrile-water+0.05% trifluoroacetic acid (“TFA”)). Pure fractions were combined and lyophilized to give 25 mg (13 μmol, 36% yield) of Fmoc-VIa-21 as a colorless solid. LRMS (ESI) analysis of the solid revealed a peak at m/z 1929.8 [M+Na]⁺, as expected for C₉₇H₁₂₇ClN₁₄O₂₄.

Piperidine (26 μL) was added to a solution of Fmoc-VIa-21 (25 mg, 13 μmol) in 2 mL of anhydrous DMA at room temperature. After 20 minutes, the reaction mixture was directly purified by reversed-phase HPLC (C18 column, 0-75% acetonitrile-water+0.05% TFA). Pure fractions were combined and lyophilized to give 8 mg of linker-drug VIa-21 (5 μmol, 39% yield) as a white solid. LRMS (ESI) analysis of the white solid revealed a peak at m/z 1685.9 [M+H]⁺, as expected for C₈₂H₁₁₇ClN₁₄O₂₂.

### 2. Synthesis of Linker-Payload VIa-11.

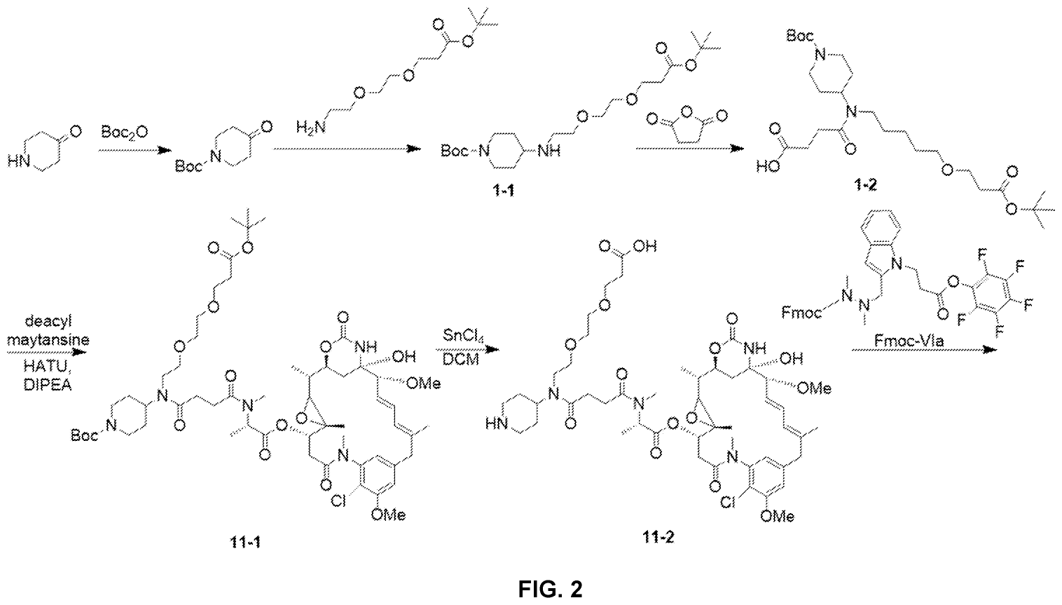

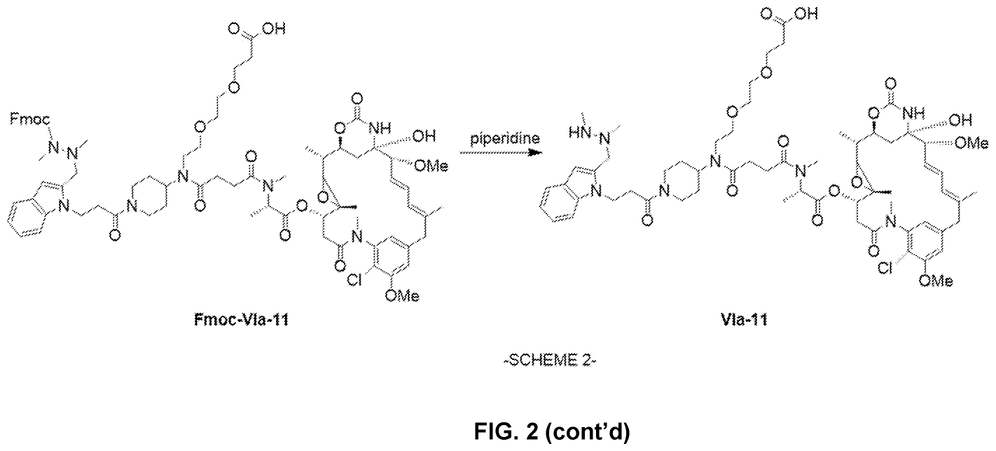

The synthesis of linker-payload VIa-11 is shown in Scheme 2 in FIG. 2. 4-Piperidinone hydrochloride monohydrate (1.53 g, 10 mmol), Boc anhydride (2.39 g, 11 mmol), sodium carbonate (1.22 g, 11.5 mmol), dioxane (10 mL), and water (1 mL) were added to a 100 mL round-bottom flask containing a magnetic stir bar. The reaction mixture was stirred at room temperature for 1 hour. The mixture was diluted with water (100 mL) and extracted with ethyl acetate (“EtOAc,” 3×100 mL) The combined organic layers were washed with brine, dried over Na₂SO₄, filtered, and concentrated under reduced pressure. The resulting material was dried in vacuo to yield tert-butyl 4-oxopiperidine-1-carboxylate as a white solid (1.74 g, 87% yield). ¹H NMR (CDCl₃) δ 3.73 (t, 4H, J=6.0), 2.46 (t, 4H, J=6.0), 1.51 (s, 9H).

Tert-butyl 4-oxopiperidine-1-carboxylate (399 mg, 2 mmol), tert-butyl 3-(2-(2-aminoethoxy)ethoxy)propanoate (550 mg, 2.4 mmol), 4 Å molecular sieves (activated powder, 200 mg), and 1,2-dichloroethane (5 mL) were added to a dried scintillation vial containing a magnetic stir bar. The mixture was stirred for 1 hour at room temperature. Sodium triacetoxyborohydride (845 mg, 4 mmol) was added to the reaction mixture. The mixture was stirred for 3 days at room temperature then partitioned between EtOAc and saturated aqueous NaHCO₃. The organic layer was washed with brine, dried over Na₂SO₄, filtered, and concentrated under reduced pressure to yield Intermediate 1-1 (tert-butyl 4-((2-(2-(3-(tert-butoxy)-3-oxopropoxy)ethoxy)ethyl)amino) piperidine-1-carboxylate) as a viscous oil (850 mg, >100% yield). MS (ESI) analysis of the oil revealed a peak at m/z 417.2, as expected.

Intermediate 1-1 (220 mg, 0.5 mmol), succinic anhydride (55 mg, 0.55 mmol), 4-(dimethylamino)pyridine (5 mg, 0.04 mmol), and dichloromethane (3 mL) were added to a dried scintillation vial containing a magnetic stir bar. The mixture was stirred for 24 hours at room temperature. The reaction mixture was partially purified by flash chromatography (eluent=50-100% EtOAc/hexanes) to yield intermediate 1-2 (13-(1-(tert-butoxycarbonyl)piperidin-4-yl)-2,2-dimethyl-4,14-dioxo-3,7,10-trioxa-13-azaheptadecan-17-oic acid) as a clear oil (117 mg).

Intermediate 1-2 (55 mg, 0.1 mmol), deacyl maytansine (65 mg, 0.1 mmol), 1-[bis(dimethylamino)methylene]-1 H-1,2,3-triazolo[4,5-b]pyridinium 3-oxide hexafluorophosphate (“HATU,” 43 mg, 0.11 mmol), dimethylformamide (“DMF,” 1 mL), and dichloromethane (“DCM,” 0.5 mL) were added to a dried scintillation vial containing a magnetic stir bar. The mixture was stirred for 8 hours at room temperature. The reaction mixture was directly purified by C18 flash chromatography (eluent=5-100% MeCN/water) to yield intermediate 11-1 as a white film (18 mg, 16% yield). MS (ESI) analysis of the film revealed a peak at m/z 1148.7, as expected.

Intermediate 11-1 (31 mg, 0.027 mmol) and dichloromethane (1 mL) were added to a dried scintillation vial containing a magnetic stir bar. The solution was cooled to 0° C. and tin(IV) tetrachloride (1.0 M solution in DCM, 0.3 mL, 0.3 mmol) was added. The reaction mixture was stirred for 1 hour at 0° C. The reaction mixture was directly purified by C18 flash chromatography (eluent=5-100% MeCN/water) to yield intermediate 11-2 as a white solid (16 mg, 60% yield). MS (ESI) analysis of the white solid revealed a peak at m/z 992.6, as expected.

Intermediate 11-2 (16 mg, 0.016 mmol), Fmoc-VIa (13 mg, 0.02 mmol, shown in Scheme 6), N,N-diisopropylethylamine (“DIPEA,” 8 μL, 0.05 mmol), and DMF (1 mL) were added to a dried scintillation vial containing a magnetic stir bar. The solution was stirred for 18 hours at room temperature. The reaction mixture was directly purified by C18 flash chromatography (eluent=5-100% MeCN/water) to yield Fmoc-VIa-11 as a white solid (18 mg, 77% yield). MS (ESI) analysis of the white solid revealed a peak at m/z 1457.9, as expected. Fmoc-VIa-11 (18 mg, 0.012 mmol), piperidine (20 μL, 0.02 mmol), and DMF (1 mL) were added to a dried scintillation vial containing a magnetic stir bar.

The solution was stirred for 20 minutes at room temperature. The reaction mixture was directly purified by C18 flash chromatography (eluent=1-60% MeCN/water) to yield the linker-drug VIa-11 as a white solid (15 mg, 98% yield). MS (ESI) analysis of the white solid revealed a peak at m/z 1236.0, as expected.

### 3. Synthesis of Linker-Payload VIb-11.

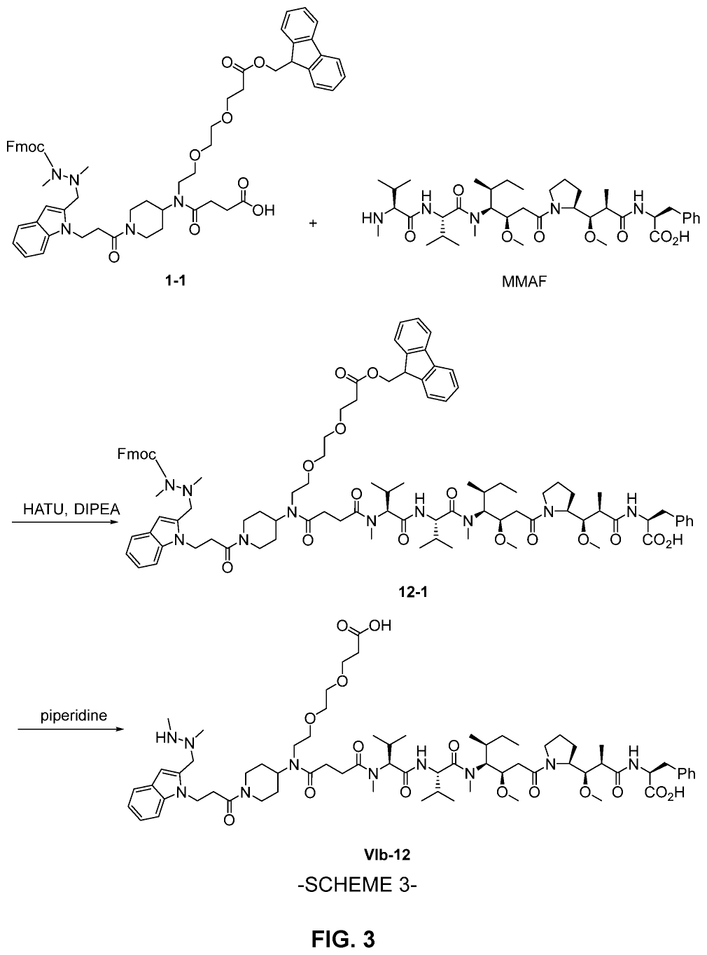

The synthesis of linker-drug VIb-11 is shown in Scheme 3 in FIG. 3.

Compound 1-1 was obtained commercially from Shanghai Medicilon and used as received. Compound 1-1 can also be prepared according to synthetic procedures disclosed in International Patent Application Publication WO 2015/081282, WO 2015/187428, and Albers, et al. Eur. J. Med. Chem. 2014, 83:3-9, each of which is incorporated herein by reference in its entirety. Monomethyl auristatin F (MMAF) was obtained from commercial sources and used without purification.

DIPEA (35 μL, 0.2 mmol) then HATU (30 mg, 0.08 mmol) were added to a solution of compound 1-1 (90 mg, 0.09 mmol) in 0.6 mL of anhydrous DMF at room temperature. The resulting mixture was stirred for 30 minutes and combined with MMAF (50 mg, 0.07 mmol). The reaction mixture was stirred at room temperature for 2 hours and purified directly by reversed-phase chromatography (C18, 40-100% acetonitrile-water+0.05% TFA) to afford 98 mg of intermediate 12-1 (0.06 mmol, 86% yield) as a yellow oil. LRMS (ESI) analysis of the white solid revealed a peak at m/z 1717.7 [M+H]⁺, as expected for C₉₈H₁₂₈N₁₀O₁₇.

Piperidine (40 μL, 0.4 mmol) was added to a solution of intermediate 12-1 (98 mg, 0.06 mmol) in 0.8 mL of anhydrous DMA at room temperature. After 30 minutes, the reaction mixture was directly purified by reversed-phase HPLC (C18 column, 10-70% acetonitrile-water+0.05% TFA). Pure fractions were combined and lyophilized to give 45 mg of linker-drug VIb-12 (0.03 mmol, 50% yield) as a white solid. LRMS (ESI) analysis of the white solid revealed a peak at m/z 1317.8 [M+H]⁺, as expected for C₆₉H₁₀₈N₁₀O₁₅.

### 4. Synthesis of Linker-Payload VIb-82.

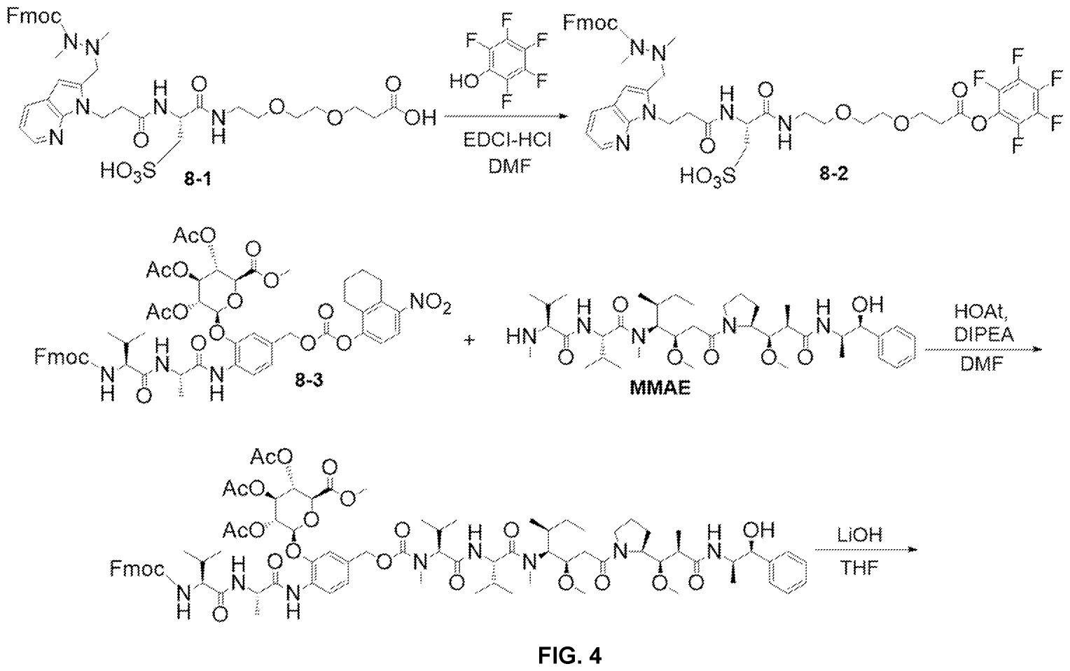

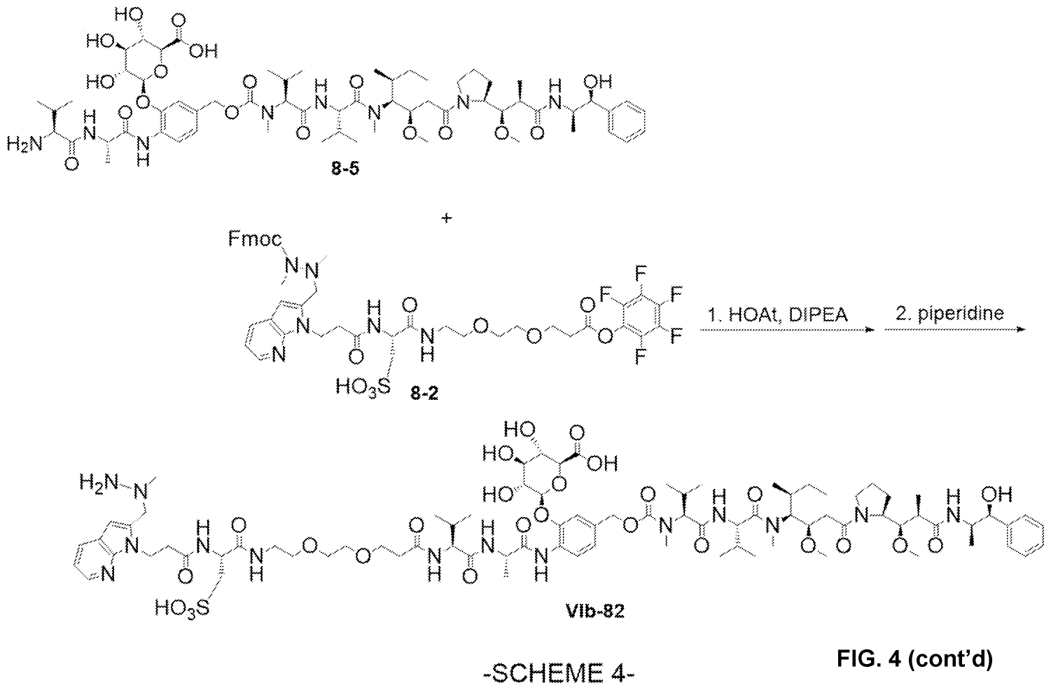

The synthesis of linker-drug VIb-82 is shown in Scheme 4 in FIG. 4.

Compound 8-1 and 2,3,4,5,6-pentafluorophenol were obtained commercially from Shanghai Medicilon and used as received. Monomethyl auristatin E (MMAE) was purchased from BroadPharm (San Diego, CA, USA). All other reagents were obtained from commercial sources and used without purification.

Compound 8-1 (1.33 g, 1.67 mmol) was combined with 2,3,4,5,6-pentafluorophenol (1.23 g, 6.68 mmol) in 6.5 mL of anhydrous DMF. This mixture was treated with 1-ethyl-3-(3-dimethylaminopropyl)carbodiimide (“EDCI-HCl,” 0.64 g, 3.34 mmol) in one portion at room temperature and stirred for 20 hours until Compound 8-1 was fully consumed, as evidenced by HPLC analysis. The reaction mixture was loaded onto a C18 column (BIOTAGE®, 60 g with a samplet) and eluted with a 0-80% gradient (10 column volumes) of ACN/water+0.05% TFA. Pure fractions were combined, concentrated on a rotary evaporator until slightly murky, and lyophilized to give intermediate 8-2 (1.40 g, 1.46 mmol, 87% yield) as a tan powder, which was stored under nitrogen at −20° C. until further use. LRMS (ESI) analysis of the tan powder revealed a peak at m/z 961.2 [M+H]⁺, as expected for C₄₄H₄₅F₅N₆O₁₁S.

In a 20 mL glass vial, MMAE (720 mg, 1.0 mmol), 5 mL of anhydrous DMF, and 0.35 mL of DIPEA (2.0 mmol) were combined at room temperature. The resulting mixture was stirred and treated with compound 8-3 (1014 mg, 1.0 mmol) as a solid in a few small portions, followed by the addition of HOAt (136 mg, 1.0 mmol) in one portion at room temperature. The reaction mixture was stirred for 6 hours until the reaction was complete, as evidenced by HPLC analysis. The reaction mixture was poured into 30 mL of water. The resulting white precipitate was separated by spinning and collected, washed with 5 mL of water, and dried briefly under high vacuum to give 1.87 g of intermediate 8-4 as a yellow solid, which was further reacted according to the disclosure below without purification.

A solution of intermediate 8-4 (1.87 g) in 15 mL of THF was cooled down to 0° C. in an ice bath and treated slowly with 1 M aqueous lithium hydroxide solution (3 mL). The reaction mixture was stirred at 0° C. for 3 hours, then warmed up to ambient temperature, treated with 3 mL of 1 M aqueous lithium hydroxide and diluted with 3 mL of methanol. The resulting mixture was stirred at room temperature for 3 hours until hydrolysis was complete, as evidenced by HPLC analysis. The reaction was then quenched by adding 1 M aqueous HCl solution to bring the reaction mixture to a pH of 7. The reaction mixture was then concentrated under reduced pressure and washed with 10 mL of methyl tert-butyl ether (“MTBE”). The aqueous layer was purified by reversed-phase chromatography (C18 column, 0-40% acetonitrile-water+0.05% TFA). Pure product fractions were combined, concentrated under reduced pressure, and lyophilized to give intermediate 8-5 as a white powder (735 mg, 0.60 mmol, 60% yield over two steps). LRMS (ESI) analysis of the white powder revealed a peak at m/z 1229.7 [M+H]⁺, as expected for C₆₁H₉₆N₈O₁₈.

DIPEA (0.21 mL, 1.2 mmol) and a solution of intermediate 8-2 (575 mg, 0.60 mmol) in 2 mL of DMA were added to a stirred solution of intermediate 8-5 (735 mg, 0.60 mmol) in 3 mL of anhydrous DMA at room temperature. 1-Hydroxy-7-azabenzotriazole (“HOAt,” 84 mg, 0.60 mmol) was then added. The resulting mixture was stirred for 30 minutes until coupling was complete, as evidenced by HPLC. The reaction mixture was then treated with 1.2 mL of piperidine at room temperature. After 15 minutes, the reaction mixture was purified by reversed-phase chromatography (C18 column, 0-40% gradient of acetonitrile-water). Pure fractions were combined, concentrated under reduced pressure and room temperature, and lyophilized to give linker-drug VIb-82 (808 mg, 0.45 mmol, 75% yield) as a white fluffy powder. LRMS (ESI) analysis of the white powder revealed a peak at 1783.9 [M+H]⁺, as expected for C₈₄H₁₃₀N₁₄O₂₆S m/z.

### 5. Synthesis of Linker-Payload VIb-81.

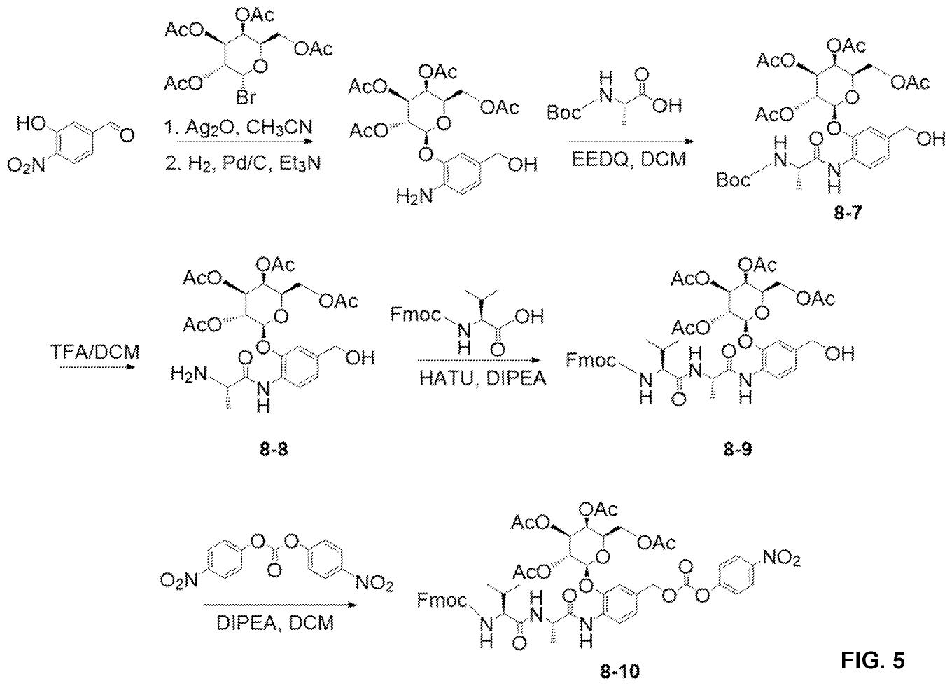

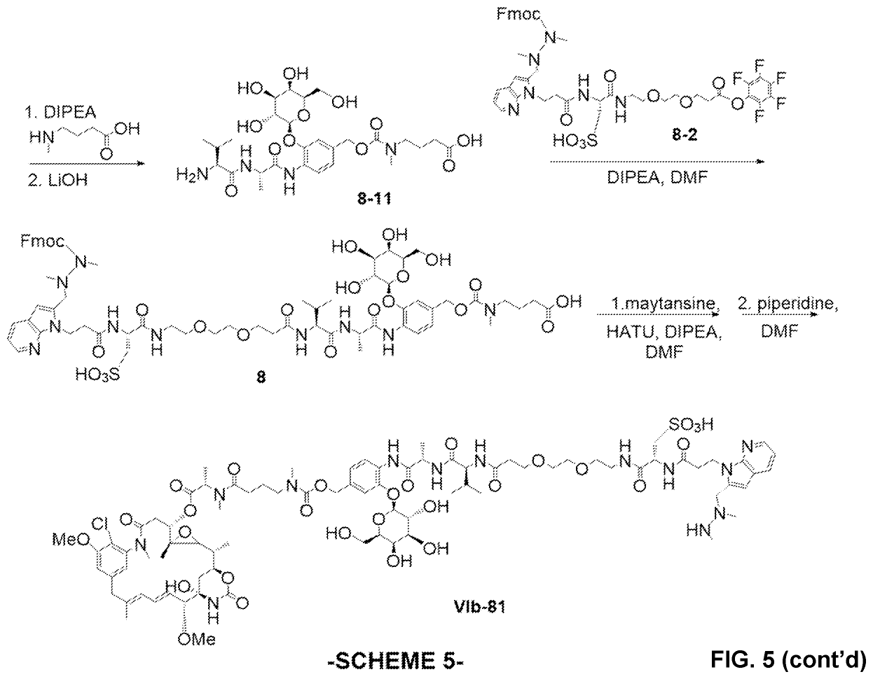

The synthesis of linker-drug VIb-81 is shown in Scheme 5 in FIG. 5. [00568]3-Hydroxy-4-nitrobenzaldehyde (204 mg, 1.22 mmol) and acetobromo-α-D-galalctose (500 mg, 1.22 mmol) were combined in a 50-mL round bottom flask with 20 mL of anhydrous acetonitrile. The solution was treated with silver(I) oxide (986 mg, 4.3 mmol) and the resulting mixture was vigorously stirred at room temperature in the dark for 24 hours. After concentrating the reaction mixture under vacuum, the residue was purified on silica gel using an ethyl acetate-hexane mixture as an eluent (0-100% gradient) to obtain 420 mg (0.85 mmol, 70% yield) of (2R,3S,4S,5R,6S)-2-(acetoxymethyl)-6-(2-amino-5-(hydroxymethyl)phenoxy)tetrahydro-2H-pyran-3,4,5-triyl triacetate as a light yellowish solid. LRMS (ESI) analysis of the yellow solid revealed a peak at 520.0 [M+Na]⁺, as expected for C₂₁H₂₃NO₁₃.

Palladium on carbon (30 mg, 10% wt.) and 30 μL of trimethylamine were added to a solution of ((2R,3S,4S,5R,6S)-2-(acetoxymethyl)-6-(5-formyl-2-nitrophenoxy)tetrahydro-2H-pyran-3,4,5-triyl triacetate (350 mg, 0.70 mmol) in 3 mL of ethyl acetate. After removal of air, the flask was equipped with a hydrogen balloon and the reaction mixture was stirred at room temperature for 48 hours. After filtration through a pad of celite, solvents were removed under vacuum and the resulting residue was dried under high vacuum to give 300 mg of (2R,3S,4S,5R,6S)-2-(acetoxymethyl)-6-(2-amino-5-(hydroxymethyl)phenoxy)tetrahydro-2H-pyran-3,4,5-triyl triacetate as white solid, which was used further in synthesis without additional purification. LRMS (ESI) analysis of the white solid revealed a peak at 470.1 [M+H]⁺, as expected for C₂₁H₂₇NO₁₁.

Boc-L-alanine (220 mg, 1.11 mmol) and (2R,3S,4S,5R,6S)-2-(acetoxymethyl)-6-(2-amino-5-(hydroxymethyl)phenoxy)tetrahydro-2H-pyran-3,4,5-triyl triacetate (520 mg, 1.16 mmol) were combined in 5 mL of anhydrous dichloromethane and 0.50 mL of anhydrous methanol. The resulting solution was treated with N-ethoxycarbonyl-2-ethoxy-1,2-dihydroquinoline (“EEDQ”, 280 mg, 1.13 mmol) at room temperature. The reaction mixture was stirred in the dark for one hour after which the solvent was removed in vacuum. The resulting intermediate 8-7 was dried under high vacuum for one hour and dissolved in 6 mL of 1:1 mixture of TFA and DCM at room temperature. The solution was allowed to stand for one hour after which solvents were removed and the resulting intermediate 8-8 was kept under high vacuum for a few hours.

In a separate 20 mL scintillation vial, Fmoc-L-valine (375 mg, 1.1 mmol) was mixed with HATU (420 mg, 1.1 mmol) and DIPEA (375 μL, 2.15 mmol) in 3 mL of anhydrous DMF. The mixture was stirred at room temperature for 30 minutes, combined with intermediate 8-8 and stirring overnight until the reaction was complete (as evidenced by LCMS analysis). The reaction mixture was directly purified on a reversed-phase C18 column (CH₃CN—H₂O/0.05% TFA, 0-80% gradient) to yield intermediate 8-9 as a mixture with minor impurities. The product was thoroughly dried under high vacuum and subjected to the next step without additional purification.

Intermediate 8-9 was dissolved in 6 mL of anhydrous DCM and treated with DIPEA (375 μL, 2.15 mmol) followed by 686 mg (2.26 mmol) of bis(4-nitrophenyl) carbonate in one portion at room temperature. The reaction mixture was stirred for one hour after which solvent was removed under vacuum. The resulting residue was purified by column chromatography on a silica gel (EtOAc-hexanes, 10-100% gradient) to yield intermediate 8-10 (640 mg, 0.62 mmol, 56% yield over four steps) as a yellowish solid. LRMS (ESI) analysis of the intermediate revealed a peak at 1049.3 [M+Na]⁺, as expected for C₅₁H₅₄N₄O₁₉.

4-(Methylamino)butanoic acid (61 mg, 0.52 mmol), 61 μL (0.35 mmol) of DIPEA, and 1 mL of anhydrous DMF were added to a 20 mL scintillation vial. The resulting solution was combined with 180 mg (0.175 mmol) of intermediate 8-10 at room temperature. The reaction mixture was stirred for one hour until intermediate 8-10 was fully consumed (as evidenced by LCMS analysis). Solvents were removed under vacuum, and the residue was dissolved in 3 mL of methanol. The obtained solution was cooled down to 0° C. and treated with 3 mL of 1M aqueous lithium hydroxide. The reaction mixture was stirred for 30 minutes, then warmed up to room temperature and concentrated under vacuum. The residue was directly purified on reversed-phase C18 column (CH₃CN—H₂O/0.05% TFA, 0-50% gradient) to obtain 90 mg (0.146 mmol, 83% yield over two steps) of intermediate 8-11 as a yellow oil. LRMS (ESI) analysis of the oil revealed a peak at 615.3 [M+H]⁺, as expected for C₂₇H₄₂N₄O₁₂.

Intermediate 8-11 (84 mg, 0.14 mmol), 1 mL of anhydrous DMF, and DIPEA (48 μL, 0.28 mmol) were added to a 20 mL scintillation vial followed by 130 mg (0.14 mmol) of intermediate 8-2. The resulting mixture was stirred for 30 minutes until coupling was complete (as evidenced by LCMS analysis). Maytansine (89 mg, 0.14 mmol) was added directly to the reaction mixture as a solution in 0.5 mL of DMF, followed by addition of HATU (52 mg, 0.14 mmol). After 30 minutes, the reaction mixture was purified on a reversed-phase C18 column (CH₃CN—H₂O/0.05% TFA, 10-95% gradient). After complete removal of solvents, the resulting intermediate 8 was dissolved in 3 mL of DMF and treated with 150 μL of piperidine at room temperature. After 30 minutes, the reaction mixture was directly purified on a reversed-phase preparative HPLC (C18 column, CH₃CN—H₂O/0.05% TFA, 0-50% gradient). Pure fractions were lyophilized to give 58 mg (0.032 mmol, 23% yield over three steps) of linker-drug VIb-81. LRMS (ESI) analysis of the lyophilized solid revealed a peak at 1801.8 [M+H]⁺, as expected for C₈₂H₁₁₈ClN₁₃O₂₈S.

### C. Bioconjugation of 5T4 ADCs

Aldehyde-tagged antibodies (15 mg/mL) were conjugated to synthesized linker-drugs (8 mol. equivalents drug:antibody) by reacting the linker-drug and aldehyde-tagged antibody for 72 hours at 37° C. in 20 mM sodium citrate, pH 5.5, 50 mM sodium chloride containing 0.85% DMA. In some cases, to improve linker-drug solubility, 0.25 TX-100 and/or up to a maximum of 10% vol/vol DMA was added. After conjugation, residual free drug was removed by diafiltration using a 115V Labscale Tangential Flow Filtration (TFF) system (Millipore, Cat. No. XX42LSS11) with a 30 kDa TFF cassette (Sigma-Aldrich, Cat. No. P3C030C00) and exchanging for 12 diavolumes (600 mL) into 20 mM sodium citrate, pH 5.5, 50 mM NaCl. Alternatively, residual free drug was removed using multiple rounds of dilution into 20 mM sodium citrate, pH 5.5, 50 mM sodium chloride and concentration using AMICON® 0.5 mL 30 kD molecular weight cut off (MWCO) centrifugal filters (Millipore Sigma, Cat. No. #UFC5030BK) or ZEBA® desalting columns (Fisher Scientific, Cat. No. P187766). To determine the drug-to-antibody ratio (DAR) of the final product, antibody-drug conjugates were examined by analytical hydrophobic interaction chromatography (HIC) or polymeric reverse phase chromatography (PLRP). The HIC column (Tosoh Bioscience, Cat. No. 14947) was run with 1.5 M ammonium sulfate and 25 mM sodium phosphate (pH 7.0) as mobile phase A and 25% isopropanol and 18.75 mM sodium phosphate (pH 7.0) as mobile phase B. The PLRP column (Agilent, Cat. No. PL1912-1802) was run with 0.1% trifluoroacetic acid in H₂O as mobile phase A and 0.1% trifluoroacetic acid in CH₃CN for mobile phase B, with the column heated to 80° C. To determine aggregation, samples were analyzed using analytical size exclusion chromatography (Tosoh Bioscience, Cat. No. 08541) using a mobile phase of 300 mM NaCl, 25 mM sodium phosphate, and 5% isopropanol (pH 6.8).

D. ADC Characterization: The Following Analyses were Performed to Characterize the Final ADC Product:

### 1. Melting Temperature Analyses

Samples for protein thermal shift experiments (melting temperature) were prepared using the APPLIED BIOSYSTEMS™ PROTEIN THERMAL SHIFT™ Dye Kit (ThermoFisher Scientific Cat. No. 4461146). Data were collected using a QUANTSTUDIO™ 3 instrument (ThermoFisher Scientific Cat. No. A28567) with PROTEIN THERMAL SHIFT™ Software v1.4 (ThermoFisher Scientific Cat. No. 466038). Briefly, PROTEIN THERMAL SHIFT™ dye (1000×) was diluted in water to 8× in deionized water. Samples were prepared on ice by combining 5 μL of PROTEIN THERMAL SHIFT™ Buffer, 12.5 μL of protein solution, and 2.5 μL of the diluted PROTEIN THERMAL SHIFT™ Dye (8×) for a total reaction solution volume of 20 μL. The reaction solution was mixed 10 times with a pipette. Tubes were then spun down at 1000 rpm for 1 minute and then incubated on ice until analysis. Samples were loaded onto the instrument plate, which were then loaded into a QUANTSTUDIO™ 3 Real-Time PCR System instrument. Data was collected using the standard Melt Curve experiment with the following conditions: Step 1 temperature: 25° C. for 2 minutes; Step 2 temperature: 99° C. for 2 minutes with continuous fast ramp mode (Step 1: 1.6° C./sec; Step 2: 0.05° C./sec). Raw melting temperature data were deconvoluted within the QUANTSTUDIO™ 3 analysis software. Table 8 below reports the measured melting temperature of each of the ADCs.

TABLE 8
Melting Temperature of ADCs

| mAb or ADC | CH2 Tₘ | Fab Tₘ |
|---|---|---|
| mAbA4 WT | 69.18 | 89.94 |
| mAbA15 WT | 70.07 | 86.38 |
| mAbA17 WT | 70.06 | 81.92 |
| mAbA4-VIa-11 | 69.03 | 89.63 |
| mAbA15-VIa-11 | 69.34 | 89.95 |
| mAbA17-VIa-11 | 69.04 | 80.9 |
| mAbA4-VIb-82 | 69.3 | 89.59 |
| mAbA15-VIb-82 | 69.74 | 86.04 |
| mAbA17-VIb-82 | 69.46 | 80.87 |
| mAbA15-VIb-12 | 69.48 | 85.94 |

### 2. Drug-to-Antibody Ratio (DAR).

Analytical Hydrophobic Interaction Chromatography (HIC)(Tosoh #14947) with UV/Vis detection. The reduced form of the final product was analyzed by reverse phase liquid chromatography (RP-HPLC)(Agilent Cat. No. PL1912-1802) with UV/Vis detection to verify DAR. RP chromatography also provided data to verify that only the heavy chain was modified and there was only one modification. Table 9 below reports the DAR for each ADC tested.

TABLE 9
Drug-to-Antibody Measurements for ADCs

| | Linker-Payload | | | | |
|---|---|---|---|---|---|
| Antibody | VIa-21 | VIa-11 | VIb-12 | VIb-82 | VIb-81 |
| mAbA4 | 1.8 | 1.8 | — | 1.8 | — |
| mAbA15 | 1.8 | 1.9 | 1.9 | 1.9 | 1.9 |
| mAbA17 | 1.9 | 1.8 | — | 1.8 | — |

Specific protocols for each analytical method identified above are detailed further below.

Analytical Hydrophobic Interaction Chromatography (HIC): Samples were analyzed on an Agilent 1100 Instrument equipped with a 4.6 mm×35 mm TSK gel butyl-NPR column from Tosoh (#14947) held at 25° C. Samples were prepared by adding 20 μL of 50 mM NaCl (pH 5.5) and 20 μL of Mobile Phase A to 20 μg of sample and were injected at room temperature. Gradient elution utilized mobile phase A (25 mM Na₃PO₄, 1.5 M (NH₄)₂SO₄, pH 7.0) and Mobile Phase B (18.75 mM Na₃PO₄, pH 7.0, 25% isopropyl alcohol) in the program reported in Table 10 at a flow rate of 1.0 mL/minute. Detection was via UV at 215 nm, 252 nm, and 280 nm.

TABLE 10
Gradient Elution Program for HIC Analyses

| Time (min) | Mobile Phase B (%) |
|---|---|
| 0.0 | 12 |
| 5.5 | 42 |
| 7.5 | 70 |
| 8.5 | 100 |
| 9.5 | 100 |
| 10 | 12 |
| 12 | 12 |

Reverse Phase HPLC: Samples were analyzed on an Agilent 1100 Instrument equipped with a 50×2.1 mm PL1912-1802 (8 μm, 1000 Å) column from Agilent held at 80° C. Samples were prepared by adding 10 μL of a mixture of 0.5 mM DTT, 50 μL of 8 M guanidine HC, 130 mM tris, and 1 mM EDTA (pH 7.6) to a 20 μg sample and adding PBS to a final volume of 100 μL. Samples were incubated at 37° C. for 30 minutes after preparation. Samples were eluted in 25% acetonitrile+0.1% TFA at a flow rate of 2.0 mL/minute and detected via UV at 215 nm, 252 nm, and 280 nm.

Size Exclusion HPLC: Samples were analyzed on an Agilent 1100 Instrument equipped with a 7.8 mm×300 mm G300 SW XL column from Tosoh (#08541) held at 25° C. Samples (1 mg/mL, 20 μg) were injected at room temperature, eluted in 25 mM NaPO4 buffer, 300 mM NaCl, pH 6.8 at a flow rate of 1.0 mL/minute, and detected via UV at 215 nm, 252 nm, and 280 nm.

LC-MRM/MS: This method determines the free drug concentration of a linker-drug by comparing the signal of the linker-drug and its known modifications, generated by RP-HPLC-MRM, in the preparation of ADCs to an internal standard curve using the linker-drug as the calibration standard and dibucaine-HCl as the internal standard.

A standard curve was generated using samples ranging from 0.457 nM to 1000 nM. A 1000 nM standard of the linker-drug was prepared by adding 0.75 μL of the 100-μM linker-drug to 74.25 μL of matrix and mixing via pipette aspiration (10×). Prepared standards were stored at −80° C. The standard was diluted 1:3 by adding 25 μL to 50 μL of matrix, mixing via pipette aspiration (10×) and vortexing to generate a 333.33 nM standard. A 111.11 nM standard was generated by diluting 25 μL of the 333.33 nM standard to 50 μL of matrix in the same manner. Further dilutions were carried out in similar fashion, resulting in eight standards with the following concentrations: 1000, 333.33, 111.11, 37.04, 12.35, 4.12, 1.37, and 0.46 nM.

ADC (50 μL) was added to each of eight centrifuge tubes. An internal standard (150 μL) was added to each to create a final volume of 200 μL in each centrifuge tube, each with a different concentration of linker-drug. All centrifuge tubes were vortexed for 5 minutes after which the solution became cloudy as the protein was denatured and precipitated, leaving the free drug in solution. All samples were incubated on ice for 30 minutes to aid precipitation then vortexed again for 10 minutes.

To separate the precipitated mAb, each sample was centrifuged (10 minutes, 15,000×g). Supernatant (75 μL) was removed and put in an autosampler vial and diluted with 75 μL water. Each sample was run in triplicate using the following HPLC/MS method below.

Samples were analyzed on an Agilent 1100 Instrument equipped with a 2.1×100 mm ZORBAX® RRHD Eclipse Plus C18 column (95 Å, 1.8 μm) from Agilent held at 50° C. Samples (1 mg/mL, 20 μg) were injected at room temperature and eluted in 1% acetonitrile+0.1% TFA at a flow rate of 0.5 mL/minute. Mass spectrometry was used for detection.

The Mass Spectrometry method was created using the “Compound Optimization” function of SCIEX's Analyst Software. The instrument outputs a set of MRM transitions for the molecule, which were then used for the LC-MS method. MRM transitions for dibucaine were also generated using “Compound Optimization.”

The transitions identified in Table 11 below are used for determining free concentration of linker-drug VIb-82 (this can vary based on the mass spectrometer). Similar transitions were used for determining free VIa-21, VIb-12, and VIa-11 concentration. The identity of each transition species is depicted below in Table 11.

TABLE 11
Transition Species for Determining Free Concentration of Linker-
Payload VIb-82

| | Q3 | Time | Transition | DP | CE | CXP |
|---|---|---|---|---|---|---|
| Q1 (Da) | (Da) | (ms) | Species | (Volts) | (Volts) | (Volts) |
| 892.536 | 718.2 | 30 | VIb-82 Parent | 1 | 29 | 26 |
| 892.536 | 501.1 | 30 | VIb-82 Parent | 1 | 35 | 18 |
| 892.536 | 725 | 30 | VIb-82 Parent | 1 | 29 | 26 |
| 898.527 | 718.2 | 30 | VIb-82 +12 Da | 1 | 29 | 30 |
| 898.527 | 737.1 | 30 | VIb-82 +12 Da | 1 | 29 | 12 |
| 898.527 | 513 | 30 | VIb-82 +12 Da | 1 | 39 | 54 |
| 883.684 | 718.1 | 30 | VIb-82 −18 Da | 136 | 31 | 30 |
| 883.684 | 501 | 30 | VIb-82 −18 Da | 136 | 37 | 20 |
| 883.684 | 725 | 30 | VIb-82 −18 Da | 136 | 27 | 12 |
| 343.968 | 271.1 | 10 | IS | 31 | 29 | 24 |
| 343.968 | 215.1 | 10 | IS | 31 | 39 | 16 |

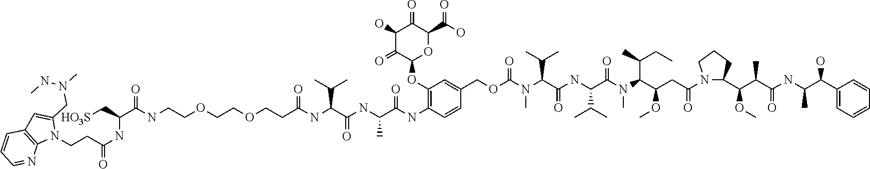 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCOCCNC(=O)[C@H](CS(=O)(=O)O)NC(=O)CCn2c(CN(C)NC)cc3cccnc32)C(C)C)c(O[C@@H]2O[C@H](C(=O)O)C(=O)[C@H](O)C2=O)c1)C(C)C]

VIb-82, Parent, C₈₄H₁₃₀N₁₄O₂₆S
Monoisotopic Mass: 1782.9001 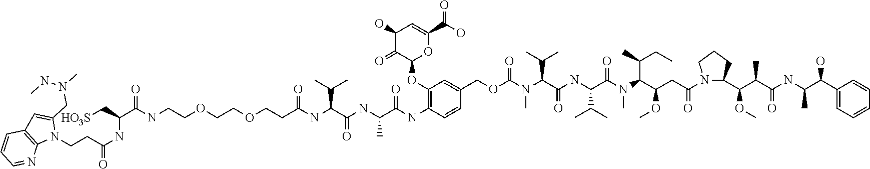
VIb-82, −18 Da, C₈₄H₁₂₈N₁₄O₂₅S
Monoisotopic Mass: 1764.8896 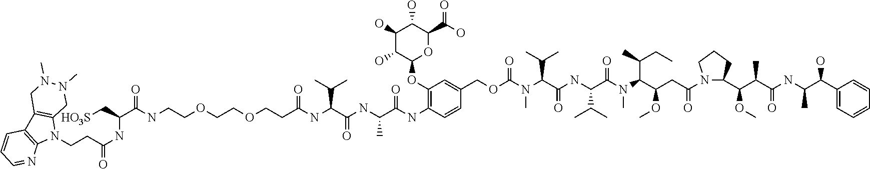 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCOCCNC(=O)[C@H](CS(=O)(=O)O)NC(=O)CCn2c3c(c4cccnc42)CN(C)N(C)C3)C(C)C)c(O[C@@H]2O[C@H](C(=O)O)[C@@H](O)[C@H](O)[C@H]2O)c1)C(C)C]
VIb-82, +12 Da, C₈₅H₁₃₀N₁₄O₂₆S
Monoisotopic Mass: 1794.9001 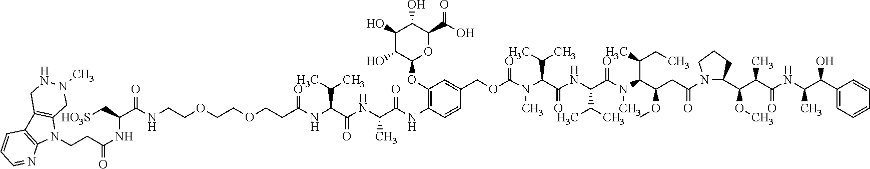 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCOCCNC(=O)[C@H](CS(=O)(=O)O)NC(=O)CCn2c3c(c4cccnc42)CNN(C)C3)C(C)C)c(O[C@@H]2O[C@H](C(=O)O)[C@@H](O)[C@H](O)[C@H]2O)c1)C(C)C]
VIb-82, −2 Da, C₈₄H₁₂₈N₁₄O₂₆S
Monoisotopic Mass: 1780.8845

To calculate free drug percent, a linear calibration curve was generated. Each transition identified in the Mass Spectrometry Method generates its own chromatogram. The peak for each transition was integrated for all standards to find peak area. The peak area for all transitions, including the “Parent,” “−18 Da,” and “+12 Da,” were added together to generate a “VIb-82 Area” value. Peak areas for all the transitions for the internal standards were likewise added to generate an “IS Area” value. A ratio of VIb-82 Area to IS Area was calculated and a scatter plot was generated with concentration on the x-axis and ratio on the y-axis. A trendline was generated from the plotted data of which the x-intercept of the trendline equation was the concentration of ADC. Free drug % was calculated using the following equation:

$$Free Drug \% = \frac{Free Drug Concentration}{Total ADC Concentration} * 100 \%$$

### Example 7: Binding of Unconjugated Antibodies to Human 5T4 (ELISA)

A. Normal Binding Unconjugated antibodies were tested for their ability to bind to human 5T4 (hu5T4) using ELISA. On Day 1, unconjugated human TPBG/5T4 protein with a c-terminal polyhistidine and Avi (AVITAG™) tag (AcroBiosystems (Newark, DE, USA), Cat. No. TPG-H52E5) was diluted 2 μg/mL in PBS, mixed thoroughly, plated 100 μL per well in a 96-well plate (Nunc MAXISORP®) and incubated overnight at 4° C.

On Day 2, plates were washed four times with 0.1% Tween®-20 in PBS (“PBS-T”). Wells were blocked with 200 μL of casein in PBS (BLOCKER™ Casein, available from ThermoFisher Scientific) and incubated for 2 hours at room temperature while shaking.

A 1:3 dilution series of IgG-ADC was prepared in PBS and added at a volume of 100 μL per well in a 96-well plate. Plates were incubated for 1 hour at room temperature while shaking.

Goat anti-human IgG Fc gamma specific HRP conjugate (Jackson Immunoresearch, cat #109-035-098) was diluted to a factor of 1:15000 in PBS.

ELISA plates were washed six times with PBS-T with a plate washer (BIOTEK® ELx405™) after which 100 μL of the goat anti-human IgG Fc gamma specific HRP conjugate was added into each well. Plates were then incubated for 30 minutes at room temperature while shaking.

ELISA plates were washed six times with PBS-T using a plate washer (BIOTEK ELx405™) after which 100 μL of 3,3′,5,5′-tetramethylbenzidine (TMB) was added to each well. Plates were allowed to develop until a dark blue color became visible. H₂SO₄ (2 N, 100 μL) was then added to quench the reaction and absorbance of each well was read on a plate reader (SPETRAMAX® M5 equipped with SOFTMAX® Pro software available from Molecular Devices).

### B. Stress Testing

1. High pH Stress Test. Several unconjugated antibodies were tested for their ability to bind to 5T4 using ELISA under basic (high pH) conditions. 1 mg/mL of antibody in 0.1% w/w H₂O₂/1×PBS was stored at room temperature for 24-48 hours in the dark after which 50 mM methionine was added. Samples were frozen for storage prior to analysis, which was carried out via ELISA as described in part A. Table 12 below reports the EC50 calculated for each antibody tested.

TABLE 12
Binding of Unconjugated Antibodies to hu5T4 under High pH

| mAb | condition | EC₅₀ (nM) |
|---|---|---|
| mAbA4 | pH 8.5 Day 0 | 0.120 |
| mAbA4 | pH 8.5 Day 1 | 0.127 |
| mAbA4 | pH 8.5 Day 4 | 0.114 |
| mAbA4 | pH 8.5 Day 7 | 0.128 |
| mAbA4 | pH 8.5 Day 14 | 0.136 |
| mAbA15 | pH 8.5 Day 0 | 0.134 |
| mAbA15 | pH 8.5 Day 1 | 0.128 |
| mAbA15 | pH 8.5 Day 4 | 0.133 |
| mAbA15 | pH 8.5 Day 7 | 0.143 |
| mAbA15 | pH 8.5 Day 14 | 0.131 |
| mAbA17 | pH 8.5 Day 0 | 0.153 |
| mAbA17 | pH 8.5 Day 1 | 0.174 |
| mAbA17 | pH 8.5 Day 4 | 0.150 |
| mAbA17 | pH 8.5 Day 7 | 0.158 |
| mAbA17 | pH 8.5 Day 14 | 0.167 |

2. Low pH Stress Test. Several unconjugated antibodies were tested for their ability to bind to 5T4 using ELISA under acidic (low pH) conditions. Solutions of 1 mg/mL to 10 mg/mL of antibody in 20 mM Tris/10 mM EDTA at a pH of 8.5 were prepared and stored at 37° C. for 1 to 2 weeks. Samples were frozen for storage prior to analysis, which was carried out via ELISA as described in part A. Table 13 below reports the EC₅₀ calculated for each antibody tested.

TABLE 13
Binding of Unconjugated Antibodies to hu5T4 under Low pH

| mAb | condition | EC₅₀ (nM) |
|---|---|---|
| mAbA4 | pH 5.5 Day 0 | 0.133 |
| mAbA4 | pH 5.5 Day 1 | 0.132 |
| mAbA4 | pH 5.5 Day 4 | 0.125 |
| mAbA4 | pH 5.5 Day 7 | 0.124 |
| mAbA4 | pH 5.5 Day 14 | 0.122 |
| mAbA15 | pH 5.5 Day 0 | 0.124 |
| mAbA15 | pH 5.5 Day 1 | 0.134 |
| mAbA15 | pH 5.5 Day 4 | 0.123 |
| mAbA15 | pH 5.5 Day 7 | 0.132 |
| mAbA15 | pH 5.5 Day 14 | 0.138 |
| mAbA17 | pH 5.5 Day 0 | 0.164 |
| mAbA17 | pH 5.5 Day 1 | 0.159 |
| mAbA17 | pH 5.5 Day 4 | 0.157 |
| mAbA17 | pH 5.5 Day 7 | 0.190 |
| mAbA17 | pH 5.5 Day 14 | 0.153 |

3. Oxidative Stress. Several unconjugated antibodies were tested for their ability to bind to 5T4 using ELISA in an oxidative environment. Solutions of 1 mg/mL to 10 mg/mL of antibody in 20 mM citrate/50 mM NaCl at a pH of 5.5 were prepared and stored at 37° C. for 1 to 2 weeks. Samples were frozen for storage prior to analysis, which was carried out via ELISA as described in part A. Table 14 below reports the EC50 calculated for each antibody tested.

TABLE 14
Binding of Unconjugated Antibodies
to hu5T4 under Oxidative Stress

| mAb | condition | EC₅₀ (nM) |
|---|---|---|
| mAbA4 | PBS Time 0 | 0.182 |
| mAbA4 | PBS time 24 hr. | 0.200 |
| mAbA4 | PBS time 48 hr. | 0.189 |
| mAbA4 | PBS + H₂O₂ time 0 | 0.225 |
| mAbA4 | PBS + H₂O₂ time 24 hr. | 0.393 |
| mAbA4 | PBS + H₂O₂ time 48 hr. | 0.386 |
| mAbA15 | PBS Time 0 | 0.229 |
| mAbA15 | PBS time 24 hr. | 0.212 |
| mAbA15 | PBS time 48 hr. | 0.203 |
| mAbA15 | PBS + H₂O₂ time 0 | 0.191 |
| mAbA15 | PBS + H₂O₂ time 24 hr. | 0.190 |
| mAbA15 | PBS + H₂O₂ time 48 hr. | 0.189 |
| mAbA17 | PBS Time 0 | 0.198 |
| mAbA17 | PBS time 24 hr. | 0.186 |
| mAbA17 | PBS time 48 hr. | 0.185 |
| mAbA17 | PBS + H₂O₂ time 0 | 0.188 |
| mAbA17 | PBS + H₂O₂ time 24 hr. | 0.183 |
| mAbA17 | PBS + H₂O₂ time 48 hr. | 0.207 |

### Example 8. Cross-Reactivity of Unconjugated Antibodies to Cyno, Rat, and Mouse 5T4 (ELISA & OCTET)

Unconjugated antibodies were tested for their ability to bind to non-human 5T4-Ts using ELISA and Octet. The ELISA protocol described in Example 7 was used, replacing the human 5T4 antigen for recombinant cyno 5T4-Fc (sourced from either R&D Systems (Minneapolis, MN, USA), Cat. No. 2280-TG-100 or LSBio (Seattle, WA, USA), Cat. No. LS-G137131), recombinant mouse 5T4-Fc (R&D Systems, Cat. No. 5049-TG-100), Recombinant Rat TPBG/5T4 with 6His-SUMO, N-terminus tags (LSBio Cat. No. G56592), Recombinant Mouse TPBG/5T4 with His, N-terminal tag (LSBio, Cat. No. G12168) antigen. For Octet analyses, a Protein A Biochip was coated with the antibody, washed with PBS, and then reacted with the antigen, as identified above. All antibodies tested bound to human, cyno, and rat 5T4. No significant binding cross-reactivity to mouse 5T4 was observed.

### Example 9. Cell Binding of Unconjugated Antibodies to 5T4-Transfected CHO and HEK293 Cells

Unconjugated antibodies were tested for their ability to bind to HEK293 and CHO cells transfected to express human, cyno, rat, or mouse 5T4. Non-transfected cells were utilized as a control to observe non-specific binding. mAbA4, mAbA15, and mAbA17 were reactive to HEK293 and CHO cells transfected to express human and cyno 5T4 but not to cells transfected to express rat or mouse 5T4.

### Example 10. In Vitro Cytotoxicity of 5T4 ADCs

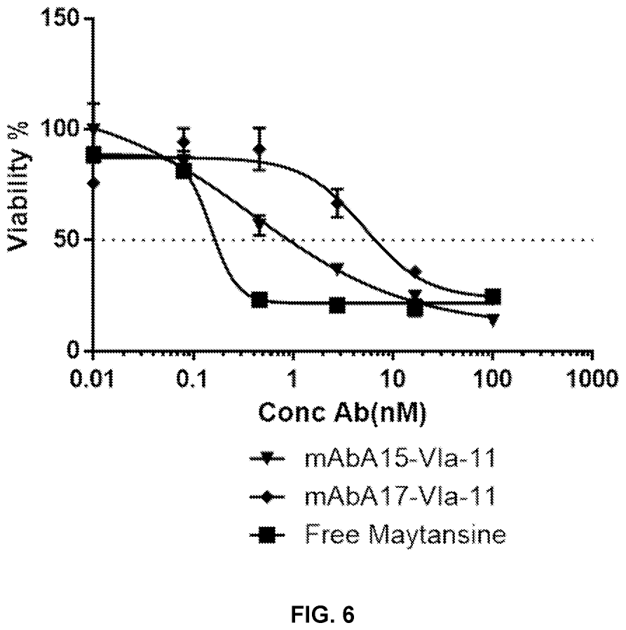

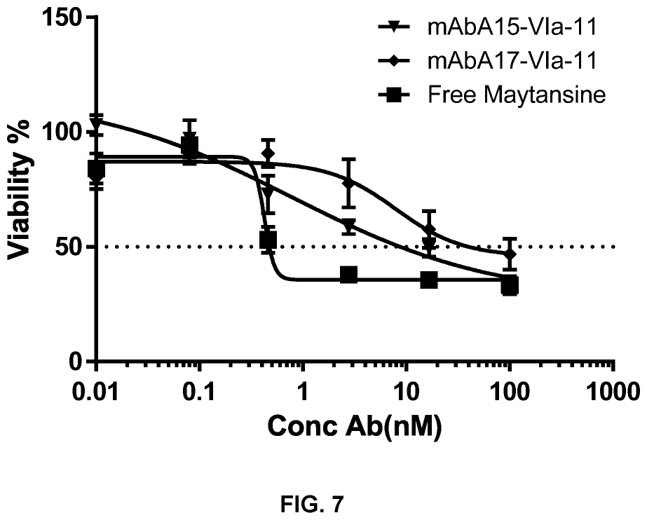

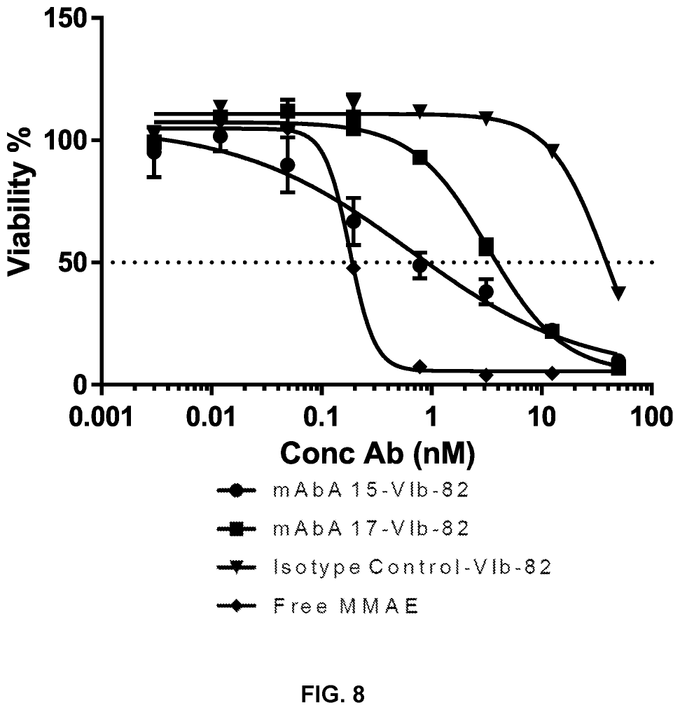

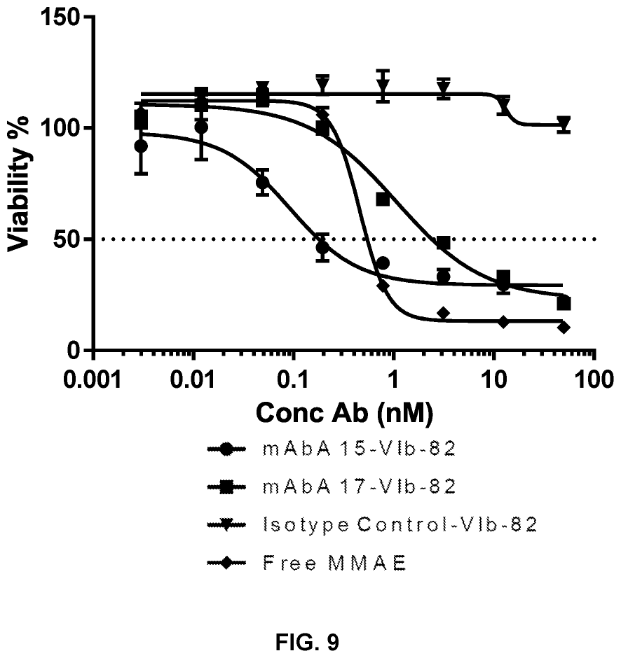

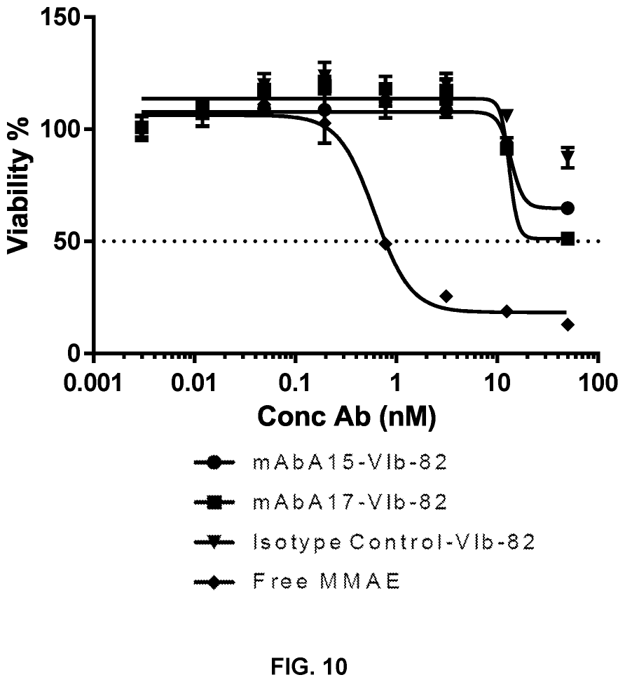

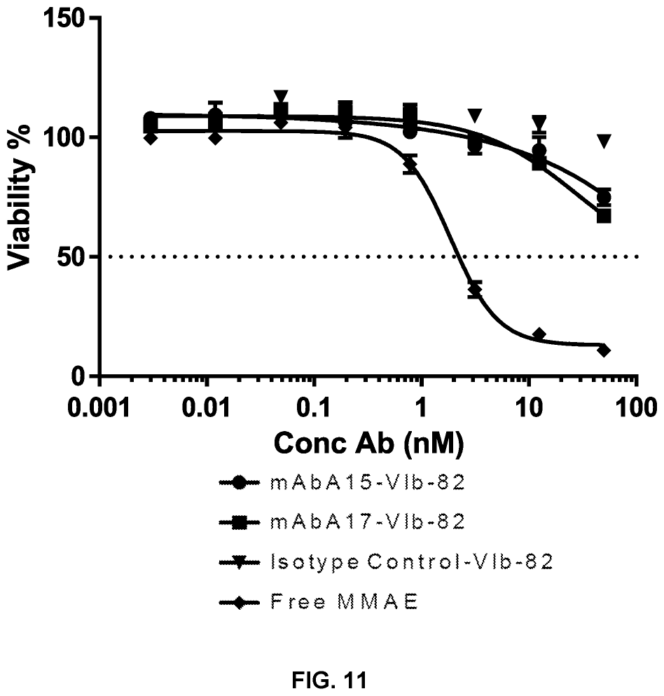

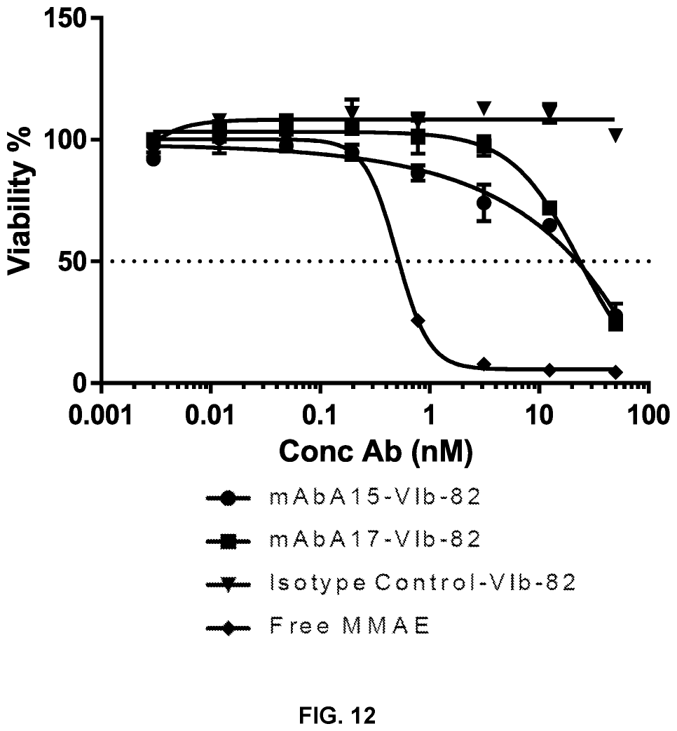

MDA-MB-468, MDA-MB-231, MCF-7, NCI-H226, or BxPC3 cells were plated at a density of 1500 cells per well in a Costar 3610 plate (Corning (Corning NY, USA), VWR Cat. No. VWR #29444-010) 100 μL of growth media and incubated overnight. A 6-fold dilution series was prepared for each ADC to be tested. 20 μL of each of the prepared ADC solution was added to the cells, resulting in a final dilution range of 100 nM to 0.02 nM, and incubated at 37° C. with 5% CO₂ for 5 days. Viability of the cells was measured using a Cell TITER-GLO® Viability Assay (Promega (Madison, WI, USA), Cat. No. G7573) and compared to untreated (control) cells.
A. VIa-11. FIG. 6 reports the cell viability of MDA-MB-468 cells treated with mAbA15-VIa-11, mAbA17-VIa-11, and mAbA17 conjugated directly to maytansine. FIG. 7 reports the cell viability of MCF-7 cells treated with mAb15-VIa-11, mAb15-VIa-11, and mAbA17 conjugated directly to maytansine.
B. VIa-82. FIG. 8 reports the cell viability of MDA-MB-468 cells treated with mAbA15-VIb-82, mAbA17-VIb-82, an isotype control, and free MMAE. FIG. 9 reports the cell viability of MCF-7 cells treated with mAbA15-VIb-82, mAbA17-VIb-82, an isotype control, and free MMAE. FIG. 10 reports the cell viability of MDA-MB-231 cells treated with various antibodies conjugated to linker-drug VIb-82. FIG. 11 reports the cell viability of NCI-H226 cells treated with various antibodies conjugated to linker-drug VIb-82. FIG. 12 provided below reports the cell viability of BxPC3 cells treated with various antibodies conjugated to linker-drug VIb-82.

### Example 11. In Vivo Efficacy of 5T4 ADCs Against Cancer Xenograft Model

MCF-7.1 Breast Cancer Xenograft Model. Female NCr NU/NU mice (n=8) were injected (flank) with 1×10⁷ of MCF-7.1 tumor cells in 50% MATRIGEL® (Corning Life Sciences) matrix. When the average tumor volumes reached 150-250 mm³, mice were randomized into respective treatment groups and received intravenous injections of vehicle, isotype control, or an ADC. ADCs were infused intravenously at a concentration of 5 mg/kg on either day 1 and day 4 after injection of MCF-7.1 cells or as a single intravenous injection of either vehicle, isotype control or test article (5 mg/kg) on Day 1. There were no adverse effects on body weight for any of the treatment groups through the course of the study.

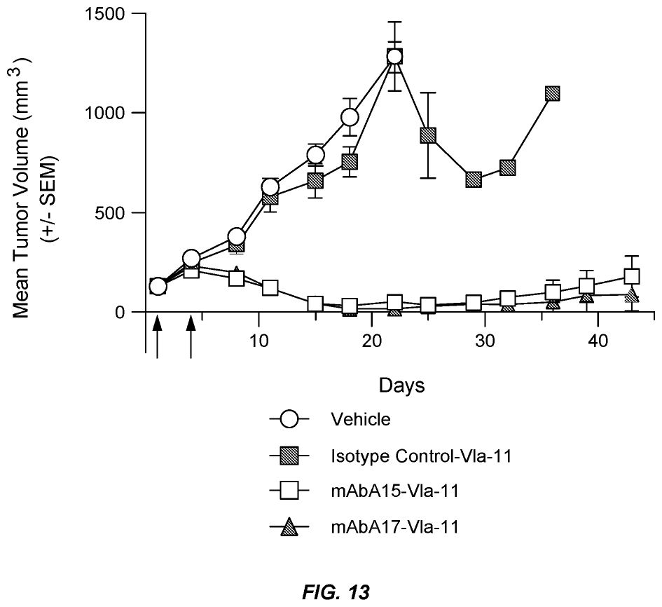

FIG. 13 represents the effect of treatment on tumor regression and suppression in MCF-7.1 xenograft mice treated with mAbA15-VIa-11 and mAbA17-VIa-11. As indicated by the arrows, mice received intravenous injections on day 1 and day 4 as indicated by the arrows on the x-axis of the graph.

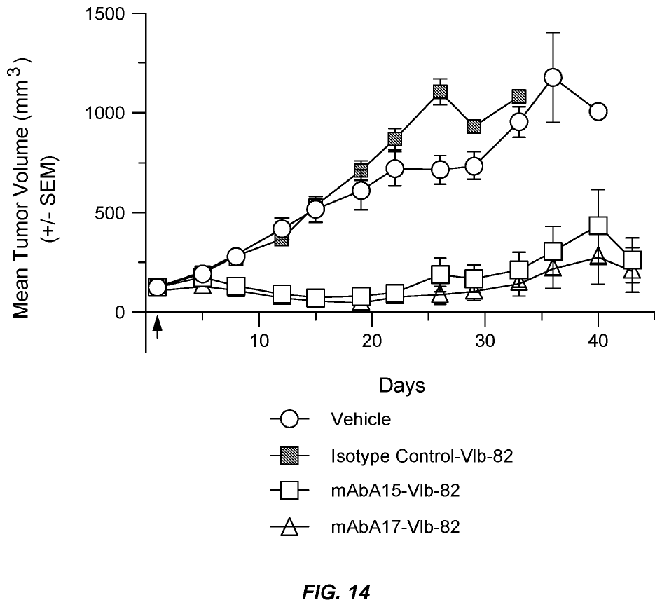

FIG. 14 represents the effect of treatment on tumor regression and suppression in MCF-7.1 xenograft mice treated with mAbA15-VIb-82 and mAbA17-VIb-82. As indicated by the arrow, mice received a single intravenous injection on Day 1 as indicated by the arrow on the x-axis of the graph.

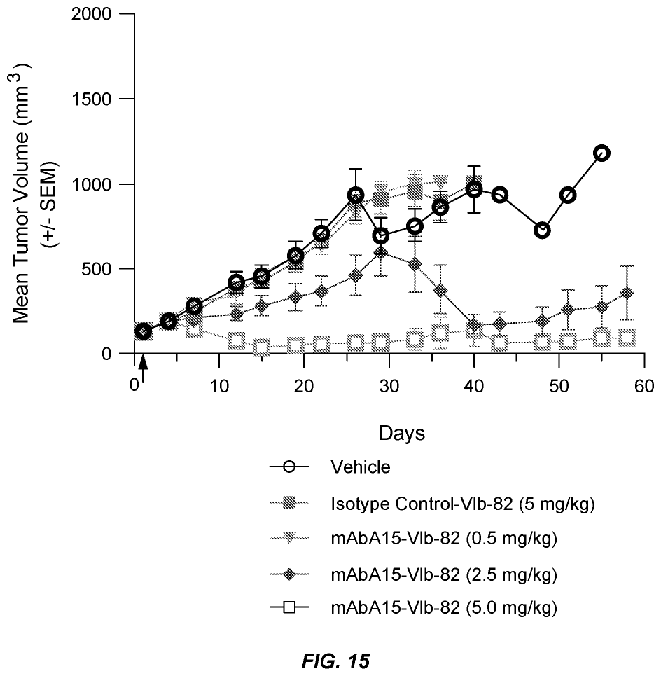

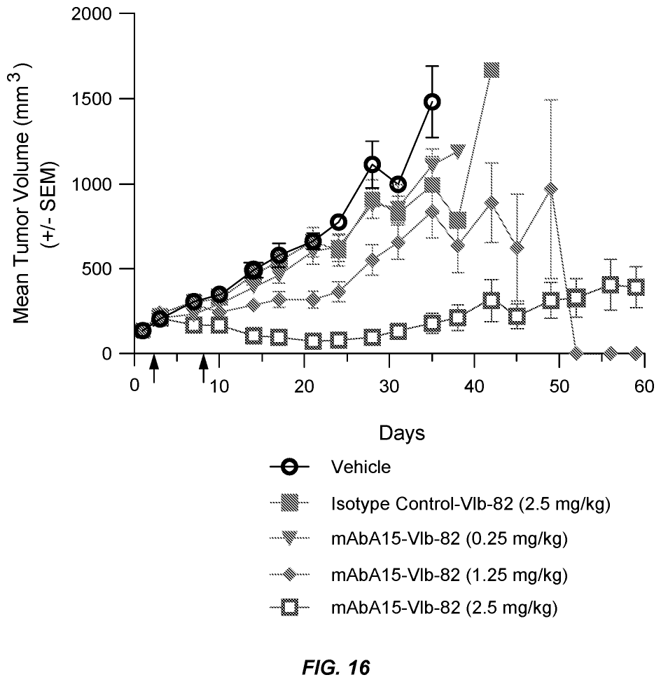

FIG. 15 represents the dose response effects of single dosing on tumor regression and suppression in MCF-7.1 Xenograft mice treated with one of three different dose strengths of mAbA15-VIb-82. As indicated by the arrow, mice received a single intravenous injection of vehicle, isotype control (5 mg/kg), or ADC (0.5 mg/kg, 2.5 mg/kg, or 5 mg/kg). FIG. 16 reports the dose response effects of weekly dosing on tumor regression and suppression in MCF-7.1 Xenograft mice treated with mAbA15-VIb-82. As indicated by the arrows, mice received a split dose on Days 1 and 8 of vehicle, isotype control (2.5 mg/kg) or ADC (0.25 mg/kg, 1.25 mg/kg, or 2.5 mg/kg).

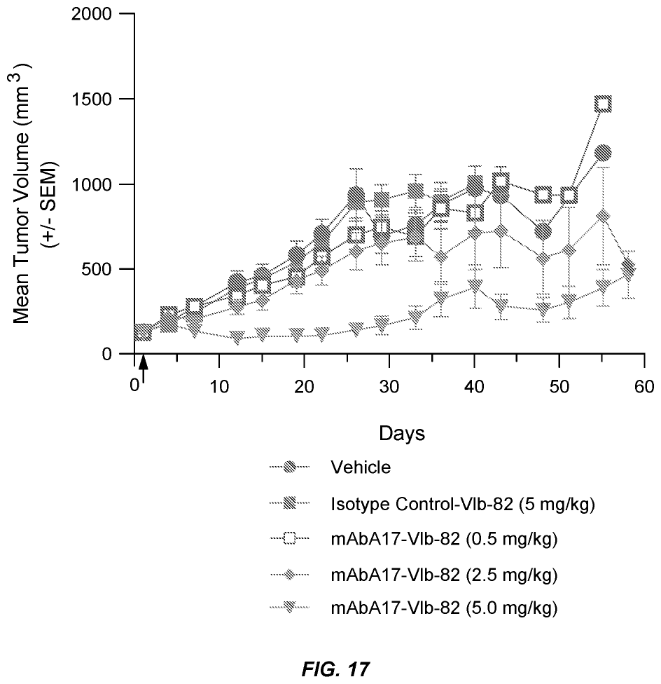

FIG. 17 reports the dose response effects of single dosing on tumor regression and suppression in MCF-7.1 Xenograft mice treated with mAbA17-VIb-82. As indicated by the arrow, mice received a single intravenous injection of vehicle, isotype control (5 mg/kg), or ADC (0.5 mg/kg, 2.5 mg/kg, or 5 mg/kg).

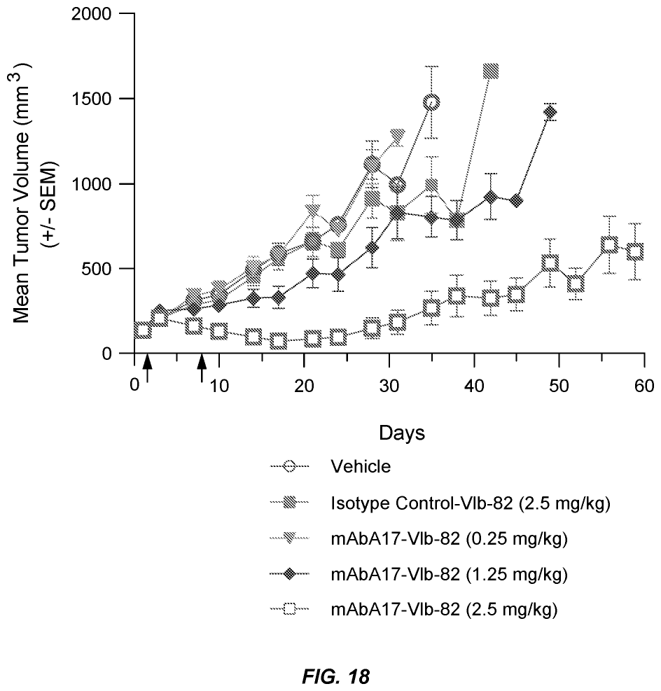

FIG. 18 reports the dose response effects of weekly dosing on tumor regression and suppression in MCF-7.1 Xenograft mice treated with mAbA17-VIb-82. As indicated by the arrows, mice received a split dose on day 1 and day 8 of vehicle, isotype control (2.5 mg/kg) or ADC (0.25 mg/kg, 1.25 mg/kg, or 2.5 mg/kg). MDA-MB-468 Breast Cancer Xenograft Model: Female SCID Beige mice (n=8) were injected (flank) with 1×10⁷ of MDA-MB-468 tumor cells in 50% MATRIGEL® (Corning Life Sciences) matrix. When the average tumor volume reached 100-150 mm³, mice were randomized into respective treatment groups and received intravenous injections of vehicle, 5 mg/kg isotype control, or 5 mg/kg ADC. There were no adverse effects on body weight for any of the treatment groups through the course of the study.

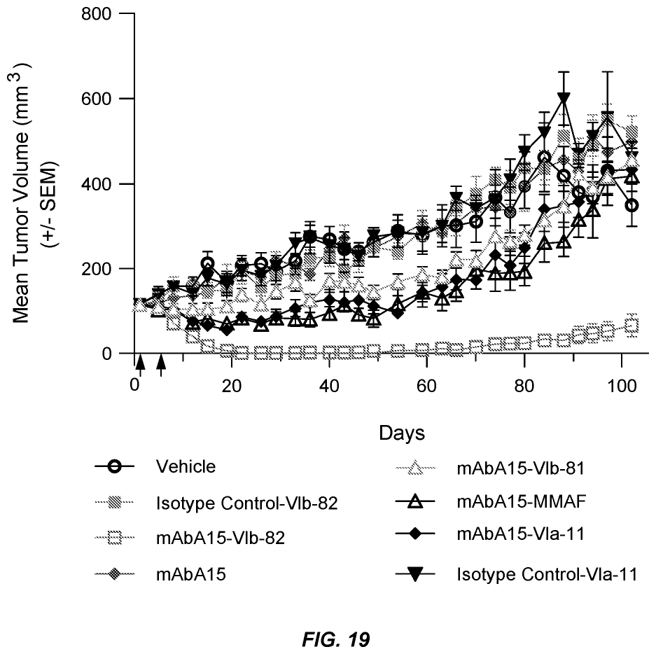

The effect of mAbA15 conjugated to linker-drug VIb-82 on tumor regression and suppression is compared to the effects of mAbA15 conjugated to linker-drug VIb-81 and directly to MMAF in FIG. 19. Mice received intravenous injections of ADC on day 1 and day 4 as indicated by the arrows on the x-axis of the graph.

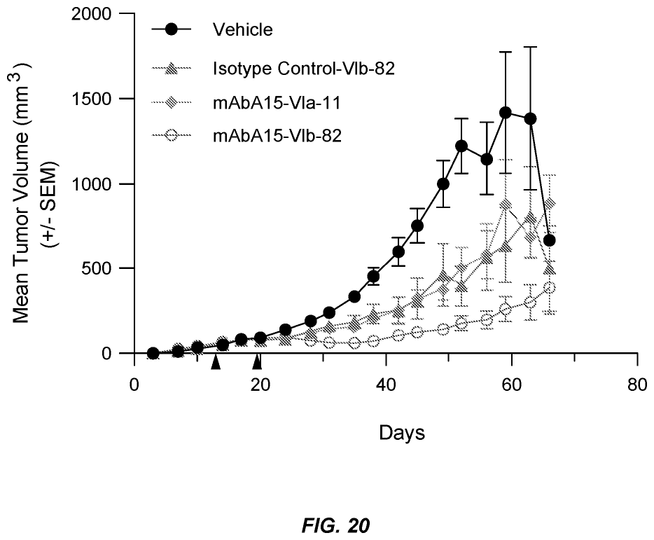

MDA-MB-231 Breast Cancer Xenograft Model: Female BalbC/nude mice (n=8) were inoculated with 1×10⁶ of MDA-MB-231 tumor cells into the mammary fat pad. When the average tumor volume reached 50-150 mm³, mice were randomized into respective treatment groups and received intravenous injections of vehicle, isotype control, or 5 mg/kg ADC. There were no adverse effects on body weight for any of the treatment groups through the course of the study. The effects of mAbA15-VIb-82 and mAbA15-VIa-11 on tumor regression and suppression are shown in FIG. 20. Mice received intravenous injections on day 1 and day 4 after mice were separated into experimental groups (the arrows on the x-axis of the graph indicate dosing on days after MCF-7 cell injection).

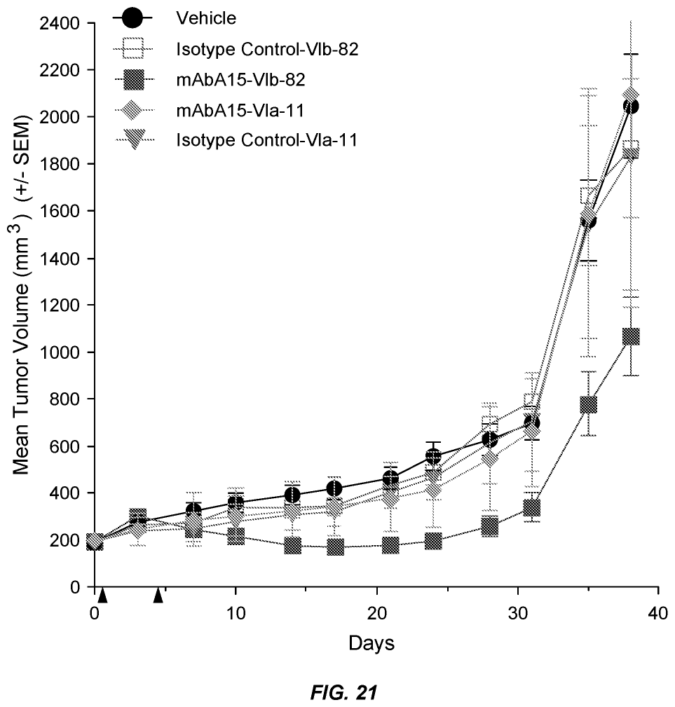

NCI-H226 Lung Cancer Xenograft Model: Female BalbC/nude mice (n=8) were inoculated with 1×10⁷ of NCI-H226 tumor cells in 1:1 MATRIGEL© matrix. When the average tumor volume reached 150-200 mm³, mice were randomized into respective treatment groups and received intravenous injections of vehicle, isotype control, or 5 mg/kg ADC. There were no adverse effects on body weight for any of the treatment groups through the course of the study. The effects of mAbA15-VIb-82 and mAbA15-VIa-11 on tumor regression and suppression are shown in FIG. 21. Mice received intravenous injections on day 1 and day 4 as indicated by the arrows on the x-axis of the graph.

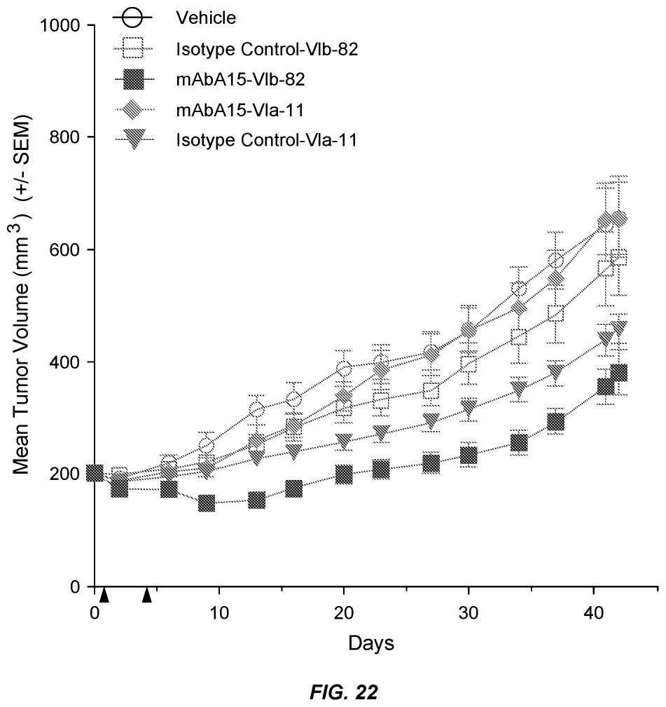

BxPC3 Pancreatic Cancer Xenograft Model: Female BalbC/nude mice (n=8) were inoculated with 1×10⁷ of BxPC3 tumor cells in 1:1 MATRIGEL® matrix. When the average tumor volume reached 150-200 mm³, mice were randomized into respective treatment groups and received intravenous injections of vehicle, isotype control, or 5 mg/kg ADC. There were no adverse effects on body weight for any of the treatment groups through the course of the study. The effects of mAbA15-VIb-82 and mAbA15-VIa-11 on tumor regression and suppression are shown in FIG. 22. Mice received intravenous injections on day 1 and day 4 as indicated by the arrows on the x-axis of the graph.

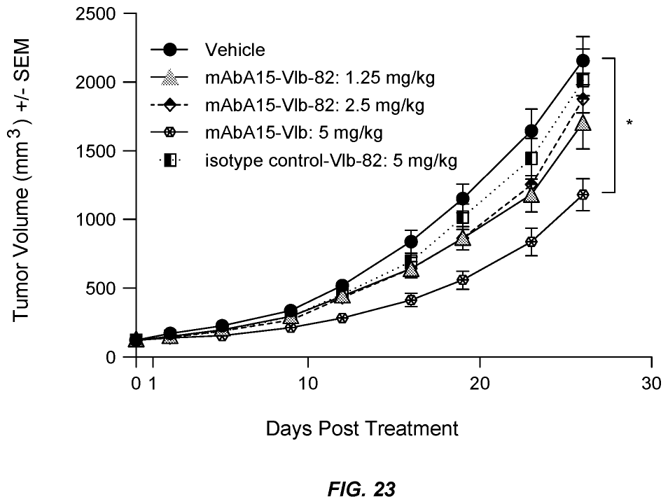

Calu-6 Lung Cancer Xenograft Model: Female BalbC/nude mice (n=10) were innoculated with 5×10⁶ of Calu-6 tumor cells in PBS. When the average tumor volume reached 100-200 mm³, mice were randomized into respective treatment groups and received intravenous injections of vehicle, isotype control, or 1.25, 2.5, or 5 mg/kg ADC. This was denoted as “day 1” of treatment. Mice received a single i.v. injection on day 1. There were no adverse effects on body weight for any of the treatment groups through the course of the study. The effects of mAbA15-VIb-82 on tumor regression and suppression are shown in FIG. 23.

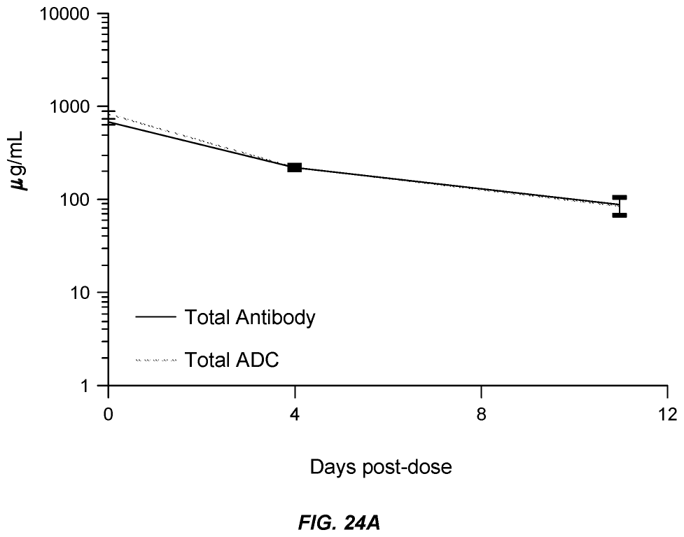

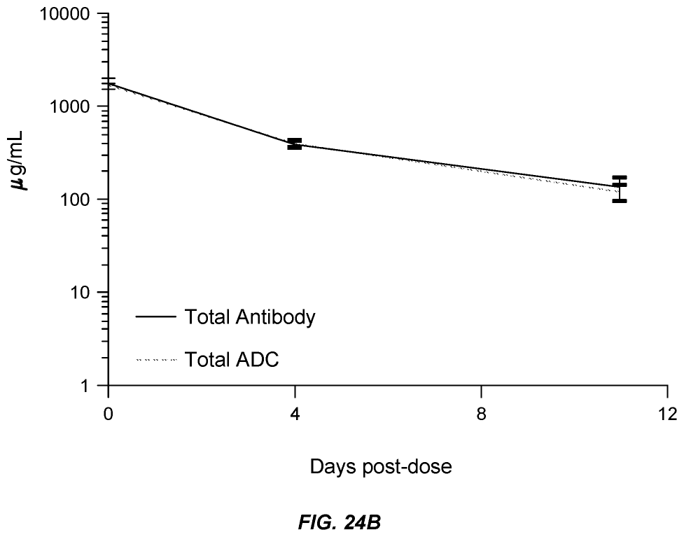

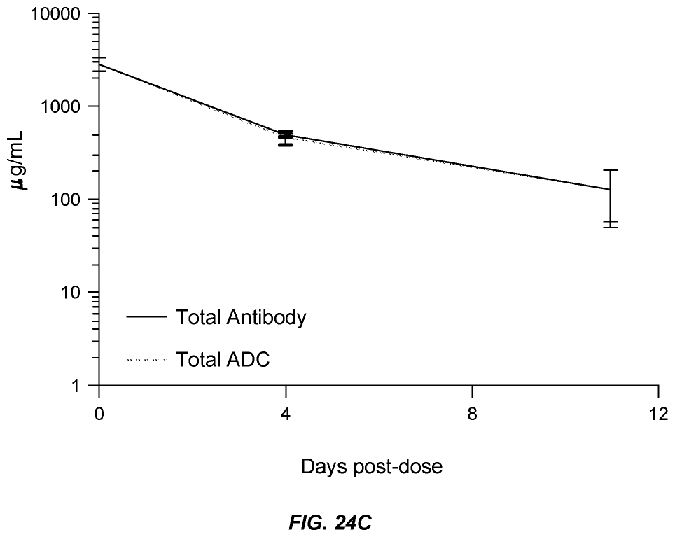

Rat Toxicokinetic (TK) Study: Male Sprague-Dawley rats (3 per group) were dosed intravenously with a single dose of 5 mg/kg of mAbA15-VIb-82 produced as described in the examples above. K2EDTA-stabilized plasma was collected at 0.5 h, 6 h and 24 h, and 2, 4, 7, 10, 14 and 21 days post-dose. Total antibody and total ADC concentrations were quantified by two ELISA methods. For total antibody measurements, conjugates were captured with an anti-human IgG-specific antibody and detected with an HRP-conjugated anti-human Fc-specific antibody. For total ADC measurements, conjugates were captured with an anti-human Fab-specific antibody and detected with a mouse anti-payload primary antibody, followed by an HRP-conjugated anti-mouse IgG-subclass 1-specific secondary antibody. Bound secondary antibody was detected using Ultra TMB One-Step ELISA substrate (ThermoFisher). After quenching the reaction with sulfuric acid, signals were read by taking the absorbance at 450 nm on a Molecular Devices Spectra Max M5 plate reader equipped with SoftMax Pro software. Data were analyzed using GraphPad Prism and Microsoft Excel software and plotted in FIGS. 24A (30 mg/kg), 24B (60 mg/kg), and 24C (90 mg/kg).

Throughout this application, various publications, patents, patent applications and other documents have been referenced. The disclosures of these publications, patents, patent applications and other documents in their entireties are hereby incorporated by reference in this application for all purposes, including in order to more fully describe the state of the art to which this the subject matter disclosed herein pertains. Although the disclosed subject matter has been described with reference to the examples provided above, it should be understood that various modifications could be made without departing from the spirit of the disclosed subject matter. Many variations will become apparent to those skilled in the art upon review of this specification.

What is claimed is:

1. An antibody-drug conjugate (ADC) of Formula (IV) comprising:
a. an antibody that binds to 5T4; and
b. one or more drugs conjugated to one or more pyridazine-pyrrolo coupling moieties via a linker 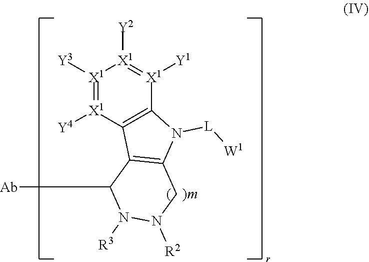
wherein:
Ab represents the antibody that binds to 5T4;
r is an integer from 1 to 10;
m is 0 or 1;
R² and R³ are each independently selected from the group consisting of hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl; or R² and R³ are cyclically linked to form a 5- or 6-membered heterocyclyl;
X¹, X², X³ and X⁴ are each independently selected from the group consisting of C, N, O and S;
Y¹, Y², Y³ and Y⁴ are each independently selected from the group consisting of hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or Y¹ and Y², Y² and Y³, or Y³ and Y⁴ are cyclically linked;
W¹ is the drug;
L represents the linker and is -(T¹V¹)ₐ-(T²V²)_{b}-(T³V³)_{c}-(T⁴V⁴)_{d}-(T⁵V⁵)ₑ—, wherein:
a, b, c, d, and e are each independently 0 or 1, wherein the sum of a, b, c, d, and e is 1 to 5;
T¹, T², T³, T⁴, and T⁵ each independently comprise C₁-C₁₂ alkyl, substituted C₁-C₁₂ alkyl, (EDA)_{w}, (PEG)ₙ, (AA)ₚ, —(CR¹³OH)ₙ—, a piperidin-4-amine (P4A-R¹²), a meta-aminobenzyl carbamate (MABC) group, a meta-aminobenzyloxy (MABO) group, a para-aminiobenzyloxy (PABO) group, a para-aminobenzyl carbamate (PABC) group, a para-aminobenzyl (PAB) group, acetal, a disulfide, a hydrazine, (AA)ₚ-MABC-(AA)ₚ, (AA)ₚ-MABO-(AA)ₚ, (AA)ₚ-PABO-(AA)ₚ, or (AA)ₚ-PABC-(AA)ₚ,
w is an integer from 1 to 20;
n is an integer from 1 to 30;
each p is independently zero or an integer from 1 to 20;
h is an integer from 1 to 12; and
each R¹² is hydrogen, alkyl, substituted alkyl, a polyethylene glycol moiety, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl;
each R¹³ is hydrogen, alkyl, substituted alkyl, aryl, or substituted aryl;
V¹, V², V³, V⁴ and V⁵ are each independently selected from the group consisting of a covalent bond, —C(═O)—, —NR¹¹—, —C(═O)NR¹¹—, —NR¹¹C(═O)—, —C(═O)O—, —OC(═O)—, —O—, —S—, —S(═O)—, —SO₂—, —SO₂NR¹¹—, —NR¹¹SO₂— and —P(═O)OH—, wherein R¹¹ is selected from the group consisting of hydrogen, alkyl, substituted alkyl, PEG, aryl, and substituted aryl.

2. The ADC of claim 1, wherein W¹ is selected from the group consisting of a maytansinoid and an auristatin.

3. The ADC of claim 1 or 2, wherein the auristatin is MMAE.

4. The ADC of any one of claims 1-3, wherein R² and R³ are each alkyl.

5. The ADC of any one of claims 1-4, wherein R² and R³ are each methyl.

6. The ADC of any one of claims 1-5, wherein each of X¹, X², X³, and X⁴ are independently selected from C and N.

7. The ADC of any one of claims 1-6, wherein the ADC is represented by Formula (V):

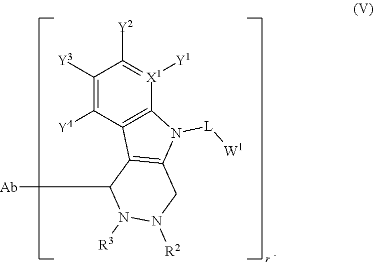

8. The ADC of any one of claims 1-7, wherein the ADC is represented by Formula (VI): 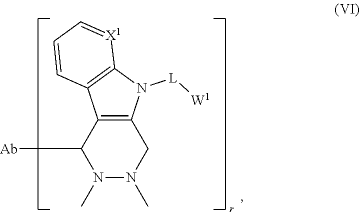
wherein X¹ is C or N.

9. The ADC of any one of claims 1-8, wherein T¹, T², T³, T⁴, and T⁵ are each independently selected from the group consisting of C₁-C₆ alkyl, P4A-R¹², (AA)ₚ, (AA)ₚ-PABC, and (PEG)ₙ, wherein p is an integer from 1 to 5, and R¹² is a polyethylene glycol or carboxylic acid-modified polyethylene glycol.

10. The ADC of any one of claims 1-9, wherein
T¹ is C₁-C₆ alkyl;
T², T³, T⁴ and T⁵ are each independently selected from (PEG)ₙ, C₁-C₆ alkyl, (AA)ₚ, P4A-R¹², (AA)ₚ-(PABO-R¹⁶)-(AA)ₚ, (AA)ₚ-(PABC-R¹⁶)-(AA)_{P}, (AA)ₚ-(PABO-R¹⁶), and (AA)ₚ-(PABC-R¹⁶), wherein R¹² is a carboxylic acid-modified polyethylene glycol and R¹⁶ is hydrogen; and
V¹, V², V³, V⁴ and V⁵ are each independently selected from —CO— and —NR¹¹—, wherein R¹¹ is selected from hydrogen and alkyl.

11. The ADC of any one of claims 1-10, wherein L is: 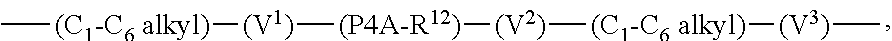
wherein:
V¹, V², and V³ are each independently selected from the group consisting of —CO— and —NR¹¹—
each R¹¹ is independently selected from group consisting of hydrogen and C₁-C₆ alkyl; and
R¹² is a carboxylic acid-modified polyethylene glycol moiety.

12. The ADC of any one of claims 1-11, wherein L is: 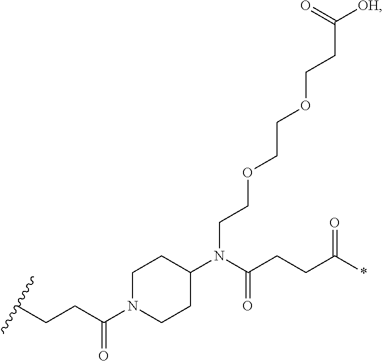 [SMILES: *CCC(=O)N1CCC(N(CCOCCOCCC(=O)O)C(=O)CCC(*)=O)CC1]
wherein: [symbol]
represents attachment to the nitrogen of the pyridazine-pyrrolo coupling moiety; and
* represents attachment to W¹.

13. An antibody-drug conjugate (ADC) of Formula (VI-1) comprising:
a. an antibody that binds to 5T4; and
b. one or more drugs conjugated to one or more pyridazine-pyrrolo coupling moieties via a linker 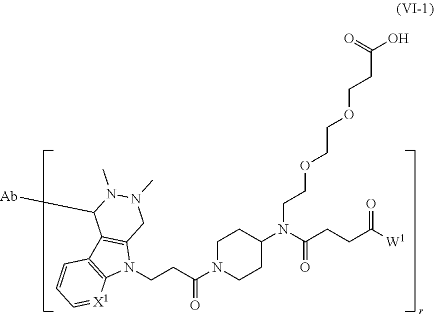
wherein:
Ab represents the antibody that binds to 5T4;
r is an integer from 1 to 10;
X¹ is C or N; and
W¹ is the drug.

14. The ADC of claim 13, wherein W¹ is selected from the group consisting of a maytansinoid and an auristatin.

15. The ADC of claim 14, wherein the auristatin in MMAE.

16. The ADC of claim 13 or 14, wherein X¹ is C and W¹ is deacylated maytansine.

17. The ADC of any one of claims 14-15, wherein X¹ is N and W¹ is MMAE.

18. The ADC of any one of claims 13-17, wherein L is: 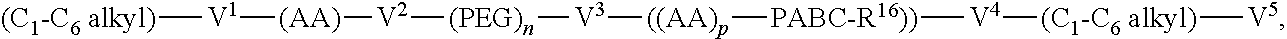
wherein:
R¹⁶ is hydrogen;
n and p are each independently an integer from 1 to 20;
V¹, V², V³, V⁴ and V⁵ are each independently selected from the group consisting of —CO— and —NR¹¹—; and
each R¹¹ is independently selected from the group consisting of hydrogen and C₁-C₆ alkyl.

19. The ADC of any one of claims 13-17, wherein L is: 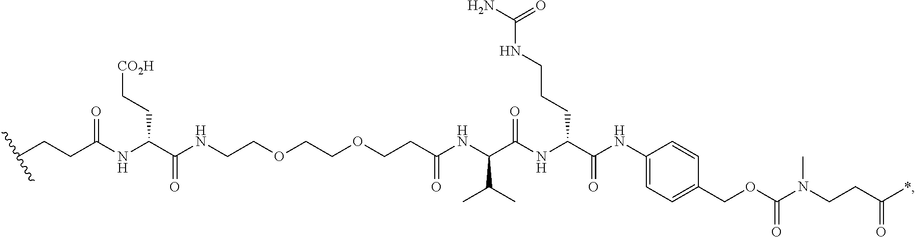
wherein: [symbol]
represents attachment to the nitrogen of the pyridazine-pyrrolo coupling moiety; and
* represents attachment to W¹.

20. An antibody-drug conjugate (ADC) comprising an antibody that binds to 5T4 conjugated to one or more drugs via one or more pyridazine-pyrrolo coupling moieties, as represented by Formula (VI-2): 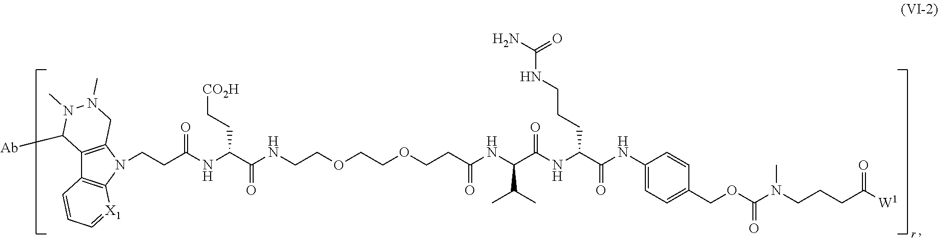
wherein:
Ab represents the antibody that binds to 5T4;
r is an integer from 1 to 10;
X¹ is C or N; and
W¹ is the drug.

21. The ADC of claim 20, wherein W¹ is selected from a maytansinoid and an auristatin.

22. The ADC of claim 21, wherein the auristatin is MMAE.

23. The ADC of claim 20 or 21 wherein X¹ is C and W¹ is deacylated maytansine.

24. An antibody-drug conjugate (ADC) of Formula (IV) comprising:
a. an antibody that binds to 5T4; and
b. one or more drugs conjugated to one or me pyridazine-pyrrolo coupling moieties via a linker 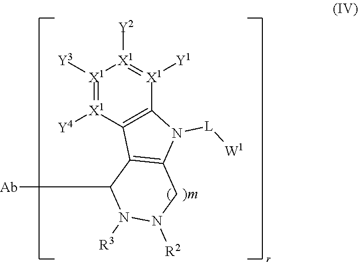
wherein:
Ab represents the antibody that binds to 5T4;
W¹ is the drug;
r is an integer from 1 to 10;
m is 0 or 1;
R² and R³ are each independently selected from the group consisting of hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl; or R² and R³ are cyclically linked to form a 5- or 6-membered heterocyclyl;
X¹, X², X³, and X⁴ are each independently selected from the group consisting of C, N, O and S;
Y¹, Y², Y³, and Y⁴ are each independently selected from the group consisting of hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl; or Y¹ and Y², Y² and Y³, or Y³ and Y⁴ are cyclically linked;
L is 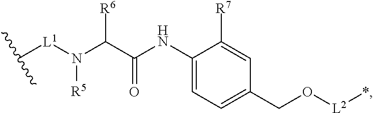
wherein: [symbol]
represents attachment to the nitrogen of the pyridazine-pyrrolo coupling moiety;
* represents attachment to W¹;
each R⁵ is independently hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl;
each R⁶ is independently selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R⁷ is a cleavable moiety;
k is an integer from 1 to 10;
L¹ comprises -(T¹-V¹)ₐ-(T²-V²)_{b}-(T³-V³)_{c}-(T⁴-V⁴)_{d}—;
L² comprises -(T⁵-V⁵)ₑ-(T⁶-V⁶)_{f}-(T⁷-V⁷)_{g}-(T⁸-V⁸)ₕ—;
each of a, b, c, d, e, f, g, and h are independently 1 or 0;
T¹, T², T³, T⁴, T⁵, T⁶, T⁷, and T⁸ are each independently selected from the group consisting of a covalent bond, C₁-C₁₂ alkyl, substituted C₁-C₁₂ alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, P4A-R¹², acetal, a hydrazine, a disulfide, and an ester;
each w is an integer from 1 to 20;
each n is an integer from 1 to 30;
each p is an integer from 1 to 20;
each m is an integer from 1 to 12;
V¹, V², V³, V⁴, V⁵, V⁶, V⁷, and V⁸ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
each q is an integer from 1 to 6;
R¹² is selected from the group consisting of hydrogen, alkyl, substituted alkyl, a polyethylene glycol moiety, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
each R¹³ is independently selected from the group consisting of hydrogen, alkyl, substituted alkyl, aryl, and substituted aryl; and
each R¹⁵ is independently selected from the group consisting of hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

25. The ADC of claim 24, wherein W¹ is selected from a maytansinoid and an auristatin.

26. The ADC of claim 25, wherein the auristatin in MMAE.

27. The ADC of any one of claims 24-26, wherein R² and R³ are each alkyl.

28. The ADC of claim 27, wherein R² and R³ are each methyl.

29. The ADC of any one of claims 24-28, wherein each of X¹, X², X³, and X⁴ are independently selected from C and N.

30. The ADC of any one of claims 24-29, represented by Formula (VI-3):

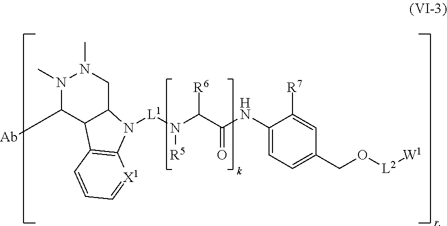

31. The ADC of claim 30, wherein R⁷ is 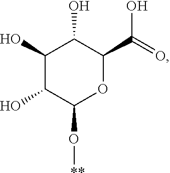
wherein** represents the point of attachment to the phenyl group in Formula (VI-3).

32. The ADC of any one of claims 24-31, represented by Formula (V-5): 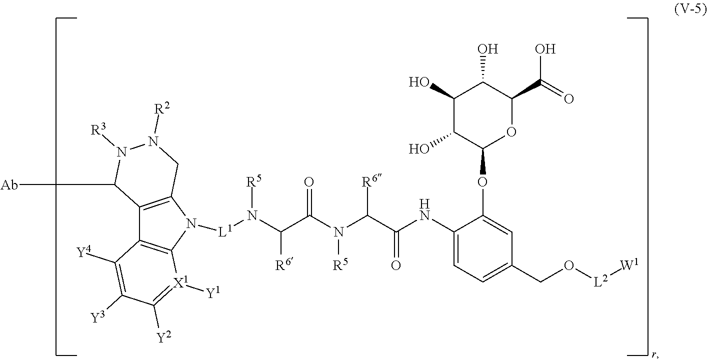
wherein R^{6′} and R^{6″} are each independently alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl.

33. The ADC of any one of claims 24-32, represented by Formula (VI-5): 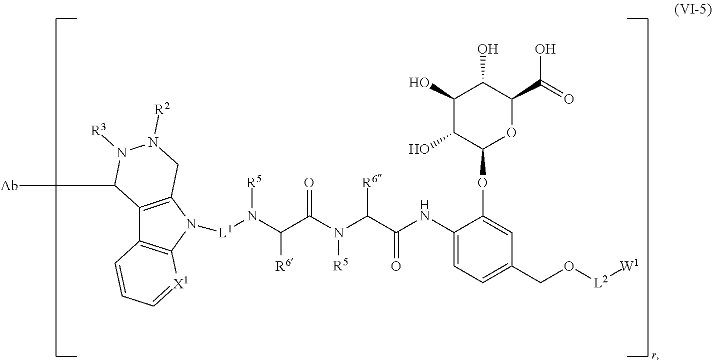
wherein X¹ is C or N, wherein R^{6′} and R^{6″} are each independently alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, or substituted heterocyclyl.

34. The ADC of any one of claims 24-33, represented by Formula (VI-6):

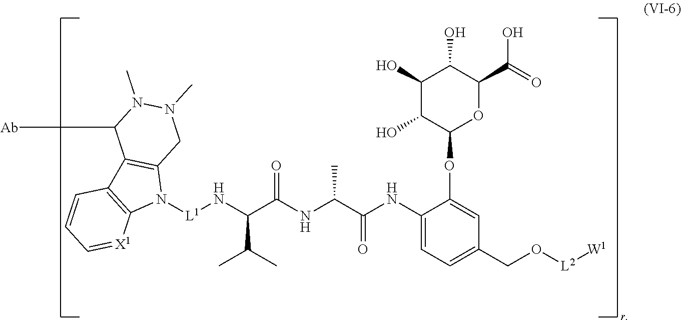

35. The ADC of any one of claims 24-34, wherein
L¹ comprises -(T¹-V¹)ₐ-(T²-V²)_{b}-(T³-V³)_{c}-(T⁴-V⁴)_{d}—;
a, b, and c are each 1;
d is 0 and therefore T⁴ and V⁴ are absent;
T¹, T², and T³ are each independently selected from the group consisting of C₁-C₁₂ alkyl, (PEG)ₙ, and (AA)ₚ,
n is an integer from 1 to 10;
p is an integer from 1-10;
V¹, V², and V³ are each independently selected from the group consisting of —C(═O)— and —NR¹¹—; and
R¹¹ is selected from hydrogen, alkyl, substituted alkyl, a polyethylene glycol moiety, aryl, and substituted aryl.

36. The ADC of any one of claims 24-35, wherein L¹ is 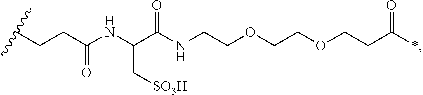
wherein [symbol] represents the point of attachment to the nitrogen of a pyridazine-pyrrolo coupling moiety and wherein * represents a point of attachment in any direction to a remaining portion of the linker.

37. The ADC of any one of claims 24-36, represented by Formula (VI-7): 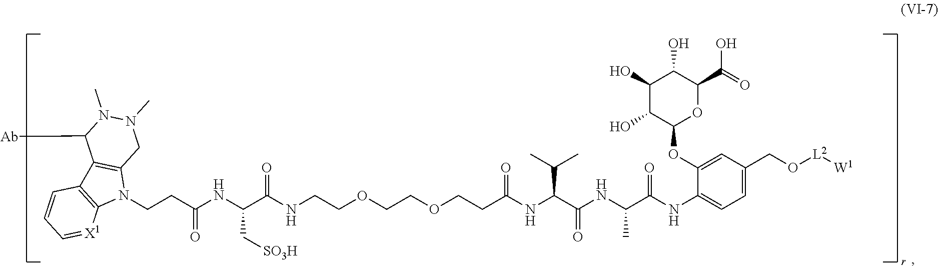
wherein X¹ is C or N.

38. The ADC of any of claims 24-37, wherein L² is a carbonyl group.

39. The ADC of any of claims 24-38, wherein X¹ is N and W¹ is MMAE.

40. The ADC of any of claims 24-39, represented by Formula (VIb-82):

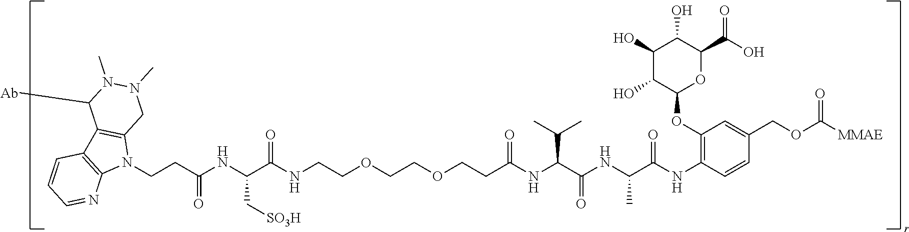

41. The ADC of any one of claims 1-40, wherein the antibody comprises:
(i) a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26; or
(ii) a VH CDR1, a VH CDR2, and a VH CDR3 as set forth in a VH comprising the amino acid sequence of SEQ ID NO:44 and a VL CDR1, a VL CDR2, and a VL CDR3 as set forth in a VL comprising the amino acid sequence of SEQ ID NO:45; or
(iii) a VH CDR1, a VH CDR2, and a VH CDR3 as set forth in a VH comprising the amino acid sequence of SEQ ID NO:62 and a VL CDR1, a VL CDR2, and a VL CDR3 as set forth in a VL comprising the amino acid sequence of SEQ ID NO:63.

42. The ADC of any one of claims 1-41, wherein the antibody comprises:
(a) a VH region comprising:
(1) a VH CDR1 having an amino acid sequence selected from the group consisting of SEQ ID NOs:1, 7, 12, 13, and 18.
(2) a VH CDR2 having an amino acid sequence selected from the group consisting of SEQ ID NOs:2, 8, 14, 19, and 24; and
(3) a VH CDR3 having an amino acid sequence selected from the group consisting of SEQ ID NOs:3, 9, 15, and 20;
and
(b) a VL region comprising:
(1) a VL CDR1 having an amino acid sequence selected from the group consisting of SEQ ID NOs:4, 10, 16, and 21;
(2) a VL CDR2 having an amino acid sequence selected from the group consisting of SEQ ID NOs:5, 11, and 22; and
(3) a VL CDR3 having an amino acid sequence selected from the group consisting of SEQ ID NOs:6, 17, and 23.

43. The ADC of any one of claims 1-42, wherein the antibody comprises:
(i) a VH region comprising a VH CDR1 comprising the amino acid sequence of SEQ ID NO:1, a VH CDR2 comprising the amino acid sequence of SEQ ID NO:2, and a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; or
(ii) a VH region comprising a VH CDR1 comprising the amino acid sequence of SEQ ID NO:7, a VH CDR2 comprising the amino acid sequence of SEQ ID NO:8, and a VH CDR3 comprising the amino acid sequence of SEQ ID NO:9; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:6; or
(iii) a VH region comprising a VH CDR1 comprising the amino acid sequence of SEQ ID NO:12, a VH CDR2 comprising the amino acid sequence of SEQ ID NO:2, and a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; or
(iv) a VH region comprising a VH CDR1 comprising the amino acid sequence of SEQ ID NO:13, a VH CDR2 comprising the amino acid sequence of SEQ ID NO:14, and a VH CDR3 comprising the amino acid sequence of SEQ ID NO:15; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:17; or
(v) a VH region comprising a VH CDR1 comprising the amino acid sequence of SEQ ID NO:18, a VH CDR2 comprising the amino acid sequence of SEQ ID NO:19, and a VH CDR3 comprising the amino acid sequence of SEQ ID NO:20; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:23; or
(vi) a VH region comprising a VH CDR1 comprising the amino acid sequence of SEQ ID NO:1, a VH CDR2 comprising the amino acid sequence of SEQ ID NO:24, and a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

44. The ADC of any one of claims 1-41, wherein the antibody comprises:
(a) a VH region comprising:
(1) a VH CDR1 having an amino acid sequence selected from the group consisting of SEQ ID NOs:27, 31, 34, 35, and 39;
(2) a VH CDR2 having an amino acid sequence selected from the group consisting of SEQ ID NOs:28, 32, 36, 40, and 43; and
(3) a VH CDR3 having an amino acid sequence selected from the group consisting of SEQ ID NOs:29, 33, 37, and 41; and
(b) a VL region comprising:
(1) a VL CDR1 having an amino acid sequence selected from the group consisting of SEQ ID NOs:4, 10, 16, and 21;
(2) a VL CDR2 having an amino acid sequence selected from the group consisting of SEQ ID NOs:5, 11, and 22; and
(3) a VL CDR3 having an amino acid sequence selected from the group consisting of SEQ ID NOs:30, 38, and 42.

45. The ADC of any one of claims 1-41, and 44, wherein the antibody comprises:
(i) a VH region comprising a VH CDR1 comprising the amino acid sequence of SEQ ID NO:27, a VH CDR2 comprising the amino acid sequence of SEQ ID NO:28, and a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30; or
(ii) a VH region comprising a VH CDR1 comprising the amino acid sequence of SEQ ID NO:31, a VH CDR2 comprising the amino acid sequence of SEQ ID NO:32, and a VH CDR3 comprising the amino acid sequence of SEQ ID NO:33; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:30; or
(iii) a VH region comprising a VH CDR1 comprising the amino acid sequence of SEQ ID NO:34, a VH CDR2 comprising the amino acid sequence of SEQ ID NO:28, and a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30; or
(iv) a VH region comprising a VH CDR1 comprising the amino acid sequence of SEQ ID NO:35, a VH CDR2 comprising the amino acid sequence of SEQ ID NO:36, and a VH CDR3 comprising the amino acid sequence of SEQ ID NO:37; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:38; or
(v) a VH region comprising a VH CDR1 comprising the amino acid sequence of SEQ ID NO:39, a VH CDR2 comprising the amino acid sequence of SEQ ID NO:40, and a VH CDR3 comprising the amino acid sequence of SEQ ID NO:41; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:42; or
(vi) a VH region comprising a VH CDR1 comprising the amino acid sequence of SEQ ID NO:27, a VH CDR2 comprising the amino acid sequence of SEQ ID NO:43, and a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30.

46. The ADC of any one of claims 1-41, wherein the antibody comprises:
(a) a VH region comprising:
(1) a VH CDR1 having an amino acid sequence selected from the group consisting of SEQ ID NOs:46, 50, 53, 13, and 57;
(2) a VH CDR2 having an amino acid sequence selected from the group consisting of SEQ ID NOs:47, 51, 54, 58, and 61; and
(3) a VH CDR3 having an amino acid sequence selected from the group consisting of SEQ ID NOs:48, 52, 55, and 59; and
(b) a VL region comprising:
(1) a VL CDR1 having an amino acid sequence selected from the group consisting of SEQ ID NOs:4, 10, 16, and 21;
(2) a VL CDR2 having an amino acid sequence selected from the group consisting of SEQ ID NOs:5, 11, and 22; and
(3) a VL CDR3 having an amino acid sequence selected from the group consisting of SEQ ID NOs:49, 56, and 60.

47. The ADC of any one of claims 1-41, and 46, wherein the antibody comprises:
(i) a VH region comprising a VH CDR1 comprising the amino acid sequence of SEQ ID NO:46, a VH CDR2 comprising the amino acid sequence of SEQ ID NO:47, and a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49; or
(ii) a VH region comprising a VH CDR1 comprising the amino acid sequence of SEQ ID NO:50, a VH CDR2 comprising the amino acid sequence of SEQ ID NO:51, and a VH CDR3 comprising the amino acid sequence of SEQ ID NO:52; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:49; or
(iii) a VH region comprising a VH CDR1 comprising the amino acid sequence of SEQ ID NO:53, a VH CDR2 comprising the amino acid sequence of SEQ ID NO:47, and a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49; or
(iv) a VH region comprising a VH CDR1 comprising the amino acid sequence of SEQ ID NO:13, a VH CDR2 comprising the amino acid sequence of SEQ ID NO:54, and a VH CDR3 comprising the amino acid sequence of SEQ ID NO:55; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:56; or
(v) a VH region comprising a VH CDR1 comprising the amino acid sequence of SEQ ID NO:57, a VH CDR2 comprising the amino acid sequence of SEQ ID NO:58, and a VH CDR3 comprising the amino acid sequence of SEQ ID NO:59; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:60; or
(vi) a VH region comprising a VH CDR1 comprising the amino acid sequence of SEQ ID NO:46, a VH CDR2 comprising the amino acid sequence of SEQ ID NO:61, and a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49.

48. The ADC of any one of claims 1-46, wherein the antibody further comprises a framework 1 (FR1), a framework 2 (FR2), a framework 3 (FR3) and/or a framework 4 (FR4) sequence as set forth in any one of SEQ ID NOs:25, 26, 44, 45, 62, and 63.

49. The ADC of any one of claims 1-48, wherein the antibody further comprises human framework sequences.

50. The ADC of any one of claims 1-49, wherein the antibody comprises:
(i) a VH comprising the amino acid sequence of SEQ ID NO:25 and a VL comprising the amino acid sequence of SEQ ID NO:26; or
(ii) a VH comprising the amino acid sequence of SEQ ID NO:44 and a VL comprising the amino acid sequence of SEQ ID NO:45; or
(iii) a VH comprising the amino acid sequence of SEQ ID NO:62 and a VL comprising the amino acid sequence of SEQ ID NO:63.

51. A pharmaceutical composition comprising an ADC of any one of claims 1-50 and a pharmaceutically acceptable excipient.

52. The pharmaceutical composition of claim 51, wherein the drug-to-antibody ratio of the antibody-drug conjugate is about 1 to about 4.

53. The pharmaceutical composition of claim 52, wherein the drug-to-antibody ratio is about 2.

54. A method for treating a cancer or a tumor in a subject comprising administering to the subject the antibody-drug conjugate of any one of claims 1-49 or the pharmaceutical composition of any one of claims 51-53.


SEQUENCE LISTING (103 sequences)

SEQ ID NO: 1 - protein, 10 aa, synthetic construct
  source 1..10: note = Antibody mAbA4 VH CDR1 (Exemplary, AbM)
GFTFSRYYIH

SEQ ID NO: 2 - protein, 17 aa, synthetic construct
  source 1..17: note = Antibody mAbA4 VH CDR2 (Exemplary, Kabat)
FISPGGSYTYYADSVKG

SEQ ID NO: 3 - protein, 10 aa, synthetic construct
  source 1..10: note = Antibody mAbA4 VH CDR3 (Exemplary, Kabat, AbM)
EYYSMGVLDY

SEQ ID NO: 4 - protein, 11 aa, synthetic construct
  source 1..11: note = Antibody mAbA4, mAbA15, mAbA17 VL CDR1 (Exemplary, Kabat, AbM)
RASQSVSSAVA

SEQ ID NO: 5 - protein, 7 aa, synthetic construct
  source 1..7: note = Antibody mAbA4, mAbA15, mAbA17 VL CDR2 (Exemplary, Kabat, AbM)
SASSLYS

SEQ ID NO: 6 - protein, 9 aa, synthetic construct
  source 1..9: note = Antibody mAbA4 VL CDR3 (Exemplary, IMGT, Kabat, AbM)
QQAYESPYT

SEQ ID NO: 7 - protein, 8 aa, synthetic construct
  source 1..8: note = Antibody mAbA4 VH CDR1 (IMGT)
GFTFSRYY

SEQ ID NO: 8 - protein, 8 aa, synthetic construct
  source 1..8: note = Antibody mAbA4 VH CDR2 (IMGT)
ISPGGSYT

SEQ ID NO: 9 - protein, 12 aa, synthetic construct
  source 1..12: note = Antibody mAbA4 VH CDR3 (IMGT)
AREYYSMGVLDY

SEQ ID NO: 10 - protein, 6 aa, synthetic construct
  source 1..6: note = Antibody mAbA4, mAbA15, mAbA17 VL CDR1 (IMGT)
QSVSSA

SEQ ID NO: 11 - skipped

SEQ ID NO: 12 - protein, 5 aa, synthetic construct
  source 1..5: note = Antibody mAbA4 VH CDR1 (Kabat)
RYYIH

SEQ ID NO: 13 - protein, 7 aa, synthetic construct
  source 1..7: note = Antibody mAbA4, mAbA17 VH CDR1 (Chothia)
GFTFSRY

SEQ ID NO: 14 - protein, 4 aa, synthetic construct
  source 1..4: note = Antibody mAbA4 VH CDR2 (Chothia)
PGGS

SEQ ID NO: 15 - protein, 8 aa, synthetic construct
  source 1..8: note = Antibody mAbA4 VH CDR3 (Chothia)
YYSMGVLD

SEQ ID NO: 16 - protein, 7 aa, synthetic construct
  source 1..7: note = Antibody mAbA4, mAbA15, mAbA17 VL CDR1 (Chothia)
SQSVSSA

SEQ ID NO: 17 - protein, 6 aa, synthetic construct
  source 1..6: note = Antibody mAbA4 VL CDR3 (Chothia)
AYESPY

SEQ ID NO: 18 - protein, 6 aa, synthetic construct
  source 1..6: note = Antibody mAbA4 VH CDR1 (Contact)
SRYYIH

SEQ ID NO: 19 - protein, 13 aa, synthetic construct
  source 1..13: note = Antibody mAbA4 VH CDR2 (Contact)
WVAFISPGGSYTY

SEQ ID NO: 20 - protein, 11 aa, synthetic construct
  source 1..11: note = Antibody mAbA4 VH CDR3 (Contact)
AREYYSMGVLD

SEQ ID NO: 21 - protein, 7 aa, synthetic construct
  source 1..7: note = Antibody mAbA4, mAbA15, mAbA17 VL CDR1 (Contact)
SSAVAWY

SEQ ID NO: 22 - protein, 10 aa, synthetic construct
  source 1..10: note = Antibody mAbA4, mAbA15, mAbA17 VL CDR2 (Contact)
LLIYSASSLY

SEQ ID NO: 23 - protein, 8 aa, synthetic construct
  source 1..8: note = Antibody mAbA4 VL CDR3 (Contact)
QQAYESPY

SEQ ID NO: 24 - protein, 10 aa, synthetic construct
  source 1..10: note = Antibody mAbA4 VH CDR2 (AbM)
FISPGGSYTY

SEQ ID NO: 25 - protein, 119 aa, synthetic construct
  source 1..119: note = Antibody mAbA4 VH Sequence
EVQLVESGGGLVQPGGSLRLSCAASGFTFSRYYIHWVRQAPGKGLEWVAFISPGGSYTYYADSVKGRFTISADTSKNTAYLQMNSLRAEDTAVYYCAREYYSMGVLDYWGQGTLVTVSS

SEQ ID NO: 26 - protein, 107 aa, synthetic construct
  source 1..107: note = Antibody mAbA4 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQAYESPYTFGQGTKVEIK

SEQ ID NO: 27 - protein, 10 aa, synthetic construct
  source 1..10: note = Antibody mAbA15 VH CDR1 (Exemplary, AbM)
GFTFSSYYIH

SEQ ID NO: 28 - protein, 17 aa, synthetic construct
  source 1..17: note = Antibody mAbA15 VH CDR2 (Exemplary, Kabat)
YITSTGSYTEYADSVKG

SEQ ID NO: 29 - protein, 12 aa, synthetic construct
  source 1..12: note = Antibody mAbA15 VH CDR3 (Exemplary, Kabat, AbM)
YDRSTRYSGLDY

SEQ ID NO: 30 - protein, 9 aa, synthetic construct
  source 1..9: note = Antibody mAbA15 VL CDR3 (Exemplary, IMGT, Kabat, AbM)
QQAYATPVT

SEQ ID NO: 31 - protein, 8 aa, synthetic construct
  source 1..8: note = Antibody mAbA15 VH CDR1 (IMGT)
GFTFSSYY

SEQ ID NO: 32 - protein, 8 aa, synthetic construct
  source 1..8: note = Antibody mAbA15 VH CDR2 (IMGT)
ITSTGSYT

SEQ ID NO: 33 - protein, 14 aa, synthetic construct
  source 1..14: note = Antibody mAbA15 VH CDR3 (IMGT)
ARYDRSTRYSGLDY

SEQ ID NO: 34 - protein, 5 aa, synthetic construct
  source 1..5: note = Antibody mAbA15 VH CDR1 (Kabat)
SYYIH

SEQ ID NO: 35 - protein, 7 aa, synthetic construct
  source 1..7: note = Antibody mAbA15 VH CDR1 (Chothia)
GFTFSSY

SEQ ID NO: 36 - protein, 4 aa, synthetic construct
  source 1..4: note = Antibody mAbA15 VH CDR2 (Chothia)
STGS

SEQ ID NO: 37 - protein, 10 aa, synthetic construct
  source 1..10: note = Antibody mAbA15 VH CDR3 (Chothia)
DRSTRYSGLD

SEQ ID NO: 38 - protein, 6 aa, synthetic construct
  source 1..6: note = Antibody mAbA15 VL CDR3 (Chothia)
AYATPV

SEQ ID NO: 39 - protein, 6 aa, synthetic construct
  source 1..6: note = Antibody mAbA15 VH CDR1 (Contact)
SSYYIH

SEQ ID NO: 40 - protein, 13 aa, synthetic construct
  source 1..13: note = Antibody mAbA15 VH CDR2 (Contact)
WVAYITSTGSYTE

SEQ ID NO: 41 - protein, 13 aa, synthetic construct
  source 1..13: note = Antibody mAbA15 VH CDR3 (Contact)
ARYDRSTRYSGLD

SEQ ID NO: 42 - protein, 8 aa, synthetic construct
  source 1..8: note = Antibody mAbA15 VL CDR3 (Contact)
QQAYATPV

SEQ ID NO: 43 - protein, 10 aa, synthetic construct
  source 1..10: note = Antibody mAbA15 VH CDR2 (AbM)
YITSTGSYTE

SEQ ID NO: 44 - protein, 121 aa, synthetic construct
  source 1..121: note = Antibody mAbA15 VH Sequence
EVQLVESGGGLVQPGGSLRLSCAASGFTFSSYYIHWVRQAPGKGLEWVAYITSTGSYTEYADSVKGRFTISADTSKNTAYLQMNSLRAEDTAVYYCARYDRSTRYSGLDYWGQGTLVTVSS

SEQ ID NO: 45 - protein, 107 aa, synthetic construct
  source 1..107: note = Antibody mAbA15 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQAYATPVTFGQGTKVEIK

SEQ ID NO: 46 - protein, 10 aa, synthetic construct
  source 1..10: note = Antibody mAbA17 VH CDR1 (Exemplary, AbM)
GFTFSRYWIH

SEQ ID NO: 47 - protein, 17 aa, synthetic construct
  source 1..17: note = Antibody mAbA17 VH CDR2 (Exemplary, Kabat)
AIFPYSGSTLYADSVKG

SEQ ID NO: 48 - protein, 13 aa, synthetic construct
  source 1..13: note = Antibody mAbA17 VH CDR3 (Exemplary, Kabat, AbM)
SPSRSILPSGLDY

SEQ ID NO: 49 - protein, 9 aa, synthetic construct
  source 1..9: note = Antibody mAbA17 VL CDR3 (Exemplary, IMGT, Kabat, AbM)
QQGDGSPYT

SEQ ID NO: 50 - protein, 8 aa, synthetic construct
  source 1..8: note = Antibody mAbA17 VH CDR1 (IMGT)
GFTFSRYW

SEQ ID NO: 51 - protein, 8 aa, synthetic construct
  source 1..8: note = Antibody mAbA17 VH CDR2 (IMGT)
IFPYSGST

SEQ ID NO: 52 - protein, 15 aa, synthetic construct
  source 1..15: note = Antibody mAbA17 VH CDR3 (IMGT)
ARSPSRSILPSGLDY

SEQ ID NO: 53 - protein, 5 aa, synthetic construct
  source 1..5: note = Antibody mAbA17 VH CDR1 (Kabat)
RYWIH

SEQ ID NO: 54 - protein, 4 aa, synthetic construct
  source 1..4: note = Antibody mAbA17 VH CDR2 (Chothia)
PYSG

SEQ ID NO: 55 - protein, 11 aa, synthetic construct
  source 1..11: note = Antibody mAbA17 VH CDR3 (Chothia)
PSRSILPSGLD

SEQ ID NO: 56 - protein, 6 aa, synthetic construct
  source 1..6: note = Antibody mAbA17 VL CDR3 (Chothia)
GDGSPY

SEQ ID NO: 57 - protein, 6 aa, synthetic construct
  source 1..6: note = Antibody mAbA17 VH CDR1 (Contact)
SRYWIH

SEQ ID NO: 58 - protein, 13 aa, synthetic construct
  source 1..13: note = Antibody mAbA17 VH CDR2 (Contact)
WVAAIFPYSGSTL

SEQ ID NO: 59 - protein, 14 aa, synthetic construct
  source 1..14: note = Antibody mAbA17 VH CDR3 (Contact)
ARSPSRSILPSGLD

SEQ ID NO: 60 - protein, 8 aa, synthetic construct
  source 1..8: note = Antibody mAbA17 VL CDR3 (Contact)
QQGDGSPY

SEQ ID NO: 61 - protein, 10 aa, synthetic construct
  source 1..10: note = Antibody mAbA17 VH CDR2 (AbM)
AIFPYSGSTL

SEQ ID NO: 62 - protein, 122 aa, synthetic construct
  source 1..122: note = Antibody mAbA17 VH Sequence
EVQLVESGGGLVQPGGSLRLSCAASGFTFSRYWIHWVRQAPGKGLEWVAAIFPYSGSTLYADSVKGRFTISADTSKNTAYLQMNSLRAEDTAVYYCARSPSRSILPSGLDYWGQGTLVTVSS

SEQ ID NO: 63 - protein, 107 aa, synthetic construct
  source 1..107: note = Antibody mAbA17 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQGDGSPYTFGQGTKVEIK

SEQ ID NO: 64 - protein, 420 aa, Homo sapiens
  source 1..420: note = full-length amino acid sequence of human 5T4
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 65 - protein, 420 aa, Macaca fascicularis
  source 1..420: note = full-length amino acid sequence of cynomolgous monkey (cyno) 5T4
MPGGCSRGPAAGDGRLRLARLALVLLGWVSSSSSTSSASSSSSSAPFLASAASAQPPLPDQCPALCECSEAARTVKCVNRNLTEVPTDLPLYVRNLFLTGNQLAVLPAGAFARRPPLAELAALNLSGSRLDEVRGGAFEHLPSLRQLDLSHNPLAYLSPFAFSGSNASISAPSPLVELILNHIVPPDDKRQNRSFEGMVAAALVAGRALQGLHLLELASNHFLYLPRDVLAQLPSLRYLDLSNNSLVSLTYVSFRNLTHLESLHLEDNALKVLHNGTLAELQGLPHVRVFLDNNPWVCDCHMADMVTWLKQTGVVQGKDRLTCAFPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 66 - protein, 426 aa, Mus musculus
  source 1..426: note = full-length amino acid sequence of mouse 5T4
MPGAGSRGPSAGDGRLRLARLALVLLGWVSASAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTSYVFLGIVLALIGAIFLLVLYLNRKGIKKWMHNIRDACRDHMEGYHYRYEINADPRLTNLSSNSDV

SEQ ID NO: 67 - protein, 222 aa, synthetic construct
  source 1..222: note = exemplary Fc region sequence
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 222 aa, synthetic construct
  source 1..222: note = exemplary variant Fc region (silent Fc)
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 330 aa, synthetic construct [sequence omitted]

SEQ ID NO: 70 - protein, 330 aa, synthetic construct
  source 1..330: note = exemplary IgG heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 107 aa, synthetic construct
  source 1..107: note = exemplary light chain (e.g., for pairing with an IgG heavy chain)
RTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 226 aa, synthetic construct
  source 1..226: note = phage display heavy chain
  VARIANT 31..35: note = X is any amino acid
  VARIANT 50..66: note = X is any amino acid
  VARIANT 99..112: note = X is any amino acid
EVQLVESGGGLVQPGGSLRLSCAASGFTFSXXXXXWVRQAPGKGLEWVAXXXXXXXXXXXXXXXXXRFTISADTSKNTAYLQMNSLRAEDTAVYYCARXXXXXXXXXXXXXXWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 73 - protein, 214 aa, synthetic construct
  source 1..214: note = phage display light chain
  VARIANT 89..97: note = X is any amino acid
DIQMTQSPSSLSASVGDRVTITCRASQSVSSAVAWYQQKPGKAPKLLIYSASSLYSGVPSRFSGSRSGTDFTLTISSLQPEDFATYYCXXXXXXXXXFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 330 aa, synthetic construct [sequence omitted]

SEQ ID NO: 75 - protein, 107 aa, synthetic construct; identical to SEQ ID NO: 71
  source 1..107: note = full length IgGs, the light chain constant region

SEQ ID NO: 76 - protein, 43 aa, Homo sapiens
  source 1..43: note = Epitope 1 of human 5T4 (H1)
SSPTSSASSFSSSAPFLASAVSAQPPLPDQCPALCECSEAART

SEQ ID NO: 77 - protein, 60 aa, Homo sapiens
  source 1..60: note = Epitope 2 of human 5T4 (H2)
VKCVNRNLTEVPTDLPAYVRNLFLTGNQLAVLPAGAFARRPPLAELAALNLSGSRLDEVR

SEQ ID NO: 78 - protein, 30 aa, Homo sapiens
  source 1..30: note = Epitope 3 of human 5T4 (H3)
AGAFEHLPSLRQLDLSHNPLADLSPFAFSG

SEQ ID NO: 79 - protein, 52 aa, Homo sapiens
  source 1..52: note = Epitope 4 of human 5T4 (H4)
SNASVSAPSPLVELILNHIVPPEDERQNRSFEGMVVAALLAGRALQGLRRLE

SEQ ID NO: 80 - protein, 47 aa, Homo sapiens
  source 1..47: note = Epitope 5 of human 5T4 (H5)
LASNHFLYLPRDVLAQLPSLRHLDLSNNSLVSLTYVSFRNLTHLESL

SEQ ID NO: 81 - protein, 28 aa, Homo sapiens
  source 1..28: note = Epitope 6 of human 5T4 (H6)
HLEDNALKVLHNGTLAELQGLPHIRVFL

SEQ ID NO: 82 - protein, 64 aa, Homo sapiens
  source 1..64: note = Epitope 7 of human 5T4 (H7)
DNNPWVCDCHMADMVTWLKETEVVQGKDRLTCAYPEKMRNRVLLELNSADLDCDPILPPSLQTS

SEQ ID NO: 83 - protein, 330 aa, synthetic construct
  source 1..330: note = Human 5T4 Epitope 1
SSPTSSASSFSSSAPFLASAVSAQPPLPDQCPALCECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTS

SEQ ID NO: 84 - protein, 330 aa, synthetic construct
  source 1..330: note = Human 5T4 Epitope 2
SAPSSSVPSSSTSPAAFLASGSAQPPPAERCPAACECSEAARTVKCVNRNLTEVPTDLPAYVRNLFLTGNQLAVLPAGAFARRPPLAELAALNLSGSRLDEVRAGAFEHLPGLRRLDLSHNPLTNLSAFAFAGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTS

SEQ ID NO: 85 - protein, 330 aa, synthetic construct
  source 1..330: note = Human 5T4 Epitope 3
SAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPSLRQLDLSHNPLADLSPFAFSGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTS

SEQ ID NO: 86 - protein, 324 aa, synthetic construct
  source 1..324: note = Human 5T4 Epitope 4
SAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLVELILNHIVPPEDERQNRSFEGMVVAALLAGRALQGLRRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTS

SEQ ID NO: 87 - protein, 330 aa, synthetic construct
  source 1..330: note = Human 5T4 Epitope 5
SAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALRSGLALRGLTRLELASNHFLYLPRDVLAQLPSLRHLDLSNNSLVSLTYVSFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTS

SEQ ID NO: 88 - protein, 330 aa, synthetic construct
  source 1..330: note = Human 5T4 Epitope 6
SAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNGTLAELQGLPHIRVFLDNNPWVCDCYMADMVAWLKETEVVPDKARLTCAFPEKMRNRGLLDLNSSDLDCDAVLPQSLQTS

SEQ ID NO: 89 - protein, 330 aa, synthetic construct
  source 1..330: note = Human 5T4 Epitope 7
SAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCHMADMVTWLKETEVVQGKDRLTCAYPEKMRNRVLLELNSADLDCDPILPPSLQTS

SEQ ID NO: 90 - protein, 457 aa, synthetic construct
  source 1..457: note = exemplary modified heavy chain antibody that binds to 5T4
EVQLVESGGGLVQPGGSLRLSCAASGFTFSRYYIHWVRQAPGKGLEWVAFISPGGSYTYYADSVKGRFTISADTSKNTAYLQMNSLRAEDTAVYYCAREYYSMGVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLCTPSRGS

SEQ ID NO: 91 - protein, 214 aa, synthetic construct
  source 1..214: note = exemplary light chain antibody that binds to 5T4
DIQMTQSPSSLSASVGDRVTITCRASQSVSSAVAWYQQKPGKAPKLLIYSASSLYSGVPSRFSGSRSGTDFTLTISSLQPEDFATYYCQQAYESPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 457 aa, synthetic construct
  source 1..457: note = exemplary modified heavy chain antibody that binds to 5T4 and includes formyl glycine residue
  MOD_RES 451: note = FORMYLATION
EVQLVESGGGLVQPGGSLRLSCAASGFTFSRYYIHWVRQAPGKGLEWVAFISPGGSYTYYADSVKGRFTISADTSKNTAYLQMNSLRAEDTAVYYCAREYYSMGVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLGTPSRGS

SEQ ID NO: 93 - protein, 457 aa, synthetic construct
  source 1..457: note = exemplary modified heavy chain 5T4-ADC
  SITE 451: note = n is an amino acid having its side chain replaced by a linker-drug
EVQLVESGGGLVQPGGSLRLSCAASGFTFSRYYIHWVRQAPGKGLEWVAFISPGGSYTYYADSVKGRFTISADTSKNTAYLQMNSLRAEDTAVYYCAREYYSMGVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLNTPSRGS

SEQ ID NO: 94 - protein, 459 aa, synthetic construct
  source 1..459: note = exemplary modified heavy chain antibody that binds to 5T4
EVQLVESGGGLVQPGGSLRLSCAASGFTFSSYYIHWVRQAPGKGLEWVAYITSTGSYTEYADSVKGRFTISADTSKNTAYLQMNSLRAEDTAVYYCARYDRSTRYSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLCTPSRGS

SEQ ID NO: 95 - protein, 214 aa, synthetic construct
  source 1..214: note = exemplary light chain antibody that binds to 5T4
DIQMTQSPSSLSASVGDRVTITCRASQSVSSAVAWYQQKPGKAPKLLIYSASSLYSGVPSRFSGSRSGTDFTLTISSLQPEDFATYYCQQAYATPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 459 aa, synthetic construct
  source 1..459: note = exemplary modified heavy chain antibody that binds to 5T4 and includes formyl glycine residue
  MOD_RES 453: note = FORMYLATION
EVQLVESGGGLVQPGGSLRLSCAASGFTFSSYYIHWVRQAPGKGLEWVAYITSTGSYTEYADSVKGRFTISADTSKNTAYLQMNSLRAEDTAVYYCARYDRSTRYSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLGTPSRGS

SEQ ID NO: 97 - protein, 459 aa, synthetic construct
  source 1..459: note = exemplary modified heavy chain 5T4-ADC
  SITE 453: note = n is an amino acid having its side chain replaced by a linker-drug
EVQLVESGGGLVQPGGSLRLSCAASGFTFSSYYIHWVRQAPGKGLEWVAYITSTGSYTEYADSVKGRFTISADTSKNTAYLQMNSLRAEDTAVYYCARYDRSTRYSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLNTPSRGS

SEQ ID NO: 98 - protein, 460 aa, synthetic construct
  source 1..460: note = exemplary modified heavy chain antibody that binds to 5T4
EVQLVESGGGLVQPGGSLRLSCAASGFTFSRYWIHWVRQAPGKGLEWVAAIFPYSGSTLYADSVKGRFTISADTSKNTAYLQMNSLRAEDTAVYYCARSPSRSILPSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLCTPSRGS

SEQ ID NO: 99 - protein, 214 aa, synthetic construct
  source 1..214: note = exemplary light chain antibody that binds to 5T4
DIQMTQSPSSLSASVGDRVTITCRASQSVSSAVAWYQQKPGKAPKLLIYSASSLYSGVPSRFSGSRSGTDFTLTISSLQPEDFATYYCQQGDGSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 460 aa, synthetic construct
  source 1..460: note = exemplary modified heavy chain antibody that binds to 5T4 and includes formyl glycine residue
  MOD_RES 454: note = FORMYLATION
EVQLVESGGGLVQPGGSLRLSCAASGFTFSRYWIHWVRQAPGKGLEWVAAIFPYSGSTLYADSVKGRFTISADTSKNTAYLQMNSLRAEDTAVYYCARSPSRSILPSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLGTPSRGS

SEQ ID NO: 101 - protein, 460 aa, synthetic construct
  source 1..460: note = exemplary modified heavy chain 5T4-ADC
  SITE 454: note = n is an amino acid having its side chain replaced by a linker-drug
EVQLVESGGGLVQPGGSLRLSCAASGFTFSRYWIHWVRQAPGKGLEWVAAIFPYSGSTLYADSVKGRFTISADTSKNTAYLQMNSLRAEDTAVYYCARSPSRSILPSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLNTPSRGS

SEQ ID NO: 102 - protein, 6 aa, synthetic construct
  source 1..6: note = synthetic peptide
  MOD_RES 2: note = FORMYLATION
LGTPSR

SEQ ID NO: 103 - protein, 6 aa, synthetic construct
  source 1..6: note = synthetic peptide
  VARIANT 2: note = X is C or S
LXTPSR